(19) United States
(12) Patent Application Publication
(10) Pub. No.: US 2024/0368104 A1
(43) Pub. Date: Nov. 7, 2024

(54) Novel Dialkoxynaphtho[2,3-C]Furan-1(3H)-One Derivatives and Pharmaceutical Composition For Preventing or Treating Respiratory Disease or SARS-COV-2 Infection Disease, Comprising Same

(71) Applicant: DONG WHA PHARM. CO., LTD., Seoul (KR)

(72) Inventors: Jae Kyung LIM, Suwon-si Gyeonggi-do (KR); Jung UK CHOI, Suwon-si Gyeonggi-do (KR); Dong Hyuk SHIN, Suwon-si Gyeonggi-do (KR); Yong Tae KIM, Yongin-si Gyeonggi-do (KR); Seung Hwan KIM, Suwon-si Gyeonggi-do (KR); Jung Hwan KIM, Yongin-si Gyeonggi-do (KR); O Jin KWON, Suwon-si Gyeonggi-do (KR); Jin Yong JUNG, Hwaseong-si Gyeonggi-do (KR); Seo Hee JEONG, Suwon-si Gyeonggi-do (KR); Yun Ha HWANG, Gunpo-si Gyeonggi-do (KR); Doc Gyun JEONG, Yongin-si Gyeonggi-do (KR); Ji Hyun YOUM, Suwon-si Gyeonggi-do (KR); Whui Jung PARK, Suwon-si Gyeonggi-do (KR)

(73) Assignee: DONG WHA PHARM. CO., LTD., Seoul (KR)

(21) Appl. No.: 18/553,532

(22) PCT Filed: Mar. 30, 2022
(86) PCT No.: PCT/IB2022/052939

§ 371 Date: Sep. 29, 2023

(30) Foreign Application Priority Data
Mar. 31, 2021 (KR) 10-2021-0041955

(51) Int. Cl. C07D 307/92; A61K 31/365; A61K 31/366; A61K 31/381; A61K 31/4365; A61K 31/438; A61K 31/4525; A61K 31/506; A61K 31/5377; A61K 31/541; C07D 405/04; C07D 405/14; C07D 407/04; C07D 409/04; C07D 413/04; C07D 413/14; C07D 417/14; C07D 487/04

(52) U.S. Cl. CPC C07D 307/92; A61K 31/365; A61K 31/366; A61K 31/381; A61K 31/4365; A61K 31/438; A61K 31/4525; A61K 31/506; A61K 31/5377; A61K 31/541; C07D 405/04; C07D 405/14; C07D 407/04; C07D 409/04; C07D 413/04; C07D 413/14; C07D 417/14; C07D 487/04

(57) ABSTRACT

The present invention provides a compound of chemical formula 1, a compound of chemical formula 3, compound [212], compound [224], or compound [228], a pharmaceutically acceptable salt thereof, a stereoisomer thereof, a hydrate thereof, or a solvate thereof, and a pharmaceutical composition comprising same. The pharmaceutical composition has excellent antiviral efficacy, thereby having a preventive or treatment effect on viral infectious disease such as SARS-COV-2, and exhibits an inhibitory effect on IL-5 expression and thus has a preventive or treatment effect on respiratory disease or allergic disease.

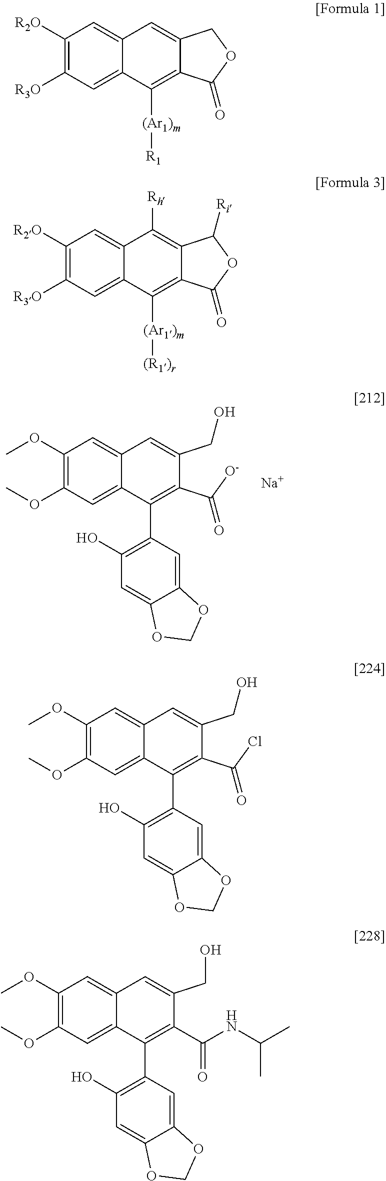

### TECHNICAL FIELD

The present invention relates to a dialkoxynaphtho[2,3-c]furan-1(3H)-one derivative and a pharmaceutical composition including the same.

### BACKGROUND ART

The Justicia genus of the Acanthaceae family consists of about 600 species. Representative Justicia genus plants include Justicia procumbens, Justicia pectoralis, Justicia gendarussa, Justicia anselliana, Justicia adhatoda, and the like. Among them, Justicia procumbens, which is a yearly plant, includes Justicidin A (9-(1,3-benzodioxol-5-yl)-4,6,7-trimethoxy-3H-benzo[f][2]benzofuran-1-one), Justicidin B (4-(1,3-benzodioxol-5-yl)-6,7-dimethoxy-1H-benzo[f][2]benzofuran-3-one), and the like as active ingredients, and grows in Korea, Japan, China, India, etc. Existing therapeutic agents for allergic diseases include steroids which suppress immune responses, but are limited to a local or temporary use due to various side effects caused by non-selective immune suppression. Anti-leukotrienes, anti-histamines and the like are the most commonly used therapeutic agents for allergic diseases, but are involved in controlling at the lower stages of the allergy-inducing mechanism, and thus have a limitation in that they have an effect of temporarily alleviating symptoms. In particular, anti-leukotrienes do not have a good medicinal effect on the treatment of asthma as compared to inhaler corticosteroid (ICS), and thus their use are limited only to patients who cannot use inhalant agents, patients who have side effects on ICS, or asthma patients with allergic rhinitis (Workshop report, global strategy for asthma management and prevention (updated 2017). The GINA reports are available on www.ginasthma.org.). Besides, there is an anti-IgE antibody therapeutic agent (Omalizumab) which is used as a therapeutic agent for severe asthma, and anti-IL-5 antibody therapeutic agents (Mepolizumab, Reslizumab) have been recently developed, but there is a need to develop a new concept of therapeutic agent effective for allergic diseases due to high costs and a limited improvement effect on severe asthma patients (Corren J, Weinstein S, Janka L, Zangrilli J, Garin M. Phase 3 study of reslizumab in patients with poorly controlled asthma: Effects across a broad range of eosinophil counts. Chest. 2016, 150 (4): 799-810, Djukanović R, Wilson S J, Kraft M, Jarjour N N, Steel M, Chung K F, Bao W, Fowler-Taylor A, Matthews J, Busse W W, Holgate S T, Fahy J V. Effects of treatment with anti-immunoglobulin E antibody omalizumab on airway inflammation in allergic asthma. Am J Respir Crit Care Med. 2004 Sep. 15; 170 (6): 583-93).

Severe acute respiratory syndrome coronavirus 2 (SARS-COV-2) refers to a severe acute respiratory syndrome coronavirus 2, which has been first known in 2019, and is classified as a positive-sense single-stranded RNA virus. The disease infected by this virus has been named Coronavirus infection disease-19 (Coronavirus disease 2019), and is also called COVID-19 for short. The World Health Organization (WHO) has officially announced a coronavirus pandemic. As of November 2020, it has been found that the number of infected people in South Korea is more than 27,000, and about 47.4 million people are infected around the world and this trend continues to spread. Currently, Remdesivir, an Ebola virus therapeutic agent, is the only one formulated as a therapeutic agent for COVID-19, but its effect has been questioned, and plasma treatment, etc., is used as an alternative therapy. In addition, clinical trials for COVID 19 have been conducted on Kaletra (main ingredient: Lopinavir), a human immunodeficiency virus (HIV) therapeutic agent, etc. Accordingly, there is an urgent need for developing a therapeutic agent for SARS-Cov-2.

### DETAILED DESCRIPTION OF THE INVENTION

### Technical Problem

One object of the present invention is to provide a 6,7-dialkoxynaphtho[2,3-c]furan-1(3H)-one compound, a pharmaceutically acceptable salt thereof, a stereoisomer thereof, a hydrate thereof, or a solvate thereof.

In addition, one object of the present invention is to provide a pharmaceutical composition including a 6,7-dialkoxynaphtho[2,3-c]furan-1(3H)-one compound.

Furthermore, one object of the present invention is to provide a pharmaceutical composition for an anti-viral purpose including a 6,7-dialkoxynaphtho[2,3-c]furan-1(3H)-one compound.

Moreover, one object of the present invention is to provide a pharmaceutical composition for preventing or treating at least one of SARS-COV-2 infection disease, influenza virus infection disease, dengue virus infection disease, Zika virus infection disease and severe fever with thrombocytopenia syndrome virus infection disease, which includes a 6,7-dialkoxynaphtho[2,3-c]furan-1(3H)-one compound.

Besides, one object of the present invention is to provide a pharmaceutical composition for preventing or treating a respiratory disease, which includes a 6,7-dialkoxynaphtho[2,3-c]furan-1(3H)-one compound.

In addition, one object of the present invention is to provide a pharmaceutical composition for preventing or treating an allergic disease, which includes a 6,7-dialkoxynaphtho[2,3-c]furan-1(3H)-one compound.

Furthermore, one object of the present invention is to provide a method for preventing or treating a virus infection disease; a respiratory disease; or an allergic disease; which includes administering a 6,7-dialkoxynaphtho[2,3-c]furan-1(3H)-one compound into a subject in need thereof.

Moreover, one object of the present invention is to provide a use of a 6, 7-dialkoxynaphtho[2,3-c]furan-1(3H)-one compound for preventing or treating a virus infection disease; a respiratory disease; or an allergic disease.

Besides, one object of the present invention is to provide a use of a 6,7-dialkoxynaphtho[2,3-c]furan-1(3H)-one compound for manufacturing a medicament for preventing or treating a virus infection disease; a respiratory disease; or an allergic disease.

### Technical Solution

Hereinafter, in the present specification, “substituted or unsubstituted” may refer to being substituted or unsubstituted with at least one of halogen, hydroxy (—OH), —C(═O)R, alkoxy, amine, alkyl, alkenyl, cycloalkyl, cycloalkenyl, heterocycloalkyl, heterocycloalkenyl, aryl, and heteroaryl.

In the present specification, R of —C(═O)R may be hydrogen, alkyl, hydroxy, alkoxy, amine, alkyl, alkenyl, cycloalkyl, cycloalkenyl, heterocycloalkyl, heterocycloalkenyl, aryl, or heteroaryl. R of —C(═O)R may be substituted or unsubstituted.

In the present specification, halogen may be F, Cl, Br or I. For example, the halogen may be F or Cl.

In the present specification, alkoxy may be linear or branched alkoxy. The alkoxy may be substituted or unsubstituted. For example, the alkoxy may be substituted or unsubstituted C₁-C₂₀ linear or branched alkoxy. The C₁-C₂₀ linear or branched alkoxy may include C₁-C₁₅. C₁-C₁₀. C₁-C₈, C₁-C₅ and/or C₁-C₃ linear or branched alkoxy. Here, the description of the alkyl group to be described later may be applied to the alkyl group included in the alkoxy.

In the present specification, “and/or” may be used as a term referring to all combinations which may be derived from a plurality of configurations.

In the present specification, amine may include —NH₂, alkylamine, cycloalkylamine, cycloalkenylamine, heterocycloalkylamine, arylamine, and/or heteroarylamine. In the present specification, the amine, in which only one alkyl is substituted for nitrogen and another substituent (for example, a phenyl group) different from alkyl is further substituted, may be also defined as alkylamine. For example, in the present specification, methylphenylamine may be interpreted as alkylamine or arylamine.

In alkylamine, cycloalkylamine, cycloalkenylamine, heterocycloalkylamine, arylamine and heteroarylamine, the description of alkyl, cycloalkyl, cycloalkenyl, heterocycloalkyl, aryl, and heteroaryl to be described later may be applied to alkyl, cycloalkyl, cycloalkenyl, heterocycloalkyl, aryl and heteroaryl.

In the present specification, alkyl may be linear or branched alkyl, and may be substituted or unsubstituted. The alkyl may be C₁-C₂₀ alkyl. The C₁-C₂₀ alkyl may include C₁-C₁₅. C₁-C₁₀, C₁-C₈, C₁-C₅ and C₁-C₃ alkyl.

Examples of the alkyl may include methyl, ethyl, n-propyl, isopropyl, n-butyl, s-butyl, t-butyl, i-butyl, 2-ethylbutyl, 3,3-dimethylbutyl, n-pentyl, i-pentyl, neopentyl, t-pentyl, cyclopentyl, 1-methylpentyl, 3-methylpentyl, 2-ethylpentyl, 4-methyl-2-pentyl, n-hexyl, 1-methylhexyl, 2-ethylhexyl, 2-butylhexyl, cyclohexyl, 4-methylcyclohexyl, 4-t-butylcyclohexyl, n-heptyl, 1-methylheptyl, 2,2-dimethylheptyl, 2-ethylheptyl, 2-butylheptyl, n-octyl, t-octyl, 2-ethyloctyl, 2-butyloctyl, 2-hexyloctyl, 3,7-dimethyloctyl, cyclooctyl, n-nonyl, n-decyl, adamantyl, 2-ethyldecyl, 2-butyldecyl, 2-hexyldecyl, 2-octyldecyl, n-undecyl, n-dodecyl, 2-ethyldodecyl, 2-butyldodecyl, 2-hexyldodecyl, 2-octyldodecyl, n-tridecyl, n-tetradecyl, n-pentadecyl, n-hexadecyl, 2-ethylhexadecyl, 2-butylhexadecyl, 2-hexylhexadecyl, 2-octylhexadecyl, n-heptadecyl, n-octadecyl, n-nonadecyl, n-icosyl, 2-ethylicosyl, 2-butylicosyl, 2-hexylicosyl, 2-octylicosyl, n-henicosyl, n-docosyl, n-tricosyl, n-tetracosyl, n-pentacosyl, n-hexacosyl, n-heptacosyl, n-octacosyl, n-nonacosyl, n-triacontyl, and the like, but an example is not limited thereto.

In the present specification, the above description of alkyl may be applied to alkenyl except for including one or more carbon-carbon double bonds.

In the present specification, cycloalkyl may be substituted or unsubstituted. The cycloalkyl may be C₃-C₂₀ cycloalkyl. The C₃-C₂₀ cycloalkyl may include C₃-C₁₅, C₃-C₁₀ and/or C₃-C₅ cycloalkyl. In the present specification, a substituent of the cycloalkyl may be bonded to an adjacent group to form a ring. Examples of the cycloalkyl may include cyclopropyl, cyclobutyl, cyclopentyl, cyclohexyl, cycloheptyl, and/or the like, but an example is not limited thereto.

In the present specification, heterocycloalkyl may be substituted or unsubstituted. The heterocycloalkyl may be monocyclic, bicyclic alkyl, or spiroalkyl. When the heterocycloalkyl is bicyclic alkyl or spiroalkyl, only one ring of the two rings may include a heteroatom, and both rings may include a heteroatom. The heterocycloalkyl may be C₂-C₂₀ heterocycloalkyl. The C₂-C₂₀ heterocycloalkyl may include C₂-C₁₅, C₂-C₁₀ and C₂-C₅ heterocycloalkyl. In the present specification, a substituent of the heterocycloalkyl may be bonded to an adjacent group to form a ring. In the present specification, the heterocycloalkyl may include at least one heteroatom selected from N, O, S, P and B in a ring. For example, in the present specification, the heterocycloalkyl may include at least one or at least two heteroatoms selected from N, O and S in a ring and, for example, may include N as a heteroatom in a ring. When the heterocycloalkyl includes two or more heteroatoms in the ring, the heteroatoms may be the same as or different from each other. Examples of the heterocycloalkyl may include pyrrolidine, tetrahydrofuran, tetrahydrothiophene, piperidine, tetrahydropyran, tetrahydrothiopyran, piperazine, morpholine, thiomorpholine, quinuclidine, and the like, but an example is not limited thereto.

In the present specification, the above description of heterocycloalkyl may be applied to heterocycloalkenyl except for including one or more carbon-carbon double bonds in a ring.

In the present specification, aryl may be substituted or unsubstituted. The aryl may be C₆-C₂₀ aryl. The C₆-C₂₀ aryl may include C₆-C₁₅, C₆-C₁₀ and/or C₆-C₈ aryl. In the present specification, a substituent of the aryl may be bonded to an adjacent group to form a ring.

In the present specification, “being bonded to an adjacent group to form a ring” may refer to being bonded to an adjacent group to form a substituted or unsubstituted hydrocarbon ring, or a substituted or unsubstituted hetero ring. The hydrocarbon ring may include an aliphatic hydrocarbon ring and an aromatic hydrocarbon ring. The hetero ring may include an aliphatic hetero ring and an aromatic hetero ring. The hydrocarbon ring and the hetero ring may be monocyclic or polycyclic. In addition, the ring formed by bonding with an adjacent group may form a bicyclic ring (including a bridge structure) and/or a spiro ring.

Hereinafter, as an example, described are a cyclohexane group in which a substituent is bonded to an adjacent group to form a benzene ring with an alkoxy group substituted, a piperidine group in which a substituent is bonded to an adjacent group to form a thiophene ring, a piperidine group in which a substituent is bonded to an adjacent group to form a spirocyclic group, a piperidine group (quinuclidine) in which a substituent is bonded to an adjacent group to form a bridge structure, a cyclohexane group in which a substituent is bonded to an adjacent group to form a benzene ring, a cyclohexane group in which a substituent is bonded to an adjacent group to form a benzene ring, a cyclohexane group in which a substituent is bonded to an adjacent group to form a benzene ring with a benzene ring substituted, a heterocyclohexane group in which a substituent is bonded to an adjacent group to form heteroaryl with —CF₃ substituted, and a tetrahydroisoquinoline group with a methyl group substituted in which a substituent is bonded to an adjacent group.

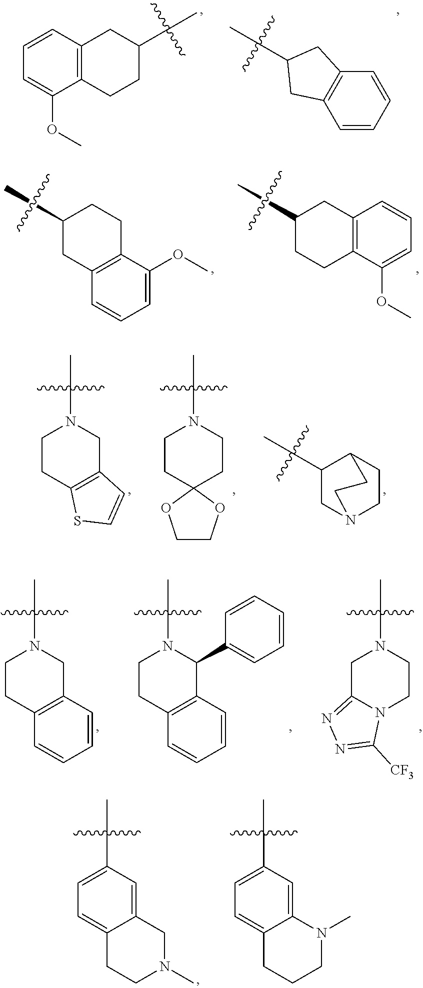

Examples of the aryl group may include phenyl, naphthyl, fluorenyl, anthracenyl, phenanthryl, biphenyl, terphenyl, quaterphenyl, quinquephenyl, sexiphenyl, triphenylenyl, pyrenyl, benzo fluoranthenyl, chrycenyl, and the like, but an example is not limited thereto.

In the present specification, [symbol] may refer to a portion to be connected.

In the present specification, the heteroaryl group may be substituted or unsubstituted. The heteroaryl group may be C₂-C₂₀ heteroaryl group. The C₂-C₂₀ heteroaryl group may include C₂-C₁₅, C₂-C₁₀, C₂-C₅, C₃-C₂₀, C₃-C₁₅. C₃-C₁₀ and/or C₃-C₅ heteroaryl group. In the present specification, the heteroaryl group may include at least one heteroatom selected from N, O, S, P and B in a ring. For example, in the present specification, the heteroaryl group may include one or more heteroatoms selected from N, O and S and, for example, may include N as a heteroatom. When the heteroaryl group includes two or more heteroatoms in the ring, the heteroatoms may be the same as or different from each other. For example, in the present specification, the heteroaryl group may be C₂-C₁₅, C₂-C₁₀, C₂-C₅, C₃-C₂₀, C₃-C₁₅. C₃-C₁₀ or C₃-C₅ heteroaryl group including N as a heteroatom.

Examples of the heteroaryl group may include benzoimidazolyl, benzofuranyl, quinolyl, naphthoquinonyl, benzodioxazolyl, quinoxalyl, benzothiophenyl, tetrathienopyridyl, piperidyl, piperidone ethylketal, tetrahydronaphthalenyl, morpholinyl, piperazinyl, pyrrolidinyl, thiophenyl, thiomorpholinyl, furanyl, imidazolnyl, pyrazolnyl, pyrimidinyl, dibenzofuranyl, quinuclidinyl, indolyl, azaindolyl, quinazolinyl, naphthopyridinyl, purinyl, isoindolyl, benzodioxinyl, dihydrobenzodioxinyl, benzothiazolinyl, tetrahydroisoindolyl, chromenyl, benzothiophenyl, benztriazolyl, benzoxadiazolyl, indazolyl, isoquinolinyl, indolinyl, azaindolinyl, pyrimidopyridinyl, pyridopyrimidinyl, imidazopyridinyl, dihydroimidazopyrimidinyl, furopyridinyl, insoindolinyl and the like, but an example is not limited thereto.

In the present specification, the above description of the aryl group may be applied to the arylene group except for being a divalent group.

In the present specification, the above description of the heteroaryl group may be applied to the heteroarylene group except for being a divalent group.

The compound of the present specification may include not only pharmaceutically acceptable salts, but also all the salts, stereoisomers, hydrates and solvates, which may be prepared by a conventional method.

In the present specification, the “hydrate” may refer to one in which a compound according to one example, a pharmaceutically acceptable salt thereof or a stereoisomer thereof and water are bound by a non-covalent intermolecular force, and may include a stoichiometric or non-stoichiometric amount of water. Specifically, the hydrate may include water at a molar ratio of about 0.25 mol to about 10 mol based on 1 mol of an active ingredient, more specifically about 0.5 mol, about 1 mol, about 1.5 mol, about 2 mol, about 2.5 mol, about 3 mol, about 5 mol, etc.

In the present specification, the “solvate” may refer to one in which a compound according to one example, a pharmaceutically acceptable salt thereof or a stereoisomer thereof and a solvent other than water are bound by an intermolecular force, and may include a stoichiometric or non-stoichiometric amount of the solvent. Specifically, the solvate may include a solvent molecule at a molar ratio of about 0.25 mol to about 10 mol based on 1 mol of an active component, more specifically about 0.5 mol, about 1 mol, about 1.5 mol, about 2 mol, about 2.5 mol, about 3 mol, about 5 mol, etc.

According to one example of the present invention, there may be provided a 6,7-dialkoxynaphtho[2,3-c]furan-1(3H)-one compound of formula 1 below, a pharmaceutically acceptable salt thereof, a stereoisomer thereof, a hydrate thereof, or a solvate thereof. The 6,7-dialkoxynaphtho[2,3-c]furan-1(3H)-one compound of formula 1 may be a 6,7-dimethoxynaphtho[2,3-c]furan-1(3H)-one compound.

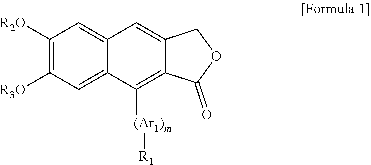

In above formula 1,
Ar₁ may be arylene group or heteroarylene group. Ar₁ may be C₆-C₁₂ arylene group or C₆-C₂₀ heteroarylene group. For example, Ar₁ may be substituted or unsubstituted C₂-C₁₂ heteroarylene group including at least one of N, O and S as a heteroatom in a ring. m may be 0 or 1.
R₁ may be hydrogen, halogen, substituted or unsubstituted C₁-C₁₀ linear or branched alkyl, substituted or unsubstituted C₂-C₂₀ heterocycloalkyl including at least one of N, O and S as a heteroatom in a ring (wherein a substituent of C₃-C₂₀ cycloalkyl or C₂-C₂₀ heterocycloalkyl may be each independently bonded to an adjacent group to form a ring), substituted or unsubstituted C₂-C₂₀ heteroaryl including at least one of N, O and S as a heteroatom in a ring, —NR₄R₅, —OR₆, —N═CHR₇ or

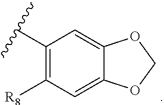

When R₁ is hydrogen or halogen, m may be 1.
When C₂-C₂₀ heteroaryl is furan or thiophene and m is 0, the furan and thiophene may each independently have at least one hydrogen substituted with C₁-C₁₀ linear or branched alkyl, substituted or unsubstituted C₆-C₂₀ aryl, substituted or unsubstituted C₂-C₂₀ heteroaryl, or —C(═O)Ra (wherein Ra is hydrogen, C₁-C₁₀ linear or branched alkyl, substituted or unsubstituted C₆-C₂₀ aryl, or substituted or unsubstituted C₂-C₂₀ heteroaryl).
R₂ and R₃ may be each independently C₁-C₁₀ linear or branched alkyl. R₂ and R₃ may be, for example, C₁-C₅ linear or branched alkyl, C₁-C₃ linear or branched alkyl, an ethyl group, or a methyl group.
R₄ to R₇ may be each independently hydrogen, halogen, C₁-C₁₀ linear or branched alkyl (wherein at least one of the hydrogen of the alkyl may be substituted with OH, substituted or unsubstituted C₃-C₁₀ cycloalkyl, substituted or unsubstituted C₃-C₁₀ cycloalkenyl, substituted or unsubstituted C₂-C₁₀ heterocycloalkyl, substituted or unsubstituted C₆-C₂₀ aryl, substituted or unsubstituted C₂-C₂₀ heteroaryl, —C(═O)OR_{b}, linear or branched alkoxy, or amine), substituted or unsubstituted C₃-C₁₀ cycloalkyl (wherein a substituent of the cycloalkyl may be bonded to an adjacent group to form a ring), substituted or unsubstituted C₂-C₁₀ heterocycloalkyl including at least one of N, O and S as a heteroatom in a ring (wherein a substituent of the heterocycloalkyl may be bonded to an adjacent group to form a ring), substituted or unsubstituted C₆-C₂₀ aryl (wherein a substituent of the aryl may be bonded to an adjacent group to form a ring), substituted or unsubstituted C₂-C₂₀ heteroaryl including at least one of N, O and S as a heteroatom in a ring, or —C(═O)R_{c}.
When R₄ or R₅ is C₁-C₁₀ linear or branched alkyl, wherein at least one of the hydrogen of the alkyl is substituted with substituted or unsubstituted C₂-C₁₀ heterocycloalkyl, the substituted or unsubstituted C₂-C₁₀ heterocycloalkyl may be substituted or unsubstituted morpholine, substituted or unsubstituted dioxolane, or substituted or unsubstituted tetrahydrofuran.
When R₄ or R₅ is C₁-C₁₀ linear or branched alkyl, wherein at least one of the hydrogen of the alkyl is substituted with substituted or unsubstituted C₂-C₂₀ heteroaryl, the substituted or unsubstituted C₂-C₂₀ heteroaryl may be substituted or unsubstituted furan, substituted or unsubstituted indole, substituted or unsubstituted thiophene, or substituted or unsubstituted pyridine.
When R₄ or R₅ is substituted or unsubstituted C₆-C₂₀ aryl, at least one of the hydrogen of the aryl may be substituted with C₁-C₁₀, C₁-C₅ or C₁-C₃ linear or branched alkyl, —CF₃, —CN, —SH or N(CH₃) C(═O)(CH₃).
When R₄ or R₅ is substituted or unsubstituted C₆-C₂₀ heteroaryl, the substituted or unsubstituted heteroaryl may be substituted or unsubstituted pyrazole, substituted or unsubstituted oxadiazole, substituted or unsubstituted benzothiazole, substituted or unsubstituted triazole, or substituted or unsubstituted thiadiazole, and at least one of the hydrogen of the heteroaryl may be substituted with C₁-C₁₀, C₁-C₅ or C₁-C₃ linear or branched alkyl, an alkylthio group, or —CN.
R_{b} may be C₁-C₁₀ linear or branched alkyl, and R_{c} may be C₁-C₁₀ linear or branched alkyl or C₁-C₁₀ linear or branched alkoxy.
R₈ may be —OC(═O)R_{d} or C₁-C₂₀ linear or branched alkoxy, and R_{d} may be C₁-C₁₀ linear or branched alkyl, C₁-C₁₀ linear or branched alkoxy, or substituted or unsubstituted C₃-C₂₀ cycloalkoxy.
Ar₁ may be substituted or unsubstituted C₆-C₁₂ arylene group, or substituted or unsubstituted C₂-C₁₂ heteroarylene group including N as a heteroatom in a ring.
Ar₁—R₁ may be represented by formula 2 below.

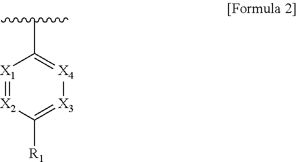

In above formula 2,
X₁ to X₄ may be each independently CH or N, and R₁ may be the same as defined in above formula 1.

Above formula 2 may be represented by formulas 2-1 to 2-8 below.

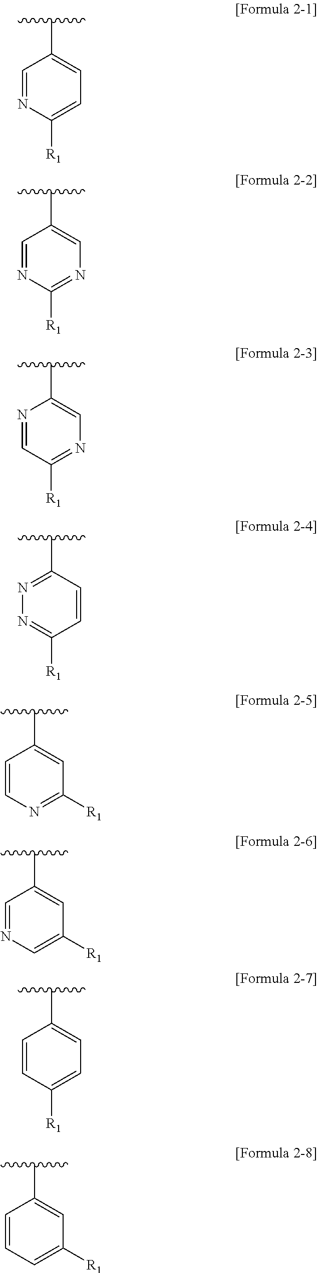

In above formulas 2-1 to 2-8, R₁ may be the same as defined in above formula 1 or 2. In above formulas 2-1 to 2-8, R₁ may be hydrogen, halogen, substituted or unsubstituted C₁-C₁₀ linear or branched alkyl, NR₄R₅, —OR₆, —N═CHR₇, substituted or unsubstituted C₂-C₂₀ heteroaryl, or substituted or unsubstituted C₂-C₁₀ heterocycloalkyl including at least one of N, O and S as a heteroatom in a ring. R₄ to R₇ may be the same as defined in formula 1. The C₂-C₁₀ heterocycloalkyl may include one N in the ring and may further include one or more heteroatoms selected from N, O and S in the ring.
R₁ may be hydrogen, halogen, substituted or unsubstituted C₁-C₁₀ linear or branched alkyl, —NR₄R₅, —OR₆, —N═CHR₇,

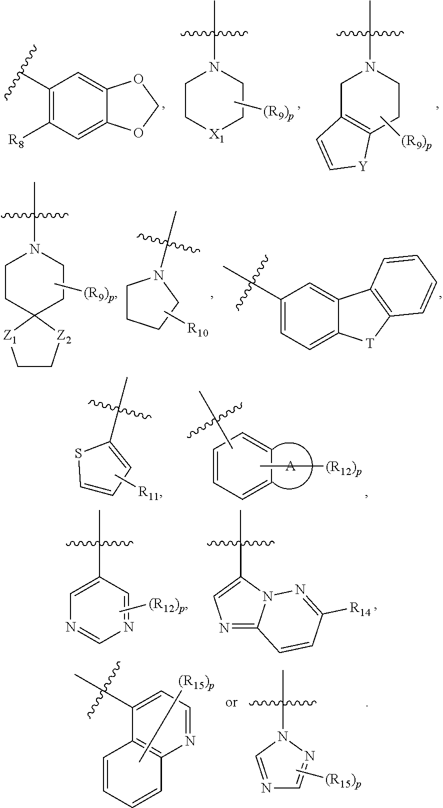

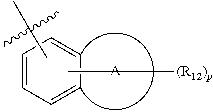

may be

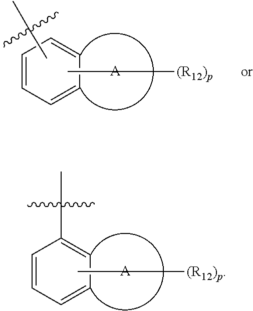

X₁ may be —CHR₁₃, —CX₂ (wherein, X is halogen), —NR₁₃, —O— or —S—. In —CX₂, X may be at least one of F, Cl, Br or I, and X may be the same as or different from each other, and X may be, for example, F.
Y, Z₁, Z₂ and T may be each independently —O— or —S—.
R₉ may be each independently hydrogen, C₁-C₁₀ linear or branched alkyl (wherein at least one of the hydrogen of R₉ may be substituted with —OH, and R₉ may be bonded to an adjacent group to form a substituted or unsubstituted ring), substituted or unsubstituted C₆-C₂₀ aryl, amine, —OH, or —C(═O)ORₑ (Rₑ is hydrogen or C₁-C₁₀ linear or branched alkyl). For example,

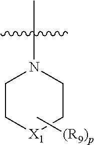

including a substituted or unsubstituted ring which is formed by R₉ being bonded to an adjacent group may be

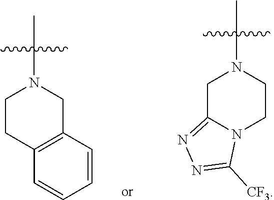 [SMILES: *N1CCc2ccccc2C1 ; *N1CCn2c(nnc2C(F)(F)F)C1]

R₁₀ may be hydrogen, C₁-C₁₀ linear or branched alkyl (wherein at least one of the hydrogen of the alkyl may be substituted with —OH, C₁-C₁₀ linear or branched alkoxy, or substituted or unsubstituted C₂-C₂₀ heterocycloalkyl), —C(═O)R_{f} (wherein R_{f} is —OH, C₁-C₁₀ linear or branched alkyl, C₁-C₁₀ linear or branched alkoxy, substituted or unsubstituted amine, substituted or unsubstituted C₂-C₂₀ heterocycloalkyl) or —OC(═O)R_{g} (wherein R_{g} is C₁-C₁₀ linear or branched alkyl and may be substituted with —OH).
R₁₁ may be —C(═O)Rₐ (wherein Rₐ is hydrogen, C₁-C₁₀ linear or branched alkyl, substituted or unsubstituted C₆-C₂₀ aryl, or substituted or unsubstituted C₂-C₂₀ heteroaryl).
When Rₐ, R_{f} and R_{g} are substituted groups, for example a hydroxy group, an amine group, halogen, an alkyl group, a cycloalkyl group, a heterocycloalkyl group, an aryl group, a heteroaryl group, —C(═O)Rₕ or —Rₕ—C(═O)Rᵢ (wherein Rₕ is C₁-C₁₀ linear or branched alkyl, substituted or unsubstituted C₆-C₂₀ aryl, or substituted or unsubstituted C₂-C₂₀ heteroaryl, and Rᵢ is C₁-C₁₀ linear or branched alkyl).
R₁₂ may be each independently hydrogen, halogen, C₁-C₁₀ linear or branched alkyl (wherein at least one of the hydrogen of the alkyl may be substituted with substituted or unsubstituted C₂-C₁₀ heterocycloalkyl), substituted or unsubstituted C₂-C₂₀ heterocycloalkyl, C₁-C₁₀ linear or branched alkoxy, —CX₃ (wherein X is halogen), or —OH. The substituted or unsubstituted C₂-C₂₀ heterocycloalkyl may be substituted or unsubstituted piperazine, or substituted or unsubstituted morpholine. In —CX₃, X may be at least one of F, Cl, Br or I, and X may be the same as or different from each other, and X may be, for example, F.
R₁₃ may be hydrogen, halogen, substituted or unsubstituted C₁-C₁₀ linear or branched alkyl (wherein at least one of the hydrogen of the alkyl may be substituted with —OH, substituted or unsubstituted C₂-C₁₀ heterocycloalkyl, and R₁₃ may be bonded to an adjacent group to form a substituted or unsubstituted ring), substituted or unsubstituted C₃-C₂₀ cycloalkyl (wherein a substituent of the C₃-C₂₀ cycloalkyl may be bonded to an adjacent group to form a ring), substituted or unsubstituted C₆-C₂₀ aryl, substituted or unsubstituted C₃-C₂₀ heteroaryl (wherein at least one of the hydrogen of the C₃-C₂₀ heteroaryl may be substituted with C₁-C₁₀ alkylthio group or halogen), —OH, or amine.
R₁₄ may be C₂-C₁₀ heterocycloalkyl including at least one of N and O as a heteroatom in a ring. For example, the C₂-C₁₀ heterocycloalkyl may be morpholine.
R₁₅ may be each independently halogen, C₁-C₁₀ linear or branched alkyl, —CX₃, C₁-C₁₀ linear or branched alkoxy, an alkylthio group, or amine. Here, X may be halogen and, for example, the halogen may be F. p may be an integer of 0 or more and 7 or less, 0 or more and 5 or less, 0 or more and 4 or less, or 0 or more and 3 or less.
A may be substituted or unsubstituted C₂-C₁₀ heterocycloalkyl including at least one of N, O and S as a heteroatom in a ring, substituted or unsubstituted C₆-C₂₀ aryl, or substituted or unsubstituted C₂-C₂₀ heteroaryl including at least one N as a heteroatom in a ring. For example, A may be C₂-C₁₀, C₂-C₇ or C₂-C₅ heteroaryl including N as a heteroatom. For example, A may be pyrrole, pyridine, pyrimidine pyran-2-one, or furan.
R₄ to R₈ may be the same as defined in formula 1.
R₁ may be hydrogen, fluorine, chlorine, —CH₃,

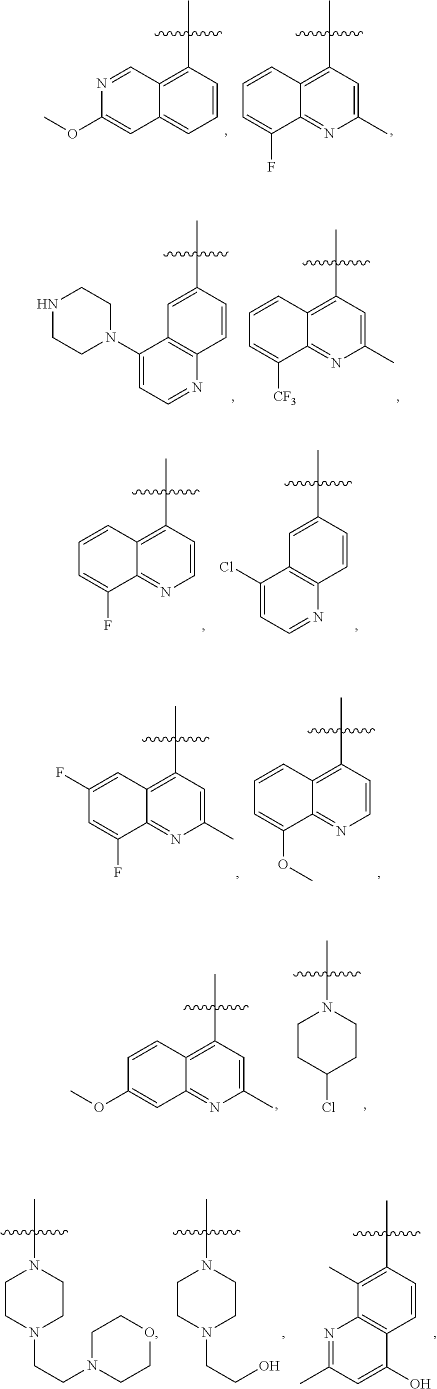

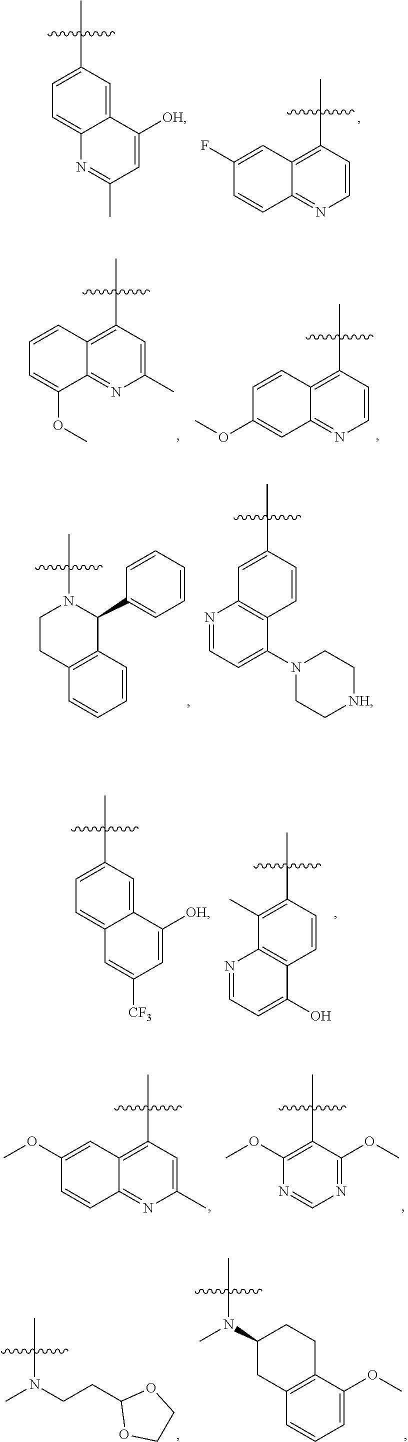

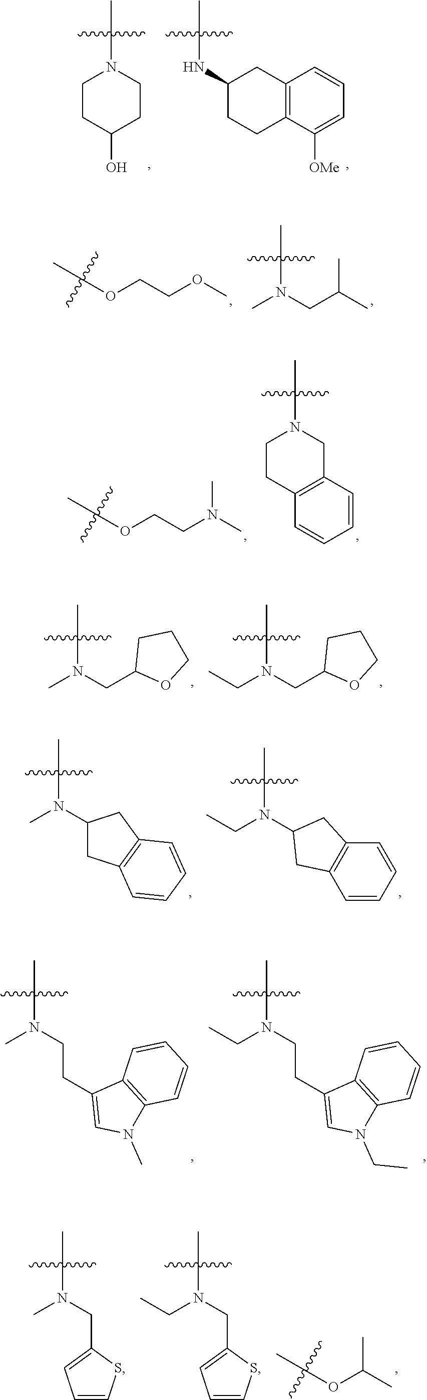

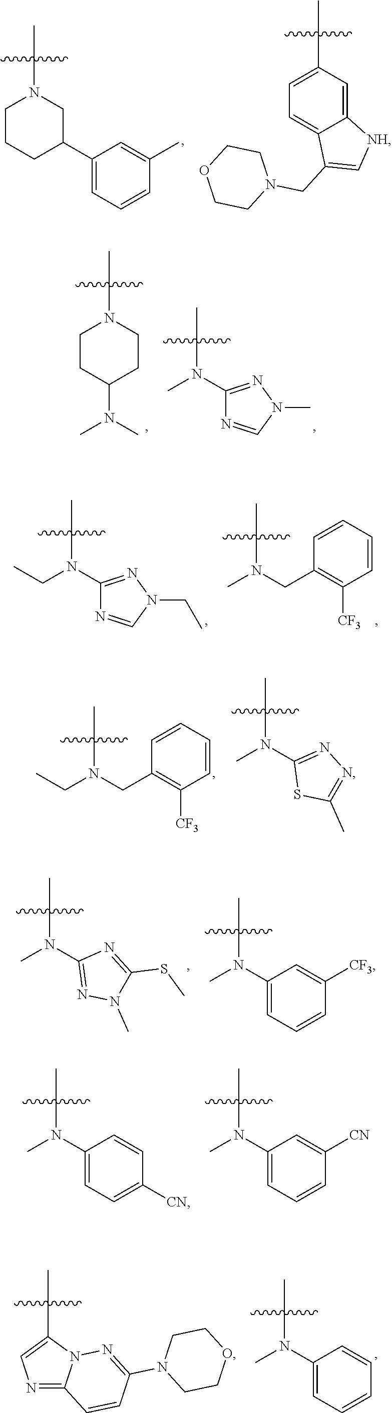

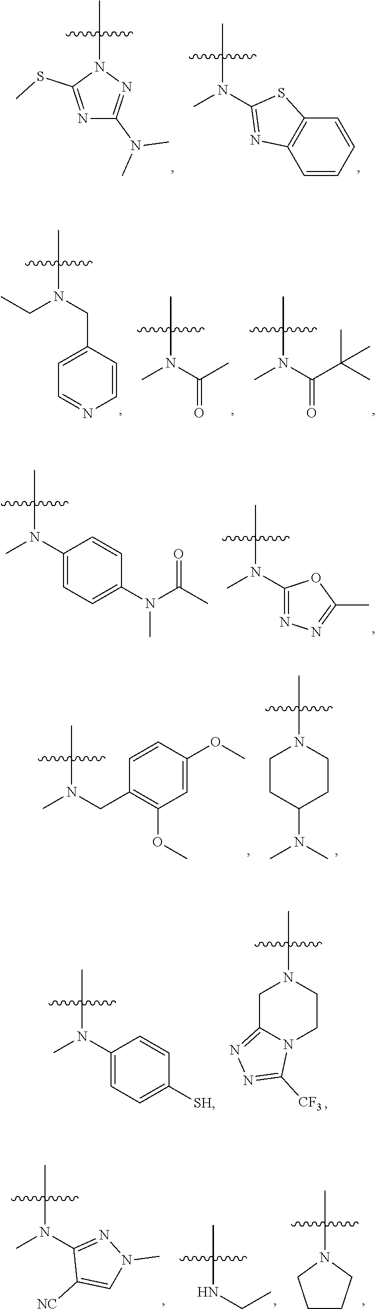

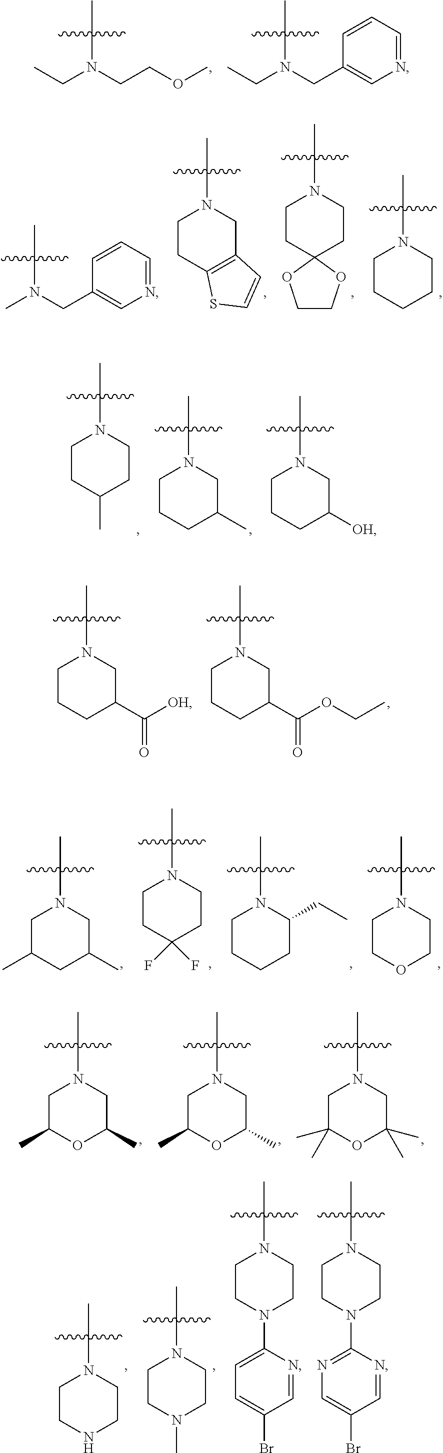

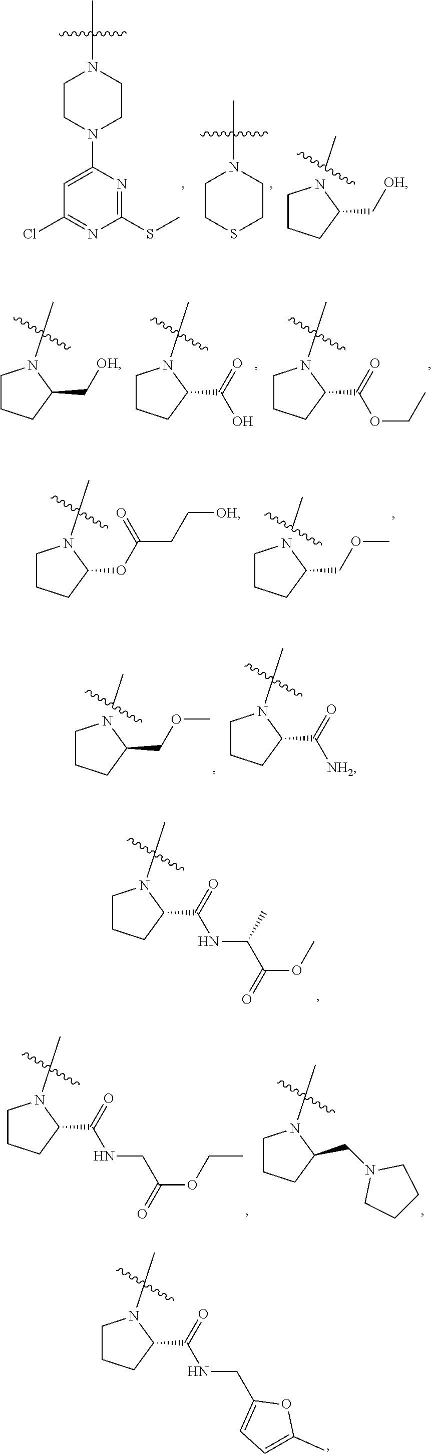

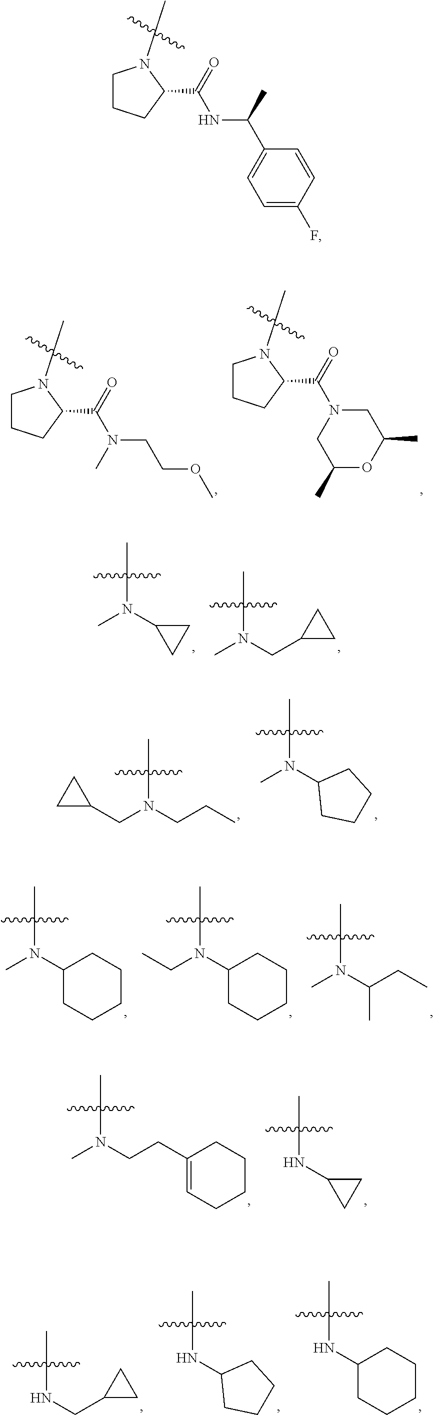

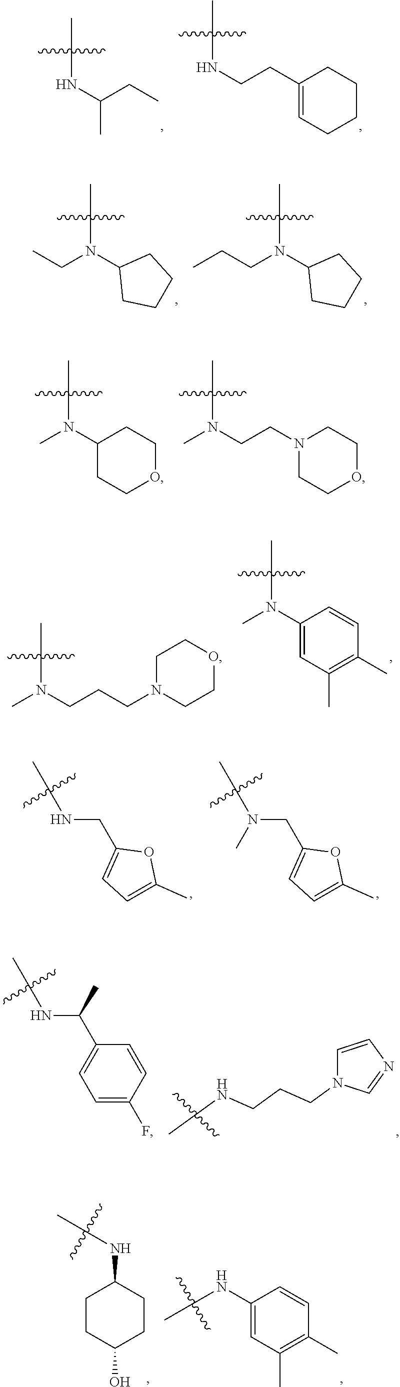

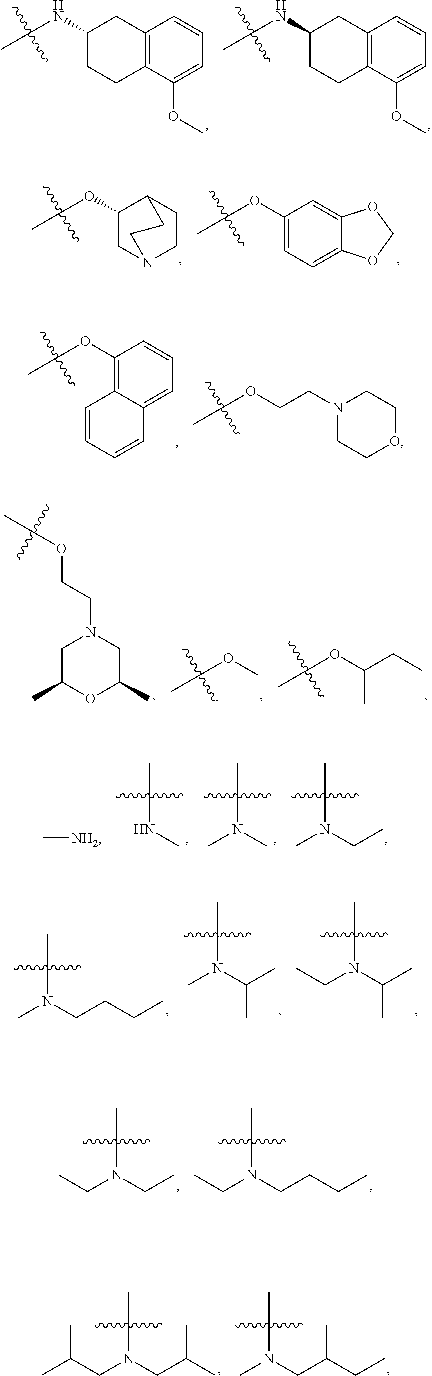

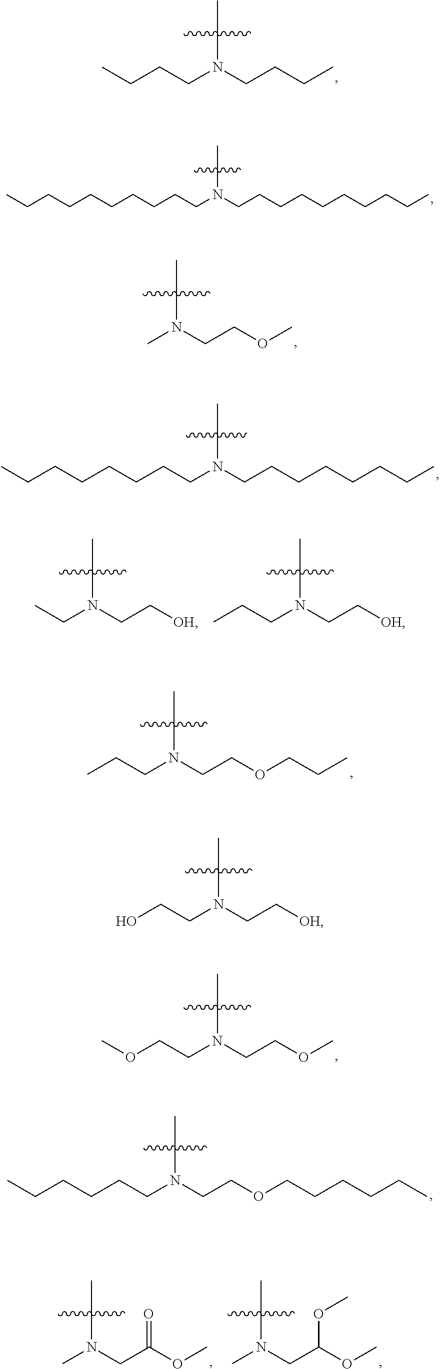

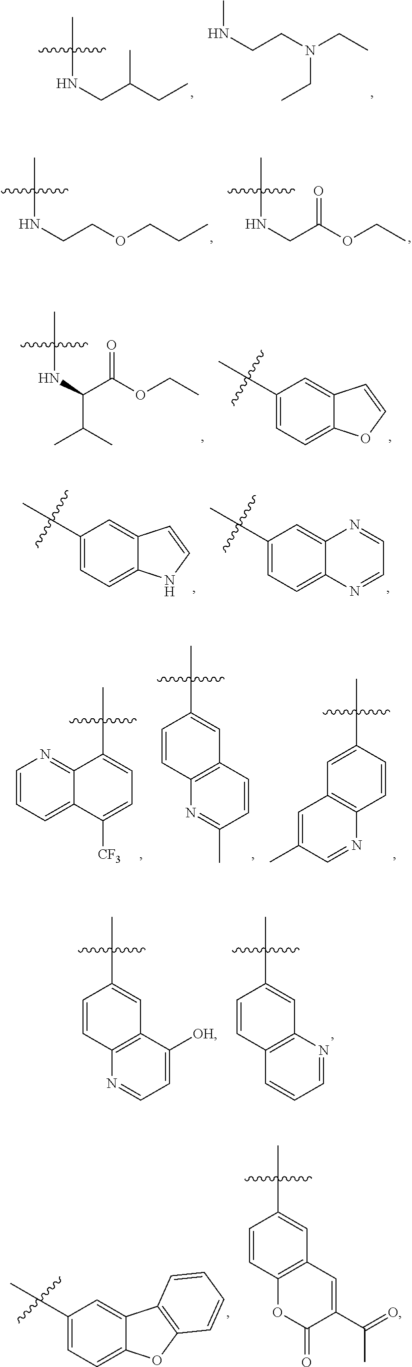

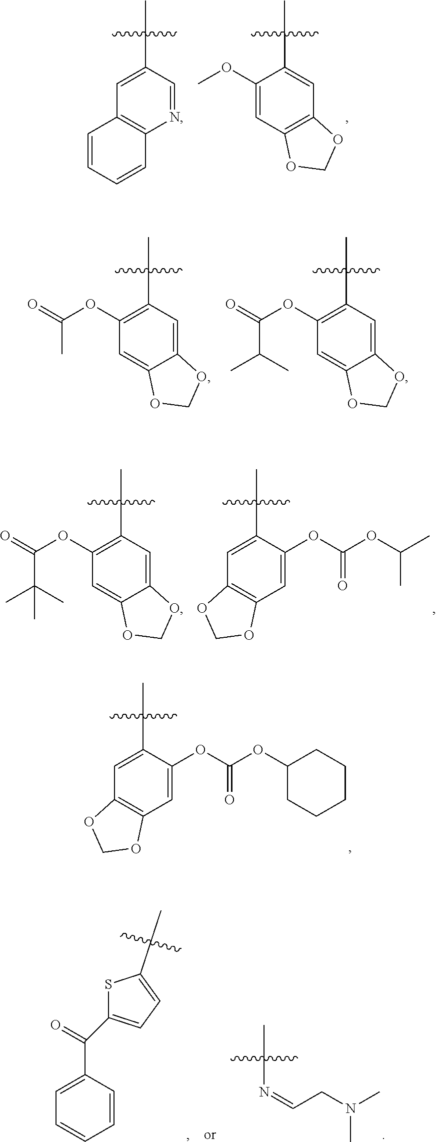

The compound represented by above formula 1 may be one selected from the group consisting of the following compounds:
[001] (S)-6,7-dimethoxy-9-((5-methoxy-1,2,3,4-tetrahydronaphthalen-2-yl)amino)naphtho[2,3-c]furan-1(3H)-one
[002] 9-((2S,6R)-2,6-dimethylmorpholino)-6,7-dimethoxynaphtho[2,3-c]furan-1(3H)-one
[003] 9-(6,7-dihydrothieno[3,2-c]pyridin-5 (4H)-yl)-6,7-dimethoxynaphtho[2,3-c]furan-1(3H)-one
[004] 9-(3,5-dimethylpiperidin-1-yl)-6,7-dimethoxynaphtho[2,3-c]furan-1(3H)-one
[005] 6,7-dimethoxy-9-((2-methoxyethyl)(methyl)amino)naphtho[2,3-c]furan-1(3H)-one
[006] 9-((2-(diethylamino)ethyl)amino)-6,7-dimethoxynaphtho[2,3-c]furan-1(3H)-one
[007] 9-(2-((2S,6R)-2,6-dimethylmorpholino)pyrimidin-5-yl)-6,7-dimethoxynaphtho[2,3-c]furan-1(3H)-one
[008] 9-(2-((2S,6R)-2,6-dimethylmorpholino)pyrimidin-5-yl)-6,7-dimethoxynaphtho[2,3-c]furan-1(3H)-one
[009] 6,7-dimethoxy-9-(6-thiomorpholinopyridin-3-yl)naphtho[2,3-c]furan-1(3H)-one
[010] 6,7-dimethoxy-9-(6-thiomorpholinopyridin-3-yl)naphtho[2,3-c]furan-1(3H)-one
[011] 9-(2-(dimethylamino)pyrimidin-5-yl)-6,7-dimethoxynaphtho[2,3-c]furan-1(3H)-one
[012] 9-(2-(6,7-dihydrothieno[3,2-c]pyridin-5 (4H)-yl)pyrimidin-5-yl)-6,7-dimethoxynaphtho[2,3-c]furan-1(3H)-one
[013] 6,7-dimethoxy-9-(2-(2-morpholinoethoxy)pyrimidin-5-yl)naphtho[2,3-c]furan-1(3H)-one
[014] 9-(2-((3,4-dimethylphenyl)amino)pyrimidin-5-yl)-6,7-dimethoxynaphtho[2,3-c]furan-1(3H)-one
[015] Ethyl 1-(5-(6,7-dimethoxy-3-oxo-1,3-dihydronaphtho[2,3-c]furan-4-yl)pyrimidin-2-yl) piperidin-3-carboxylate
[016] 9-(2-((3-(1H-imidazol-1-yl) propyl)amino)pyrimidin-5-yl)-6,7-dimethoxynaphtho[2,3-c]furan-1(3H)-one
[017] (R)-6,7-dimethoxy-9-(2-(quinuclidin-3-yloxy)pyrimidin-5-yl)naphtho[2,3-c]furan-1(3H)-one
[018] 6,7-dimethoxy-9-(2-((2-methoxyethyl)(methyl)amino)pyrimidin-5-yl)naphtho[2,3-c]furan-1(3H)-one
[019] (S)ethyl 1-(5-(6,7-dimethoxy-3-oxo-1,3-dihydronaphtho[2,3-c]furan-4-yl)pyrimidin-2-yl)pyrrolidin-2-carboxylate
[020] 9-(2-(benzo[d][1,3]dioxol-5-yloxy)pyrimidin-5-yl)-6,7-dimethoxynaphtho[2,3-c]furan-1(3H)-one
[021] 6,7-dimethoxy-9-(2-(naphthalen-1-yloxy)pyrimidin-5-yl)naphtho[2,3-c]furan-1(3H)-one
[022] (S)-6,7-dimethoxy-9-(2-((5-methoxy-1,2,3,4-tetrahydronaphthalen-2-yl)amino)pyrimidin-5-yl)naphtho[2,3-c]furan-1(3H)-one
[023] 9-(2-(isopropyl(methyl)amino)pyrimidin-5-yl)-6,7-dimethoxynaphtho[2,3-c]furan-1(3H)-one
[024] 9-(2-((S)-2-((2S,6R)-2,6-dimethylmorpholin-4-carbonyl)pyrrolidin-1-yl)pyrimidin-5-yl)-6,7-dimethoxynaphtho[2,3-c]furan-1(3H)-one
[025] 9-(2-(sec-butoxy)pyrimidin-5-yl)-6,7-dimethoxynaphtho[2,3-c]furan-1(3H)-one
[026] 6,7-dimethoxy-9-(2-methoxypyrimidin-5-yl)naphtho[2,3-c]furan-1(3H)-one
[027] Methyl
[028] (5-(6,7-dimethoxy-3-oxo-1,3-dihydronaphtho[2,3-c]furan-4-yl)pyrimidin-2-yl)-D-valinate
[029] (Z)-9-(5-((2-(dimethylamino)ethyliden)amino)pyrazin-2-yl)-6,7-dimethoxynaphtho[2,3-c]furan-1(3H)-one
[030] 9-(5-aminopyrazin-2-yl)-6,7-dimethoxynaphtho[2,3-c]furan-1(3H)-one
[031] 9-(5-(dimethylamino)pyrazin-2-yl)-6,7-dimethoxynaphtho[2,3-c]furan-1(3H)-one
[032] 6,7-dimethoxy-9-(pyrazin-2-yl)naphtho[2,3-c]furan-1(3H)-one
[033] (S)-1-(5-(6,7-dimethoxy-3-oxo-1,3-dihydronaphtho[2,3-c]furan-4-yl)pyrimidin-2-yl)-N-(2-methoxyethyl)-N-methylpyrrolidin-2-carboxamide
[034] 9-(1H-indol-5-yl)-6,7-dimethoxynaphtho[2,3-c]furan-1(3H)-one
[035] 9-(6-fluoropyridin-3-yl)-6,7-dimethoxynaphtho[2,3-c]furan-1(3H)-one
[036] 2-hydroxyethyl (5-(6,7-dimethoxy-3-oxo-1,3-dihydronaphtho[2,3-c]furan-4-yl)pyrimidin-2-yl)-L-prolinate
[037] 6,7-dimethoxy-9-(2-((2-propoxyethyl)amino)pyrimidin-5-yl)naphtho[2,3-c]furan-1(3H)-one
[038] Methyl (5-(6,7-dimethoxy-3-oxo-1,3-dihydronaphtho[2,3-c]furan-4-yl)pyrimidin-2-yl)-L-prolyl-D-alaninate
[039] (S)-1-(5-(6,7-dimethoxy-3-oxo-1,3-dihydronaphtho[2,3-c]furan-4-yl)pyrimidin-2-yl)-N—((S)-1-(4-fluorophenyl)ethyl)pyrrolidin-2- carboxamide
[040] 6,7-dimethoxy-9-(quinoxalin-6-yl)naphtho[2,3-c]furan-1(3H)-one
[041] (S)-1-(5-(6,7-dimethoxy-3-oxo-1,3-dihydronaphtho[2,3-c]furan-4-yl)pyrimidin-2-yl)-N-((5-methylfuran-2-yl)methyl)pyrrolidin-2-carboxamide
[042] 6,7-dimethoxy-9-(2-((2-propoxyethyl)(propyl)amino)pyrimidin-5-yl)naphtho[2,3-c]furan-1(3H)-one
[043] 9-(dibenzo[b,d]furan-2-yl)-6,7-dimethoxynaphtho[2,3-c]furan-1(3H)-one
[044] Ethyl (5-(6,7-dimethoxy-3-oxo-1,3-dihydronaphtho[2,3-c]furan-4-yl)pyrimidin-2-yl)glycinate
[045] Ethyl (5-(6,7-dimethoxy-3-oxo-1,3-dihydronaphtho[2,3-c]furan-4-yl)pyrimidin-2-yl)-L-proglycinate
[046] 6,7-dimethoxy-9-(quinolin-3-yl)naphtho[2,3-c]furan-1(3H)-one
[047] 9-(2-(((1r,4r)-4-hydroxycyclohexyl)amino)pyrimidin-5-yl)-6,7-dimethoxynaptho[2,3-c]furan-1(3H)-one
[048] 9-(2-(hexyl(2-(hexyloxy)ethyl)amino)pyrimidin-5-yl)-6,7-dimethoxynaphtho[2,3-c]furan-1(3H)-one
[049] (S)-1-(5-(6,7-dimethoxy-3-oxo-1,3-dihydronaphtho[2,3-c]furan-4-yl)pyrimidin-2-yl) pyrrolidin-2-carboxamide
[050] 6,7-dimethoxy-9-(2-(((5-methylfuran-2-yl)methyl)amino)pyrimidin-5-yl)naphtho[2,3-c]furan-1(3H)-one
[151] 6,7-dimethoxy-9-(2-morpholinopyrimidin-5-yl)naphtho[2,3-c]furan-1(3H)-one
[152] 9-(benzofuran-5-yl)-6,7-dimethoxynaphtho[2,3-c]furan-1(3H)-one
[053] 9-(5-benzothiophen-2-yl)-6,7-dimethoxynaphtho[2,3-c]furan-1(3H)-one
[054] 9-(2-(2-((2S,6R)-2,6-dimethylmorpholino) ethoxy)pyrimidin-5-yl)-6,7-dimethoxynaphtho[2,3-c]furan-1(3H)-one
[055] (S)-9-(2-((1-(4-fluorophenyl)ethyl)amino)pyrimidin-5-yl)-6,7-dimethoxynaphtho[2,3-c]furan-1(3H)-one
[056] 9-(3-acetyl-2-oxo-2H-chromen-6-yl)-6,7-dimethoxynaphtho[2,3-c]furan-1(3H)-one
[057] 9-(2-(3,5-dimethylpiperidin-1-yl)pyrimidin-5-yl)-6,7-dimethoxynaphtho[2,3-c]furan-1(3H)-one
[058] 9-(2-((2-(diethylamino)ethyl)amino)pyrimidin-5-yl)-6,7-dimethoxynaphtho[2,3-c]furan-1(3H)-one
[059] 6,7-dimethoxy-9-(2-(3-methylpiperidin-1-yl)pyrimidin-5-yl)naphtho[2,3-c]furan-1(3H)-one
[060] 1-(5-(6,7-dimethoxy-3-oxo-1,3-dihydronaphtho[2,3-c]furan-4-yl)pyrimidin-2-yl) piperidin-3-carboxylic acid
[061] (5-(6,7-dimethoxy-3-oxo-1,3-dihydronaphtho[2,3-c]furan-4-yl)pyrimidin-2-yl)-L-prolan
[062] 9-(2-((2S,6S)-2,6-dimethylmorpholino)pyrimidin-5-yl)-6,7-dimethoxynaphtho[2,3-c]furan-1(3H)-one
[063] 6,7-dimethoxy-9-(2-thiomorpholinopyrimidin-5-yl)naphtho[2,3-c]furan-1(3H)-one
[064] 6,7-dimethoxy-9-(2-((2-methylbutyl)amino)pyrimidin-5-yl)naphtho[2,3-c]furan-1(3H)-one
[065] 6,7-dimethoxy-9-(2-(methyl(2-methylbutyl)amino)pyrimidin-5-yl)naphtho[2,3-c]furan-1(3H)-one
[066] 9-(2-(butyl(ethyl)amino)pyrimidin-5-yl)-6,7-dimethoxynaphtho[2,3-c]furan-1(3H)-one
[067] 9-(2-((cyclopropylmethyl)amino)pyrimidin-5-yl)-6,7-dimethoxynaphtho[2,3-c]furan-1(3H)-one
[068] 9-(2-((cyclopropylmethyl)(methyl)amino)pyrimidin-5-yl)-6,7-dimethoxynaphtho[2,3-c]furan-1(3H)-one
[069] 9-(2-(cyclopropylamino)pyrimidin-5-yl)-6,7-dimethoxynaphtho[2,3-c]furan-1(3H)-one
[070] 9-(2-(cyclopropyl(methyl)amino)pyrimidin-5-yl)-6,7-dimethoxynaphtho[2,3-c]furan-1(3H)-one
[071] 9-(2-(butyl(methyl)amino)pyrimidin-5-yl)-6,7-dimethoxynaphtho[2,3-c]furan-1(3H)-one
[072] 9-(2-(bis(2-methoxyethyl)amino)pyrimidin-5-yl)-6,7-dimethoxynaphtho[2,3-c]furan-1(3H)-one
[073] 9-(2-(dibutylamino)pyrimidin-5-yl)-6,7-dimethoxynaphtho[2,3-c]furan-1(3H)-one
[074] 9-(2-(diethylamino)pyrimidin-5-yl)-6,7-dimethoxynaphtho[2,3-c]furan-1(3H)-one
[075] 9-(2-(ethyl(isopropyl)amino)pyrimidin-5-yl)-6,7-dimethoxynaphtho[2,3-c]furan-1(3H)-one hydrochloride
[076] 9-(2-(butyl(methyl)amino)pyrimidin-5-yl)-6,7-dimethoxynaphtho[2,3-c]furan-1(3H)-one hydrochloride
[077] 9-(2-((2-hydroxyethyl)(propyl)amino)pyrimidin-5-yl)-6,7-dimethoxynaphtho[2,3-c]furan-1(3H)-one hydrochloride
[078] 9-(2-(cyclohexyl(ethyl)amino)pyrimidin-5-yl)-6,7-dimethoxynaphtho[2,3-c]furan-1(3H)-one hydrochloride
[079] 9-(2-(cyclohexyl(methyl)amino)pyrimidin-5-yl)-6,7-dimethoxynaphtho[2,3-c]furan-1(3H)-one hydrochloride
[080] 9-(2-(3-hydroxy peridin-1-ylpyrimidin-5-yl)-6,7-dimethoxynaphtho[2,3-c]furan-1(3H)-one hydrochloride
[081] 9-(2-(ethyl(2-hydroxyethyl)amino)pyrimidin-5-yl)-6,7-dimethoxynaphtho[2,3-c]furan-1(3H)-one hydrochloride
[082] (R)-9-(2-(2-(hydroxymethyl)pyrrolidin-1-yl)pyrimidin-5-yl)-6,7-dimethoxynaphtho[2,3-c]furan-1(3H)-one hydrochloride
[083] (S)-9-(2-(2-(hydroxymethyl)pyrrolidin-1-yl)pyrimidin-5-yl)-6,7-dimethoxynaphtho[2,3-c]furan-1(3H)-one hydrochloride
[084] 9-(2-(cyclopentyl(methyl)amino)pyrimidin-5-yl)-6,7-dimethoxynaphtho[2,3-c]furan-1(3H)-one hydrochloride
[085] 9-(2-(bis(2-hydroxyethyl)amino)pyrimidin-5-yl)-6,7-dimethoxynaphtho[2,3-c]furan-1(3H)-one hydrochloride
[086] 9-(2-(diisobutylamino)pyrimidin-5-yl)-6,7-dimethoxynaphtho[2,3-c]furan-1(3H)-one hydrochloride
[087] 9-(2-(didecylamino)pyrimidin-5-yl)-6,7-dimethoxynaphtho[2,3-c]furan-1(3H)-one hydrochloride
[088] 6,7-dimethoxy-9-(6-methoxybenzo[d][1,3]dioxol-5-yl)naphtho[2,3-c]furan-1(3H)-one
[089] 9-(2-(1,4-dioxa-8-azaspiro[4.5]decan-8-yl)pyrimidin-5-yl)-6,7-dimethoxynaphtho[2,3-c]furan-1(3H)-one
[090] Methyl N-(5-(6,7-dimethoxy-3-oxo-1,3-dihydronaphtho[2,3-c]furan-4-yl)pyrimidin-2-yl)-N-methyl glycinate hydrochloride
[091] 9-(2-(cyclopentylamino)pyrimidin-5-yl)-6,7-dimethoxynaphtho[2,3-c]furan-1(3H)-one hydrochloride
[092] 9-(2-(cyclopentyl(ethyl)amino)pyrimidin-5-yl)-6,7-dimethoxynaphtho[2,3-c]furan-1(3H)-one hydrochloride
[093] 9-(2-(cyclopentyl(propyl)amino)pyrimidin-5-yl)-6,7-dimethoxynaphtho[2,3-c]furan-1(3H)-one hydrochloride
[094] 9-(2-((2,2-dimethoxyethyl)(methyl)amino)pyrimidin-5-yl)-6,7-dimethoxynaphtho[2,3-c]furan-1(3H)-one
[095] 9-(2-(dioctylamino)pyrimidin-5-yl)-6,7-dimethoxynaphtho[2,3-c]furan-1(3H)-one hydrochloride
[096] (S)-6,7-dimethoxy-9-(2-((5-methoxy-1,2,3,4-tetrahydronaphthalen-2-yl)(methyl)amino)pyrimidin-5-yl)naphtho[2,3-c]furan-1(3H)-one hydrochloride
[097] (S)-6,7-dimethoxy-9-(2-(2-(methoxymethyl)pyrrolidin-1-yl)pyrimidin-5-yl)naphtho[2,3-c]furan-1(3H)-one hydrochloride
[098] 6-(6,7-dimethoxy-3-oxo-1,3-dihydronaphtho[2,3-c]furan-4-yl)benzo[d][1,3]dioxol-5-yl isopropyl carbonate
[099] Cyclohexyl (6-(6,7-dimethoxy-3-oxo-1,3-dihydronaphtho[2,3-c]furan-4-yl)benzo[d][1,3]dioxol-5-yl)carbonate
[100] 9-(2-(sec-butyl(methyl)amino)pyrimidin-5-yl)-6,7-dimethoxynaphtho[2,3-c]furan-1(3H)-one hydrochloride
[101] 9-(2-((2-(cyclohex-1-en-1-yl)ethyl)(methyl)amino)pyrimidin-5-yl)-6,7-dimethoxynaphtho[2,3-c]furan-1(3H)-one hydrochloride
[102] (R)-6,7-dimethoxy-9-(2-(2-(methoxymethyl)pyrrolidin-1-yl)pyrimidin-5-yl)naphtho[2,3-c]furan-1(3H)-one hydrochloride
[103] 6,7-dimethoxy-9-(2-(methyl((5-methylfuran-2-yl)methyl)amino)pyrimidin-5-yl)naphtho[2,3-c]furan-1(3H)-one hydrochloride
[104] 9-(2-(2-(hydroxymethyl) piperidin-1-yl)pyrimidin-5-yl)-6,7-dimethoxynaphtho[2,3-c]furan-1(3H)-one hydrochloride
[105] 9-(2-((cyclopropylmethyl)(propyl)amino)pyrimidin-5-yl)-6,7-dimethoxynaphtho[2,3-c]furan-1(3H)-one hydrochloride
[106] 9-(2-(ethyl(methyl)amino)pyrimidin-5-yl)-6,7-dimethoxynaphtho[2,3-c]furan-1(3H)-one hydrochloride
[107] 6,7-dimethoxy-9-(2-(4-methylpiperidin-1-yl)pyrimidin-5-yl)naphtho[2,3-c]furan-(3H)-one hydrochloride
[108] 6,7-dimethoxy-9-(2-(methylamino)pyrimidin-5-yl)naphtho[2,3-c]furan-1(3H)-one
[109] 9-(2-((3,4-dimethylphenyl)(methyl)amino)pyrimidin-5-yl)-6,7-dimethoxynaphtho[2,3-c]furan-1(3H)-one
[110] (R)-6,7-dimethoxy-9-(2-(2-(pyrrolidin-1-ylmethyl)pyrrolidin-1-yl)pyrimidin-5-yl)naphtho[2,3-c]furan-1(3H)-one
[111] 6,7-dimethoxy-9-(2-(methyl(2-morpholinoethyl)amino)pyrimidin-5-yl)naphtho[2,3-c]furan-1(3H)-one hydrochloride
[112] 6,7-dimethoxy-9-(2-(methyl(3-morpholinopropyl)amino)pyrimidin-5-yl)naphtho[2,3-c]furan-1(3H)-one dihydrochloride
[113] (R)-9-(2-(2-ethylpiperidin-1-yl)pyrimidin-5-yl)-6,7-dimethoxynaphtho[2,3-c]furan-1(3H)-one
[114] 6,7-dimethoxy-9-(2-(2,2,6,6-tetramethylmorpholino)pyrimidin-5-yl)naphtho[2,3-c]furan-1(3H)-one
[115] 6,7-dimethoxy-9-(2-(methyl(tetrahydro-2H-pyran-4-yl)amino)pyrimidin-5-yl)naphtho[2,3-c]furan-1(3H)-one
[116] 6,7-dimethoxy-9-(2-methylquinolin-6-yl)naphtho[2,3-c]furan-1(3H)-one
[117] 9-(4-hydroxyquinolin-6-yl)-6,7-dimethoxynaphtho[2,3-c]furan-1(3H)-one
[118] 6,7-dimethoxy-9-(quinolin-7-yl)naphtho[2,3-c]furan-1(3H)-one
[119] 6,7-dimethoxy-9-(5-(trifluoromethyl) quinolin-8-yl)naphtho[2,3-c]furan-1(3H)-one
[120] 6,7-dimethoxy-9-(3-methylquinolin-6-yl)naphtho[2,3-c]furan-1(3H)-one
[121] 9-(6-((2S,6R)-2,6-dimethylmorpholino)pyridin-3-yl)-6,7-dimethoxynaphtho[2,3-c]furan-1(3H)-one
[122] 9-(6-(4,4-difluoropiperidin-1-yl)pyridin-3-yl)-6,7-dimethoxynaphtho[2,3-c]furan-1(3H)-one
[123] 6,7-dimethoxy-9-(6-(piperidin-1-yl)pyridin-3-yl)naphtho[2,3-c]furan-1(3H)-one
[124] 6,7-dimethoxy-9-(6-morpholinopyridin-3-yl)naphtho[2,3-c]furan-1(3H)-one
[125] 6,7-dimethoxy-9-(quinolin-8-yl)naphtho[2,3-c]furan-1(3H)-one
[126] 6,7-dimethoxy-9-(6-(3-methylpiperidin-1-yl)pyridin-3-yl)naphtho[2,3-c]furan-1(3H)-one
[127] 6,7-dimethoxy-9-(6-(3-methylpiperidin-1-yl)pyridin-3-yl)naphtho[2,3-c]furan-1(3H)-one
[128] 6,7-dimethoxy-9-(piperazin-1-yl)naphtho[2,3-c]furan-1(3H)-one
[129] 9-(4-(5-bromopyridin-2-yl) piperazin-1-yl)-6,7-dimethoxynaphtho[2,3-c]furan-1(3H)-one
[130] 9-(4-(6-chloro-2-(methylthio)pyrimidin-4-yl) piperazin-1-yl)-6,7-dimethoxynaphtho[2,3-c]furan-1(3H)-one
[131] 9-(4-(5-bromopyrimidin-2-yl) piperazin-1-yl)-6,7-dimethoxynaphtho[2,3-c]furan-1(3H)-one
[132] 6,7-dimethoxy-9-(quinolin-5-yl)naphtho[2,3-c]furan-1(3H)-one
[133] 6,7-dimethoxy-9-(3-methoxyisoquinolin-8-yl)naphtho[2,3-c]furan-1(3H)-one
[134] 9-(8-fluoro-2-methylquinolin-4-yl)-6,7-dimethoxynaphtho[2,3-c]furan-1(3H)-one
[135] 6,7-dimethoxy-9-(4-(piperazin-1-yl) quinolin-6-yl)naphtho[2,3-c]furan-1(3H)-one
[136] 6,7-dimethoxy-9-(2-methyl-8-(trifluoromethyl) quinolin-4-yl)naphtho[2,3-c]furan-1(3H)-one
[137] 9-(8-fluoroquinolin-4-yl)-6,7-dimethoxynaphtho[2,3-c]furan-1(3H)-one
[138] 9-(4-chloroquinolin-6-yl)-6,7-dimethoxynaphtho[2,3-c]furan-1(3H)-one
[139] 9-(6,8-difluoro-2-methylquinolin-4-yl)-6,7-dimethoxynaphtho[2,3-c]furan-1(3H)-one
[140] 6,7-dimethoxy-9-(8-methoxyquinolin-4-yl)naphtho[2,3-c]furan-1(3H)-one
[141] 6,7-dimethoxy-9-(7-methoxy-2-methylquinolin-4-yl)naphtho[2,3-c]furan-1(3H)-one
[142] 9-(6-(4-chloropiperidin-1-yl)pyridin-3-yl)-6,7-dimethoxynaphtho[2,3-c]furan-1(3H)-one
[143] 6,7-dimethoxy-9-(4-(2-morpholinoethyl) piperazin-1-yl)naphtho[2,3-c]furan-1(3H)-one
[144] 9-(4-(2-hydroxyethyl) piperazin-1-yl)-6,7-dimethoxynaphtho[2,3-c]furan-1(3H)-one
[146] 9-(4-hydroxy-2,8-dimethylquinolin-7-yl)-6,7-dimethoxynaphtho[2,3-c]furan-1(3H)-one
[147] 9-(4-hydroxy-2-methylquinolin-6-yl)-6,7-dimethoxynaphtho[2,3-c]furan-1(3H)-one
[148] 9-(6-fluoroquinolin-4-yl)-6,7-dimethoxynaphtho[2,3-c]furan-1(3H)-one
[149] 6,7-dimethoxy-9-(8-methoxy-2-methylquinolin-4-yl)naphtho[2,3-c]furan-1(3H)-one
[150] 6,7-dimethoxy-9-(7-methoxyquinolin-4-yl)naphtho[2,3-c]furan-1(3H)-one
[151] (S)-6,7-dimethoxy-9-(2-(1-phenyl-3,4-dihydroisoquinolin-2 (1H)-yl)pyrimidin-5-yl)naphtho[2,3-c]furan-1(3H)-one
[152] 6,7-dimethoxy-9-(4-(piperazin-1-yl) quinolin-7-yl)naphtho[2,3-c]furan-1(3H)-one
[154] 9-(4-hydroxy-8-methylquinolin-7-yl)-6,7-dimethoxynaphtho[2,3-c]furan-1(3H)-one
[155] 6,7-dimethoxy-9-(6-methoxy-2-methylquinolin-4-yl)naphtho[2,3-c]furan-1(3H)-one
[156] 9-(6-(6,7-dihydrothien[3,2-c]pyridin-5 (4H)-yl)pyridin-3-yl)-6,7-dimethoxynaphtho[2,3-c]furan-1(3H)-one
[157] 9-(4-(4,6-dimethoxypyrimidin-5-yl)phenyl)-6,7-dimethoxynaphtho[2,3-c]furan-1(3H)-one
[158] 9-(2-((2-(1,3-dioxolan-2-yl)ethyl)(methyl)amino)pyrimidin-5-yl)-6,7-dimethoxynaphtho[2,3-c]furan-1(3H)-one
[159] 6,7-dimethoxy-9-(6-(4-methylpiperidin-1-yl)pyridin-3-yl)naphtho[2,3-c]furan-1(3H)-one
[160] 9-(6-(diethylamino)pyridin-3-yl)-6,7-dimethoxynaphtho[2,3-c]furan-1(3H)-one
[161] 9-(6-((2-(1,3-dioxolan-2-yl)ethyl)(methyl)amino)pyridin-3-yl)-6,7-dimethoxynaphtho[2,3-c]furan-1(3H)-one
[162] 9-(6-((2,2-dimethoxyethyl)(methyl)amino)pyridin-3-yl)-6,7-dimethoxynaphtho[2,3-c]furan-1(3H)-one
[163] 9-(6-(dimethylamino)pyridin-3-yl)-6,7-dimethoxynaphtho[2,3-c]furan-1(3H)-one
[164] (S)-6,7-dimethoxy-9-(6-((5-methoxy-1,2,3,4-tetrahydronaphthalen-2-yl)(methyl)amino)pyridin-3-yl)naphtho[2,3-c]furan-1(3H)-one
[165] 9-(6-(4-hydroxypiperidin-1-yl)pyridin-3-yl)-6,7-dimethoxynaphtho[2,3-c]furan-1(3H)-one
[166] 9-(6-(1,4-dioxa-8-azaspiro[4.5]decan-8-yl)pyridin-3-yl)-6,7-dimethoxynaphtho[2,3-c]furan-1(3H)-one
[167] 6,7-dimethoxy-9-(6-(4-methylpiperazin-1-yl)pyridin-3-yl)naphtho[2,3-c]furan-1(3H)-one
[168] 6,7-dimethoxy-9-(6-((2-methoxyethyl)(methyl)amino)pyridin-3-yl)naphtho[2,3-c]furan-1(3H)-one hydrochloride
[171] 9-(6-(cyclopropyl(methyl)amino)pyridin-3-yl)-6,7-dimethoxynaphtho[2,3-c]furan-1(3H)-one
[172] (S)-6,7-dimethoxy-9-(6-((5-methoxy-1,2,3,4-tetrahydronaphthalen-2-yl)amino)pyridin-3-yl)naphtho[2,3-c]furan-1(3H)-one
[173] 6,7-dimethoxy-9-(5-(2-methoxyethoxy)pyridin-3-yl)naphtho[2,3-c]furan-1(3H)-one
[174] 9-(6-(bis(2-methoxyethyl)amino)pyridin-3-yl)-6,7-dimethoxynaphtho[2,3-c]furan-1(3H)-one
[175] 9-(6-(cyclohexyl(methyl)amino)pyridin-3-yl)-6,7-dimethoxynaphtho[2,3-c]furan-1(3H)-one
[176] 6,7-dimethoxy-9-(6-(methyl(2-methylbutyl)amino)pyridin-3-yl)naphtho[2,3-c]furan-1(3H)-one
[177] 9-(6-(cyclopentyl(methyl)amino)pyridin-3-yl)-6,7-dimethoxynaphtho[2,3-c]furan-1(3H)-one
[178] 9-(6-(ethyl(methyl)amino)pyridin-3-yl)-6,7-dimethoxynaphtho[2,3-c]furan-1(3H)-one
[179] 6,7-dimethoxy-9-(6-(methyl((5-methylfuran-2-yl)methyl)amino)pyridin-3-yl)naphtho[2,3-c]furan-1(3H)-one
[180] 9-(6-(butyl(methyl)amino)pyridin-3-yl)-6,7-dimethoxynaphtho[2,3-c]furan-1(3H)-one
[181] 9-(6-(isobutyl(methyl)amino)pyridin-3-yl)-6,7-dimethoxynaphtho[2,3-c]furan-1(3H)-one
[182] 9-(6-(ethyl(isopropyl)amino)pyridin-3-yl)-6,7-dimethoxynaphtho[2,3-c]furan-1(3H)-one
[183] 9-(6-((cyclopropylmethyl)(methyl)amino)pyridin-3-yl)-6,7-dimethoxynaphtho[2,3-c]furan-1(3H)-one
[184] 9-(2-chloropyridin-4-yl)-6,7-dimethoxynaphtho[2,3-c]furan-1(3H)-one
[185] 9-(6-chloropyridin-3-yl)-6,7-dimethoxynaphtho[2,3-c]furan-1(3H)-one
[186] 9-(2-fluoropyridin-4-yl)-6,7-dimethoxynaphtho[2,3-c]furan-1(3H)-one
[187] 9-(5-(2-(dimethylamino) ethoxy)pyridin-3-yl)-6,7-dimethoxynaphtho[2,3-c]furan-1(3H)-one
[188] 9-(6-(isopropyl(methyl)amino)pyridin-3-yl)-6,7-dimethoxynaphtho[2,3-c]furan-1(3H)-one
[189] 9-(6-(3,4-dihydroisoquinolin-2 (1H)-yl)pyridin-3-yl)-6,7-dimethoxynaphtho[2,3-c]furan-1(3H)-one
[191] 9-(6-(cyclopentyl(ethyl)amino)pyridin-3-yl)-6,7-dimethoxynaphtho[2,3-c]furan-1(3H)-one
[192] 6,7-dimethoxy-9-(6-(methyl((tetrahydrofuran-2-yl)methyl)amino)pyridin-3-yl)naphtho[2,3-c]furan-1(3H)-one
[193] 9-(6-(ethyl((tetrahydrofuran-2-yl)methyl)amino)pyridin-3-yl)-6,7-dimethoxynaphtho[2,3-c]furan-1(3H)-one
[194] 9-(6-((2,3-dihydro-1H-indan-2-yl)(methyl)amino)pyridin-3-yl)-6,7-dimethoxynaphtho[2,3-c]furan-1(3H)-one
[195] 9-(6-((2,3-dihydro-1H-indan-2-yl)(ethyl)amino)pyridin-3-yl)-6,7-dimethoxynaphtho[2,3-c]furan-1(3H)-one
[196] 6,7-dimethoxy-9-(6-(methyl(2-(1-methyl-1H-indol-3-yl)ethyl)amino)pyridin-3-yl)naphtho[2,3-c]furan-1(3H)-one
[197] 9-(6-(ethyl(2-(1-ethyl-1H-indol-3-yl)ethyl)amino)pyridin-3-yl)-6,7-dimethoxynaphtho[2,3-c]furan-1(3H)-one
[198] 6,7-dimethoxy-9-(6-(methyl(thiophen-2-ylmethyl)amino)pyridin-3-yl)naphtho[2,3-c]furan-1(3H)-one
[199] 9-(6-(ethyl(thiophen-2-ylmethyl)amino)pyridin-3-yl)-6,7-dimethoxynaphtho[2,3-c]furan-1(3H)-one
[200] 9-(5-isopropoxypyridin-3-yl)-6,7-dimethoxynaphtho[2,3-c]furan-1(3H)-one
[202] 6,7-dimethoxy-9-(6-(3-(m-tolyl) piperidin-1-yl)pyridin-3-yl)naphtho[2,3-c]furan-1(3H)-one
[203] 6,7-dimethoxy-9-(3-(morpholinomethyl)-1H-indol-6-yl)naphtho[2,3-c]furan-1(3H)-one
[205] 9-(2-(4-(dimethylamino) piperidin-1-yl)pyrimidin-5-yl)-6,7-dimethoxynaphtho[2,3-c]furan-1(3H)-one
[206] 6,7-dimethoxy-9-(6-(methyl(1-methyl-1H-1,2,4-triazol-3-yl)amino)pyridin-3-yl)naphtho[2,3-c]furan-1(3H)-one
[207] 9-(6-(ethyl(1-ethyl-1H-1,2,4-triazol-3-yl)amino)pyridin-3-yl)-6,7-dimethoxynaphtho[2,3-c]furan-1(3H)-one
[208] 6,7-dimethoxy-9-(6-(methyl(2-(trifluoromethyl)benzyl)amino)pyridin-3-yl)naphtho[2,3-c]furan-1(3H)-one
[209] 9-(6-(ethyl(2-(trifluoromethyl)benzyl)amino)pyridin-3-yl)-6,7-dimethoxynaphtho[2,3-c]furan-1(3H)-one
[213] 9-(2-((3,4-dimethylphenyl)(methyl)amino)pyrimidin-5-yl)-6,7-dimethoxynaphtho[2,3-c]furan-1(3H)-one
[214] 6,7-dimethoxy-9-(6-(methyl(5-methyl-1,3,4-thiadiazol-2-yl)amino)pyridin-3-yl)naphtho[2,3-c]furan-1(3H)-one
[215] 6,7-dimethoxy-9-(6-(methyl(1-methyl-5-(methylthio)-1H-1,2,4-triazol-3-yl)amino)pyridin-3-yl)naphtho[2,3-c]furan-1(3H)-one
[216] 6,7-dimethoxy-9-(2-(methyl(3-(trifluoromethyl)phenyl)amino)pyrimidin-5-yl)naphtho[2,3-c]furan-1(3H)-one
[218] 4-((5-(6,7-dimethoxy-3-oxo-1,3-dihydronaphtho[2,3-c]furan-4-yl)pyridin-2-yl)(methyl)amino)benzonitrile
[219] 6,7-dimethoxy-9-(6-morpholinopyridazin-3-yl)naphtho[2,3-c]furan-1(3H)-one
[220] 6,7-dimethoxy-9-(6-morpholinoimidazo[1,2-b]pyridazin-3-yl)naphtho[2,3-c]furan-1(3H)-one
[222] 6,7-dimethoxy-9-(6-(methyl(phenyl)amino)pyridin-3-yl)naphtho[2,3-c]furan-1(3H)-one
[223] 9-(6-(3-(dimethylamino)-5-(methylthio)-1H-1,2,4-triazol-1-yl)pyridin-3-yl)-6,7-dimethoxynaphtho[2,3-c]furan-1(3H)-one
[225] 9-(6-(benzo[d]thiazol-2-yl(methyl)amino)pyridin-3-yl)-6,7-dimethoxynaphtho[2,3-c]furan-1(3H)-one
[226] 9-(6-(ethyl(pyridin-4-ylmethyl)amino)pyridin-3-yl)-6,7-dimethoxynaphtho[2,3-c]furan-1(3H)-one
[229] 6,7-dimethoxy-9-(6-(methylamino)pyridin-3-yl)naphtho[2,3-c]furan-1(3H)-one
[230] N-(5-(6,7-dimethoxy-3-oxo-1,3-dihydronaphtho[2,3-c]furan-4-yl)pyridin-2-yl)-N-methylacetamide
[231] 6,7-dimethoxy-9-(6-(methyl(5-methyl-1,3,4-oxadiazol-2-yl)amino)pyridin-3-yl)naphtho[2,3-c]furan-1(3H)-one
[232] 9-(6-((2,4-dimethoxybenzyl)(methyl)amino)pyridin-3-yl)-6,7-dimethoxynaphtho[2,3-c]furan-1(3H)-one
[233] N-(5-(6,7-dimethoxy-3-oxo-1,3-dihydronaphtho[2,3-c]furan-4-yl)pyridin-2-yl)-N-methylpivalamide
[236] N-(4-((5-(6,7-dimethoxy-3-oxo-1,3-dihydronaphtho[2,3-c]furan-4-yl)pyridin-2-yl)(methyl)amino)phenyl)-N-methylacetamide
[237] 3-((5-(6,7-dimethoxy-3-oxo-1,3-dihydronaphtho[2,3-c]furan-4-yl)pyridin-2-yl)(methyl)amino)benzonitrile
[238] 9-(6-(4-(dimethylamino) piperidin-1-yl)pyridin-3-yl)-6,7-dimethoxynaphtho[2,3-c]furan-1(3H)-one
[239] 9-(6-((4-mercaptophenyl)(methyl)amino)pyridin-3-yl)-6,7-dimethoxynaphtho[2,3-c]furan-1(3H)-one
[240] 6,7-dimethoxy-9-(6-(3-(trifluoromethyl)-5,6-dihydro-[1,2,4]triazolo[4,3-a]pyrazin-7 (8H)-yl)pyridin-3-yl)naphtho[2,3-c]furan-1(3H)-one
[241] 3-((5-(6,7-dimethoxy-3-oxo-1,3-dihydronaphtho[2,3-c]furan-4-yl)pyridin-2-yl)(methyl)amino)-1-methyl-1H-pyrazol-4-carbonitrile
[242] 6,7-dimethoxy-9-(4-(piperidin-1-yl)phenyl)naphtho[2,3-c]furan-1(3H)-one
[243] 6,7-dimethoxy-9-(3-(piperidin-1-yl)phenyl)naphtho[2,3-c]furan-1(3H)-one
[244] 6,7-dimethoxy-9-(3-morpholinophenyl)naphtho[2,3-c]furan-1(3H)-one
[245] 6,7-dimethoxy-9-(4-morpholinophenyl)naphtho[2,3-c]furan-1(3H)-one
[246] 6,7-dimethoxy-9-(4-(methylamino)phenyl)naphtho[2,3-c]furan-1(3H)-one
[247] 9-(4-(dimethylamino)phenyl)-6,7-dimethoxynaphtho[2,3-c]furan-1(3H)-one
[248] 9-(4-(ethyl(methyl)amino)phenyl)-6,7-dimethoxynaphtho[2,3-c]furan-1(3H)-one
[249] 9-(4-(diethylamino)phenyl)-6,7-dimethoxynaphtho[2,3-c]furan-1(3H)-one
[250] 9-(4-(ethylamino)phenyl)-6,7-dimethoxynaphtho[2,3-c]furan-1(3H)-one
[251] 6,7-dimethoxy-9-(4-(pyrrolidin-1-yl)phenyl)naphtho[2,3-c]furan-1(3H)-one
[252] 6,7-dimethoxy-9-(3-(pyrrolidin-1-yl)phenyl)naphtho[2,3-c]furan-1(3H)-one
[253] 9-(4-(ethyl(2-methoxyethyl)amino)phenyl)-6,7-dimethoxynaphtho[2,3-c]furan-1(3H)-one
[254] 9-(4-(ethyl(pyridin-3-ylmethyl)amino)phenyl)-6,7-dimethoxynaphtho[2,3-c]furan-1(3H)-one
[255] 6,7-dimethoxy-9-(4-(methyl(pyridin-3-ylmethyl)amino)phenyl)naphtho[2,3-c]furan-1(3H)-one
[256] 6,7-dimethoxy-9-(4-((2-methoxyethyl)(methyl)amino)phenyl)naphtho[2,3-c]furan-1(3H)-one
[257] 6,7-dimethoxy-9-(3-(methylamino)phenyl)naphtho[2,3-c]furan-1(3H)-one
[258] 9-(3-(ethylamino)phenyl)-6,7-dimethoxynaphtho[2,3-c]furan-1(3H)-one
[274] 6-(6,7-dimethoxy-3-oxo-1,3-dihydronaphtho[2,3-c]furan-4-yl)benzo[d][1,3]dioxol-5-yl acetate
[275] 6-(6,7-dimethoxy-3-oxo-1,3-dihydronaphtho[2,3-c]furan-4-yl)benzo[d][1,3]dioxol-5-yl isobutrate
[276] 6-(6,7-dimethoxy-3-oxo-1,3-dihydronaphtho[2,3-c]furan-4-yl)benzo[d][1,3]dioxol-5-yl pivalate

According to one example of the present invention, there may be provided a 6,7-dialkoxynaphtho[2,3-c]furan-1(3H)-one compound of formula 3 below, a pharmaceutically acceptable salt thereof, a stereoisomer thereof, a hydrate thereof, or a solvate thereof. The 6,7-dialkoxynaphtho[2,3-c]furan-1(3H)-one compound of formula 3 may be a 6,7-dimethoxynaphtho[2,3-c]furan-1(3H)-one compound.

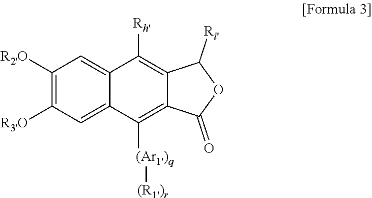

In above formula 3, Ar_{1′} may be substituted or unsubstituted C₆-C₁₂ arylene group, or substituted or unsubstituted C₂-C₁₂ heteroarylene group including at least one of N, O and S as a heteroatom in a ring. q may be 0 or 1.
R_{1′} may be each independently halogen, substituted or unsubstituted C₆-C₂₀ aryl (wherein a substituent of the C₆-C₂₀ aryl may be bonded to an adjacent group to form a ring); —NR_{4′}R_{5′}; —OR_{6′}; —C(═O)CX₃ (wherein X is halogen); —NO₂;

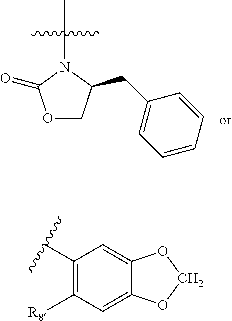

(wherein at least one of the hydrogen of

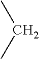

may be substituted with halogen). R_{1′} including a ring formed by a substituent of the C₆-C₂₀ aryl being bonded to an adjacent group may be substituted or unsubstituted naphthalenyl, or substituted or unsubstituted tetrahydroquinolinyl, for example,

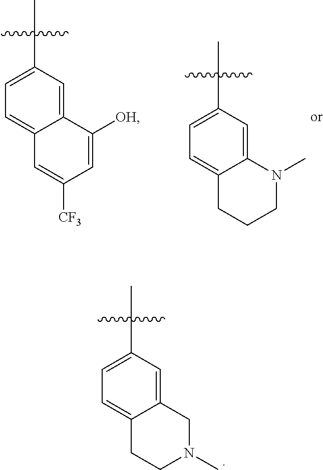 [SMILES: *c1ccc2cc(C(F)(F)F)cc(O)c2c1 ; *c1ccc2c(c1)N(C)CCC2 ; *c1ccc2c(c1)CN(C)CC2]

r may be an integer of 0 or more and 2 or less.
R_{2′} and R_{3′} may be each independently C₁-C₁₀ linear or branched alkyl.
R_{4′} to R_{6′} may be each independently C₁-C₁₀ linear or branched alkyl, or —C(═O)R_{c′}.
R_{c′} may be substituted or unsubstituted C₂-C₁₀ heterocycloalkyl including at least one of N, O and S as a heteroatom in a ring, or substituted or unsubstituted C₂-C₂₀ heteroaryl including at least one of N, O and S as a heteroatom in a ring.
R_{8′} may be hydrogen, —OC(═O)R_{d′}, —OP(═O)(OR_{d′})₂, —OP(═O)(NHR_{d′})(OR_{e′}), —OP(═O)(NHR_{d′})(R_{e′}), —OH, —NHR_{d′} or C₁-C₂₀ linear or branched alkoxy.
R_{d′} and R_{e′} may be each independently C₁-C₁₀ linear or branched alkyl (wherein at least one of the hydrogen of the alkyl may be substituted with C₂-C₁₀ heterocycloalkyl or amine), C₂-C₁₀ heterocycloalkyl, substituted or unsubstituted C₆-C₂₀ aryl, or substituted or unsubstituted C₂-C₂₀ heteroaryl.
R_{h′} and R_{i′} may be each independently hydrogen,

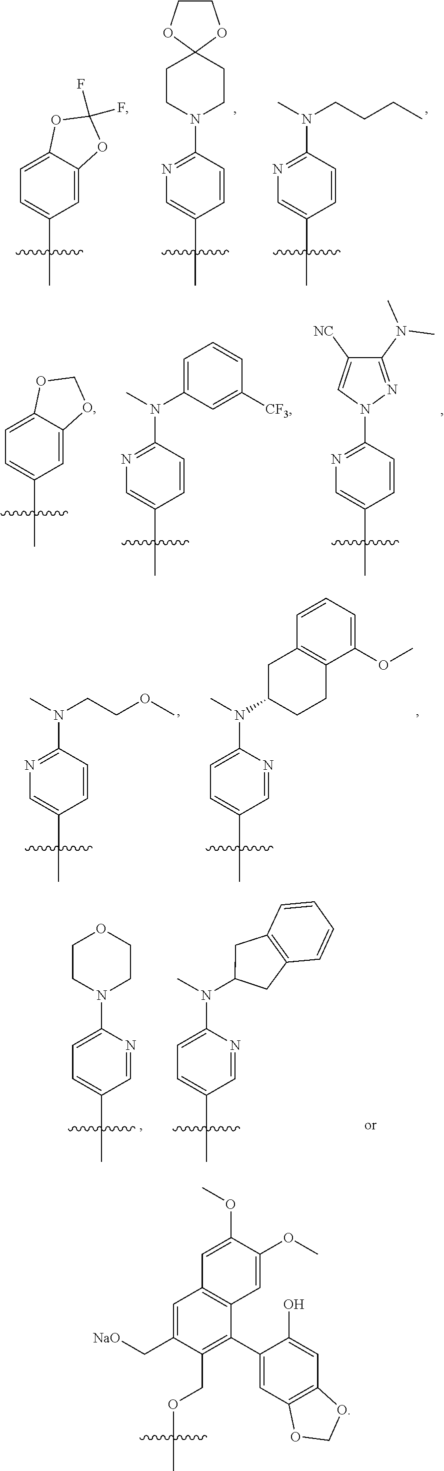

Ar_{1′}—R_{1′} may be represented by formula 4 below.

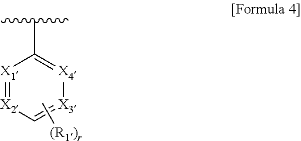

In above formula 4, X_{1′} to X_{4′} may be each independently CH or N, and R_{1′} and r may be the same as defined in above formula 3.

Above formula 4 may be represented by formulas 4-1 to 4-4 below.

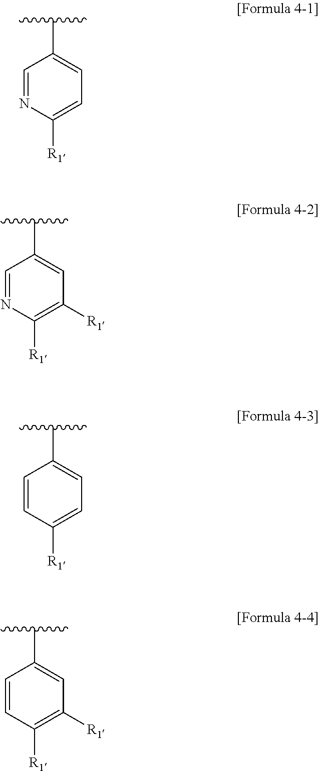

In above formulas 4-1 to 4-4, R_{1′} may be the same as defined in above formula 3 or 4.
R_{1′} may be halogen, —NR_{4′}R_{5′}, —OR_{6′}, —C(═O)CX₃ (wherein X is halogen), —NO₂,

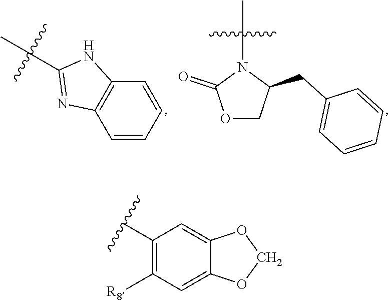

(wherein at least one of the hydrogen of

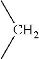

may be substituted with halogen), or

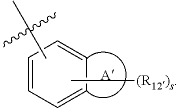

A′ may be C₆-C₁₂ aryl, or substituted or unsubstituted C₂-C₁₀ heterocycloalkyl including at least one of N, O and S as a heteroatom in a ring. For example, A′ may be substituted or unsubstituted benzene, or a substituted or unsubstituted piperidine.
R_{12″} may be each independently C₁-C₁₀ linear or branched alkyl. s may be an integer of 0 or more and 5 or less.
R_{4′} to R_{6′} and R_{8′} may be the same as defined in formula 3.
R_{1′} may be chlorine,

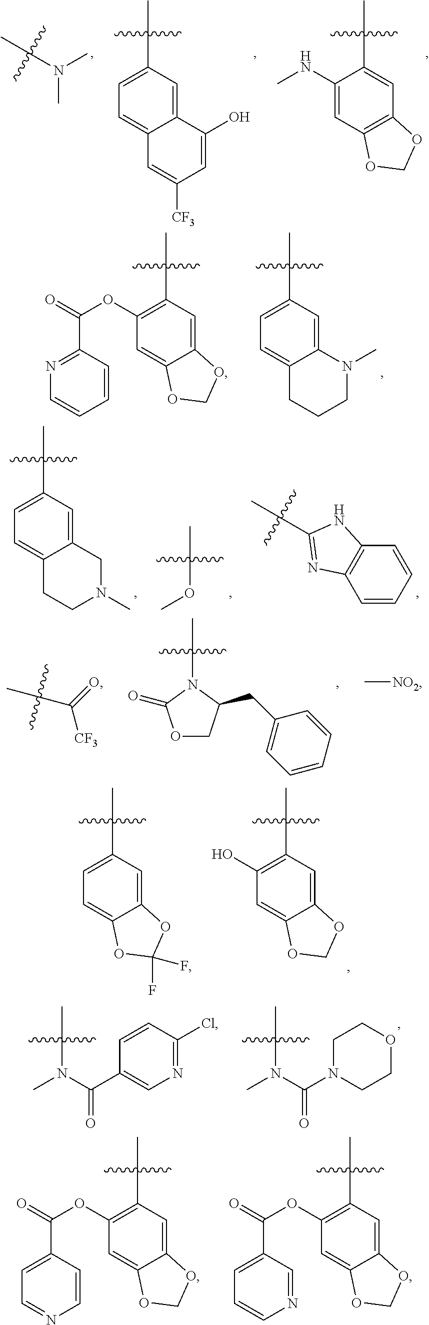

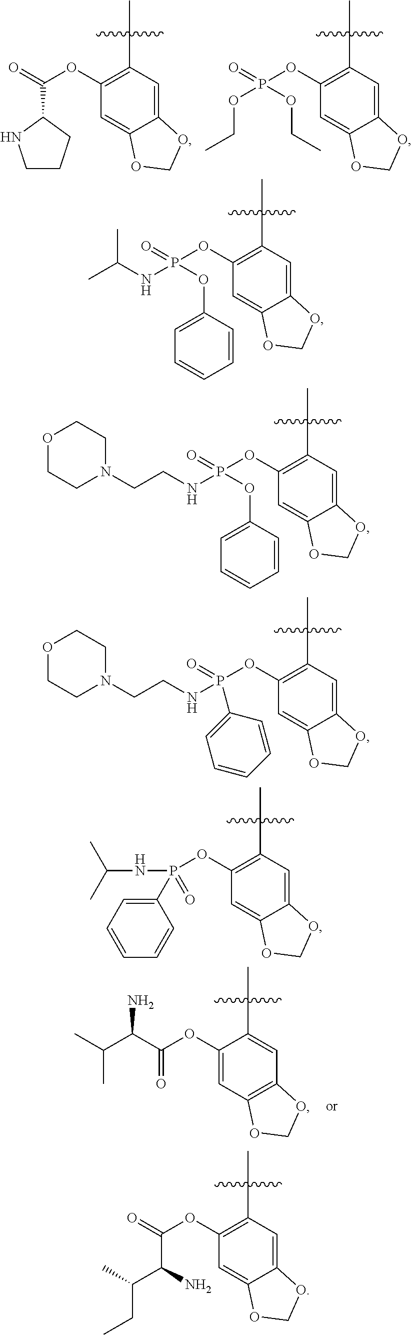

The compound represented by above formula 3 may be one selected from the group consisting of the following compounds:
[145] 9-(4-(2-hydroxyethyl) piperazin-1-yl)-6,7-dimethoxynaphtho[2,3-c]furan-1(3H)-one
[153] 9-(8-hydroxy-6-(trifluoromethyl) naphthalen-2-yl)-6,7-dimethoxynaphtho[2,3-c]furan-1(3H)-one
[169] 6-(6,7-dimethoxy-3-oxo-1,3-dihydronaphtho[2,3-c]furan-4-yl)benzo[d][1,3]dioxol-5-yl picolate
[170] 6,7-dimethoxy-9-(1-methyl-1,2,3,4-tetrahydroquinolin-7-yl)naphtho[2,3-c]furan-1(3H)-one
[190] 6,7-dimethoxy-9-(2-methyl-1,2,3,4-tetrahydroisoquinolin-7-yl)naphtho[2,3-c]furan-1(3H)-one
[201] 9-(6-chloro-5-(dimethylamino)pyridin-3-yl)-6,7-dimethoxynaphtho[2,3-c]furan-1(3H)-one
[204] 9-(5-(1H-benzo[d]imidazol-2-yl)-6-methoxypyridin-3-yl)-6,7-dimethoxynaphtho[2,3-c]furan-1(3H)-one
[210] 6,7-dimethoxy-9-(4-(2,2,2-trifluoroacetyl)phenyl)naphtho[2,3-c]furan-1(3H)-one
[211] (S)-4-benzyl-3-(5-(6,7-dimethoxy-3-oxo-1,3-dihydronaphtho[2,3-c]furan-4-yl)pyridin-2-yl) oxazolidin-2-one
[217] 9-(4-(dimethylamino)-3-nitrophenyl)-6,7-dimethoxynaphtho[2,3-c]furan-1(3H)-one
[221] 9-(2,2-difluorobenzo[d][1,3]dioxol-5-yl)-6,7-dimethoxynaphtho[2,3-c]furan-1(3H)-one
[227] 9-(4-(5-bromopyrimidin-2-yl) piperazin-1-yl)-6,7-dimethoxynaphtho[2,3-c]furan-1(3H)-one
[234] 6-chloro-N-(5-(6,7-dimethoxy-3-oxo-1,3-dihydronaphtho[2,3-c]furan-4-yl)pyridin-2-yl)-N-methylnicotinamide
[235] N-(5-(6,7-dimethoxy-3-oxo-1,3-dihydronaphtho[2,3-c]furan-4-yl)pyridin-2-yl)-N-methylmorpholin-4-carboxamide
[259] 4-(2,2-difluorobenzo[d][1,3]dioxol-5-yl)-6,7,9-trimethoxynaphtho[2,3-c]furan-1(3H)-one
[260] 4-(6-(1,4-dioxa-8-azaspiro[4.5]decan-8-yl)pyridin-3-yl)-6,7,9-trimethoxynaphtho[2,3-c]furan-1(3H)-one
[261] 4-(6-(butyl(methyl)amino)pyridin-3-yl)-6,7,9-trimethoxynaphtho[2,3-c]furan-1(3H)-one
[262] 4-(benzo[d][1,3]dioxol-5-yl)-6,7,9-trimethoxynaphtho[2,3-c]furan-1(3H)-one
[263] 6,7,9-trimethoxy-4-(6-(methyl(3-(trifluoromethyl)phenyl)amino)pyridin-3-yl)naphtho[2,3-c]furan-1(3H)-one
[264] 3-(dimethylamino)-1-(5-(6,7,9-trimethoxy-1-oxo-1,3-dihydronaphtho[2,3-c]furan-4-yl)pyridin-2-yl)-1H-pyrazol-4-carbonitrile
[265] 6,7,9-trimethoxy-4-(6-((2-methoxyethyl)(methyl)amino)pyridin-3-yl)naphtho[2,3-c]furan-1(3H)-one
[266] (S)-6,7,9-trimethoxy-4-(6-((5-methoxy-1,2,3,4-tetrahydronaphthalen-2-yl)(methyl)amino)pyridin-3-yl)naphtho[2,3-c]furan-1(3H)-one
[267] 6,7,9-trimethoxy-4-(6-morpholinopyridin-3-yl)naphtho[2,3-c]furan-1(3H)-one
[268] 4-(6-((2,3-dihydro-1H-indan-2-yl)(methyl)amino)pyridin-3-yl)-6,7,9-trimethoxynaphtho[2,3-c]furan-1(3H)-one
[269] 6-(6,7-dimethoxy-3-oxo-1,3-dihydronaphtho[2,3-c]furan-4-yl)benzo[d][1,3]dioxol-5-yl L-valinate hydrochloride
[270] 6-(6,7-dimethoxy-3-oxo-1,3-dihydronaphtho[2,3-c]furan-4-yl)benzo[d][1,3]dioxol-5-yl L-isoleucinate
[271] 6-(6,7-dimethoxy-3-oxo-1,3-dihydronaphtho[2,3-c]furan-4-yl)benzo[d][1,3]dioxol-5-yl isonicoinate
[272] 6-(6,7-dimethoxy-3-oxo-1,3-dihydronaphtho[2,3-c]furan-4-yl)benzo[d][1,3]dioxol-5-yl nicotinate
[273] 6-(6,7-dimethoxy-3-oxo-1,3-dihydronaphtho[2,3-c]furan-4-yl)benzo[d][1,3]dioxol-5-yl L-prolinate
[277] 6-(6,7-dimethoxy-3-oxo-1,3-dihydronaphtho[2,3-c]furan-4-yl)benzo[d][1,3]dioxol-5-yl diethyl phosphate
[278] 6-(6,7-dimethoxy-3-oxo-1,3-dihydronaphtho[2,3-c]furan-4-yl)benzo[d][1,3]dioxol-5-yl phenyl isopropylphosphoramidate
[279] 6-(6,7-dimethoxy-3-oxo-1,3-dihydronaphtho[2,3-c]furan-4-yl)benzo[d][1,3]dioxol-5-yl phenyl (2-morpholinoethyl)phosphoramidate
[280] 6-(6,7-dimethoxy-3-oxo-1,3-dihydronaphtho[2,3-c]furan-4-yl)benzo[d][1,3]dioxol-5-yl N-(2-morpholinoethyl)-P-phenylphosphoramidate
[281] 6-(6,7-dimethoxy-3-oxo-1,3-dihydronaphtho[2,3-c]furan-4-yl)benzo[d][1,3]dioxol-5-yl N-isopropyl-P-phenylphosphoramidate

According to another example of the present invention, there may be provided one compound selected from the group consisting of the following compounds.
[212] 1-(6-hydroxybenzo[d][1,3]dioxol-5-yl)-3-(hydroxymethyl)-6,7-dimethoxy-2-naphthoate sodium (I)
[224] 1-(6-hydroxybenzo[d][1,3]dioxol-5-yl)-3-(hydroxymethyl)-6,7-dimethoxy-2-naphthoyl chloride
[228] 1-(6-hydroxybenzo[d][1,3]dioxol-5-yl)-3-(hydroxymethyl)-N-isopropyl-6,7-dimethoxy-2-naphthamide

The compounds represented by formulas 1 and 3 and the compounds of [212], [224] and [228] of the present invention may be used in the form of pharmaceutically acceptable salt, and acid addition salt formed by pharmaceutically acceptable free acid may be useful as salt. The acid addition salt may be obtained from inorganic acids such as hydrochloric acid, nitric acid, phosphoric acid, sulfuric acid, hydrobromic acid, hydroiodic acid, nitrous acid, phosphorous acid, etc., non-toxic organic acids such as aliphatic mono- and di-carboxylates, phenyl-substituted alkanoate, hydroxy alkanoate and alkandioate, aromatic acids, and aliphatic and aromatic sulfonic acids, etc., and organic acids such as acetic acid, benzoic acid, citric acid, lactic acid, maleic acid, gluconic acid, methanesulfonic acid, 4-toluenesulfonic acid, tartaric acid, fumaric acid, etc. The types of these pharmaceutically non-toxic salts may include sulfate, pyrosulfate, bisulfate, sulfite, bisulfite, nitrate, phosphate, monohydrogen phosphate, dihydrogen phosphate, metaphosphate, pyrophosphate chloride, bromide, iodide, fluoride, acetate, propionate, decanoate, caprylate, acrylate, formate, isobutyrate, caprate, heptanoate, propiolate, oxalate, malonate, succinate, suberate, sebacate, fumarate, maleate, butyn-1,4-dioate, hexan-1,6-dioate, benzoate, chlorobenzoate, methyl benzoate, dinitro benzoate, hydroxybenzoate, methoxybenzoate, phthalate, terephthalate, benzenesulfonate, toluenesulfonate, chlorobenzenesulfonate, xylenesulfonate, phenylacetate, phenylpropionate, phenylbutyrate, citrate, lactate, β-hydroxybutyrate, glycolate, malate, tartrate, methanesulfonate, propanesulfonate, naphthalen-1-sulfonate, naphthalen-2-sulfonate, mandelate, etc., but the types of salts meant in the present invention are not limited to those listed salts.

For example, the free acid may be selected from the group consisting of citric acid, acetic acid, lactic acid, tartaric acid, maleic acid, fumaric acid, formic acid, propionic acid, oxalic acid, trifluoroacetic acid, benzoic acid, gluconic acid, methanesulfonic acid, glycolic acid, succinic acid, 4-toluenesulfonic acid, camphorsulfonic acid, glutamic acid, aspartic acid, silicylic acid, malonic acid, malic acid, benzenesulfonic acid, hydrochloric acid, bromic acid, nitric acid, sulfuric acid, and phosphoric acid.

The acid addition salt according to the present invention may be prepared by a conventional method and, for example, the acid addition salt may be prepared by dissolving the compound of formula 1 in an organic solvent such as methanol, ethanol, acetone, dichloromethane, acetonitrile, or the like and filtering and drying the resulting precipitate which is obtained by adding an organic acid or an inorganic acid thereto, or by distilling the solvent and an excess of the acid under reduced pressure and then drying and crystallizing under the organic solvent.

In addition, a pharmaceutically acceptable metal salt may be prepared by using a base. An alkali metal or alkaline earth metal salt may be obtained, for example, by dissolving a compound in an excessive amount of alkali metal hydroxide or alkaline earth metal hydroxide solution, filtering the undissolved compound salt, and evaporating and drying the filtrate. In this case, it may be pharmaceutically suitable to prepare sodium, potassium or calcium salt as the metal salt. In addition, the corresponding salt may be obtained by reacting the alkali metal or alkaline earth metal salt with an appropriate silver salt (e.g., silver nitrate).

According to one example of the present invention, there may be provided a pharmaceutical composition including the above-described compound, the pharmaceutically acceptable salt thereof, the stereoisomer thereof, the hydrate thereof or the solvate thereof as active ingredients.

According to one example of the present invention, there may be provided a pharmaceutical composition for an anti-viral purpose including the above-described compound, the pharmaceutically acceptable salt thereof, the stereoisomer thereof, the hydrate thereof or the solvate thereof as active ingredients.

The composition may be an anti-viral composition for one or more of severe acute respiratory syndrome coronavirus 2 (SARS-COV-2), influenza virus, dengue virus, Zika virus, and severe fever with thrombocytopenia syndrome virus (SFTS).

According to one example of the present invention, there may be provided a pharmaceutical composition for preventing or treating a virus infection disease, which includes the above-described compound, the pharmaceutically acceptable salt thereof, the stereoisomer thereof, the hydrate thereof or the solvate thereof as active ingredients.

The virus infection disease may be one or more of SARS-COV-2 infection disease, influenza virus infection disease, dengue virus infection disease, Zika virus infection disease, and severe fever with thrombocytopenia syndrome virus infection disease. The SARS-COV-2 infection disease may be coronavirus infection disease-19 (COVID-19). The composition may inhibit intracellular infection and proliferation of one or more of SARS-COV-2, influenza virus, dengue virus, Zika virus, and severe fever with thrombocytopenia syndrome virus.

According to one example of the present invention, there may be provided a pharmaceutical composition for preventing or treating an allergic disease, which includes the above-described compound, the pharmaceutically acceptable salt thereof, the stereoisomer thereof, the hydrate thereof or the solvate thereof as active ingredients.

The allergic disease may be at least one selected from the group consisting of rhinitis, asthma, atopic dermatitis, allergic conjunctivitis, allergic otitis media, allergic gastrointestinal infection, anaphylaxis and hives.

According to one example of the present invention, there may be provided a pharmaceutical composition for preventing or treating a respiratory disease, which includes the above-described compound, the pharmaceutically acceptable salt thereof, the stereoisomer thereof, the hydrate thereof or the solvate thereof as active ingredients.

The respiratory disease may include an allergic respiratory disease and a non-allergic respiratory disease. The respiratory disease may be at least one selected from the group consisting of cold, pneumonia, bronchitis, chronic obstructive pulmonary disease and rhinitis.

The respiratory disease may be asthma. The asthma may include allergic asthma and non-allergic asthma.

The composition may inhibit the expression of interleukin-5. The composition may exhibit an effect of preventing or treating a respiratory disease, such as improving sputum discharge capacity.

The pharmaceutical composition according to one example may further include at least one of a pharmaceutically acceptable additive, in addition to the compound represented by above formula 1, the optical isomer thereof or the pharmaceutically acceptable salt thereof. For example, the pharmaceutical composition according to one example may further include at least one of diluent or excipient such as carrier, filler, extender, binder, humectant, disintegrant, surfactant, etc. as a pharmaceutically acceptable additive.

The pharmaceutical composition according to one example may further include at least one of a pharmaceutically acceptable additive, in addition to the compound represented by above formula 3, the optical isomer thereof or the pharmaceutically acceptable salt thereof. For example, the pharmaceutical composition according to one example may further include at least one of diluent or excipient such as carrier, filler, extender, binder, humectant, disintegrant, surfactant, etc. as a pharmaceutically acceptable additive.

The pharmaceutical composition according to one example may further include at least one of a pharmaceutically acceptable additive, in addition to the compound of [212], [224] or [228], the optical isomer thereof or the pharmaceutically acceptable salt thereof. For example, the pharmaceutical composition according to one example may further include at least one of diluent or excipient such as carrier, filler, extender, binder, humectant, disintegrant, surfactant, etc. as a pharmaceutically acceptable additive.

According to one example of the present invention, the present invention may provide a method for preventing or treating a virus infection disease, which includes administering the above-described compound, the pharmaceutically acceptable salt thereof, the stereoisomer thereof, the hydrate thereof or the solvate thereof into a subject in need thereof.

According to one example of the present invention, the present invention may provide a use of the above-described compound, the pharmaceutically acceptable salt thereof, the stereoisomer thereof, the hydrate thereof or the solvate thereof for preventing or treating a virus infection disease.

According to one example of the present invention, the present invention may provide a use of the above-described compound, the pharmaceutically acceptable salt thereof, the stereoisomer thereof, the hydrate thereof or the solvate thereof for manufacturing a medicament for preventing or treating a virus infection disease.

According to one example of the present invention, the present invention may provide a method for preventing or treating a respiratory disease, which includes administering the above-described compound, the pharmaceutically acceptable salt thereof, the stereoisomer thereof, the hydrate thereof or the solvate thereof into a subject in need thereof.

According to one example of the present invention, the present invention may provide a use of the above-described compound, the pharmaceutically acceptable salt thereof, the stereoisomer thereof, the hydrate thereof or the solvate thereof for preventing or treating a respiratory disease.

According to one example of the present invention, the present invention may provide a use of the above-described compound, the pharmaceutically acceptable salt thereof, the stereoisomer thereof, the hydrate thereof or the solvate thereof for manufacturing a medicament for preventing or treating a respiratory disease.

According to one example of the present invention, the present invention may provide a method for preventing or treating an allergic disease, which includes administering the above-described compound, the pharmaceutically acceptable salt thereof, the stereoisomer thereof, the hydrate thereof or the solvate thereof into a subject in need thereof.

According to one example of the present invention, the present invention may provide a use of the above-described compound, the pharmaceutically acceptable salt thereof, the stereoisomer thereof, the hydrate thereof or the solvate thereof for preventing or treating an allergic disease.

According to one example of the present invention, the present invention may provide a use of the above-described compound, the pharmaceutically acceptable salt thereof, the stereoisomer thereof, the hydrate thereof or the solvate thereof for manufacturing a medicament for preventing or treating an allergic disease.

In the present specification, the term “subject” may refer to animals, and may be typically mammals, on which treatment using the composition of the present invention may exhibit a beneficial effect. The mammals may refer to mammals including humans, and the term “administration” may refer to providing a predetermined material to a subject through any appropriate method. It is apparent to those skilled in the art that the therapeutically effective dosage and the number of administration for active ingredient of the present invention may vary depending on a desired effect.

In the present invention, the term “mammals including humans” may mean mammals such as monkey, cow, horse, dog, cat, rabbit, rat, mouse, etc., and in particular may include humans. In addition, the subjects may include all the subjects having a symptom of respiratory disease, or having a risk of developing the symptom.

In the present specification, the term “prevention” may refer to a delay of occurrence of disease, disorder or condition. If the occurrence of disease, disorder or condition is delayed for an expected period of time, the prevention may be considered as complete.

In the present specification, the term “treatment” may refer to one which partially or completely reduces, ameliorates, alleviates, inhibits or delays the occurrence of a certain disease, disorder and/or condition, reduces a severity thereof, or reduces the occurrence of at least one symptom or property thereof.

The pharmaceutical composition of the present invention may be formulated into a dosage form of tablet, pill, powder, granule, capsule, suspension, liquid for internal use, emulsion, syrup, aerosol, solution for injection, etc., according to a conventional method for preventing and treating a virus infection disease, a respiratory disease, or an allergic disease.

The pharmaceutical composition of the present invention may be orally or parenterally administered (for example, applied or injected intravenously, subcutaneously or intraperitoneally).

In the present specification, the term “oral administration” may refer to a method of injecting a drug for ameliorating a pathological symptom into a mouth. In the present specification, the term “parenteral administration” may mean a method of administering subcutaneously, intramuscularly, intravenously and intraperitoneally through a tube, excluding administration through the mouth.

A solid preparation for oral administration may include tablets, pills, powders, granules, capsules, etc., and this solid preparation may be prepared by mixing at least one excipient, for example, starch, calcium carbonate, sucrose, lactose, gelatin, etc., in a complex composition. In addition, lubricants such as magnesium stearate and talc may be also used in addition to simple excipients. A liquid preparation for oral administration may include a suspending agent, liquid for internal use, emulsion, syrup, etc., but may also include various excipients, for example, humectant, sweetening agent, flavoring agent, preservative, etc. in addition to water and liquid paraffin, which are the frequently used simple diluents.

A preparation for parenteral administration may include a sterilized aqueous solution, non-aqueous solvent, suspending agent, emulsion, freeze-dried preparation, suppository, etc. The non-aqueous solvent and the suspending agent may include propylene glycol, polyethylene glycol, vegetable oil like olive oil, injectable ester like ethyl oleate, etc.

The pharmaceutically acceptable additive according to the present invention may be included in an amount of 0.1 to 99.9 parts by weight, specifically in an amount of about 0.1 to 50 parts by weight with regard to the composition, but an example is not limited thereto.

A dosage of the pharmaceutical composition according to the present invention may need to be a pharmaceutically effective amount. The “pharmaceutically effective amount” may mean an amount enough to prevent or treat diseases at a reasonable benefit/risk ratio applicable to medical treatment, and a level of effective dose may be variously selected by those skilled in the art according to factors such as a formulation method, a patient's condition and weight, a patient's gender, an age, a degree of disease, a drug form, an administration route and period, an excretion rate, reaction sensitivity, etc. The effective amount may vary depending on a route of disposal, a use of excipient, and possibility of being used with other drugs, as recognized by those skilled in the art.

An amount effective for preventing or treating a virus infection disease, a respiratory disease, or an allergic disease may refer to an amount capable of providing a desired outcome or an objective or subjective advantage in a subject, as a single or multiple dose alone or in combination with one or more other compositions (other therapeutic agents for a virus infection disease, a respiratory disease, an allergic disease, or the like).

A dose or dosage of the pharmaceutical composition according to the present invention may vary in a range thereof depending on a patient's weight, age, gender, health condition and diet, an administration time, an administration method, an excretion rate, and a severity of a disease, but may be taken once or several times by dividing the amount of 0.001 mg/kg to 1000 mg/kg on an adult basis.

(1) The present invention may provide a compound of formula 1 below, a pharmaceutically acceptable salt thereof, a stereoisomer thereof, a hydrate thereof or a solvate thereof:

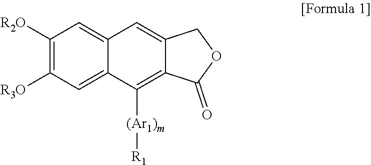

wherein in above formula 1,
Ar₁ may be substituted or unsubstituted C₆-C₁₂ arylene group, or substituted or unsubstituted C₂-C₁₂ heteroarylene group including at least one of N, O and S as a heteroatom in a ring, and m may be 0 or 1,
R₁ may be hydrogen, halogen, substituted or unsubstituted C₁-C₁₀ linear or branched alkyl, substituted or unsubstituted C₂-C₂₀ heterocycloalkyl including at least one of N, O and S as a heteroatom in a ring (wherein a substituent of C₃-C₂₀ cycloalkyl or C₂-C₂₀ heterocycloalkyl may be each independently bonded to an adjacent group to form a ring), substituted or unsubstituted C₂-C₂₀ heteroaryl including at least one of N, O and S as a heteroatom in a ring, —NR₄R₅, —OR₆, —N═CHR₇ or

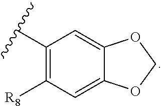

when R₁ is hydrogen or halogen, m may be 1,
when C₂-C₂₀ heteroaryl is furan or thiophene and m is 0, the furan and thiophene may each independently have at least one hydrogen substituted with C₁-C₁₀ linear or branched alkyl, substituted or unsubstituted C₆-C₂₀ aryl, substituted or unsubstituted C₂-C₂₀ heteroaryl, or —C(═O)Rₐ (wherein Rₐ is hydrogen, C₁-C₁₀ linear or branched alkyl, substituted or unsubstituted C₆-C₂₀ aryl, or substituted or unsubstituted C₂-C₂₀ heteroaryl),
R₂ and R₃ may be each independently C₁-C₁₀ linear or branched alkyl,
R₄ to R₇ may be each independently hydrogen, halogen, C₁-C₁₀ linear or branched alkyl (wherein at least one of the hydrogen of the alkyl may be substituted with OH, substituted or unsubstituted C₃-C₁₀ cycloalkyl, substituted or unsubstituted C₃-C₁₀ cycloalkenyl, substituted or unsubstituted C₂-C₁₀ heterocycloalkyl, substituted or unsubstituted C₆-C₂₀ aryl, substituted or unsubstituted C₂-C₂₀ heteroaryl, —C(═O)OR_{b}, linear or branched alkoxy, or amine), substituted or unsubstituted C₃-C₁₀ cycloalkyl (wherein a substituent of the cycloalkyl may be bonded to an adjacent group to form a ring), substituted or unsubstituted C₂-C₁₀ heterocycloalkyl including at least one of N, O and S as a heteroatom in a ring (wherein a substituent of the heterocycloalkyl may be bonded to an adjacent group to form a ring), substituted or unsubstituted C₆-C₂₀ aryl (wherein a substituent of the aryl may be bonded to an adjacent group to form a ring), substituted or unsubstituted C₂-C₂₀ heteroaryl including at least one of N, O and S as a heteroatom in a ring, or —C(═O)R_{c},
R_{b} may be C₁-C₁₀ linear or branched alkyl, and R_{c} may be C₁-C₁₀ linear or branched alkyl or C₁-C₁₀ linear or branched alkoxy, and
R₈ may be —OC(═O)R_{d} or C₁-C₂₀ linear or branched alkoxy, and Rₐ may be C₁-C₁₀ linear or branched alkyl, C₁-C₁₀ linear or branched alkoxy, or substituted or unsubstituted C₃-C₂₀ cycloalkoxy.

(2) In (1), Ar₁—R₁ may be represented by formula 2 below:

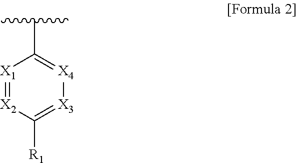

wherein in above formula 2, X₁ to X₄ may be each independently CH or N, and R₁ may be the same as defined in above formula 1.

(3) In (1) or (2), above Ar₁—R₁ or formula 2 may be represented by formulas 2-1 to 2-8 below:

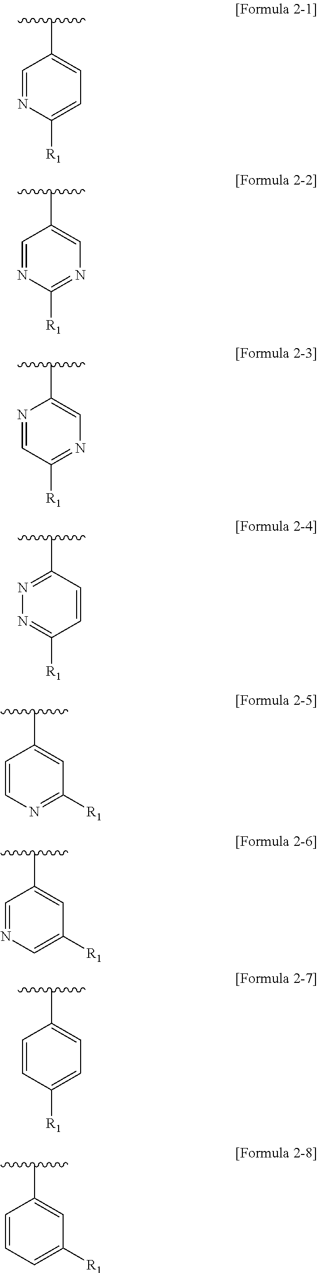

wherein in above formulas 2-1 to 2-8, R₁ may be the same as defined in above formula 2.

(4) In (1), (2) or (3), R₁ may be hydrogen, halogen, substituted or unsubstituted C₁-C₁₀ linear or branched alkyl, —NR₄R₅, —OR₆, —N═CHR₇,

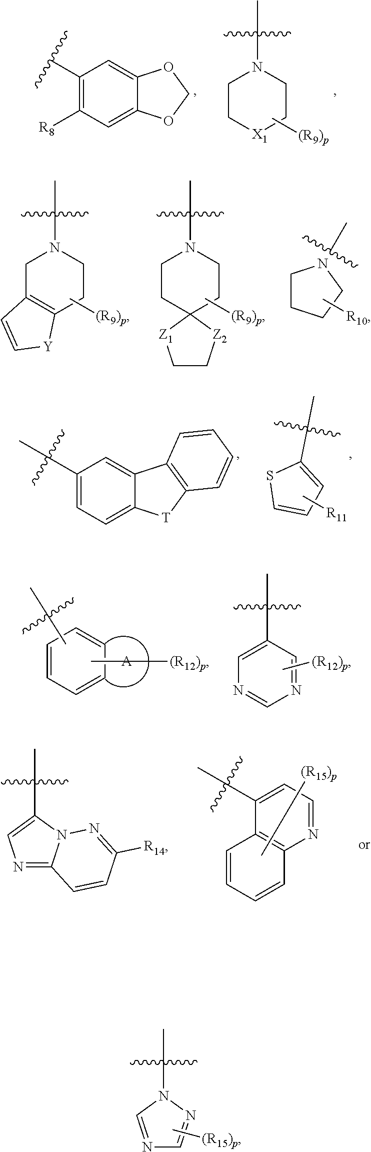

X₁ may be —CHR₁₃, —CX₂ (wherein, X is halogen), —NR₁₃, —O— or —S—,
Y, Z₁, Z₂ and T may be each independently —O— or —S—,
R₉ may be each independently hydrogen, C₁-C₁₀ linear or branched alkyl (wherein at least one of the hydrogen of R₉ may be substituted with —OH, and R₉ may be bonded to an adjacent group to form a substituted or unsubstituted ring), substituted or unsubstituted C₆-C₂₀ aryl, amine, —OH, or —C(═O)ORₑ (Rₑ is hydrogen or C₁-C₁₀ linear or branched alkyl),
R₁₀ may be hydrogen, C₁-C₁₀ linear or branched alkyl (wherein at least one of the hydrogen of the alkyl may be substituted with —OH, C₁-C₁₀ linear or branched alkoxy, or substituted or unsubstituted C₂-C₂₀ heterocycloalkyl), —C(═O)R_{f} (wherein R_{f} is —OH, C₁-C₁₀ linear or branched alkyl, C₁-C₁₀ linear or branched alkoxy, substituted or unsubstituted amine, substituted or unsubstituted C₂-C₂₀ heterocycloalkyl) or —OC(═O)R_{g} (wherein R_{g} is C₁-C₁₀ linear or branched alkyl and may be substituted with —OH),
R₁₁ may be —C(═O)Rₐ (wherein Rₐ is hydrogen, C₁-C₁₀ linear or branched alkyl, substituted or unsubstituted C₆-C₂₀ aryl, or substituted or unsubstituted C₂-C₂₀ heteroaryl),
R₁₂ may be each independently hydrogen, halogen, C₁-C₁₀ linear or branched alkyl (wherein at least one of the hydrogen of the alkyl may be substituted with substituted or unsubstituted C₂-C₁₀ heterocycloalkyl), substituted or unsubstituted C₂-C₂₀ heterocycloalkyl, C₁-C₁₀ linear or branched alkoxy, —CX₃ (wherein X is halogen), or —OH,
R₁₃ may be hydrogen, halogen, substituted or unsubstituted C₁-C₁₀ linear or branched alkyl (wherein at least one of the hydrogen of the alkyl may be substituted with —OH, substituted or unsubstituted C₂-C₁₀ heterocycloalkyl, and R₁₃ may be bonded to an adjacent group to form a substituted or unsubstituted ring), substituted or unsubstituted C₃-C₂₀ cycloalkyl (wherein a substituent of the C₃-C₂₀ cycloalkyl may be bonded to an adjacent group to form a ring), substituted or unsubstituted C₆-C₂₀ aryl, substituted or unsubstituted C₃-C₂₀ heteroaryl (wherein at least one of the hydrogen of the C₃-C₂₀ heteroaryl may be substituted with C₁-C₁₀ alkylthio group or halogen), —OH, or amine,
R₁₄ may be C₂-C₁₀ heterocycloalkyl including at least one of N and O as a heteroatom in a ring,
R₁₅ may be each independently halogen, C₁-C₁₀ linear or branched alkyl, —CX₃ (wherein X is halogen), C₁-C₁₀ linear or branched alkoxy, an alkylthio group, or amine,
p may be an integer of 0 or more and 5 or less, and
A may be substituted or unsubstituted C₂-C₁₀ heterocycloalkyl including at least one of N, O and S as a heteroatom in a ring, substituted or unsubstituted C₆-C₂₀ aryl, or substituted or unsubstituted C₂-C₂₀ heteroaryl including at least one N as a heteroatom in a ring, and R₄ to R₈ may be the same as defined in formula 1.

(5) In (1), (2), (3) or (4), R1 may be hydrogen, fluroine, chlorine, —CH₃,

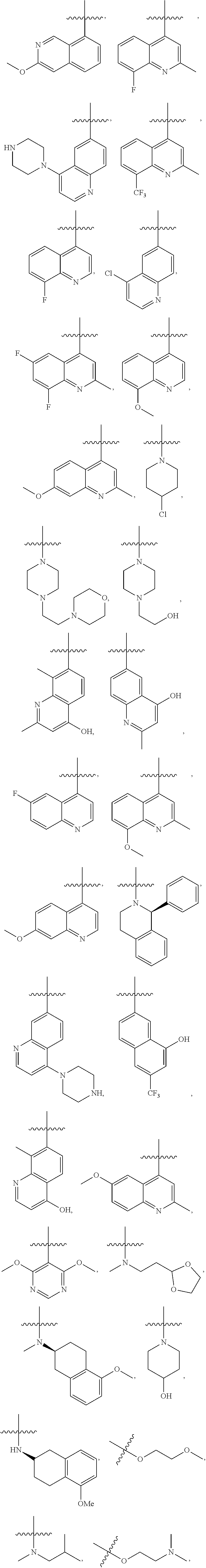

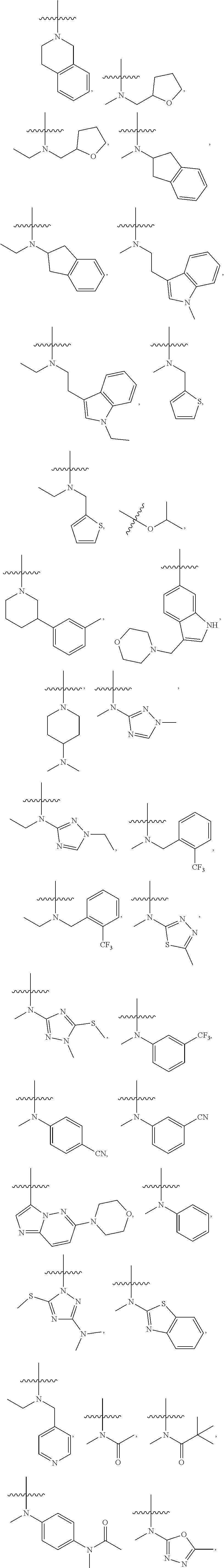

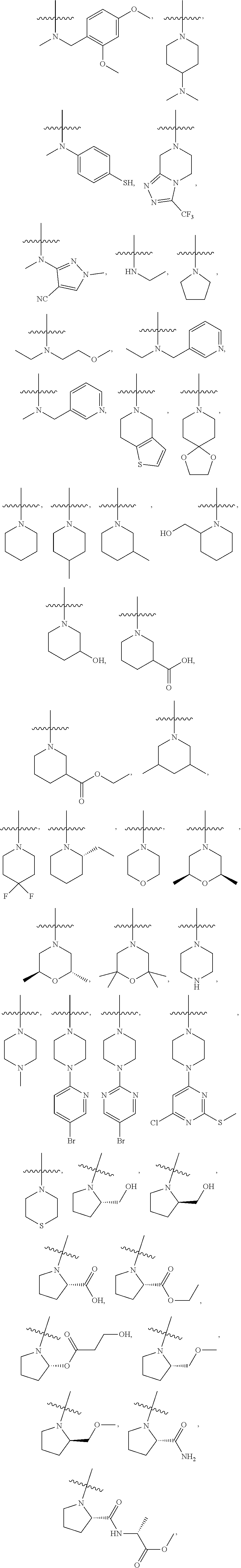

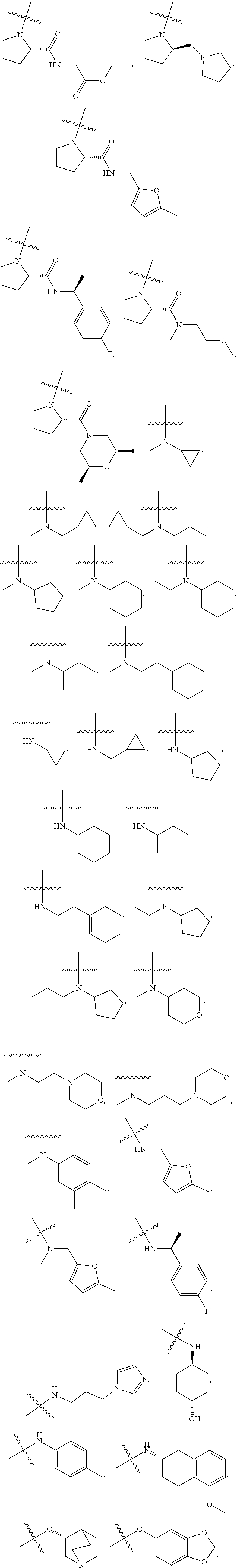

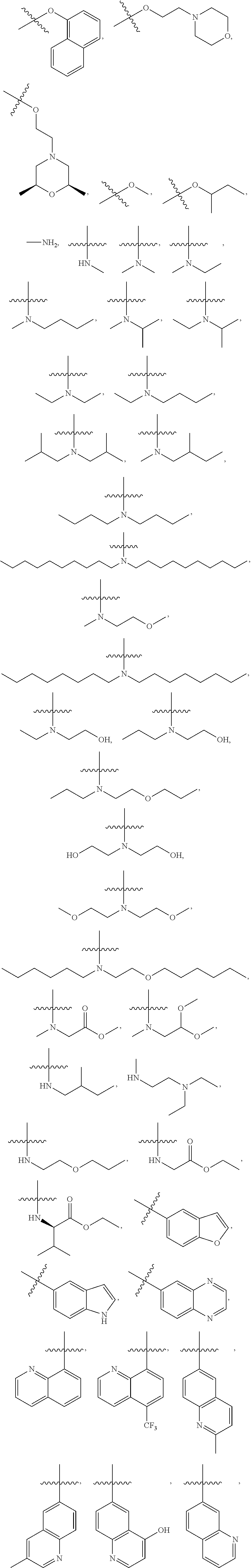

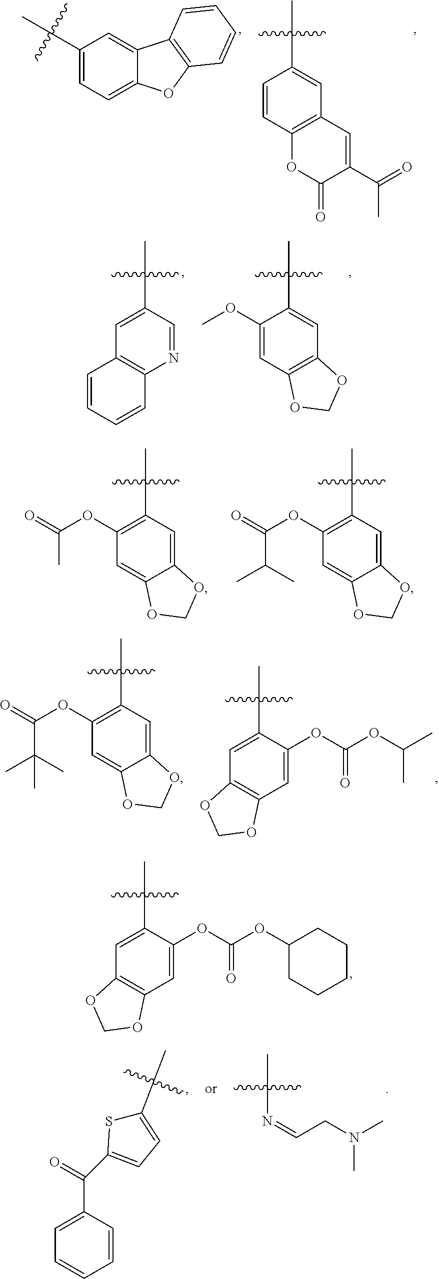

(6) In (1), (2), (3), (4) or (5), the compound represented by above formula 1 may be one selected from the group consisting of the following compounds:
[001] (S)-6,7-dimethoxy-9-((5-methoxy-1,2,3,4-tetrahydronaphthalen-2-yl)amino)naphtho[2,3-c]furan-1(3H)-one
[002] 9-((2S,6R)-2,6-dimethylmorpholino)-6,7-dimethoxynaphtho[2,3-c]furan-1(3H)-one
[003] 9-(6,7-dihydrothieno[3,2-c]pyridin-5 (4H)-yl)-6,7-dimethoxynaphtho[2,3-c]furan-1(3H)-one
[004] 9-(3,5-dimethylpiperidin-1-yl)-6,7-dimethoxynaphtho[2,3-c]furan-1(3H)-one
[005] 6,7-dimethoxy-9-((2-methoxyethyl)(methyl)amino)naphtho[2,3-c]furan-1(3H)-one
[006] 9-((2-(diethylamino)ethyl)amino)-6,7-dimethoxynaphtho[2,3-c]furan-1(3H)-one)
[007] 9-(2-((2S,6R)-2,6-dimethylmorpholino)pyrimidin-5-yl)-6,7-dimethoxynaphtho[2,3-c]furan-1(3H)-one
[008] 9-(2-((2S,6R)-2,6-dimethylmorpholino)pyrimidin-5-yl)-6,7-dimethoxynaphtho[2,3-c]furan-1(3H)-one
[009] 6,7-dimethoxy-9-(6-thiomorpholinopyridin-3-yl)naphtho[2,3-c]furan-1(3H)-one
[010] 6,7-dimethoxy-9-(6-thiomorpholinopyridin-3-yl)naphtho[2,3-c]furan-1(3H)-one
[011] 9-(2-(dimethylamino)pyrimidin-5-yl)-6,7-dimethoxynaphtho[2,3-c]furan-1(3H)-one
[012] 9-(2-(6,7-dihydrothieno[3,2-c]pyridin-5 (4H)-yl)pyrimidin-5-yl)-6,7-dimethoxynaphtho[2,3-c]furan-1(3H)-one
[013] 6,7-dimethoxy-9-(2-(2-morpholinoethoxy)pyrimidin-5-yl)naphtho[2,3-c]furan-1(3H)-one
[014] 9-(2-((3,4-dimethylphenyl)amino)pyrimidin-5-yl)-6,7-dimethoxynaphtho[2,3-c]furan-1(3H)-one
[015] Ethyl 1-(5-(6,7-dimethoxy-3-oxo-1,3-dihydronaphtho[2,3-c]furan-4-yl)pyrimidin-2-yl) piperidin-3-carboxylate
[016] 9-(2-((3-(1H-imidazol-1-yl) propyl)amino)pyrimidin-5-yl)-6,7-dimethoxynaphtho[2,3-c]furan-1(3H)-one
[017] (R)-6,7-dimethoxy-9-(2-(quinuclidin-3-yloxy)pyrimidin-5-yl)naphtho[2,3-c]furan-1(3H)-one
[018] 6,7-dimethoxy-9-(2-((2-methoxyethyl)(methyl)amino)pyrimidin-5-yl)naphtho[2,3-c]furan-1(3H)-one
[019] (S)ethyl 1-(5-(6,7-dimethoxy-3-oxo-1,3-dihydronaphtho[2,3-c]furan-4-yl)pyrimidin-2-yl)pyrrolidin-2-carboxylate
[020] 9-(2-(benzo[d][1,3]dioxol-5-yloxy)pyrimidin-5-yl)-6,7-dimethoxynaphtho[2,3-c]furan-1(3H)-one
[021] 6,7-dimethoxy-9-(2-(naphthalen-1-yloxy)pyrimidin-5-yl)naphtho[2,3-c]furan-1(3H)-one
[022] (S)-6,7-dimethoxy-9-(2-((5-methoxy-1,2,3,4-tetrahydronaphthalen-2-yl)amino)pyrimidin-5-yl)naphtho[2,3-c]furan-1(3H)-one
[023] 9-(2-(isopropyl(methyl)amino)pyrimidin-5-yl)-6,7-dimethoxynaphtho[2,3-c]furan-1(3H)-one
[024] 9-(2-((S)-2-((2S,6R)-2,6-dimethylmorpholin-4-carbonyl)pyrrolidin-1-yl)pyrimidin-5-yl)-6,7-dimethoxynaphtho[2,3-c]furan-1(3H)-one
[025] 9-(2-(sec-butoxy)pyrimidin-5-yl)-6,7-dimethoxynaphtho[2,3-c]furan-1(3H)-one
[026] 6,7-dimethoxy-9-(2-methoxypyrimidin-5-yl)naphtho[2,3-c]furan-1(3H)-one
[027] Methyl (5-(6,7-dimethoxy-3-oxo-1,3-dihydronaphtho[2,3-c]furan-4-yl)pyrimidin-2-yl)-D-valinate
[028] 9-(2-((2-(cyclohex-1-en-1-yl)ethyl)amino)pyrimidin-5-yl)-6,7-dimethoxynaphtho[2,3-c]furan-1(3H)-one
[029] (Z)-9-(5-((2-(dimethylamino)ethyliden)amino)pyrazin-2-yl)-6,7-dimethoxynaphtho[2,3-c]furan-1(3H)-one
[030] 9-(5-aminopyrazin-2-yl)-6,7-dimethoxynaphtho[2,3-c]furan-1(3H)-one
[031] 9-(5-(dimethylamino)pyrazin-2-yl)-6,7-dimethoxynaphtho[2,3-c]furan-1(3H)-one
[032] 6,7-dimethoxy-9-(pyrazin-2-yl)naphtho[2,3-c]furan-1(3H)-one
[033] (S)-1-(5-(6,7-dimethoxy-3-oxo-1,3-dihydronaphtho[2,3-c]furan-4-yl)pyrimidin-2-yl)-N-(2-methoxyethyl)-N-methylpyrrolidin-2-carboxamide
[034] 9-(1H-indol-5-yl)-6,7-dimethoxynaphtho[2,3-c]furan-1(3H)-one
[035] 9-(6-fluoropyridin-3-yl)-6,7-dimethoxynaphtho[2,3-c]furan-1(3H)-one
[036] 2-hydroxyethyl (5-(6,7-dimethoxy-3-oxo-1,3-dihydronaphtho[2,3-c]furan-4-yl)pyrimidin-2-yl)-L-prolinate
[037] 6,7-dimethoxy-9-(2-((2-propoxyethyl)amino)pyrimidin-5-yl)naphtho[2,3-c]furan-1(3H)-one
[038] Methyl (5-(6,7-dimethoxy-3-oxo-1,3-dihydronaphtho[2,3-c]furan-4-yl)pyrimidin-2-yl)-L-prolyl-D-alaniate
[039] (S)-1-(5-(6,7-dimethoxy-3-oxo-1,3-dihydronaphtho[2,3-c]furan-4-yl)pyrimidin-2-yl)-N—((S)-1-(4-fluorophenyl)ethyl)pyrrolidin-2- carboxamide
[040] 6,7-dimethoxy-9-(quinoxalin-6-yl)naphtho[2,3-c]furan-1(3H)-one
[041] (S)-1-(5-(6,7-dimethoxy-3-oxo-1,3-dihydronaphtho[2,3-c]furan-4-yl)pyrimidin-2-yl)-N-((5-methylfuran-2-yl)methyl)pyrrolidin-2-carboxamide
[042] 6,7-dimethoxy-9-(2-((2-propoxyethyl)(propyl)amino)pyrimidin-5-yl)naphtho[2,3-c]furan-1(3H)-one
[043] 9-(dibenzo[b,d]furan-2-yl)-6,7-dimethoxynaphtho[2,3-c]furan-1(3H)-one
[044] Ethyl (5-(6,7-dimethoxy-3-oxo-1,3-dihydronaphtho[2,3-c]furan-4-yl)pyrimidin-2-yl)glycinate
[045] Ethyl (5-(6,7-dimethoxy-3-oxo-1,3-dihydronaphtho[2,3-c]furan-4-yl)pyrimidin-2-yl)-L-proglycinate
[046] 6,7-dimethoxy-9-(quinolin-3-yl)naphtho[2,3-c]furan-1(3H)-one
[047] 9-(2-(((1r,4r)-4-hydroxycyclohexyl)amino)pyrimidin-5-yl)-6,7-dimethoxynaphtho[2,3-c]furan-1(3H)-one carboxamide
[048] 9-(2-(hexyl(2-(hexyloxy)ethyl)amino)pyrimidin-5-yl)-6,7-dimethoxynaphtho[2,3-c]furan-1(3H)-one
[049] (S)-1-(5-(6,7-dimethoxy-3-oxo-1,3-dihydronaphtho[2,3-c]furan-4-yl)pyrimidin-2-yl) pyrrolidin-2-carboxamide
[050] 6,7-dimethoxy-9-(2-(((5-methylfuran-2-yl)methyl)amino)pyrimidin-5-yl)naphtho[2,3-c]furan-1(3H)-one
[051] 6,7-dimethoxy-9-(2-morpholinopyrimidin-5-yl)naphtho[2,3-c]furan-1(3H)-one carboxamide
[052] 9-(benzofuran-5-yl)-6,7-dimethoxynaphtho[2,3-c]furan-1(3H)-one carboxamide
[053] 9-(5-benzothiophen-2-yl)-6,7-dimethoxynaphtho[2,3-c]furan-1(3H)-one
[054] 9-(2-(2-((2S,6R)-2,6-dimethylmorpholino) ethoxy)pyrimidin-5-yl)-6,7-dimethoxynaphtho[2,3-c]furan-1(3H)-one
[055] (S)-9-(2-((1-(4-fluorophenyl)ethyl)amino)pyrimidin-5-yl)-6,7-dimethoxynaphtho[2,3-c]furan-1(3H)-one
[056] 9-(3-acetyl-2-oxo-2H-chromen-6-yl)-6,7-dimethoxynaphtho[2,3-c]furan-1(3H)-one
[057] 9-(2-(3,5-dimethylpiperidin-1-yl)pyrimidin-5-yl)-6,7-dimethoxynaphtho[2,3-c]furan-1(3H)-one
[058] 9-(2-((2-(diethylamino)ethyl)amino)pyrimidin-5-yl)-6,7-dimethoxynaphtho[2,3-c]furan-1(3H)-one carboxamide
[059] 6,7-dimethoxy-9-(2-(3-methylpiperidin-1-yl)pyrimidin-5-yl)naphtho[2,3-c]furan-1(3H)-one
[060] 1-(5-(6,7-dimethoxy-3-oxo-1,3-dihydronaphtho[2,3-c]furan-4-yl)pyrimidin-2-yl) piperidin-3-carboxylic acid
[061] (5-(6,7-dimethoxy-3-oxo-1,3-dihydronaphtho[2,3-c]furan-4-yl)pyrimidin-2-yl)-L-prolan
[062] 9-(2-((2S,6S)-2,6-dimethylmorpholino)pyrimidin-5-yl)-6,7-dimethoxynaphtho[2,3-c]furan-1(3H)-one
[063] 6,7-dimethoxy-9-(2-thiomorpholinopyrimidin-5-yl)naphtho[2,3-c]furan-1(3H)-one
[064] 6,7-dimethoxy-9-(2-((2-methylbutyl)amino)pyrimidin-5-yl)naphtho[2,3-c]furan-1(3H)-one
[065] 6,7-dimethoxy-9-(2-(methyl(2-methylbutyl)amino)pyrimidin-5-yl)naphtho[2,3-c]furan-1(3H)-one
[066] 9-(2-(butyl(ethyl)amino)pyrimidin-5-yl)-6,7-dimethoxynaphtho[2,3-c]furan-1(3H)-one
[067] 9-(2-((cyclopropylmethyl)amino)pyrimidin-5-yl)-6,7-dimethoxynaphtho[2,3-c]furan-1(3H)-one
[068] 9-(2-((cyclopropylmethyl)(methyl)amino)pyrimidin-5-yl)-6,7-dimethoxynaphtho[2,3-c]furan-1(3H)-one
[069] 9-(2-(cyclopropylamino)pyrimidin-5-yl)-6,7-dimethoxynaphtho[2,3-c]furan-1(3H)-one
[070] 9-(2-(cyclopropyl(methyl)amino)pyrimidin-5-yl)-6,7-dimethoxynaphtho[2,3-c]furan-1(3H)-one
[071] 9-(2-(butyl(methyl)amino)pyrimidin-5-yl)-6,7-dimethoxynaphtho[2,3-c]furan-1(3H)-one
[072] 9-(2-(bis(2-methoxyethyl)amino)pyrimidin-5-yl)-6,7-dimethoxynaphtho[2,3-c]furan-1(3H)-one
[073] 9-(2-(dibutylamino)pyrimidin-5-yl)-6,7-dimethoxynaphtho[2,3-c]furan-1(3H)-one
[074] 9-(2-(diethylamino)pyrimidin-5-yl)-6,7-dimethoxynaphtho[2,3-c]furan-1(3H)-one
[075] 9-(2-(ethyl(isopropyl)amino)pyrimidin-5-yl)-6,7-dimethoxynaphtho[2,3-c]furan-1(3H)-one hydrochloride
[076] 9-(2-(butyl(methyl)amino)pyrimidin-5-yl)-6,7-dimethoxynaphtho[2,3-c]furan-1(3H)-one hydrochloride
[077] 9-(2-((2-hydroxyethyl)(propyl)amino)pyrimidin-5-yl)-6,7-dimethoxynaphtho[2,3-c]furan-1(3H)-one hydrochloride
[078] 9-(2-(cyclohexyl(ethyl)amino)pyrimidin-5-yl)-6,7-dimethoxynaphtho[2,3-c]furan-1(3H)-one hydrochloride
[079] 9-(2-(cyclohexyl(methyl)amino)pyrimidin-5-yl)-6,7-dimethoxynaphtho[2,3-c]furan-1(3H)-one hydrochloride
[080] 9-(2-(3-hydroxy peridin-1-ylpyrimidin-5-yl)-6,7-dimethoxynaphtho[2,3-c]furan-1(3H)-one hydrochloride
[081] 9-(2-(ethyl(2-hydroxyethyl)amino)pyrimidin-5-yl)-6,7-dimethoxynaphtho[2,3-c]furan-1(3H)-one hydrochloride
[082] (R)-9-(2-(2-(hydroxymethyl)pyrrolidin-1-yl)pyrimidin-5-yl)-6,7-dimethoxynaphtho[2,3-c]furan-1(3H)-one hydrochloride
[083] (S)-9-(2-(2-(hydroxymethyl)pyrrolidin-1-yl)pyrimidin-5-yl)-6,7-dimethoxynaphtho[2,3-c]furan-1(3H)-one hydrochloride
[084] 9-(2-(cyclopentyl(methyl)amino)pyrimidin-5-yl)-6,7-dimethoxynaphtho[2,3-c]furan-1(3H)-one hydrochloride
[085] 9-(2-(bis(2-hydroxyethyl)amino)pyrimidin-5-yl)-6,7-dimethoxynaphtho[2,3-c]furan-1(3H)-one hydrochloride
[086] 9-(2-(diisobutylamino)pyrimidin-5-yl)-6,7-dimethoxynaphtho[2,3-c]furan-1(3H)-one hydrochloride
[087] 9-(2-(didecylamino)pyrimidin-5-yl)-6,7-dimethoxynaphtho[2,3-c]furan-1(3H)-one hydrochloride
[088] 6,7-dimethoxy-9-(6-methoxybenzo[d][1,3]dioxol-5-yl)naphtho[2,3-c]furan-1(3H)-one
[089] 9-(2-(1,4-dioxa-8-azaspiro[4.5]decan-8-yl)pyrimidin-5-yl)-6,7-dimethoxynaphtho[2,3-c]furan-1(3H)-one
[090] Methyl N-(5-(6,7-dimethoxy-3-oxo-1,3-dihydronaphtho[2,3-c]furan-4-yl)pyrimidin-2-yl)-N-methyl glycinate hydrochloride
[091] 9-(2-(cyclopentylamino)pyrimidin-5-yl)-6,7-dimethoxynaphtho[2,3-c]furan-1(3H)-one hydrochloride
[092] 9-(2-(cyclopentyl(ethyl)amino)pyrimidin-5-yl)-6,7-dimethoxynaphtho[2,3-c]furan-1(3H)-one hydrochloride
[093] 9-(2-(cyclopentyl(propyl)amino)pyrimidin-5-yl)-6,7-dimethoxynaphtho[2,3-c]furan-1(3H)-one hydrochloride
[094] 9-(2-((2,2-dimethoxyethyl)(methyl)amino)pyrimidin-5-yl)-6,7-dimethoxynaphtho[2,3-c]furan-1(3H)-one
[095] 9-(2-(dioctylamino)pyrimidin-5-yl)-6,7-dimethoxynaphtho[2,3-c]furan-1(3H)-one hydrochloride
[096] (S)-6,7-dimethoxy-9-(2-((5-methoxy-1,2,3,4-tetrahydronaphthalen-2-yl)(methyl)amino)pyrimidin-5-yl)naphtho[2,3-c]furan-1(3H)-one hydrochloride
[097] (S)-6,7-dimethoxy-9-(2-(2-(methoxymethyl)pyrrolidin-1-yl)pyrimidin-5-yl)naphtho[2,3-c]furan-1(3H)-one hydrochloride
(6-(6,7-dimethoxy-3-oxo-1,3-dihydronaphtho[2,3-c]furan-4-yl)benzo[d][1,3]dioxol-5-yl isopropyl carbonate
[099] Cyclohexyl (6-(6,7-dimethoxy-3-oxo-1,3-dihydronaphtho[2,3-c]furan-4-yl)benzo[d][1,3]dioxol-5-yl)carbonate
[100] 9-(2-(sec-butyl(methyl)amino)pyrimidin-5-yl)-6,7-dimethoxynaphtho[2,3-c]furan-1(3H)-one hydrochloride
[101] 9-(2-((2-(cyclohex-1-en-1-yl)ethyl)(methyl)amino)pyrimidin-5-yl)-6,7-dimethoxynaphtho[2,3-c]furan-1(3H)-one hydrochloride
[102] (R)-6,7-dimethoxy-9-(2-(2-(methoxymethyl)pyrrolidin-1-yl)pyrimidin-5-yl)naphtho[2,3-c]furan-1(3H)-one hydrochloride
[103] 6,7-dimethoxy-9-(2-(methyl((5-methylfuran-2-yl)methyl)amino)pyrimidin-5-yl)naphtho[2,3-c]furan-1(3H)-one hydrochloride
[104] 9-(2-(2-(hydroxymethyl) piperidin-1-yl)pyrimidin-5-yl)-6,7-dimethoxynaphtho[2,3-c]furan-1(3H)-one hydrochloride
[105] 9-(2-((cyclopropylmethyl)(propyl)amino)pyrimidin-5-yl)-6,7-dimethoxynaphtho[2,3-c]furan-1(3H)-one hydrochloride
[106] 9-(2-(ethyl(methyl)amino)pyrimidin-5-yl)-6,7-dimethoxynaphtho[2,3-c]furan-1(3H)-one hydrochloride
[107] 6,7-dimethoxy-9-(2-(4-methylpiperidin-1-yl)pyrimidin-5-yl)naphtho[2,3-c]furan-(3H)-one hydrochloride
[108] 6,7-dimethoxy-9-(2-(methylamino)pyrimidin-5-yl)naphtho[2,3-c]furan-1(3H)-one
[109] 9-(2-((3,4-dimethylphenyl)(methyl)amino)pyrimidin-5-yl)-6,7-dimethoxynaphtho[2,3-c]furan-1(3H)-one
[110] (R)-6,7-dimethoxy-9-(2-(2-(pyrrolidin-1-ylmethyl)pyrrolidin-1-yl)pyrimidin-5-yl)naphtho[2,3-c]furan-1(3H)-one
[111] 6,7-dimethoxy-9-(2-(methyl(2-morpholinoethyl)amino)pyrimidin-5-yl)naphtho[2,3-c]furan-1(3H)-one hydrochloride
[112] 6,7-dimethoxy-9-(2-(methyl(3-morpholinopropyl)amino)pyrimidin-5-yl)naphtho[2,3-c]furan-1(3H)-one dihydrochloride
[113] (R)-9-(2-(2-ethylpiperidin-1-yl)pyrimidin-5-yl)-6,7-dimethoxynaphtho[2,3-c]furan-1(3H)-one
[114] 6,7-dimethoxy-9-(2-(2,2,6,6-tetramethylmorpholino)pyrimidin-5-yl)naphtho[2,3-c]furan-1(3H)-one
[115] 6,7-dimethoxy-9-(2-(methyl(tetrahydro-2H-pyran-4-yl)amino)pyrimidin-5-yl)naphtho[2,3-c]furan-1(3H)-one
[116] 6,7-dimethoxy-9-(2-methylquinolin-6-yl)naphtho[2,3-c]furan-1(3H)-one
[117] 9-(4-hydroxyquinolin-6-yl)-6,7-dimethoxynaphtho[2,3-c]furan-1(3H)-one
[118] 6,7-dimethoxy-9-(quinolin-7-yl)naphtho[2,3-c]furan-1(3H)-one
[119] 6,7-dimethoxy-9-(5-(trifluoromethyl) quinolin-8-yl)naphtho[2,3-c]furan-1(3H)-one
[120] 6,7-dimethoxy-9-(3-methylquinolin-6-yl)naphtho[2,3-c]furan-1(3H)-one
[121] 9-(6-((2S,6R)-2,6-dimethylmorpholino)pyridin-3-yl)-6,7-dimethoxynaphtho[2,3-c]furan-1(3H)-one
[122] 9-(6-(4,4-difluoropiperidin-1-yl)pyridin-3-yl)-6,7-dimethoxynaphtho[2,3-c]furan-1(3H)-one
[123] 6,7-dimethoxy-9-(6-(piperidin-1-yl)pyridin-3-yl)naphtho[2,3-c]furan-1(3H)-one
[124] 6,7-dimethoxy-9-(6-morpholinopyridin-3-yl)naphtho[2,3-c]furan-1(3H)-one
[125] 6,7-dimethoxy-9-(quinolin-8-yl)naphtho[2,3-c]furan-1(3H)-one
[126] 6,7-dimethoxy-9-(6-(3-methylpiperidin-1-yl)pyridin-3-yl)naphtho[2,3-c]furan-1(3H)-one
[127] 6,7-dimethoxy-9-(6-(3-methylpiperidin-1-yl)pyridin-3-yl)naphtho[2,3-c]furan-1(3H)-one
[128] 6,7-dimethoxy-9-(piperazin-1-yl)naphtho[2,3-c]furan-1(3H)-one
[129] 9-(4-(5-bromopyridin-2-yl) piperazin-1-yl)-6,7-dimethoxynaphtho[2,3-c]furan-1(3H)-one
[130] 9-(4-(6-chloro-2-(methylthio)pyrimidin-4-yl) piperazin-1-yl)-6,7-dimethoxynaphtho[2,3-c]furan-1(3H)-one
[131] 9-(4-(5-bromopyrimidin-2-yl) piperazin-1-yl)-6,7-dimethoxynaphtho[2,3-c]furan-1(3H)-one
[132] 6,7-dimethoxy-9-(quinolin-5-yl)naphtho[2,3-c]furan-1(3H)-one
[133] 6,7-dimethoxy-9-(3-methoxyisoquinolin-8-yl)naphtho[2,3-c]furan-1(3H)-one
[134] 9-(8-fluoro-2-methylquinolin-4-yl)-6,7-dimethoxynaphtho[2,3-c]furan-1(3H)-one
[135] 6,7-dimethoxy-9-(4-(piperazin-1-yl) quinolin-6-yl)naphtho[2,3-c]furan-1(3H)-one
[136] 6,7-dimethoxy-9-(2-methyl-8-(trifluoromethyl) quinolin-4-yl)naphtho[2,3-c]furan-1(3H)-one
[137] 9-(8-fluoroquinolin-4-yl)-6,7-dimethoxynaphtho[2,3-c]furan-1(3H)-one
[138] 9-(4-chloroquinolin-6-yl)-6,7-dimethoxynaphtho[2,3-c]furan-1(3H)-one
[139] 9-(6,8-difluoro-2-methylquinolin-4-yl)-6,7-dimethoxynaphtho[2,3-c]furan-1(3H)-one
[140] 6,7-dimethoxy-9-(8-methoxyquinolin-4-yl)naphtho[2,3-c]furan-1(3H)-one
[141] 6,7-dimethoxy-9-(7-methoxy-2-methylquinolin-4-yl)naphtho[2,3-c]furan-1(3H)-one
[142] 9-(6-(4-chloropiperidin-1-yl)pyridin-3-yl)-6,7-dimethoxynaphtho[2,3-c]furan-1(3H)-one
[143] 6,7-dimethoxy-9-(4-(2-morpholinoethyl) piperazin-1-yl)naphtho[2,3-c]furan-1(3H)-one
[144] 9-(4-(2-hydroxyethyl) piperazin-1-yl)-6,7-dimethoxynaphtho[2,3-c]furan-1(3H)-one
[146] 9-(4-hydroxy-2,8-dimethylquinolin-7-yl)-6,7-dimethoxynaphtho[2,3-c]furan-1(3H)-one
[147] 9-(4-hydroxy-2-methylquinolin-6-yl)-6,7-dimethoxynaphtho[2,3-c]furan-1(3H)-one
[148] 9-(6-fluoroquinolin-4-yl)-6,7-dimethoxynaphtho[2,3-c]furan-1(3H)-one
[149] 6,7-dimethoxy-9-(8-methoxy-2-methylquinolin-4-yl)naphtho[2,3-c]furan-1(3H)-one
[150] 6,7-dimethoxy-9-(7-methoxyquinolin-4-yl)naphtho[2,3-c]furan-1(3H)-one
[151] (S)-6,7-dimethoxy-9-(2-(1-phenyl-3,4-dihydroisoquinolin-2 (1H)-yl)pyrimidin-5-yl)naphtho[2,3-c]furan-1(3H)-one
[152] 6,7-dimethoxy-9-(4-(piperazin-1-yl) quinolin-7-yl)naphtho[2,3-c]furan-1(3H)-one
[154] 9-(4-hydroxy-8-methylquinolin-7-yl)-6,7-dimethoxynaphtho[2,3-c]furan-1(3H)-one
[155] 6,7-dimethoxy-9-(6-methoxy-2-methylquinolin-4-yl)naphtho[2,3-c]furan-1(3H)-one
[156] 9-(6-(6,7-dihydrothien[3,2-c]pyridin-5 (4H)-yl)pyridin-3-yl)-6,7-dimethoxynaphtho[2,3-c]furan-1(3H)-one
[157] 9-(4-(4,6-dimethoxypyrimidin-5-yl)phenyl)-6,7-dimethoxynaphtho[2,3-c]furan-1(3H)-one
[158] 9-(2-((2-(1,3-dioxolan-2-yl)ethyl)(methyl)amino)pyrimidin-5-yl)-6,7-dimethoxynaphtho[2,3-c]furan-1(3H)-one
[159] 6,7-dimethoxy-9-(6-(4-methylpiperidin-1-yl)pyridin-3-yl)naphtho[2,3-c]furan-1(3H)-one
[160] 9-(6-(diethylamino)pyridin-3-yl)-6,7-dimethoxynaphtho[2,3-c]furan-1(3H)-one
[161] 9-(6-((2-(1,3-dioxolan-2-yl)ethyl)(methyl)amino)pyridin-3-yl)-6,7-dimethoxynaphtho[2,3-c]furan-1(3H)-one
[162] 9-(6-((2,2-dimethoxyethyl)(methyl)amino)pyridin-3-yl)-6,7-dimethoxynaphtho[2,3-c]furan-1(3H)-one
[163] 9-(6-(dimethylamino)pyridin-3-yl)-6,7-dimethoxynaphtho[2,3-c]furan-1(3H)-one
[164] (S)-6,7-dimethoxy-9-(6-((5-methoxy-1,2,3,4-tetrahydronaphthalen-2-yl)(methyl)amino)pyridin-3-yl)naphtho[2,3-c]furan-1(3H)-one
[165] 9-(6-(4-hydroxypiperidin-1-yl)pyridin-3-yl)-6,7-dimethoxynaphtho[2,3-c]furan-1(3H)-one
[166] 9-(6-(1,4-dioxa-8-azaspiro[4.5]decan-8-yl)pyridin-3-yl)-6,7-dimethoxynaphtho[2,3-c]furan-1(3H)-one
[167] 6,7-dimethoxy-9-(6-(4-methylpiperazin-1-yl)pyridin-3-yl)naphtho[2,3-c]furan-1(3H)-one
[168] 6,7-dimethoxy-9-(6-((2-methoxyethyl)(methyl)amino)pyridin-3-yl)naphtho[2,3-c]furan-1(3H)-one hydrochloride
[171] 9-(6-(cyclopropyl(methyl)amino)pyridin-3-yl)-6,7-dimethoxynaphtho[2,3-c]furan-1(3H)-one
[172] (S)-6,7-dimethoxy-9-(6-((5-methoxy-1,2,3,4-tetrahydronaphthalen-2-yl)amino)pyridin-3-yl)naphtho[2,3-c]furan-1(3H)-one
[173] 6,7-dimethoxy-9-(5-(2-methoxyethoxy)pyridin-3-yl)naphtho[2,3-c]furan-1(3H)-one
[174] 9-(6-(bis(2-methoxyethyl)amino)pyridin-3-yl)-6,7-dimethoxynaphtho[2,3-c]furan-1(3H)-one
[175] 9-(6-(cyclohexyl(methyl)amino)pyridin-3-yl)-6,7-dimethoxynaphtho[2,3-c]furan-1(3H)-one
[176] 6,7-dimethoxy-9-(6-(methyl(2-methylbutyl)amino)pyridin-3-yl)naphtho[2,3-c]furan-1(3H)-one
[177] 9-(6-(cyclopentyl(methyl)amino)pyridin-3-yl)-6,7-dimethoxynaphtho[2,3-c]furan-1(3H)-one
[178] 9-(6-(ethyl(methyl)amino)pyridin-3-yl)-6,7-dimethoxynaphtho[2,3-c]furan-1(3H)-one
[179] 6,7-dimethoxy-9-(6-(methyl((5-methylfuran-2-yl)methyl)amino)pyridin-3-yl)naphtho[2,3-c]furan-1(3H)-one
[180] 9-(6-(butyl(methyl)amino)pyridin-3-yl)-6,7-dimethoxynaphtho[2,3-c]furan-1(3H)-one
[181] 9-(6-(isobutyl(methyl)amino)pyridin-3-yl)-6,7-dimethoxynaphtho[2,3-c]furan-1(3H)-one
[182] 9-(6-(ethyl(isopropyl)amino)pyridin-3-yl)-6,7-dimethoxynaphtho[2,3-c]furan-1(3H)-one
[183] 9-(6-((cyclopropylmethyl)(methyl)amino)pyridin-3-yl)-6,7-dimethoxynaphtho[2,3-c]furan-1(3H)-one
[184] 9-(2-chloropyridin-4-yl)-6,7-dimethoxynaphtho[2,3-c]furan-1(3H)-one
[185] 9-(6-chloropyridin-3-yl)-6,7-dimethoxynaphtho[2,3-c]furan-1(3H)-one
[186] 9-(2-fluoropyridin-4-yl)-6,7-dimethoxynaphtho[2,3-c]furan-1(3H)-one
[187] 9-(5-(2-(dimethylamino) ethoxy)pyridin-3-yl)-6,7-dimethoxynaphtho[2,3-c]furan-1(3H)-one
[188] 9-(6-(isopropyl(methyl)amino)pyridin-3-yl)-6,7-dimethoxynaphtho[2,3-c]furan-1(3H)-one
[189] 9-(6-(3,4-dihydroisoquinolin-2 (1H)-yl)pyridin-3-yl)-6,7-dimethoxynaphtho[2,3-c]furan-1(3H)-one
[191] 9-(6-(cyclopentyl(ethyl)amino)pyridin-3-yl)-6,7-dimethoxynaphtho[2,3-c]furan-1(3H)-one
[192] 6,7-dimethoxy-9-(6-(methyl((tetrahydrofuran-2-yl)methyl)amino)pyridin-3-yl)naphtho[2,3-c]furan-1(3H)-one
[193] 9-(6-(ethyl((tetrahydrofuran-2-yl)methyl)amino)pyridin-3-yl)-6,7-dimethoxynaphtho[2,3-c]furan-1(3H)-one
[194] 9-(6-((2,3-dihydro-1H-indan-2-yl)(methyl)amino)pyridin-3-yl)-6,7-dimethoxynaphtho[2,3-c]furan-1(3H)-one
[195] 9-(6-((2,3-dihydro-1H-indan-2-yl)(ethyl)amino)pyridin-3-yl)-6,7-dimethoxynaphtho[2,3-c]furan-1(3H)-one
[196] 6,7-dimethoxy-9-(6-(methyl(2-(1-methyl-1H-indol-3-yl)ethyl)amino)pyridin-3-yl)naphtho[2,3-c]furan-1(3H)-one
[197] 9-(6-(ethyl(2-(1-ethyl-1H-indol-3-yl)ethyl)amino)pyridin-3-yl)-6,7-dimethoxynaphtho[2,3-c]furan-1(3H)-one
[198] 6,7-dimethoxy-9-(6-(methyl(thiophen-2-ylmethyl)amino)pyridin-3-yl)naphtho[2,3-c]furan-1(3H)-one
[199] 9-(6-(ethyl(thiophen-2-ylmethyl)amino)pyridin-3-yl)-6,7-dimethoxynaphtho[2,3-c]furan-1(3H)-one
[200] 9-(5-isopropoxypyridin-3-yl)-6,7-dimethoxynaphtho[2,3-c]furan-1(3H)-one
[202] 6,7-dimethoxy-9-(6-(3-(m-tolyl) piperidin-1-yl)pyridin-3-yl)naphtho[2,3-c]furan-1(3H)-one
[203] 6,7-dimethoxy-9-(3-(morpholinomethyl)-1H-indol-6-yl)naphtho[2,3-c]furan-1(3H)-one
[205] 9-(2-(4-(dimethylamino) piperidin-1-yl)pyrimidin-5-yl)-6,7-dimethoxynaphtho[2,3-c]furan-1(3H)-one
[206] 6,7-dimethoxy-9-(6-(methyl(1-methyl-1H-1,2,4-triazol-3-yl)amino)pyridin-3-yl)naphtho[2,3-c]furan-1(3H)-one
[207] 9-(6-(ethyl(1-ethyl-1H-1,2,4-triazol-3-yl)amino)pyridin-3-yl)-6,7-dimethoxynaphtho[2,3-c]furan-1(3H)-one
[208] 6,7-dimethoxy-9-(6-(methyl(2-(trifluoromethyl)benzyl)amino)pyridin-3-yl)naphtho[2,3-c]furan-1(3H)-one
[209] 9-(6-(ethyl(2-(trifluoromethyl)benzyl)amino)pyridin-3-yl)-6,7-dimethoxynaphtho[2,3-c]furan-1(3H)-one
[213] 9-(2-((3,4-dimethylphenyl)(methyl)amino)pyrimidin-5-yl)-6,7-dimethoxynaphtho[2,3-c]furan-1(3H)-one
[214] 6,7-dimethoxy-9-(6-(methyl(5-methyl-1,3,4-thiadiazol-2-yl)amino)pyridin-3-yl)naphtho[2,3-c]furan-1(3H)-one
[215] 6,7-dimethoxy-9-(6-(methyl(1-methyl-5-(methylthio)-1H-1,2,4-triazol-3-yl)amino)pyridin-3-yl)naphtho[2,3-c]furan-1(3H)-one
[216] 6,7-dimethoxy-9-(2-(methyl(3-(trifluoromethyl)phenyl)amino)pyrimidin-5-yl)naphtho[2,3-c]furan-1(3H)-one
[218] 4-((5-(6,7-dimethoxy-3-oxo-1,3-dihydronaphtho[2,3-c]furan-4-yl)pyridin-2-yl)(methyl)amino)benzonitrile
[219] 6,7-dimethoxy-9-(6-morpholinopyridazin-3-yl)naphtho[2,3-c]furan-1(3H)-one
[220] 6,7-dimethoxy-9-(6-morpholinoimidazo[1,2-b]pyridazin-3-yl)naphtho[2,3-c]furan-1(3H)-one
[222] 6,7-dimethoxy-9-(6-(methyl(phenyl)amino)pyridin-3-yl)naphtho[2,3-c]furan-1(3H)-one
[223] 9-(6-(3-(dimethylamino)-5-(methylthio)-1H-1,2,4-triazol-1-yl)pyridin-3-yl)-6,7-dimethoxynaphtho[2,3-c]furan-1(3H)-one
[225] 9-(6-(benzo[d]thiazol-2-yl(methyl)amino)pyridin-3-yl)-6,7-dimethoxynaphtho[2,3-c]furan-1(3H)-one
[226] 9-(6-(ethyl(pyridin-4-ylmethyl)amino)pyridin-3-yl)-6,7-dimethoxynaphtho[2,3-c]furan-1(3H)-one
[229] 6,7-dimethoxy-9-(6-(methylamino)pyridin-3-yl)naphtho[2,3-c]furan-1(3H)-one
[230] N-(5-(6,7-dimethoxy-3-oxo-1,3-dihydronaphtho[2,3-c]furan-4-yl)pyridin-2-yl)-N-methylacetamide
[231] 6,7-dimethoxy-9-(6-(methyl(5-methyl-1,3,4-oxadiazol-2-yl)amino)pyridin-3-yl)naphtho[2,3-c]furan-1(3H)-one
[232] 9-(6-((2,4-dimethoxybenzyl)(methyl)amino)pyridin-3-yl)-6,7-dimethoxynaphtho[2,3-c]furan-1(3H)-one
[233] N-(5-(6,7-dimethoxy-3-oxo-1,3-dihydronaphtho[2,3-c]furan-4-yl)pyridin-2-yl)-N-methylpivalamide
[236] N-(4-((5-(6,7-dimethoxy-3-oxo-1,3-dihydronaphtho[2,3-c]furan-4-yl)pyridin-2-yl)(methyl)amino)phenyl)-N-methylacetamide
[237] 3-((5-(6,7-dimethoxy-3-oxo-1,3-dihydronaphtho[2,3-c]furan-4-yl)pyridin-2-yl)(methyl)amino)benzonitrile
[238] 9-(6-(4-(dimethylamino) piperidin-1-yl)pyridin-3-yl)-6,7-dimethoxynaphtho[2,3-c]furan-1(3H)-one
[239] 9-(6-((4-mercaptophenyl)(methyl)amino)pyridin-3-yl)-6,7-dimethoxynaphtho[2,3-c]furan-1(3H)-one
[240] 6,7-dimethoxy-9-(6-(3-(trifluoromethyl)-5,6-dihydro-[1,2,4]triazolo[4,3-a]pyrazin-7 (8H)-yl)pyridin-3-yl)naphtho[2,3-c]furan-1(3H)-one
[241] 3-((5-(6,7-dimethoxy-3-oxo-1,3-dihydronaphtho[2,3-c]furan-4-yl)pyridin-2-yl)(methyl)amino)-1-methyl-1H-pyrazol-4-carbonitrile
[242] 6,7-dimethoxy-9-(4-(piperidin-1-yl)phenyl)naphtho[2,3-c]furan-1(3H)-one
[243] 6,7-dimethoxy-9-(3-(piperidin-1-yl)phenyl)naphtho[2,3-c]furan-1(3H)-one
[244] 6,7-dimethoxy-9-(3-morpholinophenyl)naphtho[2,3-c]furan-1(3H)-one
[245] 6,7-dimethoxy-9-(4-morpholinophenyl)naphtho[2,3-c]furan-1(3H)-one
[246] 6,7-dimethoxy-9-(4-(methylamino)phenyl)naphtho[2,3-c]furan-1(3H)-one
[247] 9-(4-(dimethylamino)phenyl)-6,7-dimethoxynaphtho[2,3-c]furan-1(3H)-one
[248] 9-(4-(ethyl(methyl)amino)phenyl)-6,7-dimethoxynaphtho[2,3-c]furan-1(3H)-one
[249] 9-(4-(diethylamino)phenyl)-6,7-dimethoxynaphtho[2,3-c]furan-1(3H)-one
[250] 9-(4-(ethylamino)phenyl)-6,7-dimethoxynaphtho[2,3-c]furan-1(3H)-one
[251] 6,7-dimethoxy-9-(4-(pyrrolidin-1-yl)phenyl)naphtho[2,3-c]furan-1(3H)-one
[252] 6,7-dimethoxy-9-(3-(pyrrolidin-1-yl)phenyl)naphtho[2,3-c]furan-1(3H)-one
[253] 9-(4-(ethyl(2-methoxyethyl)amino)phenyl)-6,7-dimethoxynaphtho[2,3-c]furan-1(3H)-one
[254] 9-(4-(ethyl(pyridin-3-ylmethyl)amino)phenyl)-6,7-dimethoxynaphtho[2,3-c]furan-1(3H)-one
[255] 6,7-dimethoxy-9-(4-(methyl(pyridin-3-ylmethyl)amino)phenyl)naphtho[2,3-c]furan-1(3H)-one
[256] 6,7-dimethoxy-9-(4-((2-methoxyethyl)(methyl)amino)phenyl)naphtho[2,3-c]furan-1(3H)-one
[257] 6,7-dimethoxy-9-(3-(methylamino)phenyl)naphtho[2,3-c]furan-1(3H)-one
[258] 9-(3-(ethylamino)phenyl)-6,7-dimethoxynaphtho[2,3-c]furan-1(3H)-one
[274] 6-(6,7-dimethoxy-3-oxo-1,3-dihydronaphtho[2,3-c]furan-4-yl)benzo[d][1,3]dioxol-5-yl acetate
[275] 6-(6,7-dimethoxy-3-oxo-1,3-dihydronaphtho[2,3-c]furan-4-yl)benzo[d][1,3]dioxol-5-yl isobutrate
[276] 6-(6,7-dimethoxy-3-oxo-1,3-dihydronaphtho[2,3-c]furan-4-yl)benzo[d][1,3]dioxol-5-yl pivalate

(7) The present invention may provide a compound of formula 3 below, a pharmaceutically acceptable salt thereof, a stereoisomer thereof, a hydrate thereof or a solvate thereof:

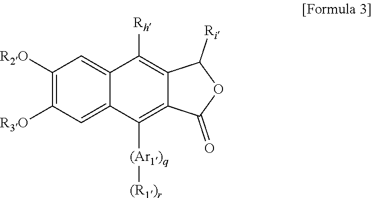

wherein in above formula 3,
Ar_{1′} may be substituted or unsubstituted C₆-C₁₂ arylene group, or substituted or unsubstituted C₂-C₁₂ heteroarylene group including at least one of N, O and S as a heteroatom in a ring, and q may be 0 or 1,
R_{1′} may be each independently halogen, substituted or unsubstituted C₆-C₂₀ aryl (wherein a substituent of the C₆-C₂₀ aryl may be bonded to an adjacent group to form a ring);
—NR_{4′}R_{5′}; —OR_{6′}; —C(═O)CX₃ (wherein X is halogen); —NO₂;

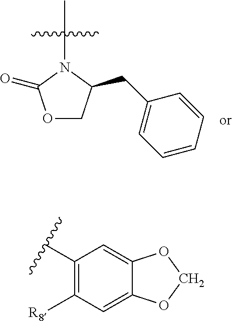

(wherein at least one of the hydrogen of

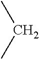

may be substituted with halogen), and r may be an integer of 0 or more and 2 or less,
R_{2′} and R_{3′} may be each independently C₁-C₁₀ linear or branched alkyl,
R_{4′} to R_{6′} may be each independently C₁-C₁₀ linear or branched alkyl, or —C(═O)R_{c′},
R_{c′} may be substituted or unsubstituted C₂-C₁₀ heterocycloalkyl including at least one of N, O and S as a heteroatom in a ring, or substituted or unsubstituted C₂-C₂₀ heteroaryl including at least one of N, O and S as a heteroatom in a ring,
R₈′ may be hydrogen, —OC(═O)R_{d′}, —OP(═O)(OR_{d′})₂, —OP(═O)(NHR_{d′})(OR_{e′}), —OP(═O)(NHR_{d′})(R_{e′}), —OH, —NHR_{d′} or C₁-C₂₀ linear or branched alkoxy,
R_{d′} and R_{e′} may be each independently C₁-C₁₀ linear or branched alkyl (wherein at least one of the hydrogen of the alkyl may be substituted with C₂-C₁₀ heterocycloalkyl or amine), C₂-C₁₀ heterocycloalkyl, substituted or unsubstituted C₆-C₂₀ aryl, or substituted or unsubstituted C₂-C₂₀ heteroaryl, and
R_{h′} and R_{i′} may be each independently hydrogen,

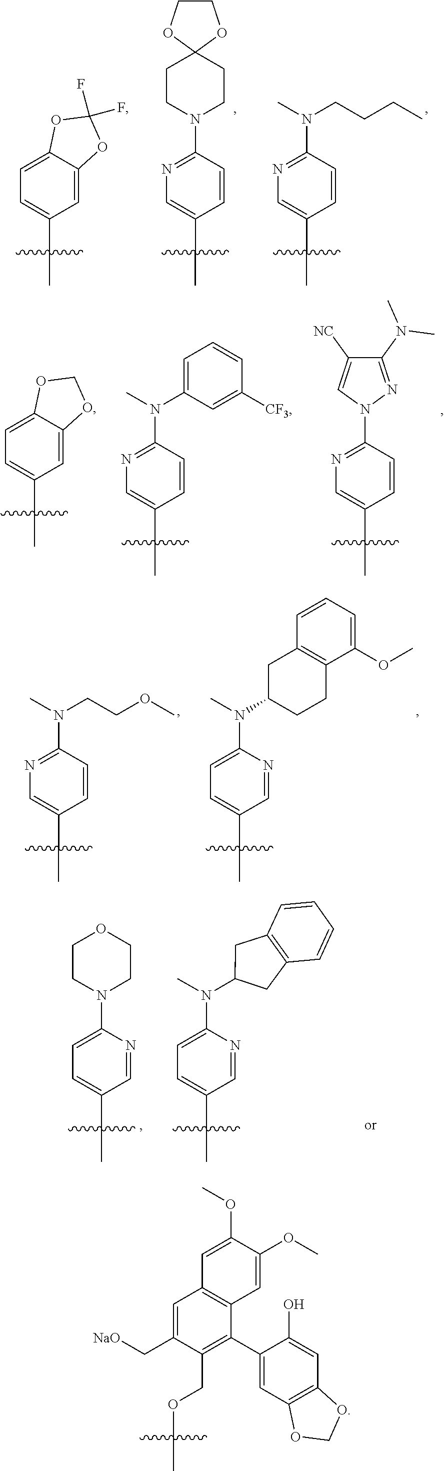

(8) In (7), (Ar_{1′})_{q}—(R_{1′})ᵣ may be represented by formula 4 below:

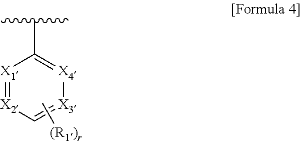

wherein in above formula 4, X_{1′} to X_{4′} may be each independently CH or N, and R_{1′} and r may be the same as defined in above formula 3.

(9) In (7) or (8), (Ar_{1′})_{q}—(R_{1′})ᵣ or formula 4 may be represented by formulas 4-1 to 4-4 below:

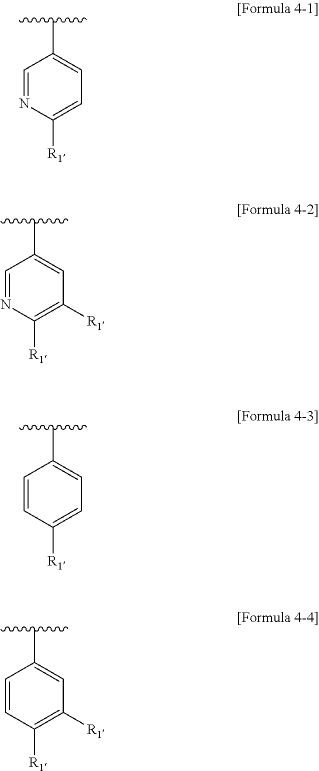

wherein in above formulas 4-1 to 4-4, Rr may be the same as defined in above formula 3.

(10) In (7), (8) or (9), Rr may be halogen, —NR_{4′}R_{5′}, —OR_{6′}, —C(═O)CX₃ (wherein X is halogen), —NO₂

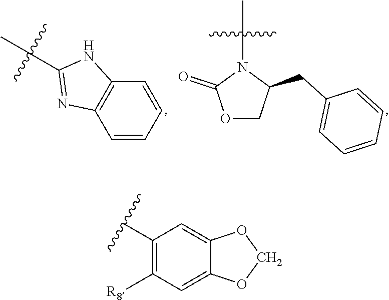

(wherein at least one of the hydrogen of

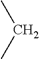

may be substituted with halogen), or

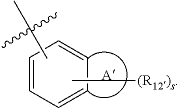

R_{12′} may be each independently C₁-C₁₀ linear or branched alkyl, —CX₃ (wherein X is halogen) or —OH,
s may be an integer of 0 or more and 5 or less,
A′ may be C₆-C₁₂ aryl or C₂-C₁₀ heterocycloalkyl including at least one of N, O and S as a heteroatom in a ring,
R_{4′} to R_{6′} and R_{8′} may be the same as defined in formula 3.

(11) In (7), (8), (9) or (10), R_{1′} may be chlorine,

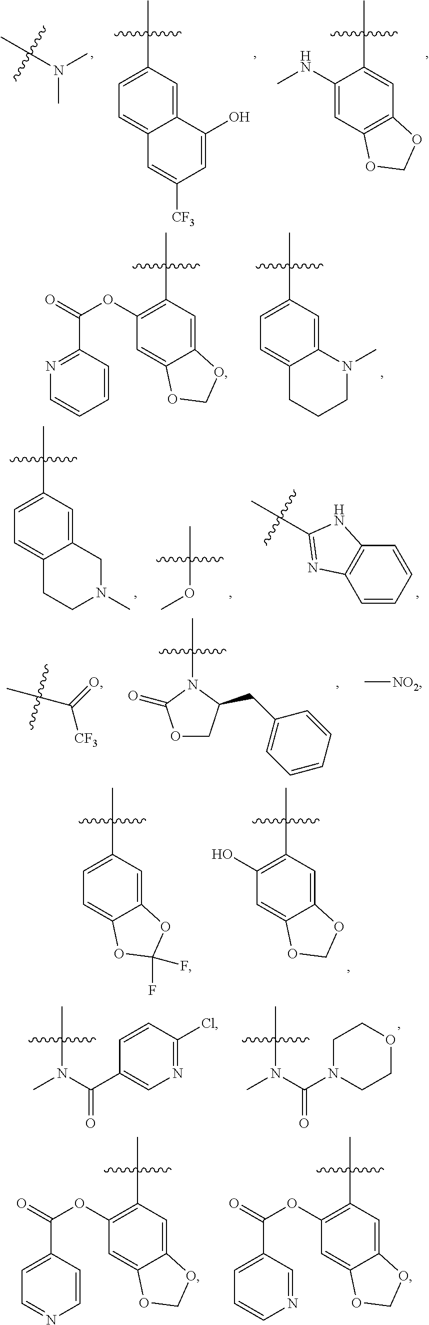

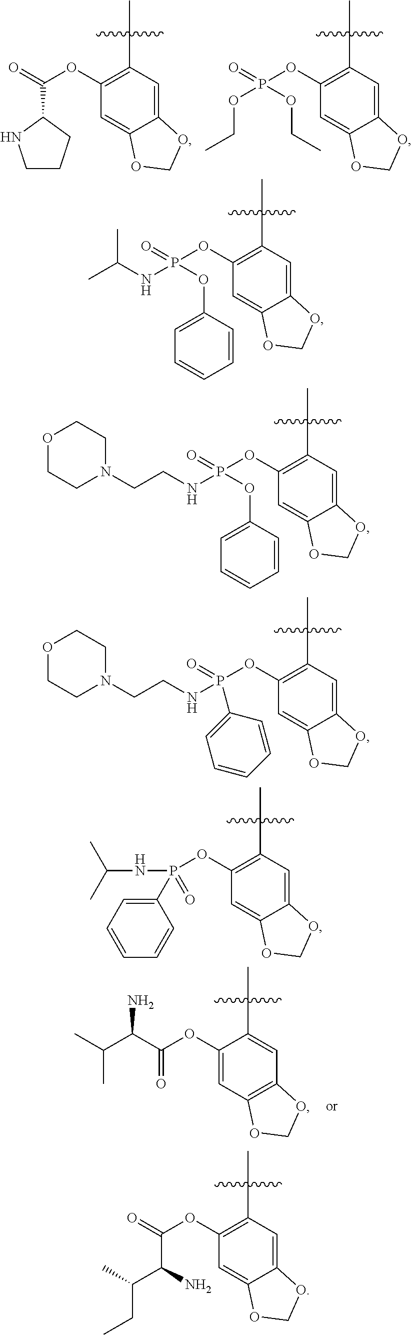

(12) In (7), (8), (9), (10) or (11), the compound represented by above formula 3 may be one selected from the group consisting of the following compounds:
[145] 9-(4-(2-hydroxyethyl) piperazin-1-yl)-6,7-dimethoxynaphtho[2,3-c]furan-1(3H)-one
[153] 9-(8-hydroxy-6-(trifluoromethyl) naphthalen-2-yl)-6,7-dimethoxynaphtho[2,3-c]furan-1(3H)-one
[169] 6-(6,7-dimethoxy-3-oxo-1,3-dihydronaphtho[2,3-c]furan-4-yl)benzo[d][1,3]dioxol-5-yl picolate
[170] 6,7-dimethoxy-9-(1-methyl-1,2,3,4-tetrahydroquinolin-7-yl)naphtho[2,3-c]furan-1(3H)-one
[190] 6,7-dimethoxy-9-(2-methyl-1,2,3,4-tetrahydroisoquinolin-7-yl)naphtho[2,3-c]furan-1(3H)-one
[201] 9-(6-chloro-5-(dimethylamino)pyridin-3-yl)-6,7-dimethoxynaphtho[2,3-c]furan-1(3H)-one
[204] 9-(5-(1H-benzo[d]imidazol-2-yl)-6-methoxypyridin-3-yl)-6,7-dimethoxynaphtho[2,3-c]furan-1(3H)-one
[210] 6,7-dimethoxy-9-(4-(2,2,2-trifluoroacetyl)phenyl)naphtho[2,3-c]furan-1(3H)-one
[211] (S)-4-benzyl-3-(5-(6,7-dimethoxy-3-oxo-1,3-dihydronaphtho[2,3-c]furan-4-yl)pyridin-2-yl) oxazolidin-2-one
[207] 9-(4-(dimethylamino)-3-nitrophenyl)-6,7-dimethoxynaphtho[2,3-c]furan-1(3H)-one
[221] 9-(2,2-difluorobenzo[d][1,3]dioxol-5-yl)-6,7-dimethoxynaphtho[2,3-c]furan-1(3H)-one
[227] 9-(4-(5-bromopyrimidin-2-yl) piperazin-1-yl)-6,7-dimethoxynaphtho[2,3-c]furan-1(3H)-one
[234] 6-chloro-N-(5-(6,7-dimethoxy-3-oxo-1,3-dihydronaphtho[2,3-c]furan-4-yl)pyridin-2-yl)-N-methylnicotinamide
[235] N-(5-(6,7-dimethoxy-3-oxo-1,3-dihydronaphtho[2,3-c]furan-4-yl)pyridin-2-yl)-N-methylmorpholin-4-carboxamide
[259] 4-(2,2-difluorobenzo[d][1,3]dioxol-5-yl)-6,7,9-trimethoxynaphtho[2,3-c]furan-1(3H)-one
[260] 4-(6-(1,4-dioxa-8-azaspiro[4.5]decan-8-yl)pyridin-3-yl)-6,7,9-trimethoxynaphtho[2,3-c]furan-1(3H)-one
[261] 4-(6-(butyl(methyl)amino)pyridin-3-yl)-6,7,9-trimethoxynaphtho[2,3-c]furan-1(3H)-one
[262] 4-(benzo[d][1,3]dioxol-5-yl)-6,7,9-trimethoxynaphtho[2,3-c]furan-1(3H)-one
[263] 6,7,9-trimethoxy-4-(6-(methyl(3-(trifluoromethyl)phenyl)amino)pyridin-3-yl)naphtho[2,3-c]furan-1(3H)-one
[264] 3-(dimethylamino)-1-(5-(6,7,9-trimethoxy-1-oxo-1,3-dihydronaphtho[2,3-c]furan-4-yl)pyridin-2-yl)-1H-pyrazol-4-carbonitrile
[265] 6,7,9-trimethoxy-4-(6-((2-methoxyethyl)(methyl)amino)pyridin-3-yl)naphtho[2,3-c]furan-1(3H)-one
[266] (S)-6,7,9-trimethoxy-4-(6-((5-methoxy-1,2,3,4-tetrahydronaphthalen-2-yl)(methyl)amino)pyridin-3-yl)naphtho[2,3-c]furan-1(3H)-one
[267] 6,7,9-trimethoxy-4-(6-morpholinopyridin-3-yl)naphtho[2,3-c]furan-1(3H)-one
[268] 4-(6-((2,3-dihydro-1H-indan-2-yl)(methyl)amino)pyridin-3-yl)-6,7,9-trimethoxynaphtho[2,3-c]furan-1(3H)-one
[269] 6-(6,7-dimethoxy-3-oxo-1,3-dihydronaphtho[2,3-c]furan-4-yl)benzo[d][1,3]dioxol-5-yl L-valinate hydrochloride
[270] 6-(6,7-dimethoxy-3-oxo-1,3-dihydronaphtho[2,3-c]furan-4-yl)benzo[d][1,3]dioxol-5-yl L-isoleucinate
[271] 6-(6,7-dimethoxy-3-oxo-1,3-dihydronaphtho[2,3-c]furan-4-yl)benzo[d][1,3]dioxol-5-yl isonicoinate
[272] 6-(6,7-dimethoxy-3-oxo-1,3-dihydronaphtho[2,3-c]furan-4-yl)benzo[d][1,3]dioxol-5-yl nicotinate
[273] 6-(6,7-dimethoxy-3-oxo-1,3-dihydronaphtho[2,3-c]furan-4-yl)benzo[d][1,3]dioxol-5-yl L-prolinate
[277] 6-(6,7-dimethoxy-3-oxo-1,3-dihydronaphtho[2,3-c]furan-4-yl)benzo[d][1,3]dioxol-5-yl diethyl phosphate
[278] 6-(6,7-dimethoxy-3-oxo-1,3-dihydronaphtho[2,3-c]furan-4-yl)benzo[d][1,3]dioxol-5-yl phenyl isopropylphosphoramidate
[279] 6-(6,7-dimethoxy-3-oxo-1,3-dihydronaphtho[2,3-c]furan-4-yl)benzo[d][1,3]dioxol-5-yl phenyl (2-morpholinoethyl)phosphoramidate
[280] 6-(6,7-dimethoxy-3-oxo-1,3-dihydronaphtho[2,3-c]furan-4-yl)benzo[d][1,3]dioxol-5-yl N-(2-morpholinoethyl)-P-phenylphosphoramidate
[281] 6-(6,7-dimethoxy-3-oxo-1,3-dihydronaphtho[2,3-c]furan-4-yl)benzo[d][1,3]dioxol-5-yl N-isopropyl-P-phenylphosphoramidate

(13) The present invention may provide a compound below, a pharmaceutically acceptable salt thereof, a stereoisomer thereof, a hydrate thereof or a solvate thereof:
[212] 1-(6-hydroxybenzo[d][1,3]dioxol-5-yl)-3-(hydroxymethyl)-6,7-dimethoxy-2-naphthoate sodium (I)
[224] 1-(6-hydroxybenzo[d][1,3]dioxol-5-yl)-3-(hydroxymethyl)-6,7-dimethoxy-2-naphthoyl chloride
[228] 1-(6-hydroxybenzo[d][1,3]dioxol-5-yl)-3-(hydroxymethyl)-N-isopropyl-6,7-dimethoxy-2-naphthamide

(14) In (1), (2), (3), (4), (5), (6), (7), (8), (9), (10), (11), (12) or (13), there may be provided the compound, the pharmaceutically acceptable salt thereof, the stereoisomer thereof, the hydrate thereof or the solvate thereof, wherein the salt is acid addition salt formed by pharmaceutically acceptable free acid.

(15) In (14), the free acid may be selected from the group consisting of citric acid, acetic acid, lactic acid, tartaric acid, maleic acid, fumaric acid, formic acid, propionic acid, oxalic acid, trifluoroacetic acid, benzoic acid, gluconic acid, methanesulfonic acid, glycolic acid, succinic acid, 4-toluenesulfonic acid, camphorsulfonic acid, glutamic acid, aspartic acid, silicylic acid, malonic acid, malic acid, benzenesulfonic acid, hydrochloric acid, bromic acid, nitric acid, sulfuric acid, and phosphoric acid.

(16) The present invention may provide a pharmaceutical composition for an anti-viral purpose including the compound, the pharmaceutically acceptable salt thereof, the stereoisomer thereof, the hydrate thereof or the solvate thereof according to (1), (2), (3), (4), (5), (6), (7), (8), (9), (10), (11), (12), (13), (14) or (15) as an active ingredient.

(17) In (16), the composition may be an anti-viral composition for one or more of SARS-COV-2, influenza virus, avian influenza virus, dengue virus, Zika virus, and severe fever with thrombocytopenia syndrome virus (SFTS).

(18) The present invention may provide a pharmaceutical composition for preventing or treating a virus infection disease, including the compound, the pharmaceutically acceptable salt thereof, the stereoisomer thereof, the hydrate thereof or the solvate thereof according to (1), (2), (3), (4), (5), (6), (7), (8), (9), (10), (11), (12), (13), (14) or (15) as an active ingredient.

(19) In (18), the virus infection disease may be one or more of SARS-COV-2 infection disease, influenza virus infection disease, avian influenza virus infection disease, dengue virus infection disease, Zika virus infection disease, and severe fever with thrombocytopenia syndrome virus infection disease.

(20) In (18), the virus infection disease may be COVID-19. In (19), the SARS-COV-2 infection disease may be COVID-19.

(21) In (18), (19) or (20), the composition may inhibit intracellular infection and proliferation of one or more of SARS-COV-2, influenza virus, avian influenza virus, dengue virus, Zika virus, and severe fever with thrombocytopenia syndrome virus.

(22) The present invention may provide a pharmaceutical composition for preventing or treating a respiratory disease, including the compound, the pharmaceutically acceptable salt thereof, the stereoisomer thereof, the hydrate thereof or the solvate thereof according to (1), (2), (3), (4), (5), (6), (7), (8), (9), (10), (11), (12), (13), (14) or (15) as an active ingredient.

(23) In (21), the respiratory disease may be at least one selected from the group consisting of asthma, cold, pneumonia, bronchitis, chronic obstructive pulmonary disease and rhinitis.

(24) In (22) or (23), the composition may inhibit the expression of interleukin-5.

(25) The present invention may provide a pharmaceutical composition for preventing or treating an allergic disease, including the compound, the pharmaceutically acceptable salt thereof, the stereoisomer thereof, the hydrate thereof or the solvate thereof according to (1), (2), (3), (4), (5), (6), (7), (8), (9), (10), (11), (12), (13), (14) or (15) as an active ingredient.

(26) In (25), the allergic disease may be at least one selected from the group consisting of rhinitis, asthma, atopic dermatitis, allergic conjunctivitis, allergic otitis media, allergic gastrointestinal infection, anaphylaxis and hives.

(27) The present invention may provide a method for preventing or treating a virus infection disease, which includes administering the compound, the pharmaceutically acceptable salt thereof, the stereoisomer thereof, the hydrate thereof or the solvate thereof according to (1), (2), (3), (4), (5), (6), (7), (8), (9), (10), (11), (12), (13), (14) or (15) into a subject in need thereof.

(28) The present invention may provide a use of the compound, the pharmaceutically acceptable salt thereof, the stereoisomer thereof, the hydrate thereof or the solvate thereof according to (1), (2), (3), (4), (5), (6), (7), (8), (9), (10), (11), (12), (13), (14) or (15) for preventing or treating a virus infection disease.

(29) The present invention may provide a use of the compound, the pharmaceutically acceptable salt thereof, the stereoisomer thereof, the hydrate thereof or the solvate thereof according to (1), (2), (3), (4), (5), (6), (7), (8), (9), (10), (11), (12), (13), (14) or (15) for manufacturing a medicament for preventing or treating a virus infection disease.

(30) The present invention may provide a method for preventing or treating a respiratory disease, which includes administering the compound, the pharmaceutically acceptable salt thereof, the stereoisomer thereof, the hydrate thereof or the solvate thereof according to (1), (2), (3), (4), (5), (6), (7), (8), (9), (10), (11), (12), (13), (14) or (15) into a subject in need thereof.

(31) The present invention may provide a use of the compound, the pharmaceutically acceptable salt thereof, the stereoisomer thereof, the hydrate thereof or the solvate thereof according to (1), (2), (3), (4), (5), (6), (7), (8), (9), (10), (11), (12), (13), (14) or (15) for preventing or treating a respiratory disease.

(32) The present invention may provide a use of the compound, the pharmaceutically acceptable salt thereof, the stereoisomer thereof, the hydrate thereof or the solvate thereof according to (1), (2), (3), (4), (5), (6), (7), (8), (9), (10), (11), (12), (13), (14) or (15) for manufacturing a medicament for preventing or treating a respiratory disease.

(33) The present invention may provide a method for preventing or treating an allergic disease, which includes administering the compound, the pharmaceutically acceptable salt thereof, the stereoisomer thereof, the hydrate thereof or the solvate thereof according to (1), (2), (3), (4), (5), (6), (7), (8), (9), (10), (11), (12), (13), (14) or (15) into a subject in need thereof.

(31) The present invention may provide a use of the compound, the pharmaceutically acceptable salt thereof, the stereoisomer thereof, the hydrate thereof or the solvate thereof according to (1), (2), (3), (4), (5), (6), (7), (8), (9), (10), (11), (12), (13), (14) or (15) for preventing or treating an allergic disease.

(32) The present invention may provide a use of the compound, the pharmaceutically acceptable salt thereof, the stereoisomer thereof, the hydrate thereof or the solvate thereof according to (1), (2), (3), (4), (5), (6), (7), (8), (9), (10), (11), (12), (13), (14) or (15) for manufacturing a medicament for preventing or treating an allergic disease.

The numerical values described in the present specification as above should be interpreted to include a range of equivalents thereof, unless otherwise stated.

Matters mentioned in each of the composition, use; and preventive and/or therapeutic method according to the present invention are applied the same, if not contradictory to each other.

### Advantageous Effects

The 6,7-dimethoxynaphtho[2,3-c]furan-1(3H)-one compound according to one example of the present invention can exhibit an effect of inhibiting the intracellular infection and proliferation of various viruses including SARS-COV-2, thereby showing an excellent effect of preventing or treating a wide range of virus infection diseases. In addition, the compound according to one example can have an excellent effect of inhibiting interleukin-5 mainly involved in the induction of allergy and asthma, thereby providing an excellent effect of preventing or treating a respiratory disease.

### DESCRIPTION OF DRAWINGS

FIGS. 1 to 18 are data on SARS-COV-2 virus inhibitory ability and cytotoxicity of the example compounds (in FIGS. 1 to 18, ▴ represents CC₅₀ and ● represents IC₅₀).

FIG. 19 is data showing 60 substances which inhibit IL-4 secretion by 75% or more in the example compounds.

FIG. 20 is data showing the IL-4 secretion inhibitory ability when RBL-2H3 cells are treated with the finally selected eight example compounds at 10 and 3 uM.

FIG. 21 is data showing the eosinophil reducing ability of an example compound in an Balb/c mouse asthma model.

FIG. 22 is data showing the IgE, IL-4, and IL-5 secretion inhibitory activity of the example compounds in the Balb/c mouse asthma model.

### MODE FOR INVENTION

Hereinafter, the present invention will be described in more detail through preparation examples and examples. However, these preparation examples and examples are provided only for the purpose of illustrating the present invention, and thus the present invention is not limited thereto.

Furthermore, the present invention may include the compound represented by above formula 1, the compound represented by compound 3, the compound of [212], the compound of [224], or the compound of [228]; and the pharmaceutically acceptable salt thereof; as well as the solvate; the stereoisomer; the hydrate, and the like, which may be prepared therefrom.

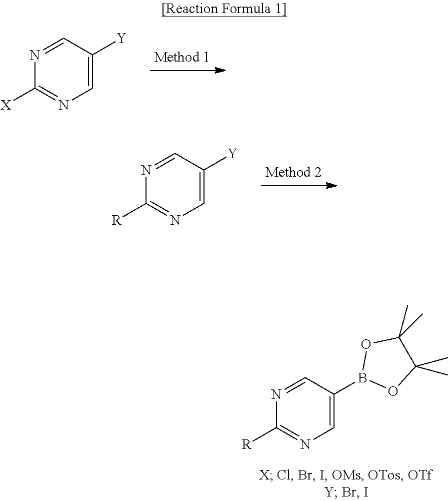

In Method 1, an R group may be introduced into a pyrimidine compound substituted with X by using anhydrous potassium carbonate, anhydrous sodium carbonate, sodium hydride, etc., in an organic solvent such as acetonitrile, tetrahydrofuran, dimethylformamide, dioxane, etc. R may be defined in the same manner as in R₁ described above.

In Reaction Formula 1, the pyrimidine compound may be replaced with an aryl group or a heteroaryl group and, for example, the pyrimidine compound may be replaced with the above-described —(Ar₁—R₁), etc.

In Method 2, an intermediate compound was synthesized through a Suzuki-Miyaura coupling reaction as shown in Reaction Formula 2 below. Borate was synthesized by heating and stirring a dichloromethane complex of Pd(dppf)Cl₂ and potassium acetate in a dioxane solvent at 60-110° C. for 3 to 48 hours.

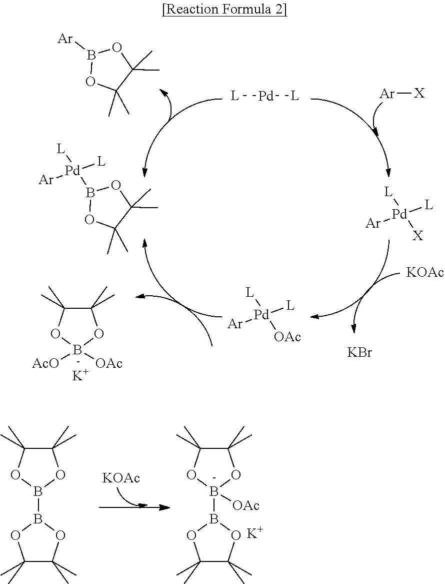

Ar of Method 2 may be —(Ar₁—R₁) in formula 1 described above.

In order to synthesize a target material, the compound synthesized in above Reaction Formula 2 was subjected to Suzuki coupling as shown in Reaction Formula 3 below, so as to synthesize compound 1 as the target material.

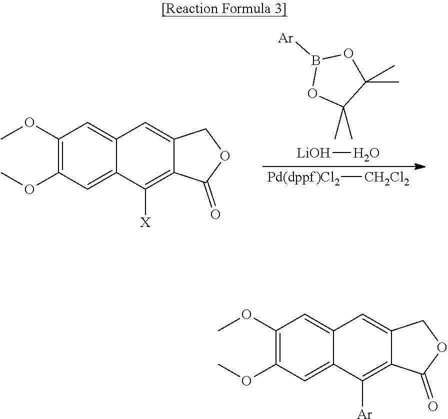

X: OTf, Br, I, Oms, OTs

In Reaction Formula 3, a compound in which X is OTf was mainly used. The reactants, Pd(dppf)Cl₂—CH₂Cl₂, and lithium hydroxide hydrate were heated in a dioxane solvent at 80-110° C. for 8 to 48 hours to synthesize the compound of one Example.

Hereinafter, the present invention will be described in more detail through examples. The following Examples are provided only for the purpose of illustrating the present invention, and thus the scope of the present invention is not limited thereto.

### Preparation of Intermediate Compound

An intermediate was synthesized according to Reaction Formula 1 described above.

### Intermediate Synthesis Example 1: Synthesis of 4-(2-((5-bromopyridi-2-yl)oxy)ethyl)morpholine

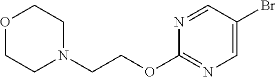 [SMILES: Brc1cnc(OCCN2CCOCC2)nc1]

After 2 g (10.33 mmol) of 5-bromo-2-chloro pyrimidine and 4.07 g (31.02 mmol) of 2-morpholinoethan-1-ol were dissolved in 30 mL of tetrahydrofuran, 1.24 g (28.41 mmol) of sodium hydride was added thereto at 20° C. A reaction temperature was raised up to 25° C. and reacted for five hours. The reaction was quenched by addition of 50 mL of purified water and extracted with DCM (dichloromethane 3×30 mL). The organic layer was washed with brine (2×10 mL), dried over anhydrous Na₂SO₄, filtered, and concentrated. The residue was recrystallized with DCM/hexane to obtain 1.884 g (6.53 mmol, 63.2%) of 4-(2-((5-bromopyridi-2-yl)oxy)ethyl)morpholine as a title compound. Mass (M+H⁺): 290.0

### Intermediate Synthesis Example 2: Synthesis of 5-bromo-N-(cyclopropylmethyl)pyrimidine-2-amine

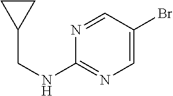 [SMILES: Brc1cnc(NCC2CC2)nc1]

After 2 g (10.33 mmol) of 5-bromo-2-chloro pyrimidine and 0.808 g (11.36 mmol) of cyclopropyl methylamine were dissolved in 30 mL of acetonitrile, 4.3 g (31.11 mmol) of anhydrous potassium carbonate was added thereto at 20° C. The resulting mixture was heated and stirred under reflux for eight hours. After cooling, the residual inorganic material was filtered, removed, and distilled under reduced pressure. After 15 mL of purified water was added to the residue, the resulting mixture was extracted twice with 15 ml of dichloromethane, dried over anhydrous sodium sulfate, filtered, washed, and distilled under reduced pressure to obtain 2.32 g (10.17 mmol, 98.3%) of 5-bromo-N-(cyclopropylmethyl)pyrimidin-2-amine. Mass (M+H⁺): 230.0

### Intermediate Synthesis Example 3: Synthesis of 5-bromo-N-(cyclopropylmethyl)-N-methylpyrimidin-2-amine

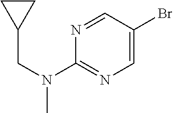

After 1.2 g (5.26 mmol) of 5-bromo-N-(cyclopropylmethyl)pyrimidin-2-amine was dissolved in 30 mL of tetrahydrofuran, 1.49 g (10.49 mmol) of iodomethane was added at 20° C. After a reaction temperature was lowered to 0° C., 0.918 g (21.04 mmol) of sodium hydride was added thereto, and the reaction was performed at the same temperature for five hours. After 20 mL of purified water was added to complete the reaction and perform distillation under reduced pressure, 20 ml of purified water was added to the residue and extracted with dichloromethane (DCM)(3×30 mL). The organic layer was washed with brine (2×10 mL), dried over anhydrous Na₂SO₄, filtered, and distilled under reduced pressure to obtain 1.25 g (5.16 mmol, 98.4%) of 5-bromo-N-(cyclopropylmethyl)-N-methylpyrimidine-2-amine as a title compound. Mass (M+H⁺): 244.1

### Intermediate Synthesis Example 4: Synthesis of N-(cyclopropylmethyl)-N-methyl-5-(4,4,5,5-tetramethyl-1,3,2-dioxaborolan-2-yl)pyrimidin-2-amine

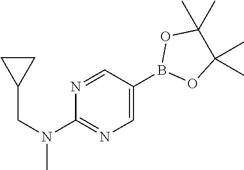 [SMILES: CN(CC1CC1)c1ncc(B2OC(C)(C)C(C)(C)O2)cn1]

After 1.2 g (4.95 mmol) of 5-bromo-N-(cyclopropylmethyl)-N-methylpyrimidin-2-amine synthesized in Intermediate Synthesis Example 3 was dissolved in 20 ml of dioxane, 1.5 g (5.90 mmol) of bis(panacolato)diborane, 1.46 g (14.87 mmol) of KOAc, and 0.4 g (0.48 mmol) of Pd(dppf)Cl₂—CH₂Cl₂ were added thereto. After bubbling the reaction solvent with nitrogen, a reaction temperature was raised up to 80° C., and then the reaction was performed for three hours. After cooling to 25° C., 20 ml of ethyl acetate was injected and then stirred. The reaction solution was passed through a celite pad, filtered and distilled under reduced pressure. The residue was purified by silica gel column chromatography to obtain 1.2 g (4.14 mmol, 83.7%) of N-(cyclopropylmethyl)-N-methyl-5-(4,4,5,5-tetramethyl-1,3,2-dioxaborolan-2-yl)pyrimidin-2-amine as a title compound. Mass (M+H⁺): 289.2

### Preparation of Example Compound

Intermediates used in the following examples were prepared by using intermediates prepared similarly to the method for preparing intermediates described above.

### [Example 1] Synthesis of (S)-6,7-dimethoxy-9-((5-methoxy-1,2,3,4-tetrahydronaphthalen-2-yl)amino)naphtho[2,3-c]furan-1(3H)-one

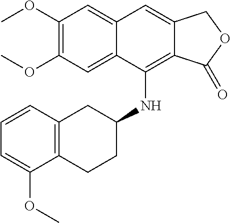 [SMILES: COc1cc2cc3c(c(N[C@H]4CCc5c(cccc5OC)C4)c2cc1OC)C(=O)OC3]

After 0.5 g (1.27 mmol) of 6,7-dimethoxy-3-oxo-1,3-dihydronaphtho[2,3-c]furan-4-yl trifluoromethanesulfonate was dissolved in 30 ml of 1,4-dioxane, 0.42 g (1.27 mmol) of(S)-2-amino-5-methoxytetralin(S)-mandelate salt and 0.528 g (3.82 mmol) of anhydrous potassium carbonate were added thereto, after which a temperature was raised up to 95° C. and then the resulting mixture was reacted for 15 hours. After cooling down to 25° C., 30 ml of ethyl acetate (EA) was added thereto, and the resulting mixture was stirred. After filtration and washing with ethyl acetate, distillation was performed under reduced pressure, and the residue was purified by silica gel column chromatography to obtain 275 mg (0.65 mmol, 51.6%) of(S)-6,7-dimethoxy-9-((5-methoxy-1,2,3,4-tetrahydronaphthalen-2-yl)amino)naphtho[2,3-c]furan-1(3H)-one as a title compound.

Mass (M+H⁺): 420.2

¹H-NMR (500 Mhz, DMSO-d6): 0.82-0.83 (m, 1H), 1.20 (s, 1H), 1.70-1.75 (m, 1H), 2.10-2.15 (m, 1H), 2.74-2.82 (m, 2H), 3.08-3.09 (m, 1H), 3.71 (s, 3H), 3.83 (s, 3H), 3.87 (s, 3H), 4.21-4.25 (m, 1H), 5.31 (s, 2H), 6.35 (d, J=4.45 Hz, 1H), 6.64 (d, J=7.75 Hz, 1H), 6.71 (d, J=8.05 Hz, 1H), 7.04 (t, J=8.0 Hz, 1H), 7.20 (s, 1H), 7.29 (s, 1H), 7.47 (s, 1H)

### [Example 2] Synthesis of 9-((2S,6R)-2,6-dimethylmorpholino)-6,7-dimethoxynaphtho[2,3-c]furan-1(3H)-one

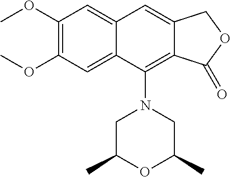 [SMILES: COc1cc2cc3c(c(N4C[C@@H](C)O[C@@H](C)C4)c2cc1OC)C(=O)OC3]

After 1.0 g (2.54 mmol) of 6,7-dimethoxy-3-oxo-1,3-dihydronaphtho[2,3-c]furan-4-yl trifluoromethanesulfonate was dissolved in 30 ml of acetonitrile, 0.322 g (2.79 mmol) of cis-dimethylmorpholine and 0.80 g (5.78 mmol) of anhydrous potassium carbonate were added thereto, after which a temperature was raised up to 80° C. and then the resulting mixture was reacted for 20 hours. After cooling down to 25° C., 30 ml of ethyl acetate was added thereto, and the resulting mixture was stirred. After filtration and washing with ethyl acetate, distillation was performed under reduced pressure, and the residue was purified by silica gel column chromatography to obtain 511 mg (1.42 mmol, 55.9%) of 9-((2S,6R)-2,6-dimethylmorpholino)-6,7-dimethoxynaphtho[2,3-c]furan-1(3H)-one as a title compound.

Mass (M+H⁺): 358.2

¹H-NMR (500 Mhz, DMSO-d6): δ 1.08 (s, 6H), 2.86 (d, J=11.15 Hz, 2H), 3.27-3.29 (m, 2H), 3.89-3.91 (m, 8H), 5.35 (s, 2H), 7.37 (s, 1H), 7.64 (s, 1H), 7.78 (s, 1H)

### [Example 3] Synthesis of 9-(6,7-dihydrothieno[3,2-c]pyridin-5 (4H)-yl)-6,7-dimethoxynaphtho[2,3-c]furan-1(3H)-one

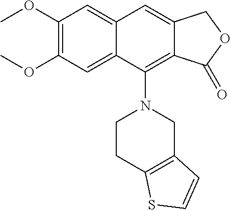 [SMILES: COc1cc2cc3c(c(N4CCc5sccc5C4)c2cc1OC)C(=O)OC3]

After 1.0 g (2.54 mmol) of 6,7-dimethoxy-3-oxo-1,3-dihydronaphtho[2,3-c]furan-4-yl trifluoromethanesulfonate was dissolved in 30 ml of acetonitrile, 0.49 g (2.79 mmol) of 4,5,6,7-tetrahydrothieno[3,2-c]pyridine hydrochloride and 1.056 g (7.64 mmol) of anhydrous potassium carbonate were added thereto, after which a temperature was raised up to 80° C. and then the resulting mixture was reacted for 2 days. After cooling down to 25° C., 30 ml of ethyl acetate was added thereto, and the resulting mixture was stirred. After filtration and washing with ethyl acetate, distillation was performed under reduced pressure, and the residue was purified by silica gel column chromatography to obtain 353 mg (0.92 mmol, 36.2%) of 9-((2S,6R)-2,6-dimethylmorpholino)-6,7-dimethoxynaphtho[2,3-c]furan-1(3H)-one as a title compound.

Mass (M+H⁺): 381.45

¹H-NMR (500 Mhz, DMSO-d6): δ 2.94 (s, 2H), 3.50-3.57 (m, 2H), 3.64 (s, 3H), 3.84 (s, 3H), 4.24-4.41 (m, 2H), 5.36 (s, 2H), 6.78 (d, J=4.9 Hz, 1H), 7.27 (d, 1H), 7.34 (s, 1H), 7.57 (s, 1H), 7.63 (s, 1H)

### [Example 4] Synthesis of 9-(3,5-dimethylpiperidin-1-yl)-6,7-dimethoxynaphtho[2,3-c]furan-1(3H)-one

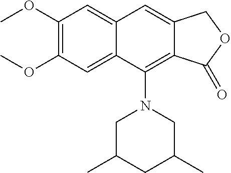 [SMILES: COc1cc2cc3c(c(N4CC(C)CC(C)C4)c2cc1OC)C(=O)OC3]

After 1.0 g (2.54 mmol) of 6,7-dimethoxy-3-oxo-1,3-dihydronaphtho[2,3-c]furan-4-yl trifluoromethanesulfonate was dissolved in 30 ml of acetonitrile, 0.317 g (2.79 mmol) of 3,5-methylpiperidine and 1.056 g (7.64 mmol) of anhydrous potassium carbonate were added thereto, after which a temperature was raised up to 80° C. and then the resulting mixture was reacted for two days. After cooling down to 25° C., 30 ml of ethyl acetate was added thereto, and the resulting mixture was stirred. After filtration and washing with ethyl acetate, distillation was performed under reduced pressure, and the residue was purified by silica gel column chromatography to obtain 690 mg (1.94 mmol, 76.3%) of 9-(3,5-dimethylpiperidin-1-yl)-6,7-dimethoxynaphtho[2,3-c]furan-1(3H)-one as a title compound.

Mass (M+H⁺): 356.2

¹H-NMR (500 Mhz, DMSO-d6): δ 0.73-0.81 (m, 6H), 1.85-1.87 (m, 4H), 2.91-2.92 (m, 2H), 3.01 (t, J=10.9 Hz, 2H), 3.77 (s, 3H), 3.84 (s, 3H), 5.31 (s, 2H), 7.30 (s, 1H), 7.51 (s, 1H), 7.61 (s, 1H)

### [Example 5] Synthesis of 6,7-dimethoxy-9-((2-methoxyethyl)(methyl)amino)naphtho[2,3-c]furan-1(3H)-one

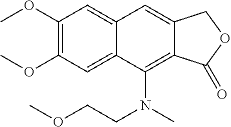 [SMILES: COCCN(C)c1c2c(cc3cc(OC)c(OC)cc13)COC2=O]

After 1.0 g (2.54 mmol) of 6,7-dimethoxy-3-oxo-1,3-dihydronaphtho[2,3-c]furan-4-yl trifluoromethanesulfonate was dissolved in 30 ml of acetonitrile, 0.249 g (2.79 mmol) of 2-methoxy-N-methylethan-1-amine and 1.056 g (7.64 mmol) of anhydrous potassium carbonate were added thereto, after which a temperature was raised up to 80° C. and then the resulting mixture was reacted for three days. After cooling down to 25° C., 30 ml of ethyl acetate was added thereto, and the resulting mixture was stirred. After filtration and washing with ethyl acetate, distillation was performed under reduced pressure, and the residue was purified by silica gel column chromatography to obtain 460 mg (1.38 mmol, 54.3%) of 6,7-dimethoxy-9-((2-methoxyethyl)(methyl)amino)naphtho[2,3-c]furan-1(3H)-one as a title compound.

Mass (M+H⁺): 332.2

¹H-NMR (500 Mhz, DMSO-d6): δ 2.92 (s, 3H), 3.12 (s, 3H), 3.23 (s, 4H), 3.82 (s, 3H), 3.84 (s, 3H), 5.32 (s, 2H), 7.30 (s, 1H), 7.58 (s, 1H), 7.86 (s, 1H)

### [Example 6] Synthesis of 9-((2-(diethylamino)ethyl)amino)-6,7-dimethoxynaphtho[2,3-c]furan-1(3H)-one

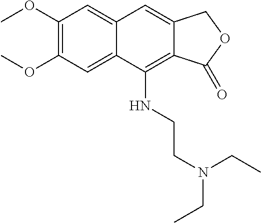 [SMILES: CCN(CC)CCNc1c2c(cc3cc(OC)c(OC)cc13)COC2=O]

After 1.0 g (2.54 mmol) of 6,7-dimethoxy-3-oxo-1,3-dihydronaphtho[2,3-c]furan-4-yl trifluoromethanesulfonate was dissolved in 30 ml of acetonitrile, 0.325 g (2.79 mmol) of N1,N1-diethylethan-1,2-diamine and 1.056 g (7.64 mmol) of anhydrous potassium carbonate were added thereto, after which a temperature was raised up to 80° C. and then the resulting mixture was reacted for one day. After cooling down to 25° C., 30 ml of ethyl acetate was added thereto, and the resulting mixture was stirred. After filtration and washing with ethyl acetate, distillation was performed under reduced pressure, and the residue was purified by silica gel column chromatography to obtain 505 mg (1.40 mmol, 55.1%) of 9-((2-(diethylamino)ethyl)amino)-6,7-dimethoxynaphtho[2,3-c]furan-1(3H)-one as a title compound.

Mass (M+H⁺): 359.2

¹H-NMR (500 Mhz, DMSO-d6): δ 0.85 (t, J=7.15 Hz, 6H), 2.39-2.42 (m, 4H), 2.54 (t, J=6.0 Hz, 2H), 3.65-3.68 (m, 2H), 3.82 (d, J=2.3 Hz, 6H), 5.21 (s, 2H), 6.86-6.88 (m, 1H), 7.00 (s, 1H), 7.19 (s, 1H), 7.49 (s, 1H)

### [Example 7] Synthesis of 6,7-dimethoxy-9-(2-(4-methylpiperazin-1-yl)pyrimidin-5-yl)naphtho[2,3-c]furan-1(3H)-one

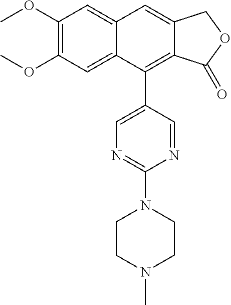 [SMILES: COc1cc2cc3c(c(-c4cnc(N5CCN(C)CC5)nc4)c2cc1OC)C(=O)OC3]

After 1.13 g of 6,7-dimethoxy-3-oxo-1,3-dihydronaphtho[2,3-c]furan-4-yl trifluoromethanesulfonate was dissolved in 30 ml of 1,4-dioxane, lithium hydroxy hydrate and Pd(dppf)Cl₂, and CH₂Cl₂ were added thereto and stirred. After 2-(4-methylpiperazin-1-yl)-5-(4,4,5,5-tetramethyl-1,3,2-dioxaborolan-2-yl)pyrimidine was added, nitrogen bubbling was performed on the solvent to remove oxygen and substitute nitrogen, after which a temperature was raised up to 60° C. and the resulting mixture was reacted for four hours. After cooling down to 25° C., 30 ml of ethyl acetate was added thereto, and the resulting mixture was stirred. After filtration and washing with ethyl acetate, distillation was performed under reduced pressure, and the residue was purified by silica gel column chromatography to obtain 370 mg (0.71 mmol, 52%) of 6,7-dimethoxy-9-(2-(4-methylpiperazin-1-yl)pyrimidin-5-yl)naphtho[2,3-c]furan-1(3H)-one as a title compound.

Mass (M+H⁺): 421.2

¹H-NMR (500 Mhz, CDCl3): 2.86-2.94 (m, 5H), 3.57 (d, J=12.5 Hz, 2H), 3.89-3.94 (m, 5H), 4.06 (s, 3H), 5.02 (d, J=12.05 Hz, 2H), 5.41 (s, 2H), 7.08 (s, 1H), 7.21 (s, 1H), 7.76 (s, 1H), 8.43 (s, 2H)

### [Example 8] Synthesis of 9-(2-((2S,6R)-2,6-dimethylmorpholino)pyrimidin-5-yl)-6,7-dimethoxynaphtho[2,3-c]furan-1(3H)-one

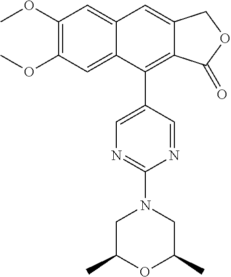 [SMILES: COc1cc2cc3c(c(-c4cnc(N5C[C@@H](C)O[C@@H](C)C5)nc4)c2cc1OC)C(=O)OC3]

The compound of Example 8 was synthesized by the same method as that of the compound of Example 7 except for using (2S,6R)-2,6-dimethyl-4-(5-(4,4,5,5-tetramethyl-1,3,2-dioxaborolan-2-yl)pyrimidin-2-yl) morpholine instead of 2-(4-methylpiperazin-1-yl)-5-(4,4,5,5-tetramethyl-1,3,2-dioxaborolan-2-yl)pyrimidine.

Mass (M+H⁺): 436.2

¹H-NMR (500 Mhz, DMSO-d6): δ 1.15 (t, J=13.75 Hz, 6H), 2.58-2.61 (m, 2H), 3.59-3.62 (m, 2H), 3.73 (s, 3H), 3.92 (s, 3H), 4.59 (d, J=11.45 Hz, 2H), 5.42 (s, 2H), 7.07 (s, 1H), 7.50 (s, 1H), 7.94 (s, 1H), 8.41 (s, 2H)

### [Example 9] Synthesis of 6,7-dimethoxy-9-(2-(piperidin-1-yl)pyrimidin-5-yl)naphtho[2,3-c]furan-1(3H)-one

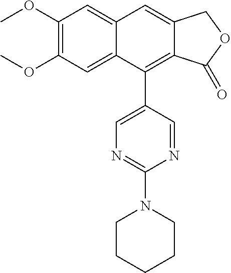 [SMILES: COc1cc2cc3c(c(-c4cnc(N5CCCCC5)nc4)c2cc1OC)C(=O)OC3]

The compound of Example 9 was synthesized by the same method as that of the compound of Example 7 except for using 2-(piperidin-1-yl)-5-(4,4,5,5-tetramethyl-1,3,2-dioxaborolan-2-yl)pyrimidine instead of 2-(4-methylpiperazin-1-yl)-5-(4,4,5,5-tetramethyl-1,3,2-dioxaborolan-2-yl)pyrimidine.

Mass (M+H⁺): 406.2

¹H-NMR (500 Mhz, CDCl₃): δ 1.68 (s, 6H), 3.88 (s, 7H), 4.03 (s, 3H), 5.36 (s, 2H), 7.19 (d J=6.3 Hz, 2H), 7.69 (s, 1H), 8.36 (s, 2H)

### [Example 10] Synthesis of 6,7-dimethoxy-9-(6-thiomorpholinopyridin-3-yl)naphtho[2,3-c]furan-1(3H)-one

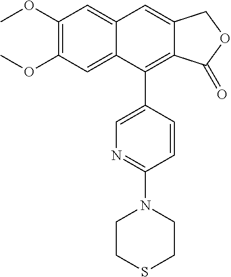 [SMILES: COc1cc2cc3c(c(-c4ccc(N5CCSCC5)nc4)c2cc1OC)C(=O)OC3]

The compound of Example 10 was synthesized by the same method as that of the compound of Example 7 except for using 4-(5-(4,4,5,5-tetramethyl-1,3,2-dioxaborolan-2-yl)pyridin-2-yl)thiomorpholine instead of 2-(4-methylpiperazin-1-yl)-5-(4,4,5,5-tetramethyl-1,3,2-dioxaborolan-2-yl)pyrimidine.

Mass (M+H⁺): 423.2

¹H-NMR (500 Mhz, CDCl₃): δ 2.76 (s, 4H), 3.85 (s, 3H), 4.04 (s, 3H), 4.07 (s, 4H), 5.37 (s, 2H), 7.13-7.25 (m, 4H), 7.70 (s, 1H), 8.22 (s, 1H)

### [Example 11] Synthesis of 9-(2-(dimethylamino)pyrimidin-5-yl)-6,7-dimethoxynaphtho[2,3-c]furan-1(3H)-one

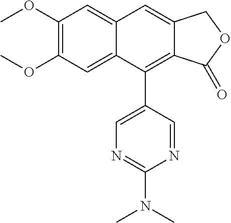 [SMILES: COc1cc2cc3c(c(-c4cnc(N(C)C)nc4)c2cc1OC)C(=O)OC3]

The compound of Example 11 was synthesized by the same method as that of the compound of Example 7 except for using N,N-dimethyl-5-(4,4,5,5-tetramethyl-1,3,2-dioxaborolan-2-yl)pyrimidin-2-amine instead of 2-(4-methylpiperazin-1-yl)-5-(4,4,5,5-tetramethyl-1,3,2-dioxaborolan-2-yl)pyrimidine.

Mass (M+H⁺): 366.1

¹H-NMR (500 Mhz, DMSO-d6): δ 3.13 (s, 6H), 3.66 (s, 3H), 3.86 (s, 3H), 5.36 (s, 2H), 7.01 (s, 1H), 7.44 (s, 1H), 7.87 (s, 1H), 8.31 (s, 2H)

### [Example 12] Synthesis of 9-(2-(6,7-dihydrothieno[3,2-c]pyridin-5 (4H)-yl)pyrimidin-5-yl)-6,7-dimethoxynaphtho[2,3-c]furan-1(3H)-one

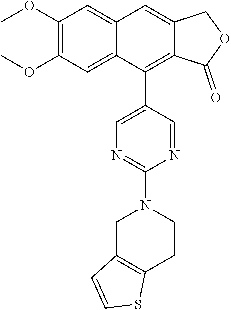 [SMILES: COc1cc2cc3c(c(-c4cnc(N5CCc6sccc6C5)nc4)c2cc1OC)C(=O)OC3]

The compound of Example 12 was synthesized by the same method as that of the compound of Example 7 except for using 5-(5-(4,4,5,5-tetramethyl-1,3,2-dioxaborolan-2-yl)pyrimidin-2-yl)-4,5,6,7-tetrahydrothieno[3,2-c]pyridine instead of 2-(4-methylpiperazin-1-yl)-5-(4,4,5,5-tetramethyl-1,3,2-dioxaborolan-2-yl)pyrimidine.

Mass (M+H⁺): 362.2

¹H-NMR (500 Mhz, CDCl3): 3.03 (t, J=5.7 Hz, 2H), 3.25 (s, 3H), 4.06 (s, 3H), 4.33 (br, 2H), 4.99 (s, 2H), 5.40 (s, 2H), 6.88 (d, J=5.15 Hz, 1H), 7.16 (t, J=5.15 Hz, 1H), 7.25 (s, 1H), 7.73 (s, 1H), 8.46 (s, 2H)

### [Example 13] Synthesis of 6,7-dimethoxy-9-(2-(2-morpholinoethoxy)pyrimidin-5-yl)naphtho[2,3-c]furan-1(3H)-one

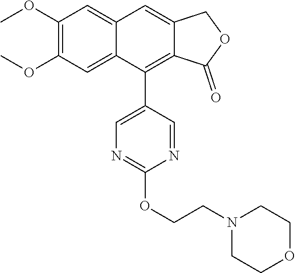 [SMILES: COc1cc2cc3c(c(-c4cnc(OCCN5CCOCC5)nc4)c2cc1OC)C(=O)OC3]

The compound of Example 13 was synthesized by the same method as that of the compound of Example 7 except for using 4-(2-((5-(4,4,5,5-tetramethyl-1,3,2-dioxaborolan-2-yl)pyrimidin-2-yl)oxy)ethyl)morpholine instead of 2-(4-methylpiperazin-1-yl)-5-(4,4,5,5-tetramethyl-1,3,2-dioxaborolan-2-yl)pyrimidine.

Mass (M+H⁺): 452.2

¹H-NMR (500 Mhz, CD3OD): 2.63-2.64 (m, 4H), 2.88 (t, J=5.45 Hz, 2H), 3.70 (t, J=4.75 Hz, 4H), 3.79 (s, 3H), 4.00 (s, 3H), 4.66 (t, J=5.45 Hz, 2H), 5.45 (s, 2H), 7.03 (s, 1H), 7.45 (s, 1H), 7.97 (s, 1H), 8.60 (s, 2H)

### [Example 14] Synthesis of 9-(2-((3,4-dimethylphenyl)amino)pyrimidin-5-yl)-6,7-dimethoxynaphtho[2,3-c]furan-1(3H)-one

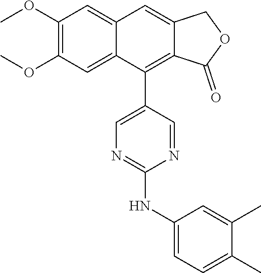

The compound of Example 14 was synthesized by the same method as that of the compound of Example 7 except for using N-(3,4-dimethylphenyl)-5-(4,4,5,5-tetramethyl-1,3,2-dioxaborolan-2-yl)pyrimidin-2-amine instead of 2-(4-methylpiperazin-1-yl)-5-(4,4,5,5-tetramethyl-1,3,2-dioxaborolan-2-yl)pyrimidine.

Mass (M+H⁺): 442.2

1H-NMR (500 Mhz, DMSO-d6): δ 2.15 (s, 3H), 2.18 (s, 3H), 3.74 (s, 3H), 3.93 (s, 3H), 5.44 (s, 2H), 7.03 (d, J=0.65 Hz, 1H), 7.09 (s, 1H), 7.52 (s, 2H), 7.56 (d, J=8.1 Hz, 1H), 7.96 (s, 1H), 8.48 (s, 2H), 9.63 (s, 1H)

### [Example 15] Synthesis of ethyl 1-(5-(6,7-dimethoxy-3-oxo-1,3-dihydronaphtho[2,3-c]furan-4-yl)pyrimidin-2-yl) piperidin-3-carboxylate

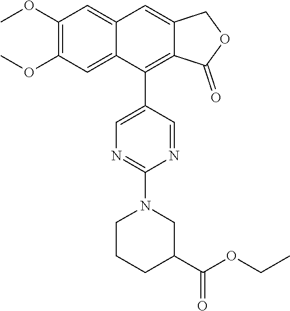 [SMILES: CCOC(=O)C1CCCN(c2ncc(-c3c4c(cc5cc(OC)c(OC)cc35)COC4=O)cn2)C1]

The compound of Example 15 was synthesized by the same method as that of the compound of Example 7 except for using ethyl 1-(5-(4,4,5,5-tetramethyl-1,3,2-dioxaborolan-2-yl)pyrimidin-2-yl) piperidin-3-carboxylate instead of 2-(4-methylpiperazin-1-yl)-5-(4,4,5,5-tetramethyl-1,3,2-dioxaborolan-2-yl)pyrimidine.

Mass (M+H⁺): 478.2

¹H-NMR (500 Mhz, DMSO-d6): 1.15 (t, J=6.4 HZ, 3H), 1.48-1.53 (m, 1H), 1.68-1.74 (m, 2H), 1.99-2.02 (m, 1H), 2.53-2.57 (m, 1H), 3.13-3.26 (m, 2H), 3.74 (s, 3H), 3.92 (s, 3H), 4.05-4.09 (m, 2H), 4.45-4.48 (m, 1H), 4.69-4.72 (m, 1H), 5.45 (s, 2H), 7.07 (s, 1H), 7.50 (s, 1H), 7.94 (s, 1H), 8.40 (s, 2H)

### [Example 16] Synthesis of 9-(2-((3-(1H-imidazol-1-yl) propyl)amino)pyrimidin-5-yl)-6,7-dimethoxynaphtho[2,3-c]furan-1(3H)-one

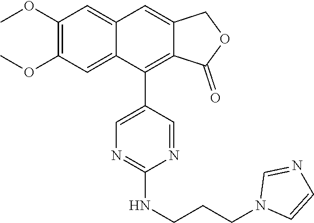 [SMILES: COc1cc2cc3c(c(-c4cnc(NCCCn5ccnc5)nc4)c2cc1OC)C(=O)OC3]

The compound of Example 16 was synthesized by the same method as that of the compound of Example 7 except for using N-(3-(1H-imidazol-1-yl) propyl)-5-(4,4,5,5-tetramethyl-1,3,2-dioxaborolan-2-yl)pyrimidin-2-amine instead of 2-(4-methylpiperazin-1-yl)-5-(4,4,5,5-tetramethyl-1,3,2-dioxaborolan-2-yl)pyrimidine.

Mass (M+H⁺): 446.2

¹H-NMR (500 Mhz, DMSO-d6): 1.99-2.03 (m, 2H), 3.30-3.34 (m, 2H), 3.74 (s, 3H), 3.92 (s, 3H), 4.03-4.07 (m, 2H), 5.42 (s, 2H), 6.86 (s, 1H), 7.08 (s, 1H), 7.19 (s, 1H), 7.50 (s, 2H), 7.64 (s, 1H), 7.93 (s, 1H), 8.31 (s, 2H)

### [Example 17] Synthesis of (R)-6,7-dimethoxy-9-(2-(quinuclidin-3-yloxy)pyrimidin-5-yl)naphtho[2,3-c]furan-1(3H)-one

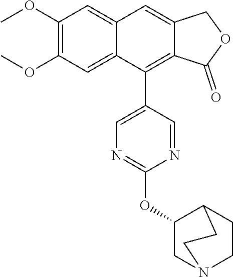 [SMILES: COc1cc2cc3c(c(-c4cnc(O[C@H]5CN6CCC5CC6)nc4)c2cc1OC)C(=O)OC3]

The compound of Example 17 was synthesized by the same method as that of the compound of Example 7 except for using (R)-3-((5-(4,4,5,5-tetramethyl-1,3,2-dioxaborolan-2-yl)pyrimidin-2-yl)oxy) quinuclidine instead of 2-(4-methylpiperazin-1-yl)-5-(4,4,5,5-tetramethyl-1,3,2-dioxaborolan-2-yl)pyrimidine.

Mass (M+H⁺): 448.2

¹H-NMR (500 Mhz, DMSO-d6): 1.43-1.45 (m, 1H), 1.61-1.69 (m, 2H), 1.91-1.95 (m, 1H), 2.18 (s, 1H), 2.68-2.87 (m, 6H), 3.31-3.34 (m, 1H), 3.72 (s, 3H), 3.92 (s, 3H), 5.45 (s, 2H), 6.98 (s, 1H), 7.54 (s, 1H), 8.00 (s, 1H), 8.63 (s, 2H)

### [Example 18] Synthesis of 6,7-dimethoxy-9-(2-((2-methoxyethyl)(methyl)amino)pyrimidin-5-yl)naphtho[2,3-c]furan-1(3H)-one

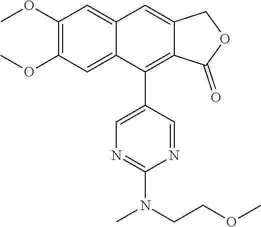

The compound of Example 18 was synthesized by the same method as that of the compound of Example 7 except for using N-(2-methoxyethyl)-N-methyl-5-(4,4,5,5-tetramethyl-1,3,2-dioxaborolan-2-yl)pyrimidin-2-amine instead of 2-(4-methylpiperazin-1-yl)-5-(4,4,5,5-tetramethyl-1,3,2-dioxaborolan-2-yl)pyrimidine.

Mass (M+H⁺): 410.2

¹H-NMR (500 Mhz, DMSO-d6): δ 3.20 (s, 3H), 3.26-3.33 (m, 3H), 3.57 (t, J=5.75 Hz, 2H), 3.73 (s, 3H), 3.84-3.87 (m, 2H), 3.92 (s, 3H), 5.42 (s, 2H), 7.07 (s, 1H), 7.50 (s, 1H), 7.93 (s, 1H), 8.38 (s, 2H)

### [Example 19] Synthesis of ethyl (5-(6,7-dimethoxy-3-oxo-1,3-dihydronaphtho[2,3-c]furan-4-yl)pyrimidin-2-yl)-L-prolinate

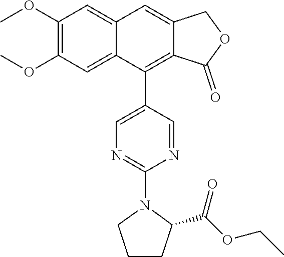 [SMILES: CCOC(=O)[C@@H]1CCCN1c1ncc(-c2c3c(cc4cc(OC)c(OC)cc24)COC3=O)cn1]

The compound of Example 19 was synthesized by the same method as that of the compound of Example 7 except for using ethyl (5-(4,4,5,5-tetramethyl-1,3,2-dioxaborolan-2-yl)pyrimidin-2-yl)-D-propionate instead of 2-(4-methylpiperazin-1-yl)-5-(4,4,5,5-tetramethyl-1,3,2-dioxaborolan-2-yl)pyrimidine.

Mass (M+H⁺): 464.2

¹H-NMR (500 Mhz, CD3OD): 1.24 (t, J=6.85 Hz, 3H), 2.08-2.15 (m, 3H), 2.40-2.45 (m, 1H), 3.76-3.83 (m, 4H), 3.99 (s, 3H), 4.16-4.20 (m, 2H), 4.54 (s, 2H), 5.42 (s, 2H), 7.12 (s, 1H), 7.41 (s, 1H), 7.90 (s, 1H), 8.36 (s, 2H)

### [Example 20] Synthesis of 9-(2-(benzo[d][1,3]dioxol-5-yloxy)pyrimidin-5-yl)-6,7-dimethoxynaphtho[2,3-c]furan-1(3H)-one

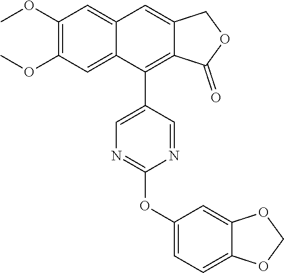 [SMILES: COc1cc2cc3c(c(-c4cnc(Oc5ccc6c(c5)OCO6)nc4)c2cc1OC)C(=O)OC3]

The compound of Example 20 was synthesized by the same method as that of the compound of Example 7 except for using 2-(benzo[d][1,3]dioxol-5-yloxy)-5-(4,4,5,5-tetramethyl-1,3,2-dioxaborolan-2-yl)pyrimidine instead of 2-(4-methylpiperazin-1-yl)-5-(4,4,5,5-tetramethyl-1,3,2-dioxaborolan-2-yl)pyrimidine.

Mass (M+H⁺): 459.1

¹H-NMR (500 Mhz, DMSO-d6): δ 3.74 (s, 3H), 3.93 (s, 3H), 5.45 (s, 2H), 6.05 (s, 2H), 6.73 (dd, J=8.35, 2.35 Hz, 1H), 6.93 (s, 1H), 6.95-6.96 (m, 1H), 6.99 (s, 1H), 7.55 (s, 1H), 8.02 (s, 1H), 8.67 (s, 2H)

### [Example 21] Synthesis of 6,7-dimethoxy-9-(2-(naphthalen-1-yloxy)pyrimidin-5-yl)naphtho[2,3-c]furan-1(3H)-one

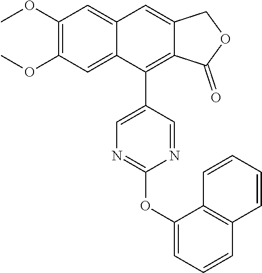 [SMILES: COc1cc2cc3c(c(-c4cnc(Oc5cccc6ccccc56)nc4)c2cc1OC)C(=O)OC3]

The compound of Example 21 was synthesized by the same method as that of the compound of Example 7 except for using 2-(naphthalen-1-ylmethyl)-5-(4,4,5,5-tetramethyl-1,3,2-dioxaborolan-2-yl)pyrimidine instead of 2-(4-methylpiperazin-1-yl)-5-(4,4,5,5-tetramethyl-1,3,2-dioxaborolan-2-yl)pyrimidine.

Mass (M+H⁺): 465.1

¹H-NMR (500 Mhz, DMSO-d6): δ 3.74 (s, 3H), 3.93 (s, 3H), 5.45 (s, 2H), 6.97 (s, 1H), 7.47-7.58 (m, 5H), 7.86 (t, J=8.15 Hz, 2H), 8.01 (d, J=7.7 Hz, 2H), 8.69 (s, 2H)

### [Example 22] Synthesis of (S)-6,7-dimethoxy-9-(2-((5-methoxy-1,2,3,4-tetrahydronaphthalen-2-yl)amino)pyrimidin-5-yl)naphtho[2,3-c]furan-1(3H)-one

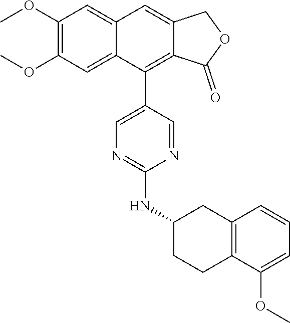

The compound of Example 22 was synthesized by the same method as that of the compound of Example 7 except for using (S)—N-(5-methoxy-1,2,3,4-tetrahydronaphthalen-2-yl)-5-(4,4,5,5-tetramethyl-1,3,2-dioxaborolan-2-yl)pyrimidin-2-amine instead of 2-(4-methylpiperazin-1-yl)-5-(4,4,5,5-tetramethyl-1,3,2-dioxaborolan-2-yl)pyrimidine.

Mass (M+H⁺): 498.2

¹H-NMR (500 Mhz, DMSO-d6): 1.60-1.71 (m, 1H), 2.13-2.16 (m, 1H), 2.50-2.55 (m, 2H), 2.71-2.78 (m, 1H), 2.86-2.91 (m, 1H), 3.07-3.10 (m, 1H), 3.75 (s, 6H), 3.93 (s, 3H), 5.42 (s, 2H), 6.66-6.68 (m, 1H), 6.71-6.73 (m, 1H), 7.03-7.07 (m, 1H), 7.10 (s, 1H), 7.42-7.43 (m, 1H), 7.50 (s, 1H), 7.93 (s, 1H), 8.33 (s, 2H)

### [Example 23] Synthesis of 9-(2-(isopropyl(methyl)amino)pyrimidin-5-yl)-6,7-dimethoxynaphtho[2,3-c]furan-1(3H)-one

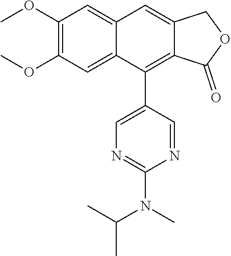 [SMILES: COc1cc2cc3c(c(-c4cnc(N(C)C(C)C)nc4)c2cc1OC)C(=O)OC3]

The compound of Example 23 was synthesized by the same method as that of the compound of Example 7 except for using N-isopropyl-N-methyl-5-(4,4,5,5-tetramethyl-1,3,2-dioxaborolan-2-yl)pyrimidin-2-amine instead of 2-(4-methylpiperazin-1-yl)-5-(4,4,5,5-tetramethyl-1,3,2-dioxaborolan-2-yl)pyrimidine.

Mass (M+H⁺): 394.2

¹H-NMR (500 Mhz, DMSO-d6): 1.18 (d, J=6.75 Hz, 6H), 3.01 (s, 3H), 3.74 (s, 3H), 3.92 (s, 3H), 5.05-5.11 (m, 1H), 5.42 (s, 2H), 7.10 (s, 1H), 7.50 (s, 1H), 7.92 (s, 1H), 8.37 (s, 2H)

### [Example 24] Synthesis of 9-(2-((S)-2-((2S,6R)-2,6-dimethylmorpholin-4-carbonyl)pyrrolidin-1-yl)pyrimidin-5-yl)-6,7-dimethoxynaphtho[2,3-c]furan-1(3H)-one

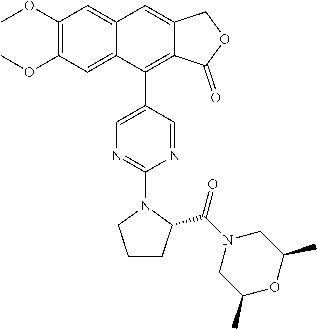

It was synthesized by the same method.

Mass (M+H⁺): 533.3

¹H-NMR (500 Mhz, DMSO-d6): δ 1.05-1.10 (m, 6H), 1.90-1.98 (m, 3H), 2.23-2.40 (m, 2H), 2.76-2.84 (m, 1H), 3.30-3.41 (m, 2H), 3.68-3.73 (m, 5H), 3.92-4.18 (m, 5H), 5.00-5.13 (m, 1H), 5.42 (s, 2H), 7.05 (s, 1H), 7.50 (s, 1H), 7.93 (s, 1H), 8.32-8.39 (m, 2H)

### [Example 25] Synthesis of 9-(2-(sec-butoxy)pyrimidin-5-yl)-6,7-dimethoxynaphtho[2,3-c]furan-1(3H)-one

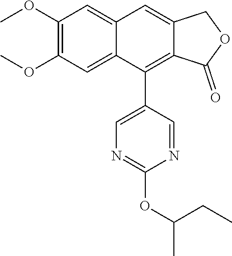 [SMILES: CCC(C)Oc1ncc(-c2c3c(cc4cc(OC)c(OC)cc24)COC3=O)cn1]

The compound of Example 25 was synthesized by the same method as that of the compound of Example 7 except for using 2-(sec-butoxy)-5-(4,4,5,5-tetramethyl-1,3,2-dioxaborolan-2-yl)pyrimidine instead of 2-(4-methylpiperazin-1-yl)-5-(4,4,5,5-tetramethyl-1,3,2-dioxaborolan-2-yl)pyrimidine.

Mass (M+H⁺): 395.2

¹H-NMR (500 Mhz, DMSO-d6): 0.92-0.96 (m, 3H), 1.33 (d, J=6.15 Hz, 3H), 1.67-1.77 (m, 2H), 3.72 (s, 3H), 3.93 (s, 3H), 5.10-5.14 (m, 1H), 5.45 (s, 2H), 6.99 (s, 1H), 7.53 (s, 1H), 8.00 (s, 1H), 8.62 (s, 2H)

### [Example 26] Synthesis of 6,7-dimethoxy-9-(2-methoxypyrimidin-5-yl)naphtho[2,3-c]furan-1(3H)-one

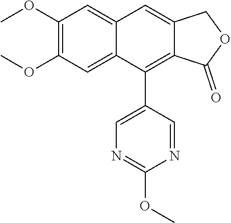 [SMILES: COc1ncc(-c2c3c(cc4cc(OC)c(OC)cc24)COC3=O)cn1]

The compound of Example 26 was synthesized by the same method as that of the compound of Example 7 except for using 2-methoxy-5-(4,4,5,5-tetramethyl-1,3,2-dioxaborolan-2-yl)pyrimidine instead of 2-(4-methylpiperazin-1-yl)-5-(4,4,5,5-tetramethyl-1,3,2-dioxaborolan-2-yl)pyrimidine.

Mass (M+H⁺): 353.1

¹H-NMR (500 Mhz, DMSO-d6): δ 3.72 (s, 3H), 3.93 (s, 3H), 4.00 (s, 3H), 5.45 (s, 2H), 6.97 (s, 1H), 7.54 (s, 1H), 8.01 (s, 1H), 8.65 (s, 2H)

### [Example 27] Synthesis of methyl (5-(6,7-dimethoxy-3-oxo-1,3-dihydronaphtho[2,3-c]furan-4-yl)pyrimidin-2-yl)-D-valinate

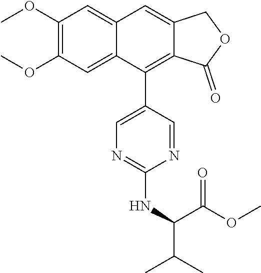

The compound of Example 27 was synthesized by the same method as that of the compound of Example 7 except for using methyl (5-(4,4,5,5-tetramethyl-1,3,2-dioxaborolan-2-yl)pyrimidin-2-yl)-D-valinate instead of 2-(4-methylpiperazin-1-yl)-5-(4,4,5,5-tetramethyl-1,3,2-dioxaborolan-2-yl)pyrimidine.

Mass (M+H⁺): 452.2

¹H-NMR (500 Mhz, DMSO-d6): δ 1.00 (dd, J=23.45, 6.85 Hz, 6H), 2.15-2.18 (m, 1H), 3.61 (s, 3H), 3.73 (s, 3H), 3.93 (s, 3H), 4.30-4.33 (m, 1H), 5.45 (s, 2H), 7.04 (s, 1H), 7.50 (s, 1H), 7.63 (d, J=7.4 Hz, 1H), 7.93 (s, 1H), 8.34 (s, 2H)

### [Example 28] Synthesis of methyl 9-(2-((2-(cyclohex-1-en-1-yl)ethyl)amino)pyrimidin-5-yl)-6,7-dimethoxynaphtho[2,3-c]furan-1(3H)-one

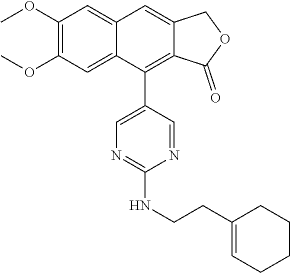 [SMILES: COc1cc2cc3c(c(-c4cnc(NCCC5=CCCCC5)nc4)c2cc1OC)C(=O)OC3]

The compound of Example 28 was synthesized by the same method as that of the compound of Example 7 except for using N-(2-(cyclohex-1-en-1-yl)ethyl)-5-(4,4,5,5-tetramethyl-1,3,2-dioxaborolan-2-yl)pyrimidin-2-amine instead of 2-(4-methylpiperazin-1-yl)-5-(4,4,5,5-tetramethyl-1,3,2-dioxaborolan-2-yl)pyrimidine.

Mass (M+H⁺): 446.2

¹H-NMR (500 Mhz, DMSO-d6): δ 1.47-1.54 (m, 4H), 1.91-1.94 (m, 4H), 2.18-2.19 (m, 2H), 3.4-3.42 (m, 2H), 3.73 (s, 3H), 3.92 (s, 3H), 5.42 (s, 2H), 7.07 (s, 1H), 7.23-7.26 (m, 1H), 7.50 (s, 1H), 8.29 (s, 2H)

### [Example 29] Synthesis of (Z)-9-(5-((2-(dimethylamino)ethyliden)amino)pyrazin-2-yl)-6,7-dimethoxynaphtho[2,3-c]furan-1(3H)-one

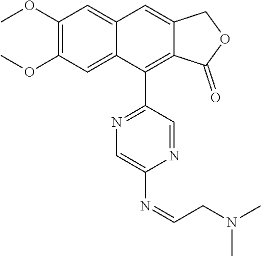

The compound of Example 29 was synthesized by the same method as that of the compound of Example 7 except for using (Z)—N,N-dimethyl-2-((5-(4,4,5,5-tetramethyl-1,3,2-dioxaborolan-2-yl)pyrazin-2-yl)imino) ethan-1-amine instead of 2-(4-methylpiperazin-1-yl)-5-(4,4,5,5-tetramethyl-1,3,2-dioxaborolan-2-yl)pyrimidine.

Mass (M+H⁺): 393.2

¹H-NMR (500 Mhz, DMSO-d6): δ 3.06 (s, 3H), 3.14 (s, 3H), 3.25 (s, 2H), 3.66 (s, 3H), 3.93 (s, 3H), 5.45 (s, 2H), 7.06 (s, 1H), 7.51 (s, 1H), 7.99 (s, 1H), 8.29 (s, 2H), 8.65 (s, 1H)

### [Example 30] Synthesis of 9-(5-aminopyrazin-2-yl)-6,7-dimethoxynaphtho[2,3-c]furan-1(3H)-one

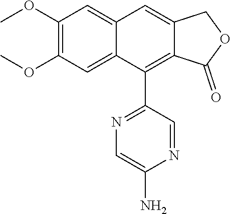 [SMILES: COc1cc2cc3c(c(-c4cnc(N)cn4)c2cc1OC)C(=O)OC3]

The compound of Example 30 was synthesized by the same method as that of the compound of Example 7 except for using 5-(4,4,5,5-tetramethyl-1,3,2-dioxaborolan-2-yl)pyrazin-2-amine instead of 2-(4-methylpiperazin-1-yl)-5-(4,4,5,5-tetramethyl-1,3,2-dioxaborolan-2-yl)pyrimidine.

Mass (M+H⁺): 338.2

¹H-NMR (500 Mhz, DMSO-d6): δ 3.68 (s, 3H), 3.9 (s, 3H), 5.42 (s, 2H), 6.59 (s, 2H), 7.11 (s, 1H), 7.48 (s, 1H), 7.93 (s, 1H), 8.00 (s, 2H)

### [Example 31] Synthesis of 9-(5-(dimethylamino)pyrazin-2-yl)-6,7-dimethoxynaphtho[2,3-c]furan-1(3H)-one

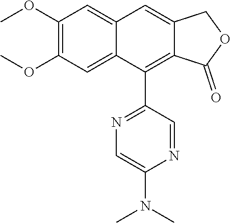 [SMILES: COc1cc2cc3c(c(-c4cnc(N(C)C)cn4)c2cc1OC)C(=O)OC3]

The compound of Example 31 was synthesized by the same method as that of the compound of Example 7 except for using N,N-dimethyl-5-(4,4,5,5-tetramethyl-1,3,2-dioxaborolan-2-yl)pyrazin-2-amine instead of 2-(4-methylpiperazin-1-yl)-5-(4,4,5,5-tetramethyl-1,3,2-dioxaborolan-2-yl)pyrimidine.

Mass (M+H⁺): 366.2

¹H-NMR (500 Mhz, DMSO-d6): δ 3.15 (s, 6H), 3.67 (s, 3H), 3.92 (s, 3H), 5.43 (s, 2H), 7.14 (s, 1H), 7.49 (s, 1H), 7.95 (s, 1H), 8.17 (s, 1H), 8.29 (s, 1H)

### [Example 32] Synthesis of 6,7-dimethoxy-9-(pyrazin-2-yl)naphtho[2,3-c]furan-1(3H)-one

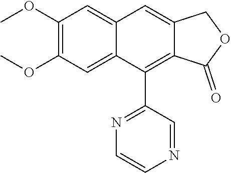 [SMILES: COc1cc2cc3c(c(-c4cnccn4)c2cc1OC)C(=O)OC3]

The compound of Example 32 was synthesized by the same method as that of the compound of Example 7 except for using 2-(4,4,5,5-tetramethyl-1,3,2-dioxaborolan-2-yl)pyrazine instead of 2-(4-methylpiperazin-1-yl)-5-(4,4,5,5-tetramethyl-1,3,2-dioxaborolan-2-yl)pyrimidine.

Mass (M+H⁺): 323.1

¹H-NMR (500 Mhz, DMSO-d6): δ 3.63 (s, 3H), 3.93 (s, 3H), 5.49 (s, 2H), 6.86 (s, 1H), 7.55 (s, 1H), 8.07 (s, 1H), 8.74 (s, 1H), 8.80 (s, 1H), 8.79 (s, 1H)

### [Example 33] Synthesis of (S)-1-(5-(6,7-dimethoxy-3-oxo-1,3-dihydronaphtho[2,3-c]furan-4-yl)pyrimidin-2-yl)-N-(2-meth oxyethyl)-N-methylpyrrolidin-2-carboxamide

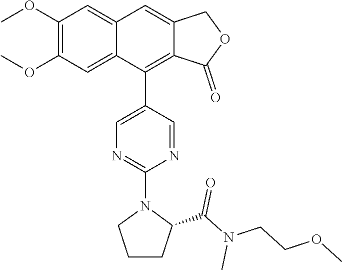

The compound of Example 33 was synthesized by the same method as that of the compound of Example 7 except for using (R)—N-(2-methoxyethyl)-N-methyl-1-(5-(4,4,5,5-tetramethyl-1,3,2-dioxaborolan-2-yl)pyrimidin-2-yl)pyrrolidin-2-carboxamide instead of 2-(4-methylpiperazin-1-yl)-5-(4,4,5,5-tetramethyl-1,3,2-dioxaborolan-2-yl)pyrimidine.

Mass (M+H⁺): 507.3

¹H-NMR (500 Mhz, DMSO-d6): δ 1.07 (s, 2H), 1.8-2.1 (m, 3H), 2.25-2.30 (m, 1H), 2.82 (s, 1H), 3.04 (s, 1H), 3.13 (s, 2H), 3.19 (s, 2H), 3.30-3.40 (m, 1H), 3.55-3.56 (m, 1H), 3.68-3.71 (m, 3H), 3.73 (s, 3H), 3.92 (s, 3H), 4.99-5.01 (m, 1H), 5.42 (s, 2H), 7.06 (s, 1H), 7.50 (s, 1H), 7.93 (s, 1H), 8.30 (s, 2H), 8.41 (s, 1H)

### [Example 34] Synthesis of 9-(1H-indol-5-yl)-6,7-dimethoxynaphtho[2,3-c]furan-1(3H)-one

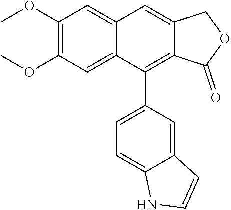 [SMILES: COc1cc2cc3c(c(-c4ccc5[nH]ccc5c4)c2cc1OC)C(=O)OC3]

The compound of Example 34 was synthesized by the same method as that of the compound of Example 7 except for using 5-(4,4,5,5-tetramethyl-1,3,2-dioxaborolan-2-yl)-1H-indole instead of 2-(4-methylpiperazin-1-yl)-5-(4,4,5,5-tetramethyl-1,3,2-dioxaborolan-2-yl)pyrimidine.

Mass (M+H⁺): 360.1

¹H-NMR (500 Mhz, DMSO-d6): δ 3.52 (s, 3H), 3.92 (s, 3H), 5.41 (s, 2H), 6.45 (s, 1H), 6.95-7.03 (m, 2H), 7.38 (s, 1H), 7.42-7.48 (m, 2H), 7.9 (s, 1H), 11.19 (s, 1H)

### [Example 35] Synthesis of 9-(6-fluoropyridin-3-yl)-6,7-dimethoxynaphtho[2,3-c]furan-1(3H)-one

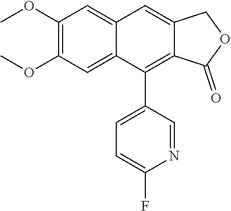 [SMILES: COc1cc2cc3c(c(-c4ccc(F)nc4)c2cc1OC)C(=O)OC3]

The compound of Example 35 was synthesized by the same method as that of the compound of Example 7 except for using 2-fluoro-5-(4,4,5,5-tetramethyl-1,3,2-dioxaborolan-2-yl)pyridine instead of 2-(4-methylpiperazin-1-yl)-5-(4,4,5,5-tetramethyl-1,3,2-dioxaborolan-2-yl)pyrimidine.

Mass (M+H⁺): 340.1

¹H-NMR (500 Mhz, DMSO-d6): δ 3.66 (s, 3H), 3.93 (s, 3H), 5.45 (s, 2H), 6.84 (s, 1H), 7.33 (d, J=8.6 Hz, 1H), 7.54-8.04 (m, 2H), 8.23 (s, 1H)

### [Example 36] Synthesis of 2-hydroxyethyl (5-(6,7-dimethoxy-3-oxo-1,3-dihydronaphtho[2,3-c]furan-4-yl)pyrimidin-2-yl)-L-prolinate

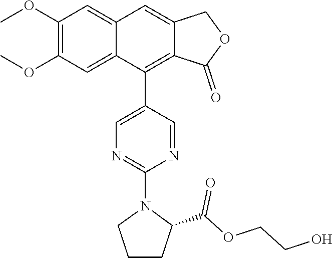 [SMILES: COc1cc2cc3c(c(-c4cnc(N5CCC[C@H]5C(=O)OCCO)nc4)c2cc1OC)C(=O)OC3]

The compound of Example 36 was synthesized by the same method as that of the compound of Example 7 except for using 2-hydroxyethyl (5-(4,4,5,5-tetramethyl-1,3,2-dioxaborolan-2-yl)pyrimidin-2-yl)-D-prolinate instead of 2-(4-methylpiperazin-1-yl)-5-(4,4,5,5-tetramethyl-1,3,2-dioxaborolan-2-yl)pyrimidine.

Mass (M+H⁺): 480.2

¹H-NMR (500 Mhz, DMSO-d6): δ 1.95-2.10 (m, 2H), 2.30-2.40 (m, 1H), 3.50-3.53 (m, 2H), 3.6-3.7 (m, 2H), 3.72 (s, 3H), 3.92 (s, 3H), 3.97-4.01 (m, 1H), 4.07-4.10 (m, 1H), 4.60-4.62 (m, 1H), 4.70-4.75 (m, 1H), 5.42 (s, 2H), 7.03 (s, 1H), 7.51 (s, 1H), 7.94 (s, 1H), 8.36 (s, 1H), 8.43 (s, 1H)

### [Example 37] Synthesis of 6,7-dimethoxy-9-(2-((2-propoxyethyl)amino)pyrimidin-5-yl)naphtho[2,3-c]furan-1(3H)-one

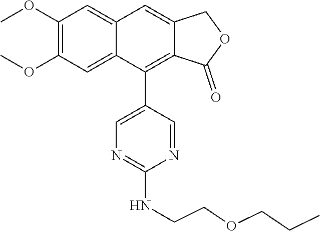 [SMILES: CCCOCCNc1ncc(-c2c3c(cc4cc(OC)c(OC)cc24)COC3=O)cn1]

The compound of Example 37 was synthesized by the same method as that of the compound of Example 7 except for using N-(2-proxyethyl)-5-(4,4,5,5-tetramethyl-1,3,2-dioxaborolan-2-yl)pyrimidin-2-amine instead of 2-(4-methylpiperazin-1-yl)-5-(4,4,5,5-tetramethyl-1,3,2-dioxaborolan-2-yl)pyrimidine.

Mass (M+H⁺): 424.2

¹H-NMR (500 Mhz, DMSO-d6): δ 0.77-0.80 (m, 3H), 1.43-1.46 (m, 2H), 3.25-3.32 (m, 2H), 3.40-3.48 (m, 4H), 3.68 (s, 3H), 3.87 (s, 3H), 5.36 (s, 2H), 7.02 (s, 1H), 7.25-7.26 (m, 1H), 7.45 (s, 1H), 7.87 (s, 1H), 8.25 (s, 2H)

### [Example 38] Synthesis of methyl (5-(6,7-dimethoxy-3-oxo-1,3-dihydronaphtho[2,3-c]furan-4-yl)pyrimidin-2-yl)-L-prolyl-D-alaniate

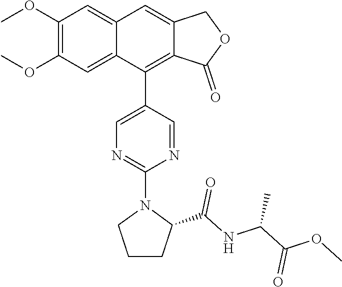

The compound of Example 38 was synthesized by the same method as that of the compound of Example 7 except for using methyl (5-(4,4,5,5-tetramethyl-1,3,2-dioxaborolan-2-yl)pyrimidin-2-yl)-L-propyl-D-alaniate instead of 2-(4-methylpiperazin-1-yl)-5-(4,4,5,5-tetramethyl-1,3,2-dioxaborolan-2-yl)pyrimidine.

Mass (M+H⁺): 521.2

¹H-NMR (500 Mhz, DMSO-d6): δ 1.22 (d, J=7.15 Hz, 3H), 1.95-1.99 (m, 3H), 2.20-2.25 (m, 1H), 3.55 (s, 3H), 3.60-3.65 (m, 1H), 3.72 (s, 3H), 3.75-3.80 (m, 1H), 3.92 (s, 3H), 4.26-4.32 (m, 1H), 4.52-4.54 (m, 1H), 5.42 (s, 2H), 7.06 (s, 1H), 7.50 (s, 1H), 7.94 (s, 1H), 8.28 (d, J=6.6 Hz, 1H), 8.30-8.43 (m, 2H)

### [Example 39] Synthesis of (S)-1-(5-(6,7-dimethoxy-3-oxo-1,3-dihydronaphtho[2,3-c]furan-4-yl)pyrimidin-2-yl)-N—((S)-1-(4-fluorophenyl)ethyl)pyrrolidin-2-carboxamide

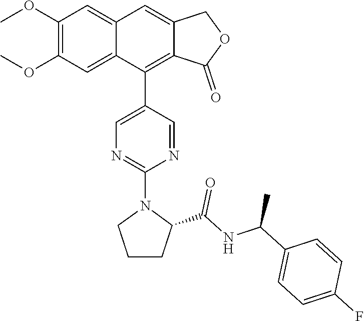 [SMILES: COc1cc2cc3c(c(-c4cnc(N5CCC[C@H]5C(=O)N[C@@H](C)c5ccc(F)cc5)nc4)c2cc1OC)C(=O)OC3]

The compound of Example 39 was synthesized by the same method as that of the compound of Example 7 except for using (S)—N—((S)-1-(4-fluorophenyl)ethyl)-1-(5-(4,4,5,5-tetramethyl-1,3,2-dioxaborolan-2-yl)pyrimidin-2-yl)pyrrolidin-2-carboxamide instead of 2-(4-methylpiperazin-1-yl)-5-(4,4,5,5-tetramethyl-1,3,2-dioxaborolan-2-yl)pyrimidine.

Mass (M+H⁺): 557.3

¹H-NMR (500 Mhz, DMSO-d6): δ 1.32 (d, J=6.85 Hz, 3H), 1.92-193 (m, 3H), 2.20-2.30 (m, 1H), 3.63-3.66 (m, 5H), 3.93 (s, 3H), 4.52 (d, J=8.6 Hz, 1H), 4.90 (t, J=8.0 Hz, 1H), 5.43 (s, 2H), 7.04-7.07 (m, 3H), 7.29 (br, 2H), 7.51 (s, 1H), 7.95 (s, 1H), 8.35 (d, J=8.0 Hz, 1H), 8.36-8.42 (m, 2H)

### [Example 40] Synthesis of 6,7-dimethoxy-9-(quinoxalin-6-yl)naphtho[2,3-c]furan-1(3H)-one

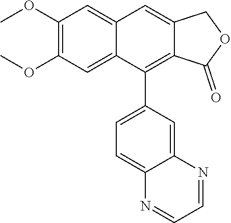 [SMILES: COc1cc2cc3c(c(-c4ccc5nccnc5c4)c2cc1OC)C(=O)OC3]

The compound of Example 40 was synthesized by the same method as that of the compound of Example 7 except for using (R)—N—((R)-1-(4-fluorophenyl)ethyl)-1-(5-(4,4,5,5-tetramethyl-1,3,2-dioxaborolan-2-yl)pyrimidin-2-yl)pyrrolidin-2-carboxamide instead of 2-(4-methylpiperazin-1-yl)-5-(4,4,5,5-tetramethyl-1,3,2-dioxaborolan-2-yl)pyrimidine.

Mass (M+H⁺): 373.1

¹H-NMR (500 Mhz, DMSO-d6): δ 3.49 (s, 3H), 3.89 (s, 3H), 5.43 (s, 2H), 6.82 (s, 1H), 7.51 (s, 1H), 7.78-7.80 (m, 1H), 7.99 (s, 1H), 8.03 (s, 1H), 8.16 (d, J=8.85 Hz, 1H), 8.95-8.97 (m, 2H)

### [Example 41] Synthesis of (S)-1-(5-(6,7-dimethoxy-3-oxo-1,3-dihydronaphtho[2,3-c]furan-4-yl)pyrimidin-2-yl)-N-((5-meth ylfuran-2-yl)methyl)pyrrolidin-2-carboxamide

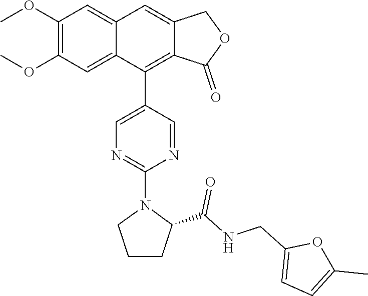

The compound of Example 41 was synthesized by the same method as that of the compound of Example 7 except for using (S)—N-((5-methylfuran-2-yl)methyl)-1-(5-(4,4,5,5-tetramethyl-1,3,2-dioxaborolan-2-yl)pyrimidin-2-yl)pyrrolidin-2-carboxamide instead of 2-(4-methylpiperazin-1-yl)-5-(4,4,5,5-tetramethyl-1,3,2-dioxaborolan-2-yl)pyrimidine.

Mass (M+H⁺): 529.2

¹H-NMR (500 Mhz, DMSO-d6): δ 1.90 (br, 3H), 2.09 (s, 3H), 2.18 (br, 1H), 3.52-3.71 (m, 5H), 3.87 (s, 3H), 4.11 (br, 2H), 4.49 (d, J=8.6 Hz, 1H), 5.37 (s, 2H), 5.84 (s, 1H), 5.97 (s, 1H), 7.01 (s, 1H), 7.45 (s, 1H), 7.88 (s, 1H), 8.30-8.40 (br, 3H)

### [Example 42] Synthesis of 6,7-dimethoxy-9-(2-((2-propoxyethyl)(propyl)amino)pyrimidin-5-yl)naphtho[2,3-c]furan-1(3H)-one

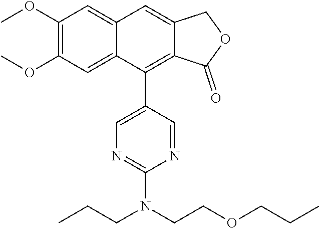 [SMILES: CCCOCCN(CCC)c1ncc(-c2c3c(cc4cc(OC)c(OC)cc24)COC3=O)cn1]

The compound of Example 42 was synthesized by the same method as that of the compound of Example 7 except for using N-(2-propoxyethyl)-N-propyl-5-(4,4,5,5-tetramethyl-1,3,2-dioxaborolan-2-yl)pyrazin-2-amine instead of 2-(4-methylpiperazin-1-yl)-5-(4,4,5,5-tetramethyl-1,3,2-dioxaborolan-2-yl)pyrimidine.

Mass (M+H⁺): 466.3

¹H-NMR (500 Mhz, DMSO-d6): δ 0.82-0.89 (m, 6H), 1.45-1.52 (m, 2H), 1.60-1.68 (m, 2H), 3.35 (t, J=6.3 Hz, 2H), 3.59 (t, J=6.3 Hz, 4H), 3.73 (s, 3h), 3.78 (t, J=5.7 Hz, 2h), 3.92 (s, 3h), 5.54 (s, 2H), 7.08 (s, 1H), 7.50 (s, 1H), 7.93 (s, 1H), 8.38 (s, 2H)

### [Example 43] Synthesis of 9-(dibenzo[b,d]furan-2-yl)-6,7-dimethoxynaphtho[2,3-c]furan-1(3H)-one

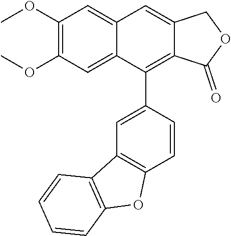 [SMILES: COc1cc2cc3c(c(-c4ccc5oc6ccccc6c5c4)c2cc1OC)C(=O)OC3]

The compound of Example 43 was synthesized by the same method as that of the compound of Example 7 except for using 2-(dibenzo[b,d]furan-3-yl)-4,4,5,5-tetramethyl-1,3,2-dioxaborolane instead of 2-(4-methylpiperazin-1-yl)-5-(4,4,5,5-tetramethyl-1,3,2-dioxaborolan-2-yl)pyrimidine.

Mass (M+H⁺): 411.1

¹H-NMR (500 Mhz, DMSO-d6): δ 3.53 (s, 3H), 3.90 (s, 3H), 5.46 (s, 2H), 6.93 (s, 1H), 7.38 (t, J=7.45 Hz, 1H), 7.48 (dd, J=8.6, 1.7 Hz, 1H), 7.1-7.54 (m, 2H), 7.74 (d, J=8.6 Hz, 1H), 7.81 (d, J=8.05 Hz, 1H), 7.99 (s, 1H), 8.12-8.15 (m, 2H)

### [Example 44] Synthesis of ethyl (5-(6,7-dimethoxy-3-oxo-1,3-dihydronaphtho[2,3-c]furan-4-yl)pyrimidin-2-yl)glycinate

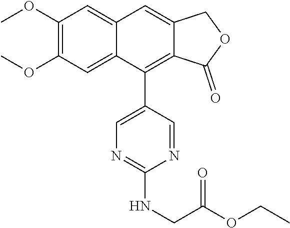 [SMILES: CCOC(=O)CNc1ncc(-c2c3c(cc4cc(OC)c(OC)cc24)COC3=O)cn1]

The compound of Example 44 was synthesized by the same method as that of the compound of Example 7 except for using ethyl (5-(4,4,5,5-tetramethyl-1,3,2-dioxaborolan-2-yl)pyrimidin-2-yl)glycinate instead of 2-(4-methylpiperazin-1-yl)-5-(4,4,5,5-tetramethyl-1,3,2-dioxaborolan-2-yl)pyrimidine.

Mass (M+H⁺): 424.1

¹H-NMR (500 Mhz, DMSO-d6): δ 1.11 (t, J=6.70 Hz, 3H), 3.67 (s, 3H), 3.88 (s, 3H), 4.02-4.06 (m, 4H), 5.37 (s, 2H), 6.98 (s, 1H), 7.46 (s, 1H), 7.66 (t, J=6.3 Hz, 1H), 7.89 (s, 1H), 8.29 (s, 2H)

### [Example 45] Synthesis of ethyl (5-(6,7-dimethoxy-3-oxo-1,3-dihydronaphtho[2,3-c]furan-4-yl)pyrimidin-2-yl)-L-proglycinate

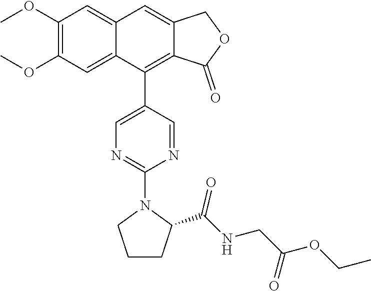 [SMILES: CCOC(=O)CNC(=O)[C@@H]1CCCN1c1ncc(-c2c3c(cc4cc(OC)c(OC)cc24)COC3=O)cn1]

The compound of Example 45 was synthesized by the same method as that of the compound of Example 7 except for using ethyl (5-(4,4,5,5-tetramethyl-1,3,2-dioxaborolan-2-yl)pyrimidin-2-yl)-L-prolyglycinate instead of 2-(4-methylpiperazin-1-yl)-5-(4,4,5,5-tetramethyl-1,3,2-dioxaborolan-2-yl)pyrimidine.

Mass (M+H⁺): 521.2

¹H-NMR (500 Mhz, DMSO-d6): δ 1.14 (dd, J=9.75, 4.55 Hz, 3H), 1.95-2.02 (m, 3H), 2.22-2.24 (m, 1H), 3.50-3.55 (m, 1H), 3.73 (s, 3H), 3.76-3.78 (m, 3H), 3.92 (s, 3H), 4.00-4.05 (m, 2H), 4.56 (t, J=1.7 Hz, 1H), 5.42 (s, 2H), 7.06 (s, 1H), 7.50 (s, 1H), 7.94 (s, 1H), 8.30-8.40 (m, 3H)

### [Example 46] Synthesis of 6,7-dimethoxy-9-(quinolin-3-yl)naphtho[2,3-c]furan-1(3H)-one

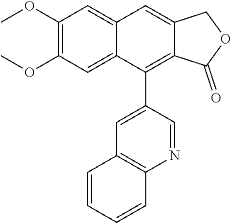 [SMILES: COc1cc2cc3c(c(-c4cnc5ccccc5c4)c2cc1OC)C(=O)OC3]

The compound of Example 46 was synthesized by the same method as that of the compound of Example 7 except for using 3-(4,4,5,5-tetramethyl-1,3,2-dioxaborolan-2-yl) quinoline instead of 2-(4-methylpiperazin-1-yl)-5-(4,4,5,5-tetramethyl-1,3,2-dioxaborolan-2-yl)pyrimidine.

Mass (M+H⁺): 372.2

¹H-NMR (500 Mhz, DMSO-d6): δ 3.57 (s, 3H), 3.94 (s, 3H), 5.49 (s, 2H), 6.94 (s, 1H), 7.57 (s, 1H), 7.66-7.68 (m, 1H), 7.81-7.83 (m, 1H), 8.04-8.05 (m, 2H), 8.11 (d, J=8.6 Hz, 1H), 8.44 (d, J=1.7 Hz, 1H), 8.86 (d, J=2.3 Hz, 1H)

### [Example 47] Synthesis of 9-(2-(((1r,4r)-4-hydroxycyclohexyl)amino)pyrimidin-5-yl)-6,7-dimethoxynaphtho[2,3-c]furan-1(3H)-one

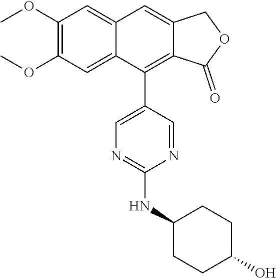 [SMILES: COc1cc2cc3c(c(-c4cnc(N[C@H]5CC[C@H](O)CC5)nc4)c2cc1OC)C(=O)OC3]

The compound of Example 47 was synthesized by the same method as that of the compound of Example 7 except for using (1r,4r)-4-((5-(4,4,5,5-tetramethyl-1,3,2-dioxaborolan-2-yl)pyrimidin-2-yl)amino)cyclohexan-1-ol instead of 2-(4-methylpiperazin-1-yl)-5-(4,4,5,5-tetramethyl-1,3,2-dioxaborolan-2-yl)pyrimidine.

Mass (M+H⁺): 436.2

¹H-NMR (500 Mhz, DMSO-d6): δ 1.31-1.28 (m, 4H), 1.78 (d, J=12.0 Hz, 2H), 1.88 (d, J=11.2 Hz, 2H), 3.30-3.40 (m, 1H), 3.65-3.67 (m, 1H), 3.69 (s, 3H), 3.87 (s, 3H), 4.47 (d, J=4.25 Hz, 1H), 5.36 (s, 2H), 7.04 (s, 1H), 7.18 (d, J=8.0 Hz, 1H), 7.44 (s, 1H), 7.87 (s, 1H), 8.24 (s, 2H)

### [Example 48] Synthesis of 9-(2-(hexyl(2-(hexyloxy)ethyl)amino)pyrimidin-5-yl)-6,7-dimethoxynaphtho[2,3-c]furan-1(3H)-one

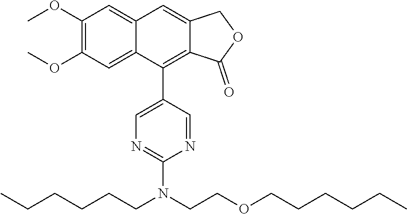 [SMILES: CCCCCCOCCN(CCCCCC)c1ncc(-c2c3c(cc4cc(OC)c(OC)cc24)COC3=O)cn1]

The compound of Example 48 was synthesized by the same method as that of the compound of Example 7 except for using N-hexyl-N-(2-(hexyloxy)ethyl)-5-(4,4,5,5-tetramethyl-1,3,2-dioxaborolan-2-yl)pyrimidin-2-amine instead of 2-(4-methylpiperazin-1-yl)-5-(4,4,5,5-tetramethyl-1,3,2-dioxaborolan-2-yl)pyrimidine.

Mass (M+H⁺): 550.4

¹H-NMR (500 Mhz, DMSO-d6): δ 0.80-0.84 (m, 6H), 1.20-1.22 (m, 5H), 1.25-1.27 (m, 7H), 1.42-1.50 (m, 2H), 1.6-1.65 (m, 2H), 3.38 (t, 2H), 3.55-3.60 (m, 4H), 3.73 (s, 3H), 3.75-3.78 (m, 2H), 3.92 (s, 3H), 5.42 (s, 2H), 7.08 (s, 1H), 7.50 (s, 1H), 7.93 (s, 1H), 8.37 (s, 2H)

### [Example 49] Synthesis of (S)-1-(5-(6,7-dimethoxy-3-oxo-1,3-dihydronaphtho[2,3-c]furan-4-yl)pyrimidin-2-yl)pyrrolidin-2-carboxamide

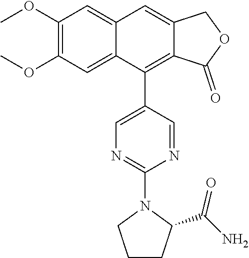 [SMILES: COc1cc2cc3c(c(-c4cnc(N5CCC[C@H]5C(N)=O)nc4)c2cc1OC)C(=O)OC3]

The compound of Example 49 was synthesized by the same method as that of the compound of Example 7 except for using (1S)-2-(5-(4,4,5,5-tetramethyl-1,3,2-dioxaborolan-2-yl)pyrimidin-2-yl)cyclopentan-1-carboxamide instead of 2-(4-methylpiperazin-1-yl)-5-(4,4,5,5-tetramethyl-1,3,2-dioxaborolan-2-yl)pyrimidine.

Mass (M+H⁺): 435.2

¹H-NMR (500 Mhz, DMSO-d6): δ 1.19 (s, 2H), 1.93-1.98 (m, 3H), 2.20-2.23 (m, 1H), 3.73 (s, 3H), 3.92 (s, 3H), 4.44-4.46 (m, 1H), 5.42 (s, 2H), 7.07 (s, 1H), 7.39 (s, 1H), 7.50 (s, 1H), 7.93 (s, 1H), 8.40 (br, 2H)

### [Example 50] Synthesis of 6,7-dimethoxy-9-(2-(((5-methylfuran-2-yl)methyl)amino)pyrimidin-5-yl)naphtho[2,3-c]furan-1(3H)-one

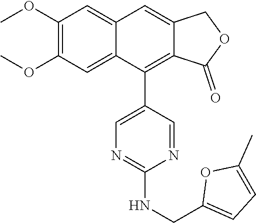 [SMILES: COc1cc2cc3c(c(-c4cnc(NCc5ccc(C)o5)nc4)c2cc1OC)C(=O)OC3]

The compound of Example 50 was synthesized by the same method as that of the compound of Example 7 except for using N-((5-methylfuran-2-yl)methyl)-5-(4,4,5,5-tetramethyl-1,3,2-dioxaborolan-2-yl)pyrimidin-2-amine instead of 2-(4-methylpiperazin-1-yl)-5-(4,4,5,5-tetramethyl-1,3,2-dioxaborolan-2-yl)pyrimidine.

Mass (M+H⁺): 432.2

¹H-NMR (500 Mhz, DMSO-d6): δ 2.21 (s, 3H) 3.73 (s, 3H), 3.93 (s, 3H), 4.50 (d, J=5.75 Hz, 2H), 5.45 (s, 2H), 5.95 (d, J=1.15 Hz, 1H), 6.13 (d, J=2.85 Hz, 1H), 7.06 (s, 1H), 7.54 (s, 1H), 7.76 (t, J=6.3 Hz, 1H), 7.93 (s, 1H), 8.83 (s, 2H)

### [Example 51] Synthesis of 6,7-dimethoxy-9-(2-morpholinopyrimidin-5-yl)naphtho[2,3-c]furan-1(3H)-one

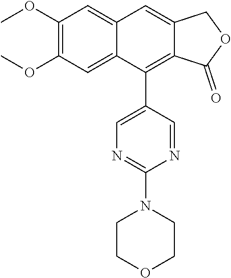 [SMILES: COc1cc2cc3c(c(-c4cnc(N5CCOCC5)nc4)c2cc1OC)C(=O)OC3]

The compound of Example 51 was synthesized by the same method as that of the compound of Example 7 except for using 4-(5-(4,4,5,5-tetramethyl-1,3,2-dioxaborolan-2-yl)pyrimidin-2-yl)morpholine instead of 2-(4-methylpiperazin-1-yl)-5-(4,4,5,5-tetramethyl-1,3,2-dioxaborolan-2-yl)pyrimidine.

Mass (M+H⁺): 408.1

¹H-NMR (500 Mhz, DMSO-d6): δ 3.64-3.65 (m, 4H), 3.68 (s, 3H), 3.72-3.74 (m, 4H), 3.87 (s, 3H), 5.37 (s, 2H), 7.02 (s, 1H), 7.45 (s, 1H), 7.89 (s, 1H), 8.38 (s, 2H)

### [Example 52] Synthesis of 9-(benzofuran-5-yl)-6,7-dimethoxynaphtho[2,3-c]furan-1(3H)-one

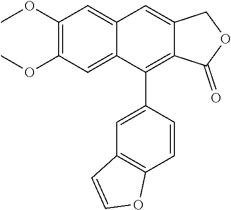 [SMILES: COc1cc2cc3c(c(-c4ccc5occc5c4)c2cc1OC)C(=O)OC3]

The compound of Example 52 was synthesized by the same method as that of the compound of Example 7 except for using 2-(benzofuran-5-yl)-4,4,5,5-tetramethyl-1,3,2-dioxaborolane instead of 2-(4-methylpiperazin-1-yl)-5-(4,4,5,5-tetramethyl-1,3,2-dioxaborolan-2-yl)pyrimidine.

Mass (M+H⁺): 361.1

¹H-NMR (500 Mhz, DMSO-d6): δ 3.57 (s, 3H), 3.90 (s, 3H), 5.43 (s, 2H), 6.88 (s, 1H), 6.99-7.00 (m, 1H), 7.25 (dd, J=8.6, 1.7 Hz, 1H), 7.50 (s, 1H), 7.61 (s, 1H), 7.69 (d, J=8.6 Hz, 1H), 7.95 (s, 1H), 8.05 (s, 1H)

### [Example 53] Synthesis of 9-(5-benzothiophen-2-yl)-6,7-dimethoxynaphtho[2,3-c]furan-1(3H)-one

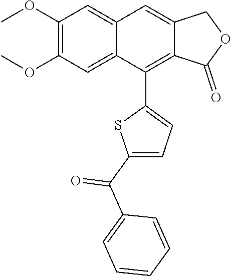 [SMILES: COc1cc2cc3c(c(-c4ccc(C(=O)c5ccccc5)s4)c2cc1OC)C(=O)OC3]

The compound of Example 53 was synthesized by the same method as that of the compound of Example 7 except for using phenyl (5-(4,4,5,5-tetramethyl-1,3,2-dioxaborolan-2-yl)thiophen-2-yl) methanone instead of 2-(4-methylpiperazin-1-yl)-5-(4,4,5,5-tetramethyl-1,3,2-dioxaborolan-2-yl)pyrimidine.

Mass (M+H⁺): 431.1

¹H-NMR (500 Mhz, DMSO-d6): δ 3.71 (s, 3H), 3.94 (s, 3H), 5.46 (s, 2H), 7.09 (s, 1H), 7.34 (d, J=3.7 Hz, 1H), 7.55 (s, 1H), 7.57-7.60 (m, 2H), 7.67-7.68 (m, 1H), 7.84 (d, J=3.75 Hz, 1H), 7.89-7.91 (m, 2H), 8.06 (s, 1H)

### [Example 54] Synthesis of 9-(2-(2-((2S,6R)-2,6-dimethylmorpholino) ethoxy)pyrimidin-5-yl)-6,7-dimethoxynaphtho[2,3-c]furan-1(3H)-one

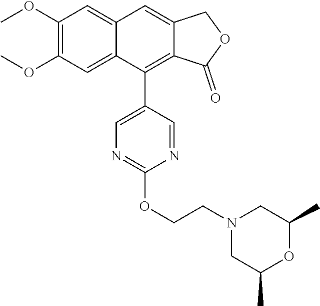

The compound of Example 54 was synthesized by the same method as that of the compound of Example 7 except for using (2S,6R)-2,6-dimethyl-4-(2-((5-(4,4,5,5-tetramethyl-1,3,2-dioxaborolan-2-yl)pyrimidin-2-yl)oxy)ethyl)morpholine instead of 2-(4-methylpiperazin-1-yl)-5-(4,4,5,5-tetramethyl-1,3,2-dioxaborolan-2-yl)pyrimidine.

Mass (M+H⁺): 480.3

¹H-NMR (500 Mhz, DMSO-d6): δ 1.02 (d, J=6.3 Hz, 6H), 1.71 (t, J=10.3 Hz, 2H), 2.71 (t, J=5.75 Hz, 2H), 2.83 (d, J=10.0 Hz, 2H), 3.53-3.54 (m, 2H), 3.72 (s, 3H), 3.93 (s, 3H), 4.49 (t, J=5.7 Hz, 2H), 5.45 (s, 2H), 6.97 (s, 1H), 7.54 (s, 1H), 8.01 (s, 1H), 8.63 (s, 2H)

### [Example 55] Synthesis of (S)-9-(2-((1-(4-fluorophenyl)ethyl)amino)pyrimidin-5-yl)-6,7-dimethoxynaphtho[2,3-c]furan-1(3H)-one

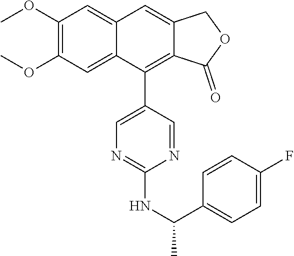 [SMILES: COc1cc2cc3c(c(-c4cnc(N[C@@H](C)c5ccc(F)cc5)nc4)c2cc1OC)C(=O)OC3]

The compound of Example 55 was synthesized by the same method as that of the compound of Example 7 except for using (S)—N-(1-(4-fluorophenyl)ethyl)-5-(4,4,5,5-tetramethyl-1,3,2-dioxaborolan-2-yl)pyrimidin-2-amine instead of 2-(4-methylpiperazin-1-yl)-5-(4,4,5,5-tetramethyl-1,3,2-dioxaborolan-2-yl)pyrimidine.

Mass (M+H⁺): 460.1

¹H-NMR (500 Mhz, DMSO-d6): δ 1.07-1.14 (m, 3H), 3.64 (s, 3H), 3.86 (s, 3H), 5.12-5.14 (m, 1H), 5.35 (s, 2H), 7.05 (s, 1H), 7.39-7.43 (m, 4H), 7.86-7.87 (m, 2H), 8.23 (s, 2H)

### [Example 56] Synthesis of 9-(3-acetyl-2-oxo-2H-chromen-6-yl)-6,7-dimethoxynaphtho[2,3-c]furan-1(3H)-one

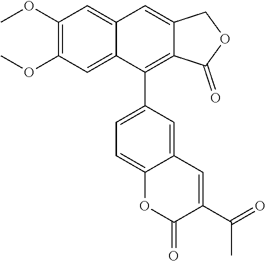 [SMILES: COc1cc2cc3c(c(-c4ccc5oc(=O)c(C(C)=O)cc5c4)c2cc1OC)C(=O)OC3]

The compound of Example 56 was synthesized by the same method as that of the compound of Example 7 except for using 3-acetyl-6-(4,4,5,5-tetramethyl-1,3,2-dioxaborolan-2-yl)-2H-chroman-2-one instead of 2-(4-methylpiperazin-1-yl)-5-(4,4,5,5-tetramethyl-1,3,2-dioxaborolan-2-yl)pyrimidine.

Mass (M+H⁺): 431.1

¹H-NMR (500 Mhz, DMSO-d6): δ 2.60 (s, 3H), 3.61 (s, 3H), 3.93 (s, 3H), 5.45 (s, 2H), 6.87 (s, 1H), 7.54 (s, 1H), 7.59 (d, J=8.6 Hz, 1H), 7.73 (dd, J=8.6, 2.0 Hz, 1H), 7.97 (d, J=2.3 Hz, 1H), 8.00 (s, 1H), 8.67 (s, 2H)

### [Example 57] Synthesis of 9-(2-(3,5-dimethylpiperidin-1-yl)pyrimidin-5-yl)-6,7-dimethoxynaphtho[2,3-c]furan-1(3H)-one

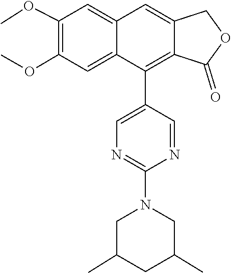 [SMILES: COc1cc2cc3c(c(-c4cnc(N5CC(C)CC(C)C5)nc4)c2cc1OC)C(=O)OC3]

The compound of Example 57 was synthesized by the same method as that of the compound of Example 7 except for using 2-(3,5-dimethylpiperidin-1-yl)-5-(4,4,5,5-tetramethyl-1,3,2-dioxaborolan-2-yl)pyrimidine instead of 2-(4-methylpiperazin-1-yl)-5-(4,4,5,5-tetramethyl-1,3,2-dioxaborolan-2-yl)pyrimidine.

Mass (M+H⁺): 434.2

¹H-NMR (500 Mhz, DMSO-d6): δ 0.74-0.82 (m, 2H), 0.86 (d, J=6.6 Hz, 6H), 1.53-1.55 (m, 2H), 2.32 (t, J=12.3 Hz, 2H), 3.68 (s, 3H), 3.87 (s, 3H), 4.70-4.72 (m, 2H), 5.37 (s, 2H), 7.03 (s, 1H), 7.45 (s, 1H), 7.88 (s, 1H), 8.32 (s, 2H)

### [Example 58] Synthesis of 9-(2-((2-(diethylamino)ethyl)amino)pyrimidin-5-yl)-6,7-dimethoxynaphtho[2,3-c]furan-1(3H)-one

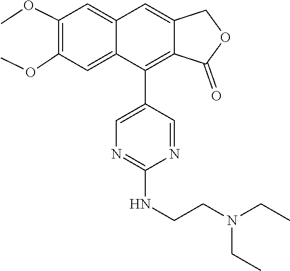 [SMILES: CCN(CC)CCNc1ncc(-c2c3c(cc4cc(OC)c(OC)cc24)COC3=O)cn1]

The compound of Example 58 was synthesized by the same method as that of the compound of Example 7 except for using N1,N1-diethyl-N2-(5-(4,4,5,5-tetramethyl-1,3,2-dioxaborolan-2-yl)pyrimidin-2-yl) ethan-1,2-diamine instead of 2-(4-methylpiperazin-1-yl)-5-(4,4,5,5-tetramethyl-1,3,2-dioxaborolan-2-yl)pyrimidine.

Mass (M+H⁺): 464.2

¹H-NMR (500 Mhz, DMSO-d6): δ 0.89-0.92 (m, 6H), 2.41-2.50 (m, 6H), 3.35 (d, J=5.7 Hz, 2H), 3.68 (s, 3H), 3.87 (s, 3H), 5.37 (s, 2H), 7.02-7.06 (m, 2H), 7.45 (s, 1H), 7.87 (s, 1H), 8.25 (s, 2H)

### [Example 59] Synthesis of 6,7-dimethoxy-9-(2-(3-methylpiperidin-1-yl)pyrimidin-5-yl)naphtho[2,3-c]furan-1(3H)-one

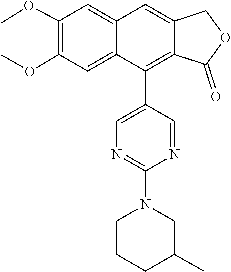 [SMILES: COc1cc2cc3c(c(-c4cnc(N5CCCC(C)C5)nc4)c2cc1OC)C(=O)OC3]

The compound of Example 59 was synthesized by the same method as that of the compound of Example 7 except for using 2-(3-methylpiperidin-1-yl)-5-(4,4,5,5-tetramethyl-1,3,2-dioxaborolan-2-yl)pyrimidine instead of 2-(4-methylpiperazin-1-yl)-5-(4,4,5,5-tetramethyl-1,3,2-dioxaborolan-2-yl)pyrimidine. Mass (M+H⁺): 420.2

¹H-NMR (500 Mhz, DMSO-d6): δ 0.85-0.87 (m, 3H), 1.12-1.14 (m, 1H), 1.40-1.43 (m, 1H), 1.50-1.55 (m, 1H), 1.65-1.67 (m, 1H), 1.75-1.77 (m, 1H), 2.55 (t, J=11.75 Hz, 1H), 2.85 (t, J=12.25 Hz, 1H), 3.69 (s, 3H), 3.87 (s, 3H), 4.59-4.62 (m, 2H), 5.37 (s, 2H), 7.03 (s, 1H), 7.45 (s, 1H), 7.88 (s, 1H), 8.32 (s, 2H)

### [Example 60] Synthesis of 1-(5-(6,7-dimethoxy-3-oxo-1,3-dihydronaphtho[2,3-c]furan-4-yl)pyrimidin-2-yl) piperidin-3-carboxylic acid

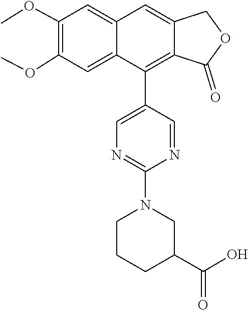 [SMILES: COc1cc2cc3c(c(-c4cnc(N5CCCC(C(=O)O)C5)nc4)c2cc1OC)C(=O)OC3]

The compound of Example 60 was synthesized by the same method as that of the compound of Example 7 except for using 1-(5-(4,4,5,5-tetramethyl-1,3,2-dioxaborolan-2-yl)pyrimidin-2-yl) piperidin-3-carboxylic acid instead of 2-(4-methylpiperazin-1-yl)-5-(4,4,5,5-tetramethyl-1,3,2-dioxaborolan-2-yl)pyrimidine.

Mass (M+H⁺): 450.2

¹H-NMR (500 Mhz, DMSO-d6): δ 1.14-1.15 (m, 1H), 1.40-1.42 (m, 1H), 1.53-1.55 (m, 1H), 1.67-1.69 (m, 1H), 1.95-1.97 (m, 1H), 2.25-2.27 (m, 1H), 2.93-2.99 (m, 2H), 3.68 (s, 3H), 3.87 (s, 3H), 4.52-4.55 (m, 1H), 4.74-4.76 (m, 1H), 5.36 (s, 2H), 7.03 (s, 1H), 7.44 (s, 1H), 7.87 (s, 1H), 8.32 (s, 2H)

### [Example 61] Synthesis of (5-(6,7-dimethoxy-3-oxo-1,3-dihydronaphtho[2,3-c]furan-4-yl)pyrimidin-2-yl)-L-proline

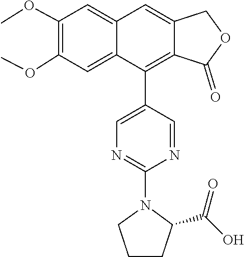 [SMILES: COc1cc2cc3c(c(-c4cnc(N5CCC[C@H]5C(=O)O)nc4)c2cc1OC)C(=O)OC3]

The compound of Example 61 was synthesized by the same method as that of the compound of Example 7 except for using (1R)-2-(5-(4,4,5,5-tetramethyl-1,3,2-dioxaborolan-2-yl)pyrimidin-2-yl)cyclopentan-1-carboxylic acid instead of 2-(4-methylpiperazin-1-yl)-5-(4,4,5,5-tetramethyl-1,3,2-dioxaborolan-2-yl)pyrimidine.

Mass (M+H⁺): 436.2

¹H-NMR (500 Mhz, DMSO-d6): δ 1.11-1.23 (m, 3H), 1.90-2.01 (m, 3H), 3.70 (s, 3H), 3.88 (s, 3H), 4.47-4.91 (m, 1H), 5.39 (s, 2H), 7.02 (s, 1H), 7.48 (s, 1H), 7.91 (s, 1H), 8.34-8.39 (m, 2H)

### [Example 62] Synthesis of 9-(2-((2S,6S)-2,6-dimethylmorpholino)pyrimidin-5-yl)-6,7-dimethoxynaphtho[2,3-c]furan-1(3H)

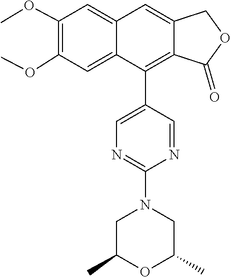 [SMILES: COc1cc2cc3c(c(-c4cnc(N5C[C@H](C)O[C@@H](C)C5)nc4)c2cc1OC)C(=O)OC3]

The compound of Example 62 was synthesized by the same method as that of the compound of Example 7 except for using (2S,6S)-2,6-dimethyl-4-(5-(4,4,5,5-tetramethyl-1,3,2-dioxaborolan-2-yl)pyrimidin-2-yl) morpholine instead of 2-(4-methylpiperazin-1-yl)-5-(4,4,5,5-tetramethyl-1,3,2-dioxaborolan-2-yl)pyrimidine.

Mass (M+H⁺): 436.2

¹H-NMR (500 Mhz, CDCl₃): δ 1.29 (d, J=6.3 Hz, 6H), 3.63-3.64 (m, 2H), 3.89 (s, 3H), 4.03 (d, J=13.15 Hz, 2H), 4.05 (s, 3H), 4.16-4.17 (m, 2H), 5.39 (s, 2H), 7.19 (d, J=11.45 Hz, 2H), 7.72 (s, 1H), 8.40 (s, 2H)

### [Example 63] Synthesis of 6,7-dimethoxy-9-(2-thiomorpholinopyrimidin-5-yl)naphtho[2,3-c]furan-1(3H)-one

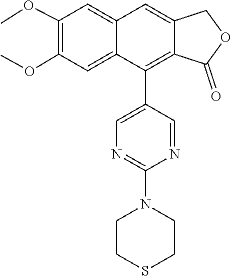 [SMILES: COc1cc2cc3c(c(-c4cnc(N5CCSCC5)nc4)c2cc1OC)C(=O)OC3]

The compound of Example 63 was synthesized by the same method as that of the compound of Example 7 except for using 4-(5-(4,4,5,5-tetramethyl-1,3,2-dioxaborolan-2-yl)pyrimidin-2-yl)thiomorpholine instead of 2-(4-methylpiperazin-1-yl)-5-(4,4,5,5-tetramethyl-1,3,2-dioxaborolan-2-yl)pyrimidine.

Mass (M+H⁺): 424.2

¹H-NMR (500 Mhz, DMSO-d6): δ 2.64-2.68 (m, 4H), 3.74 (s, 3H), 3.92 (s, 3H), 4.13-4.15 (m, 4H), 5.42 (s, 2H), 7.08 (s, 1H), 7.51 (s, 1H), 7.94 (s, 1H), 8.42 (s, 2H)

### [Example 64] Synthesis of 6,7-dimethoxy-9-(2-((2-methylbutyl)amino)pyrimidin-5-yl)naphtho[2,3-c]furan-1(3H)-one

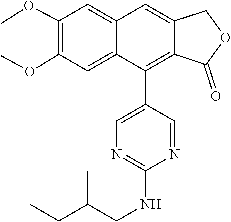 [SMILES: CCC(C)CNc1ncc(-c2c3c(cc4cc(OC)c(OC)cc24)COC3=O)cn1]

The compound of Example 64 was synthesized by the same method as that of the compound of Example 7 except for using N-(2-methylbutyl)-5-(4,4,5,5-tetramethyl-1,3,2-dioxaborolan-2-yl)pyrimidin-2-amine instead of 2-(4-methylpiperazin-1-yl)-5-(4,4,5,5-tetramethyl-1,3,2-dioxaborolan-2-yl)pyrimidine.

Mass (M+H⁺): 408.2

¹H-NMR (500 Mhz, DMSO-d6): δ 0.87-0.90 (m, 6H), 1.12-1.18 (m, 1H), 1.44-1.47 (m, 1H), 1.65-1.69 (m, 1H), 3.15-3.20 (m, 2H), 3.73 (s, 3H), 3.88 (s, 3H), 5.41 (s, 2H), 7.08 (s, 1H), 7.40 (t, J=6.0 Hz, 1H), 7.49 (s, 1H), 7.92 (s, 1H), 8.28 (s, 2H)

### [Example 65] Synthesis of 6,7-dimethoxy-9-(2-(methyl(2-methylbutyl)amino)pyrimidin-5-yl)naphtho[2,3-c]furan-1(3H)-one

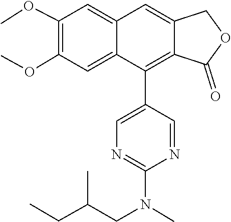 [SMILES: CCC(C)CN(C)c1ncc(-c2c3c(cc4cc(OC)c(OC)cc24)COC3=O)cn1]

The compound of Example 65 was synthesized by the same method as that of the compound of Example 7 except for using N-methyl-N-(2-methylbutyl)-5-(4,4,5,5-tetramethyl-1,3,2-dioxaborolan-2-yl)pyrimidin-2-amine instead of 2-(4-methylpiperazin-1-yl)-5-(4,4,5,5-tetramethyl-1,3,2-dioxaborolan-2-yl)pyrimidine.

Mass (M+H⁺): 422.2

¹H-NMR (500 Mhz, DMSO-d6): δ 0.78-0.86 (m, 6H), 1.02-1.14 (m, 3H), 3.10 (s, 3H), 3.50-3.53 (m, 2H), 3.66 (s, 3H), 3.86 (s, 3H), 5.36 (s, 2H), 7.00 (s, 1H), 7.44 (s, 1H), 7.86 (s, 1H), 8.30 (s, 2H)

### [Example 66] Synthesis of 9-(2-(butyl(ethyl)amino)pyrimidin-5-yl)-6,7-dimethoxynaphtho[2,3-c]furan-1(3H)-one

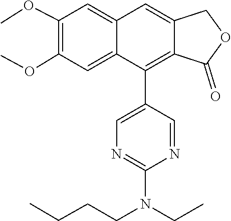 [SMILES: CCCCN(CC)c1ncc(-c2c3c(cc4cc(OC)c(OC)cc24)COC3=O)cn1]

The compound of Example 66 was synthesized by the same method as that of the compound of Example 7 except for using N-butyl-N-ethyl-5-(4,4,5,5-tetramethyl-1,3,2-dioxaborolan-2-yl)pyrimidin-2-amine instead of 2-(4-methylpiperazin-1-yl)-5-(4,4,5,5-tetramethyl-1,3,2-dioxaborolan-2-yl)pyrimidine.

Mass (M+H⁺): 422.2

¹H-NMR (500 Mhz, DMSO-d6): δ 0.80-0.88 (m, 3H), 1.10-1.12 (m, 3H), 1.21-1.30 (m, 2H), 1.54-1.55 (m, 2H), 3.55-3.61 (m, 4H), 3.69 (s, 3H), 3.88 (s, 3H), 5.37 (s, 2H), 7.04 (s, 1H), 7.45 (s, 1H), 7.88 (s, 1H), 8.32 (s, 2H)

### [Example 67] Synthesis of 9-(2-((cyclopropylmethyl)amino)pyrimidin-5-yl)-6,7-dimethoxynaphtho[2,3-c]furan-1(3H)-one

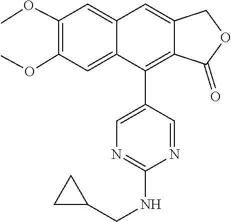 [SMILES: COc1cc2cc3c(c(-c4cnc(NCC5CC5)nc4)c2cc1OC)C(=O)OC3]

The compound of Example 67 was synthesized by the same method as that of the compound of Example 7 except for using N-(cyclopropylmethyl)-5-(4,4,5,5-tetramethyl-1,3,2-dioxaborolan-2-yl)pyrimidin-2-amine instead of 2-(4-methylpiperazin-1-yl)-5-(4,4,5,5-tetramethyl-1,3,2-dioxaborolan-2-yl)pyrimidine.

Mass (M+H⁺): 392.2

¹H-NMR (500 Mhz, DMSO-d6): δ 0.18 (d, J=4.6 Hz, 2H), 0.37 (d, J=8.25 Hz, 2H), 1.01-1.05 (m, 1H), 3.15-3.23 (m, 2H), 3.68 (s, 3H), 3.87 (s, 3H), 5.36 (s, 2H), 7.03 (s, 1H), 7.41-7.44 (m, 2H), 7.86 (s, 1H), 8.24 (s, 2H)

### [Example 68] Synthesis of 9-(2-((cyclopropylmethyl)(methyl)amino)pyrimidin-5-yl)-6,7-dimethoxynaphtho[2,3-c]furan-1(3H)-one

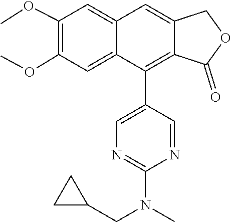 [SMILES: COc1cc2cc3c(c(-c4cnc(N(C)CC5CC5)nc4)c2cc1OC)C(=O)OC3]

The compound of Example 68 was synthesized by the same method as that of the compound of Example 7 except for using N-(cyclopropylmethyl)-N-methyl-5-(4,4,5,5-tetramethyl-1,3,2-dioxaborolan-2-yl)pyrimidin-2-amine instead of 2-(4-methylpiperazin-1-yl)-5-(4,4,5,5-tetramethyl-1,3,2-dioxaborolan-2-yl)pyrimidine.

Mass (M+H⁺): 406.2

¹H-NMR (500 Mhz, CDCl₃): 0.26-0.35 (m, 2H), 0.47-0.56 (m, 2H), 1.12-1.22 (m, 1H), 3.31 (s, 3H), 3.62 (d, J=6.6 Hz, 2H), 3.85 (s, 3H), 4.06 (s, 3H), 5.35 (s, 2H), 7.17 (d, J=4.85 Hz, 2H), 7.68 (s, 1H), 8.38 (s, 2H)

### [Example 69] Synthesis of 9-(2-(cyclopropylamino)pyrimidin-5-yl)-6,7-dimethoxynaphtho[2,3-c]furan-1(3H)-one

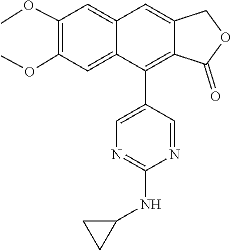 [SMILES: COc1cc2cc3c(c(-c4cnc(NC5CC5)nc4)c2cc1OC)C(=O)OC3]

The compound of Example 69 was synthesized by the same method as that of the compound of Example 7 except for using N-cyclopropyl-5-(4,4,5,5-tetramethyl-1,3,2-dioxaborolan-2-yl)pyrimidin-2-amine instead of 2-(4-methylpiperazin-1-yl)-5-(4,4,5,5-tetramethyl-1,3,2-dioxaborolan-2-yl)pyrimidine.

Mass (M+H⁺): 378.2

¹H-NMR (500 Mhz, DMSO-d6): δ 0.47 (s, 2H), 0.63 (s, 2H), 2.73 (s, 1H), 3.68 (s, 3H), 3.86 (s, 3H), 5.41 (s, 2H), 7.02 (s, 1H), 7.44 (s, 1H), 7.53 (s, 1H), 7.91 (s, 1H), 8.27 (s, 2H)

### [Example 70] Synthesis of 9-(2-(cyclopropyl(methyl)amino)pyrimidin-5-yl)-6,7-dimethoxynaphtho[2,3-c]furan-1(3H)-one

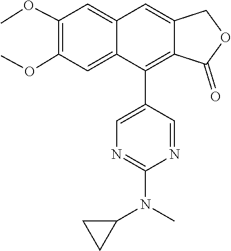 [SMILES: COc1cc2cc3c(c(-c4cnc(N(C)C5CC5)nc4)c2cc1OC)C(=O)OC3]

The compound of Example 70 was synthesized by the same method as that of the compound of Example 7 except for using N-cyclopropyl-N-methyl-5-(4,4,5,5-tetramethyl-1,3,2-dioxaborolan-2-yl)pyrimidin-2-amine instead of 2-(4-methylpiperazin-1-yl)-5-(4,4,5,5-tetramethyl-1,3,2-dioxaborolan-2-yl)pyrimidine.

Mass (M+H⁺): 392.2

¹H-NMR (500 Mhz, DMSO-d6): δ 0.65 (s, 2H), 0.79-0.81 (m, 2H), 2.83-2.84 (m, 1H), 3.11 (s, 3H), 3.69 (s, 3H), 3.89 (s, 3H), 5.41 (s, 2H), 7.03 (s, 1H), 7.47 (s, 1H), 7.90 (s, 1H), 8.39 (s, 2H)

### [Example 71] Synthesis of 9-(2-(butyl(methyl)amino)pyrimidin-5-yl)-6,7-dimethoxynaphtho[2,3-c]furan-1(3H)-one

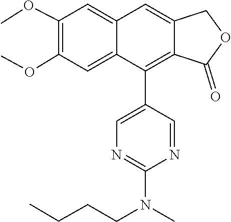 [SMILES: CCCCN(C)c1ncc(-c2c3c(cc4cc(OC)c(OC)cc24)COC3=O)cn1]

The compound of Example 71 was synthesized by the same method as that of the compound of Example 7 except for using N-butyl-N-methyl-5-(4,4,5,5-tetramethyl-1,3,2-dioxaborolan-2-yl)pyrimidin-2-amine instead of 2-(4-methylpiperazin-1-yl)-5-(4,4,5,5-tetramethyl-1,3,2-dioxaborolan-2-yl)pyrimidine.

Mass (M+H⁺): 408.2

¹H-NMR (500 Mhz, CDCl₃): 0.91-0.94 (m, 3H), 1.30-1.37 (m, 2H), 1.6-1.65 (m, 2H), 3.21 (s, 3H), 3.65-3.70 (m, 2H), 3.84 (s, 3H), 4.01 (s, 3H), 5.34 (s, 2H), 7.16 (d, J=6.85 Hz, 2H), 7.66 (s, 1H), 8.35 (s, 2H)

### [Example 72] Synthesis of 9-(2-(bis(2-methoxyethyl)amino)pyrimidin-5-yl)-6,7-dimethoxynaphtho[2,3-c]furan-1(3H)-one

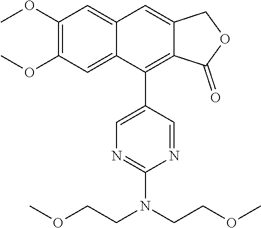

The compound of Example 72 was synthesized by the same method as that of the compound of Example 7 except for using N,N-bis(2-methoxyethyl)-5-(4,4,5,5-tetramethyl-1,3,2-dioxaborolan-2-yl)pyrimidin-2-amine instead of 2-(4-methylpiperazin-1-yl)-5-(4,4,5,5-tetramethyl-1,3,2-dioxaborolan-2-yl)pyrimidine.

Mass (M+H⁺): 454.2

¹H-NMR (500 Mhz, CDCl₃): δ 3.37 (s, 6H), 3.68 (s, 4H), 3.89 (s, 3H), 4.04 (s, 7H), 5.37 (s, 2H), 7.13-7.22 (m, 2H), 7.71 (s, 1H), 8.44 (s, 2H)

### [Example 73] Synthesis of 9-(2-(dibutylamino)pyrimidin-5-yl)-6,7-dimethoxynaphtho[2,3-c]furan-1(3H)-one

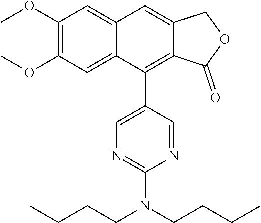 [SMILES: CCCCN(CCCC)c1ncc(-c2c3c(cc4cc(OC)c(OC)cc24)COC3=O)cn1]

The compound of Example 73 was synthesized by the same method as that of the compound of Example 7 except for using N,N-dibutyl-5-(4,4,5,5-tetramethyl-1,3,2-dioxaborolan-2-yl)pyrimidin-2-amine instead of 2-(4-methylpiperazin-1-yl)-5-(4,4,5,5-tetramethyl-1,3,2-dioxaborolan-2-yl)pyrimidine.

Mass (M+H⁺): 450.3

¹H-NMR (500 Mhz, CDCl₃): δ 1.019 (t, J=6.85 Hz 6H), 1.41-1.48 (m, 4H), 1.69-1.75 (m, 4H), 3.69 (t, J=7.45, 4H), 3.79 (s, 3H), 4.01 (s, 3H), 5.42 (s, 2H), 7.23 (s, 1H), 7.28 (s, 1H), 7.74 (s, 1H), 8.43 (s, 2H)

### [Example 74] Synthesis of 9-(2-(diethylamino)pyrimidin-5-yl)-6,7-dimethoxynaphtho[2,3-c]furan-1(3H)-one

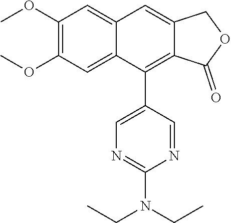 [SMILES: CCN(CC)c1ncc(-c2c3c(cc4cc(OC)c(OC)cc24)COC3=O)cn1]

The compound of Example 74 was synthesized by the same method as that of the compound of Example 7 except for using N,N-diethyl-5-(4,4,5,5-tetramethyl-1,3,2-dioxaborolan-2-yl)pyrimidin-2-amine instead of 2-(4-methylpiperazin-1-yl)-5-(4,4,5,5-tetramethyl-1,3,2-dioxaborolan-2-yl)pyrimidine.

Mass (M+H⁺): 394.2

¹H-NMR (500 Mhz, CDCl₃): δ 1.50 (s, 6H), 1.62 (s, 4H), 3.85 (s, 3H), 4.00 (s, 3H), 5.33 (s, 2H), 7.14 (s, 1H), 7.17 (s, 1H), 7.66 (s, 1H), 8.36 (s, 2H)

### [Example 75] Synthesis of 9-(2-(ethyl(isopropyl)amino)pyrimidin-5-yl)-6,7-dimethoxynaphtho[2,3-c]furan-1(3H)-one hydrochloride

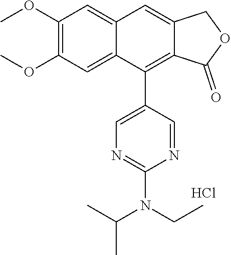 [SMILES: CCN(c1ncc(-c2c3c(cc4cc(OC)c(OC)cc24)COC3=O)cn1)C(C)C ; Cl]

The compound of Example 75 was synthesized by the same method as that of the compound of Example 7 except for using N-ethyl-N-isopropyl-5-(4,4,5,5-tetramethyl-1,3,2-dioxaborolan-2-yl)pyrimidin-2-amine instead of 2-(4-methylpiperazin-1-yl)-5-(4,4,5,5-tetramethyl-1,3,2-dioxaborolan-2-yl)pyrimidine.

Mass (M+H⁺): 408.1

¹H-NMR (500 Mhz, DMSO-d6): δ 1.12-1.21 (m, 9H), 3.47-3.54 (m, 2H), 3.69 (s, 3H), 3.87 (s, 3H), 4.93-4.97 (m, 1H), 5.36 (s, 2H), 7.06 (s, 1H), 7.44 (s, 1H), 7.88 (s, 1H), 8.37 (s, 2H)

### [Example 76] Synthesis of 9-(2-(butyl(methyl)amino)pyrimidin-5-yl)-6,7-dimethoxynaphtho[2,3-c]furan-1(3H)-one hydrochloride

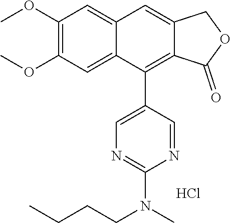 [SMILES: CCCCN(C)c1ncc(-c2c3c(cc4cc(OC)c(OC)cc24)COC3=O)cn1 ; Cl]

The compound of Example 76 was synthesized by the same method as that of the compound of Example 7 except for using N-butyl-N-methyl-5-(4,4,5,5-tetramethyl-1,3,2-dioxaborolan-2-yl)pyrimidin-2-amine instead of 2-(4-methylpiperazin-1-yl)-5-(4,4,5,5-tetramethyl-1,3,2-dioxaborolan-2-yl)pyrimidine.

Mass (M+H⁺): 408.2

¹H-NMR (500 Mhz, DMSO-d6): δ 0.83-0.89 (m, 3H), 1.24-1.32 (m, 2H), 1.52-1.59 (m, 2H), 3.10 (s, 3H), 3.60 (s, 2H), 3.67 (s, 3H), 3.87 (s, 3H), 5.36 (s, 2H), 7.02 (s, 1H), 7.44 (s, 1H), 7.87 (s, 1H), 8.33 (s, 2H)

### [Example 77] Synthesis of 9-(2-((2-hydroxyethyl)(propyl)amino)pyrimidin-5-yl)-6,7-dimethoxynaphtho[2,3-c]furan-1(3H)-one hydrochloride

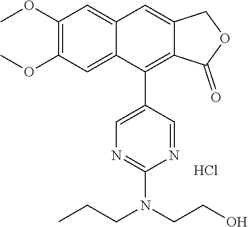 [SMILES: CCCN(CCO)c1ncc(-c2c3c(cc4cc(OC)c(OC)cc24)COC3=O)cn1 ; Cl]

The compound of Example 77 was synthesized by the same method as that of the compound of Example 7 except for using 2-(propyl (5-(4,4,5,5-tetramethyl-1,3,2-dioxaborolan-2-yl)pyrimidin-2-yl)amino) ethan-1-ol instead of 2-(4-methylpiperazin-1-yl)-5-(4,4,5,5-tetramethyl-1,3,2-dioxaborolan-2-yl)pyrimidine.

Mass (M+H⁺): 424.2

¹H-NMR (500 Mhz, DMSO-d6): δ 0.84 (t, J=7.45 Hz, 3H), 1.57-1.62 (m, 2H), 3.55-3.60 (m, 4H), 3.65-3.66 (m, 2H), 3.70 (s, 3H), 3.88 (s, 3H), 5.38 (s, 2H), 7.06 (s, 1H), 7.46 (s, 1H), 7.90 (s, 1H), 8.38 (s, 2H)

### [Example 78] Synthesis of 9-(2-(cyclohexyl(ethyl)amino)pyrimidin-5-yl)-6,7-dimethoxynaphtho[2,3-c]furan-1(3H)-one hydrochloride

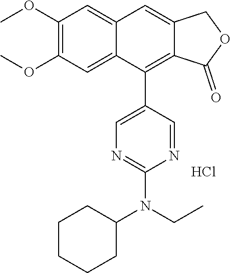 [SMILES: CCN(c1ncc(-c2c3c(cc4cc(OC)c(OC)cc24)COC3=O)cn1)C1CCCCC1 ; Cl]

The compound of Example 78 was synthesized by the same method as that of the compound of Example 7 except for using N-cyclohexyl-N-ethyl-5-(4,4,5,5-tetramethyl-1,3,2-dioxaborolan-2-yl)pyrimidin-2-amine instead of 2-(4-methylpiperazin-1-yl)-5-(4,4,5,5-tetramethyl-1,3,2-dioxaborolan-2-yl)pyrimidine.

Mass (M+H⁺): 448.3

¹H-NMR (500 Mhz, DMSO-d6): δ 1.12-1.14 (m, 4H), 1.29-1.31 (m, 2H), 1.51-1.53 (m, 3H), 1.68-1.72 (m, 4H), 3.53 (s, 2H), 3.71 (s, 3H), 3.88 (s, 3H), 4.55 (s, 1H), 5.37 (s, 2H), 7.06 (s, 1H), 7.46 (s, 1H), 7.88 (s, 1H), 8.40 (s, 2H)

### [Example 79] Synthesis of 9-(2-(cyclohexyl(methyl)amino)pyrimidin-5-yl)-6,7-dimethoxynaphtho[2,3-c]furan-1(3H)-one hydrochloride

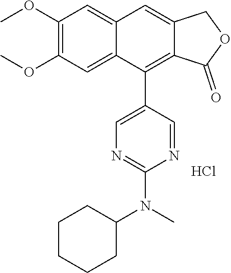 [SMILES: COc1cc2cc3c(c(-c4cnc(N(C)C5CCCCC5)nc4)c2cc1OC)C(=O)OC3 ; Cl]

The compound of Example 79 was synthesized by the same method as that of the compound of Example 7 except for using N-cyclohexyl-N-methyl-5-(4,4,5,5-tetramethyl-1,3,2-dioxaborolan-2-yl)pyrimidin-2-amine instead of 2-(4-methylpiperazin-1-yl)-5-(4,4,5,5-tetramethyl-1,3,2-dioxaborolan-2-yl)pyrimidine.

Mass (M+H⁺): 434.2

¹H-NMR (500 Mhz, DMSO-d6): δ 1.10-1.13 (m, 1H), 1.27-1.35 (m, 2H), 1.48-1.58 (m, 3H), 1.63-1.65 (m, 2H), 1.73-1.75 (m, 2H), 3.01 (s, 3H), 3.69 (s, 3H), 3.87 (s, 3H), 4.55-4.61 (m, 1H), 5.37 (s, 2H), 7.05 (s, 1H), 7.45 (s, 1H), 7.89 (s, 1H), 8.38 (s, 2H)

### [Example 80] Synthesis of 9-(2-(3-hydroxy peridin-1-ylpyrimidin-5-yl)-6,7-dimethoxynaphtho[2,3-c]furan-1(3H)-one hydrochloride

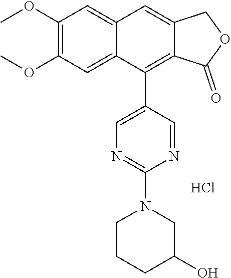 [SMILES: COc1cc2cc3c(c(-c4cnc(N5CCCC(O)C5)nc4)c2cc1OC)C(=O)OC3 ; Cl]

The compound of Example 80 was synthesized by the same method as that of the compound of Example 7 except for using 1-(5-(4,4,5,5-tetramethyl-1,3,2-dioxaborolan-2-yl)pyrimidin-2-yl) piperidin-3-ol instead of 2-(4-methylpiperazin-1-yl)-5-(4,4,5,5-tetramethyl-1,3,2-dioxaborolan-2-yl)pyrimidine.

Mass (M+H⁺): 422.2

¹H-NMR (500 Mhz, DMSO-d6): δ 1.39-1.41 (m, 2H), 1.72 (s, 1H), 1.86-1.89 (m, 1H), 3.02-3.06 (m, 1H), 3.19 (s, 1H), 3.52 (s, 1H), 3.69 (s, 3H), 3.86 (s, 3H), 4.23 (d, J=12.3 Hz, 1H), 4.38 (d, J=10.9 Hz, 1H), 5.36 (s, 2H), 7.03 (s, 1H), 7.44 (s, 1H), 7.88 (s, 1H), 8.38 (s, 2H)

### [Example 81] Synthesis of 9-(2-(ethyl(2-hydroxyethyl)amino)pyrimidin-5-yl)-6,7-dimethoxynaphtho[2,3-c]furan-1(3H)-one hydrochloride

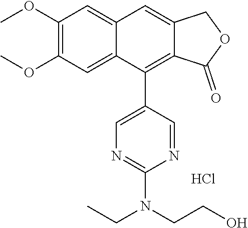 [SMILES: CCN(CCO)c1ncc(-c2c3c(cc4cc(OC)c(OC)cc24)COC3=O)cn1 ; Cl]

The compound of Example 81 was synthesized by the same method as that of the compound of Example 7 except for using 2-(ethyl(5-(4,4,5,5-tetramethyl-1,3,2-dioxaborolan-2-yl)pyrimidin-2-yl)amino) ethan-1-ol instead of 2-(4-methylpiperazin-1-yl)-5-(4,4,5,5-tetramethyl-1,3,2-dioxaborolan-2-yl)pyrimidine.

Mass (M+H⁺): 410.2

¹H-NMR (500 Mhz, DMSO-d6): δ 1.09-1.14 (m, 3H), 3.57-3.67 (m, 6H), 3.70 (s, 3H), 3.88 (s, 3H), 5.42 (s, 2H), 7.06 (s, 1H), 7.46 (s, 1H), 7.89 (s, 1H), 8.38 (s, 2H)

### [Example 82] Synthesis of (R)-9-(2-(2-(hydroxymethyl)pyrrolidin-1-yl)pyrimidin-5-yl)-6,7-dimethoxynaphtho[2,3-c]furan-1(3H)-one hydrochloride

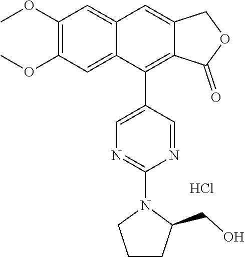 [SMILES: COc1cc2cc3c(c(-c4cnc(N5CCC[C@@H]5CO)nc4)c2cc1OC)C(=O)OC3 ; Cl]

The compound of Example 82 was synthesized by the same method as that of the compound of Example 7 except for using (R)-(1-(5-(4,4,5,5-tetramethyl-1,3,2-dioxaborolan-2-yl)pyrimidin-2-yl)pyrrolidin-2-yl) methanol instead of 2-(4-methylpiperazin-1-yl)-5-(4,4,5,5-tetramethyl-1,3,2-dioxaborolan-2-yl)pyrimidine.

Mass (M+H⁺): 422.2

¹H-NMR (500 Mhz, DMSO-d6): δ 1.86-2.03 (m, 4H), 3.37-3.40 (m, 1H), 3.47-3.52 (m, 1H), 3.55-3.63 (m, 2H), 3.70 (s, 3H), 3.87 (s, 3H), 4.19-4.20 (m, 1H), 5.38 (s, 2H), 7.06 (s, 1H), 7.46 (s, 1H), 7.90 (s, 1H), 8.43 (s, 2H)

### [Example 83] Synthesis of (S)-9-(2-(2-(hydroxymethyl)pyrrolidin-1-yl)pyrimidin-5-yl)-6,7-dimethoxynaphtho[2,3-c]furan-1(3H)-one hydrochloride

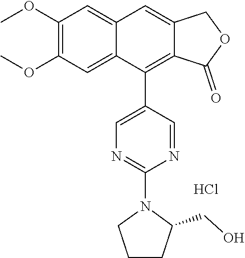 [SMILES: COc1cc2cc3c(c(-c4cnc(N5CCC[C@H]5CO)nc4)c2cc1OC)C(=O)OC3 ; Cl]

The compound of Example 83 was synthesized by the same method as that of the compound of Example 7 except for using (S)-(1-(5-(4,4,5,5-tetramethyl-1,3,2-dioxaborolan-2-yl)pyrimidin-2-yl)pyrrolidin-2-yl) methanol instead of 2-(4-methylpiperazin-1-yl)-5-(4,4,5,5-tetramethyl-1,3,2-dioxaborolan-2-yl)pyrimidine.

Mass (M+H⁺): 422.2

¹H-NMR (500 Mhz, DMSO-d6): § 1.86-2.02 (m, 4H), 3.36-3.40 (m, 1H), 3.47-3.52 (m, 1H), 3.55-3.64 (m, 2H), 3.70 (s, 3H), 3.87 (s, 3H), 4.18-4.19 (m, 1H), 5.38 (s, 2H), 7.06 (s, 1H), 7.46 (s, 1H), 7.90 (s, 1H), 8.42 (s, 2H)

### [Example 84] Synthesis of 9-(2-(cyclopentyl(methyl)amino)pyrimidin-5-yl)-6,7-dimethoxynaphtho[2,3-c]furan-1(3H)-one hydrochloride

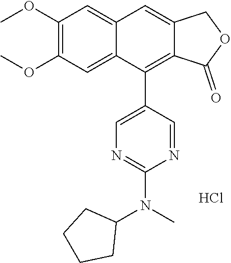 [SMILES: COc1cc2cc3c(c(-c4cnc(N(C)C5CCCC5)nc4)c2cc1OC)C(=O)OC3 ; Cl]

The compound of Example 84 was synthesized by the same method as that of the compound of Example 7 except for using N-cyclopentyl-N-methyl-5-(4,4,5,5-tetramethyl-1,3,2-dioxaborolan-2-yl)pyrimidin-2-amine instead of 2-(4-methylpiperazin-1-yl)-5-(4,4,5,5-tetramethyl-1,3,2-dioxaborolan-2-yl)pyrimidine.

Mass (M+H⁺): 420.2

¹H-NMR (500 Mhz, DMSO-d6): δ 1.50-1.62 (m, 4H), 1.64-1.69 (m, 2H), 1.75-1.81 (m, 2H), 3.00 (s, 3H), 3.69 (s, 3H), 3.87 (s, 3H), 5.14-5.21 (m, 1H), 5.37 (s, 2H), 7.05 (s, 1H), 7.45 (s, 1H), 7.89 (s, 1H), 8.38 (s, 2H)

### [Example 85] Synthesis of 9-(2-(bis(2-hydroxyethyl)amino)pyrimidin-5-yl)-6,7-dimethoxynaphtho[2,3-c]furan-1(3H)-one hydrochloride

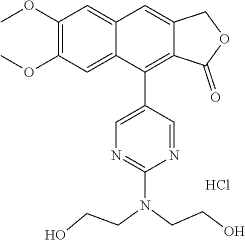 [SMILES: COc1cc2cc3c(c(-c4cnc(N(CCO)CCO)nc4)c2cc1OC)C(=O)OC3 ; Cl]

The compound of Example 85 was synthesized by the same method as that of the compound of Example 7 except for using 2,2′-((5-(4,4,5,5-tetramethyl-1,3,2-dioxaborolan-2-yl)pyrimidin-2-yl) azanediyl)bis(ethan-1-ol) instead of 2-(4-methylpiperazin-1-yl)-5-(4,4,5,5-tetramethyl-1,3,2-dioxaborolan-2-yl)pyrimidine.

Mass (M+H⁺): 426.2

¹H-NMR (500 Mhz, DMSO-d6): δ 3.59 (s, 4H), 3.69 (s, 7H), 3.88 (s, 3H), 4.74 (s, 2H), 5.37 (s, 2H), 7.04 (s, 1H), 7.45 (s, 1H), 7.88 (s, 1H), 8.33 (s, 2H)

### [Example 86] Synthesis of 9-(2-(diisobutylamino)pyrimidin-5-yl)-6,7-dimethoxynaphtho[2,3-c]furan-1(3H)-one hydrochloride

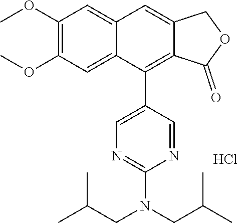 [SMILES: COc1cc2cc3c(c(-c4cnc(N(CC(C)C)CC(C)C)nc4)c2cc1OC)C(=O)OC3 ; Cl]

The compound of Example 86 was synthesized by the same method as that of the compound of Example 7 except for using N,N-diisobutyl-5-(4,4,5,5-tetramethyl-1,3,2-dioxaborolan-2-yl)pyrimidin-2-amine instead of 2-(4-methylpiperazin-1-yl)-5-(4,4,5,5-tetramethyl-1,3,2-dioxaborolan-2-yl)pyrimidine.

Mass (M+H⁺): 450.3

¹H-NMR (500 Mhz, DMSO-d6): δ 0.82 (d, J=6.55 Hz, 12H), 2.08-2.17 (m, 2H), 3.44 (d, J=7.15 Hz, 4H), 3.66 (s, 3H), 3.85 (s, 3H), 5.36 (s, 2H), 7.00 (s, 1H), 7.44 (s, 1H), 7.87 (s, 1H), 8.32 (s, 2H)

### [Example 87] Synthesis of 9-(2-(didecylamino)pyrimidin-5-yl)-6,7-dimethoxynaphtho[2,3-c]furan-1(3H)-one hydrochloride

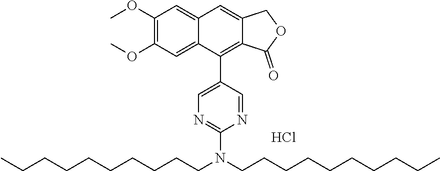 [SMILES: CCCCCCCCCCN(CCCCCCCCCC)c1ncc(-c2c3c(cc4cc(OC)c(OC)cc24)COC3=O)cn1 ; Cl]

The compound of Example 87 was synthesized by the same method as that of the compound of Example 7 except for using N,N-didecyl-5-(4,4,5,5-tetramethyl-1,3,2-dioxaborolan-2-yl)pyrimidin-2-amine instead of 2-(4-methylpiperazin-1-yl)-5-(4,4,5,5-tetramethyl-1,3,2-dioxaborolan-2-yl)pyrimidine.

Mass (M+H⁺): 618.5

¹H-NMR (500 Mhz, DMSO-d6): δ 0.73-0.75 (m, 6H), 1.16-1.22 (m, 28H), 1.54 (s, 4H), 3.52 (s, 4H), 3.67 (s, 3H), 3.86 (s, 3H), 5.36 (s, 2H), 7.03 (s, 1H), 7.44 (s, 1H), 7.87 (s, 1H), 8.33 (s, 2H)

### [Example 88] Synthesis of 6,7-dimethoxy-9-(6-methoxybenzo[d][1,3]dioxol-5-yl)naphtho[2,3-c]furan-1(3H)-one

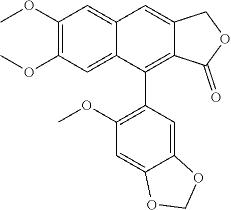 [SMILES: COc1cc2cc3c(c(-c4cc5c(cc4OC)OCO5)c2cc1OC)C(=O)OC3]

The compound of Example 88 was synthesized by the same method as that of the compound of Example 7 except for using 2-(6-methoxybenzo[d][1,3]dioxol-5-yl)-4,4,5,5-tetramethyl-1,3,2-dioxaborolane instead of 2-(4-methylpiperazin-1-yl)-5-(4,4,5,5-tetramethyl-1,3,2-dioxaborolan-2-yl)pyrimidine.

Mass (M+H⁺): 395.1

¹H-NMR (500 Mhz, DMSO-d6): 3.50 (s, 3H), 3.63 (s, 3H), 3.91 (s, 3H), 5.39-5.43 (m, 2H), 6.05 (d, J=4.0 Hz, 2H), 6.70 (s, 1H), 6.83 (s, 1H), 6.92 (s, 1H), 7.44 (s, 1H), 7.87 (s, 1H)

### [Example 89] Synthesis of 9-(2-(1,4-dioxa-8-azaspiro[4.5]decan-8-yl)pyrimidin-5-yl)-6,7-dimethoxynaphtho[2,3-c]furan-1(3H)-one

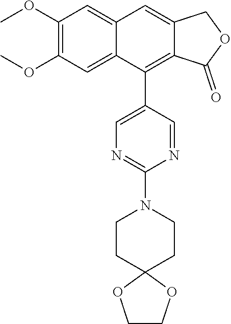 [SMILES: COc1cc2cc3c(c(-c4cnc(N5CCC6(CC5)OCCO6)nc4)c2cc1OC)C(=O)OC3]

The compound of Example 89 was synthesized by the same method as that of the compound of Example 7 except for using 8-(5-(4,4,5,5-tetramethyl-1,3,2-dioxaborolan-2-yl)pyrimidin-2-yl)-1,4-dioxa-8-azaspiro[4.5]decane instead of 2-(4-methylpiperazin-1-yl)-5-(4,4,5,5-tetramethyl-1,3,2-dioxaborolan-2-yl)pyrimidine.

Mass (M+H⁺): 464.2

¹H-NMR (500 Mhz, DMSO-d6): δ 1.69 (t, J=5.7 Hz, 4H), 3.74 (s, 3H), 3.89-3.92 (m, 11H), 5.42 (s, 2H), 7.08 (s, 1H), 7.50 (s, 1H), 7.93 (s, 1H), 8.39 (s, 2H)

### [Example 90] Synthesis of methyl N-(5-(6,7-dimethoxy-3-oxo-1,3-dihydronaphtho[2,3-c]furan-4-yl)pyrimidin-2-yl)-N-methyl glycinate hydrochloride

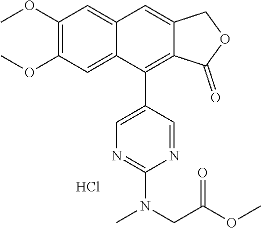

The compound of Example 90 was synthesized by the same method as that of the compound of Example 7 except for using methyl N-methyl-N-(5-(4,4,5,5-tetramethyl-1,3,2-dioxaborolan-2-yl)pyrimidin-2-yl)glycinate instead of 2-(4-methylpiperazin-1-yl)-5-(4,4,5,5-tetramethyl-1,3,2-dioxaborolan-2-yl)pyrimidine.

Mass (M+H⁺): 424.2

¹H-NMR (500 Mhz, DMSO-d6): δ 3.24 (s, 3H), 3.64 (s, 3H), 3.72 (s, 3H), 3.92 (s, 3H), 4.48 (d, J=9.15 Hz, 2H), 5.42 (s, 2H), 7.02 (s, 1H), 7.51 (s, 1H), 7.94 (s, 1H), 8.40-8.44 (m, 2H)

### [Example 91] Synthesis of 9-(2-(cyclopentylamino)pyrimidin-5-yl)-6,7-dimethoxynaphtho[2,3-c]furan-1(3H)-one hydrochloride

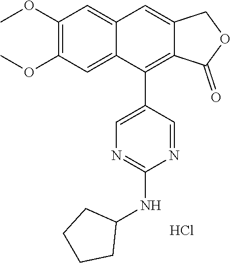 [SMILES: COc1cc2cc3c(c(-c4cnc(NC5CCCC5)nc4)c2cc1OC)C(=O)OC3 ; Cl]

The compound of Example 91 was synthesized by the same method as that of the compound of Example 7 except for using N-cyclopentyl-5-(4,4,5,5-tetramethyl-1,3,2-dioxaborolan-2-yl)pyrimidin-2-amine instead of 2-(4-methylpiperazin-1-yl)-5-(4,4,5,5-tetramethyl-1,3,2-dioxaborolan-2-yl)pyrimidine.

Mass (M+H⁺): 406.2

¹H-NMR (500 Mhz, DMSO-d6): δ 1.56-1.57 (m, 4H), 1.71-1.72 (m, 2H), 1.95-2.05 (m, 2H), 3.77 (s, 3H), 3.92 (s, 3H), 4.29 (t, J=6.6 Hz, 1H), 5.44 (s, 2H), 7.15 (s, 1H), 7.52 (s, 1H), 7.97 (s, 1H), 8.52 (s, 2H)

### [Example 92] Synthesis of 9-(2-(cyclopentyl(ethyl)amino)pyrimidin-5-yl)-6,7-dimethoxynaphtho[2,3-c]furan-1(3H)-one hydrochloride

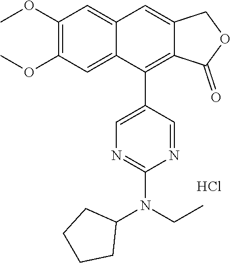 [SMILES: CCN(c1ncc(-c2c3c(cc4cc(OC)c(OC)cc24)COC3=O)cn1)C1CCCC1 ; Cl]

The compound of Example 92 was synthesized by the same method as that of the compound of Example 7 except for using N-cyclopentyl-N-ethyl-5-(4,4,5,5-tetramethyl-1,3,2-dioxaborolan-2-yl)pyrimidin-2-amine instead of 2-(4-methylpiperazin-1-yl)-5-(4,4,5,5-tetramethyl-1,3,2-dioxaborolan-2-yl)pyrimidine.

Mass (M+H⁺): 434.2

¹H-NMR (500 Mhz, DMSO-d6): § 1.19 (t, J=6.85 Hz, 3H), 1.57-1.59 (m, 4H), 1.72-1.73 (m, 2H), 1.89-1.90 (m, 2H), 3.53-3.56 (m, 2H), 3.74 (s, 3H), 3.92 (s, 3H), 5.00-5.03 (m, 1H), 5.42 (s, 2H), 7.11 (s, 1H), 7.50 (s, 1H), 7.93 (s, 1H), 8.42 (s, 2H)

### [Example 93] Synthesis of 9-(2-(cyclopentyl(propyl)amino)pyrimidin-5-yl)-6,7-dimethoxynaphtho[2,3-c]furan-1(3H)-one hydrochloride

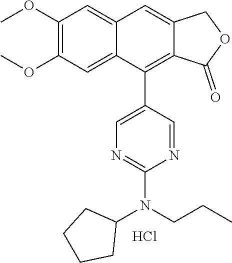 [SMILES: CCCN(c1ncc(-c2c3c(cc4cc(OC)c(OC)cc24)COC3=O)cn1)C1CCCC1 ; Cl]

The compound of Example 93 was synthesized by the same method as that of the compound of Example 7 except for using N-cyclopentyl-N-propyl-5-(4,4,5,5-tetramethyl-1,3,2-dioxaborolan-2-yl)pyrimidin-2-amine instead of 2-(4-methylpiperazin-1-yl)-5-(4,4,5,5-tetramethyl-1,3,2-dioxaborolan-2-yl)pyrimidine.

Mass (M+H⁺): 448.2

¹H-NMR (500 Mhz, DMSO-d6): δ 0.85 (s, 3H), 1.54-1.91 (m, 12H), 3.70 (s, 3H), 3.88 (s, 3H), 4.89-4.95 (m, 1H), 5.38 (s, 2H), 5.75-5.96 (br, 3H), 7.06 (s, 1H), 7.46 (s, 1H), 7.89 (s, 1H), 8.37 (s, 2H)

### [Example 94] Synthesis of 9-(2-((2,2-dimethoxyethyl)(methyl)amino)pyrimidin-5-yl)-6,7-dimethoxynaphtho[2,3-c]furan-1(3H)-one

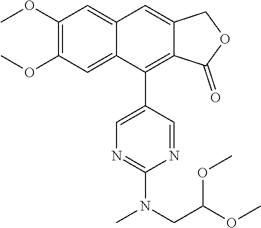

The compound of Example 94 was synthesized by the same method as that of the compound of Example 7 except for using N-(2,2-dimethoxyethyl)-N-methyl-5-(4,4,5,5-tetramethyl-1,3,2-dioxaborolan-2-yl)pyrimidin-2-amine of instead 2-(4-methylpiperazin-1-yl)-5-(4,4,5,5-tetramethyl-1,3,2-dioxaborolan-2-yl)pyrimidine.

Mass (M+H⁺): 440.2

¹H-NMR (500 Mhz, DMSO-d6): δ 3.20 (s, 3H), 3.30 (s, 6H), 3.73-3.76 (m, 5H), 3.92 (s, 3H), 4.63 (t, J=5.15 Hz, 1H), 5.42 (s, 2H), 7.06 (s, 1H), 7.50 (s, 1H), 7.93 (s, 1H), 8.40 (s, 2H)

### [Example 95] Synthesis of 9-(2-(dioctylamino)pyrimidin-5-yl)-6,7-dimethoxynaphtho[2,3-c]furan-1(3H)-one hydrochloride

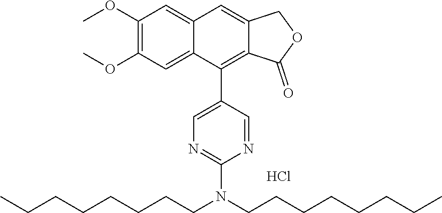 [SMILES: CCCCCCCCN(CCCCCCCC)c1ncc(-c2c3c(cc4cc(OC)c(OC)cc24)COC3=O)cn1 ; Cl]

The compound of Example 95 was synthesized by the same method as that of the compound of Example 7 except for using N,N-dioctyl-5-(4,4,5,5-tetramethyl-1,3,2-dioxaborolan-2-yl)pyrimidin-2-amine instead of 2-(4-methylpiperazin-1-yl)-5-(4,4,5,5-tetramethyl-1,3,2-dioxaborolan-2-yl)pyrimidine.

Mass (M+H⁺): 562.4

¹H-NMR (500 Mhz, DMSO-d6): δ 0.79-0.82 (m, 6H), 1.21-1.27 (m, 24H), 1.62-1.63 (m, 4H), 3.72 (s, 3H), 3.91 (s, 3H), 5.41 (s, 2H), 7.07 (s, 1H), 7.48 (s, 1H), 7.92 (s, 1H), 8.37 (s, 2H)

### [Example 96] Synthesis of (S)-6,7-dimethoxy-9-(2-((5-methoxy-1,2,3,4-tetrahydronaphthalen-2-yl)(methyl)amino)pyrimidin-5-yl)naphtho[2,3-c]furan-1(3H)-one hydrochloride

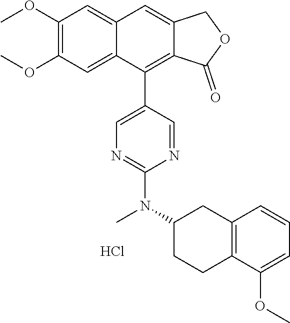

The compound of Example 96 was synthesized by the same method as that of the compound of Example 7 except for using (S)—N-(5-methoxy-1,2,3,4-tetrahydronaphthalen-2-yl)-N-methyl-5-(4,4,5,5-tetramethyl-1,3,2-dioxaborolan-2-yl)pyrimidin-2-amine instead of 2-(4-methylpiperazin-1-yl)-5-(4,4,5,5-tetramethyl-1,3,2-dioxaborolan-2-yl)pyrimidine.

Mass (M+H⁺): 512.3

¹H-NMR (500 Mhz, DMSO-d6): δ 1.97-1.99 (m, 2H), 2.59-2.61 (m, 1H), 2.79-2.83 (m, 1H), 2.93-3.00 (m, 2H), 3.12 (s, 3H), 3.74 (s, 6H), 3.92 (s, 3H), 4.97-4.99 (m, 1H), 5.41 (s, 2H), 6.71 (dd, J=24.65, 8.3 Hz, 2H), 7.05-7.10 (m, 2H), 7.50 (s, 1H), 7.92 (s, 1H), 8.39 (s, 2H)

### [Example 97] Synthesis of (S)-6,7-dimethoxy-9-(2-(2-(methoxymethyl)pyrrolidin-1-yl)pyrimidin-5-yl)naphtho[2,3-c]furan-1(3H)-one hydrochloride

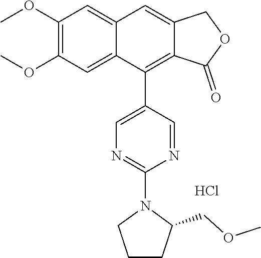

The compound of Example 97 was synthesized by the same method as that of the compound of Example 7 except for using (S)-2-(2-(methoxymethyl)pyrrolidin-1-yl)-5-(4,4,5,5-tetramethyl-1,3,2-dioxaborolan-2-yl)pyrimidine instead of 2-(4-methylpiperazin-1-yl)-5-(4,4,5,5-tetramethyl-1,3,2-dioxaborolan-2-yl)pyrimidine.

Mass (M+H⁺): 436.2

¹H-NMR (500 Mhz, DMSO-d6): δ 1.88-1.99 (m, 7H), 3.32 (t, J=8.05, 1H), 3.45-3.51 (m, 1H), 3.54-3.56 (m, 2H), 3.70 (s, 3H), 3.88 (s, 3H), 4.26-4.28 (m, 1H), 5.38 (s, 2H), 7.04 (s, 1H), 7.46 (s, 1H), 7.90 (s, 1H), 8.39 (s, 2H)

### [Example 98] Synthesis of 6-(6,7-dimethoxy-3-oxo-1,3-dihydronaphtho[2,3-c]furan-4-yl)benzo[d][1,3]dioxol-5-yl isopropyl carbonate

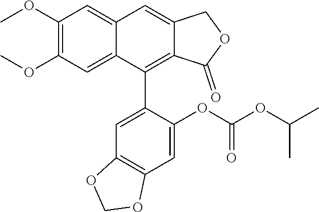 [SMILES: COc1cc2cc3c(c(-c4cc5c(cc4OC(=O)OC(C)C)OCO5)c2cc1OC)C(=O)OC3]

The compound of Example 98 was synthesized by the same method as that of the compound of Example 7 except for using isopropyl (6-(4,4,5,5-tetramethyl-1,3,2-dioxaborolan-2-yl)benzo[d][1,3]dioxol-5-yl)carbonate instead of 2-(4-methylpiperazin-1-yl)-5-(4,4,5,5-tetramethyl-1,3,2-dioxaborolan-2-yl)pyrimidine.

Mass (M+H⁺): 467.2

¹H-NMR (500 Mhz, DMSO-d6): δ 0.62 (d, J=6.0 Hz, 3H), 0.88 (d, J=6.3 Hz, 3H), 3.62 (s, 3H), 3.86 (s, 3H), 4.39-4.41 (m, 1H), 5.35-5.41 (m, 2H), 6.12 (s, 2H), 6.80 (s, 1H), 6.84 (s, 1H), 7.02 (s, 1H), 7.42 (s, 1H), 7.89 (s, 1H)

### [Example 99] Synthesis of cyclohexyl (6-(6,7-dimethoxy-3-oxo-1,3-dihydronaphtho[2,3-c]furan-4-yl)benzo[d][1,3]dioxol-5-yl)carbonate

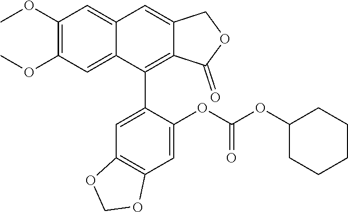 [SMILES: COc1cc2cc3c(c(-c4cc5c(cc4OC(=O)OC4CCCCC4)OCO5)c2cc1OC)C(=O)OC3]

The compound of Example 99 was synthesized by the same method as that of the compound of Example 7 except for using cyclohexyl (6-(4,4,5,5-tetramethyl-1,3,2-dioxaborolan-2-yl)benzo[d][1,3]dioxol-5-yl)carbonate instead of 2-(4-methylpiperazin-1-yl)-5-(4,4,5,5-tetramethyl-1,3,2-dioxaborolan-2-yl)pyrimidine.

Mass (M+H⁺): 507.2

¹H-NMR (500 Mhz, DMSO-d6): δ 0.70-0.73 (m, 1H), 0.93-1.12 (m, 6H), 1.18-1.20 (m, 1H), 1.27-1.30 (m, 1H), 1.41-1.42 (m, 1H), 3.59 (s, 3H), 3.84 (s, 3H), 4.17-4.18 (m, 1H), 5.36 (s, 2H), 6.10 (s, 2H), 6.78 (s, 1H), 6.83 (s, 1H), 7.00 (s, 1H), 7.40 (s, 1H), 7.88 (s, 1H)

### [Example 100] Synthesis of 9-(2-(sec-butyl(methyl)amino)pyrimidin-5-yl)-6,7-dimethoxynaphtho[2,3-c]furan-1(3H)-one hydrochloride

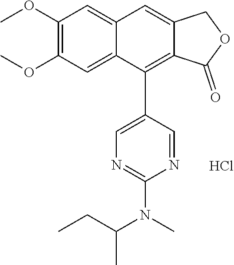 [SMILES: CCC(C)N(C)c1ncc(-c2c3c(cc4cc(OC)c(OC)cc24)COC3=O)cn1 ; Cl]

The compound of Example 101 was synthesized by the same method as that of the compound of Example 7 except for using N-(sec-butyl)-N-methyl-5-(4,4,5,5-tetramethyl-1,3,2-dioxaborolan-2-yl)pyrimidin-2-amine instead of 2-(4-methylpiperazin-1-yl)-5-(4,4,5,5-tetramethyl-1,3,2-dioxaborolan-2-yl)pyrimidine.

Mass (M+H⁺): 408.2

¹H-NMR (500 Mhz, DMSO-d6): δ 0.82 (t, J=7.45 Hz, 3H), 1.17 (d, J=6.85 Hz, 3H), 1.51-1.65 (m, 2H), 3.00 (s, 3H), 3.74 (s, 3H), 3.90 (s, 3H), 4.86-4.91 (m, 1H), 5.42 (s, 2H), 7.10 (s, 1H), 7.51 (s, 1H), 7.94 (s, 1H), 8.44 (s, 2H)

### [Example 101] Synthesis of 9-(2-((2-(cyclohex-1-en-1-yl)ethyl)(methyl)amino)pyrimidin-5-yl)-6,7-dimethoxynaphtho[2,3-c]furan-1(3H)-one hydrochloride

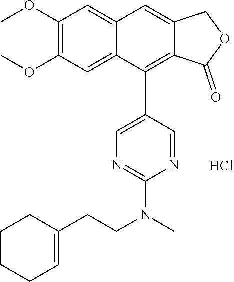 [SMILES: COc1cc2cc3c(c(-c4cnc(N(C)CCC5=CCCCC5)nc4)c2cc1OC)C(=O)OC3 ; Cl]

The compound of Example 101 was synthesized by the same method as that of the compound of Example 7 except for using N-(2-(cyclohex-1-en-1-yl)ethyl)-N-methyl-5-(4,4,5,5-tetramethyl-1,3,2-dioxaborolan-2-yl)pyrimidin-2-amine instead of 2-(4-methylpiperazin-1-yl)-5-(4,4,5,5-tetramethyl-1,3,2-dioxaborolan-2-yl)pyrimidine.

Mass (M+H⁺): 460.3

¹H-NMR (500 Mhz, DMSO-d6): δ 1.13 (t, J=7.15 Hz, 1H), 1.42-1.45 (m, 2H), 1.49-1.53 (m, 2H), 1.88 (br, 2H), 1.93-1.97 (m, 3H), 2.22 (t, J=6.85 Hz, 2H), 3.73 (s, 3H), 3.77 (t, J=7.15 Hz, 2H), 3.92 (s, 3H), 3.99 (q, J=7.15 Hz, 1H), 5.39 (s, 1H), 5.42 (s, 2H), 7.06 (s, 1H), 7.50 (s, 1H), 7.93 (s, 1H), 8.39 (s, 2H)

### [Example 102] Synthesis of (R)-6,7-dimethoxy-9-(2-(2-(methoxymethyl)pyrrolidin-1-yl)pyrimidin-5-yl)naphtho[2,3-c]furan-1(3H)-one hydrochloride

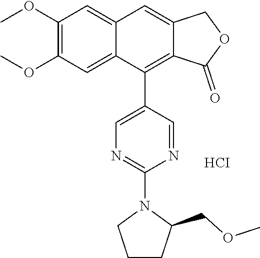

The compound of Example 102 was synthesized by the same method as that of the compound of Example 7 except for using (R)-2-(2-(methoxymethyl)pyrrolidin-1-yl)-5-(4,4,5,5-tetramethyl-1,3,2-dioxaborolan-2-yl)pyrimidine instead of 2-(4-methylpiperazin-1-yl)-5-(4,4,5,5-tetramethyl-1,3,2-dioxaborolan-2-yl)pyrimidine.

Mass (M+H⁺): 436.2

1H-NMR (500 Mhz, DMSO-d6): δ 1.95-2.00 (m, 6H), 3.37 (t, J=8.6 Hz, 1H), 3.52-3.61 (m, 4H), 3.74 (s, 3H), 3.92 (s, 3H), 4.32-4.34 (m, 1H), 5.43 (s, 2H), 7.09 (s, 1H), 7.51 (s, 1H), 7.95 (s, 1H), 8.46 (s, 2H)

### [Example 103] Synthesis of 6,7-dimethoxy-9-(2-(methyl((5-methylfuran-2-yl)methyl)amino)pyrimidin-5-yl)naphtho[2,3-c]furan-1(3H)-one hydrochloride

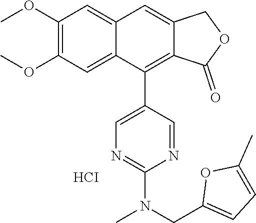 [SMILES: COc1cc2cc3c(c(-c4cnc(N(C)Cc5ccc(C)o5)nc4)c2cc1OC)C(=O)OC3 ; "HCI"]

The compound of Example 103 was synthesized by the same method as that of the compound of Example 7 except for using N-methyl-N-((5-methylfuran-2-yl)methyl)-5-(4,4,5,5-tetramethyl-1,3,2-dioxaborolan-2-yl)pyrimidin-2-amine instead of 2-(4-methylpiperazin-1-yl)-5-(4,4,5,5-tetramethyl-1,3,2-dioxaborolan-2-yl)pyrimidine.

Mass (M+H⁺): 446.2

1H-NMR (500 Mhz, DMSO-d6): δ 2.20 (d, J=0.55 Hz, 3H), 3.16 (s, 3H), 3.73 (s, 3H), 3.92 (s, 3H), 4.86 (br, 2H), 5.42 (s, 2H), 5.97-5.98 (m, 1H), 6.20 (d, J=2.9 Hz, 1H), 7.06 (s, 1H), 7.51 (s, 1H), 7.94 (s, 1H), 8.43 (s, 2H)

### [Example 104] Synthesis of 9-(2-(2-(hydroxymethyl) piperidin-1-yl)pyrimidin-5-yl)-6,7-dimethoxynaphtho[2,3-c]furan-1(3H)-one hydrochloride

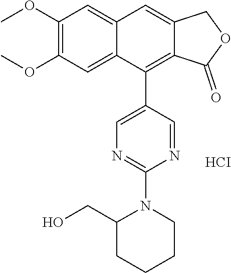

The compound of Example 104 was synthesized by the same method as that of the compound of Example 7 except for using (1-(5-(4,4,5,5-tetramethyl-1,3,2-dioxaborolan-2-yl)pyrimidin-2-yl) piperidin-2-yl) methanol instead of 2-(4-methylpiperazin-1-yl)-5-(4,4,5,5-tetramethyl-1,3,2-dioxaborolan-2-yl)pyrimidine.

Mass (M+H⁺): 436.2

1H-NMR (500 Mhz, DMSO-d6): δ 1.39-1.72 (m, 6H), 1.93-1.96 (m, 1H), 2.95-3.00 (m, 1H), 3.13 (s, 1H), 3.51-3.54 (m, 1H), 3.64-3.68 (m, 1H), 3.75 (s, 3H), 3.94 (s, 3H), 5.42 (s, 2H), 7.10 (s, 1H), 7.51 (s, 1H), 7.94 (s, 1H), 8.41 (s, 2H)

### [Example 105] Synthesis of 9-(2-((cyclopropylmethyl)(propyl)amino)pyrimidin-5-yl)-6,7-dimethoxynaphtho[2,3-c]furan-1(3H)-one hydrochloride

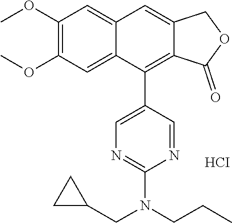

The compound of Example 105 was synthesized by the same method as that of the compound of Example 7 except for using N-(cyclopropylmethyl)-N-propyl-5-(4,4,5,5-tetramethyl-1,3,2-dioxaborolan-2-yl)pyrimidin-2-amine instead of 2-(4-methylpiperazin-1-yl)-5-(4,4,5,5-tetramethyl-1,3,2-dioxaborolan-2-yl)pyrimidine.

Mass (M+H⁺): 434.2

1H-NMR (500 Mhz, DMSO-d6): δ 0.30-0.32 (m, 2H), 0.45-0.47 (m, 2H), 0.89 (t, J=7.45 Hz, 3H), 1.13-1.16 (m, 1H), 1.62-1.68 (m, 2H), 3.54 (d, J=6.6 Hz, 2H), 3.60-3.62 (m, 2H), 3.74 (s, 3H), 3.92 (s, 3H), 5.42 (s, 2H), 7.10 (s, 1H), 7.50 (s, 1H), 7.93 (s, 1H), 8.38 (s, 2H)

### [Example 106] Synthesis of 9-(2-(ethyl(methyl)amino)pyrimidin-5-yl)-6,7-dimethoxynaphtho[2,3-c]furan-1(3H)-one hydrochloride

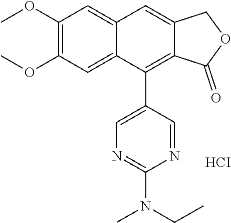

The compound of Example 106 was synthesized by the same method as that of the compound of Example 7 except for using N-ethyl-N-methyl-5-(4,4,5,5-tetramethyl-1,3,2-dioxaborolan-2-yl)pyrimidin-2-amine instead of 2-(4-methylpiperazin-1-yl)-5-(4,4,5,5-tetramethyl-1,3,2-dioxaborolan-2-yl)pyrimidine.

Mass (M+H⁺): 380.2

1H-NMR (500 Mhz, DMSO-d6): δ 1.16 (t, J=6.85 Hz, 3H), 3.18 (s, 3H), 3.70-3.74 (m, 5H), 3.92 (s, 3H), 5.42 (s, 2H), 7.09 (s, 1H), 7.51 (s, 1H), 7.94 (s, 1H), 8.43 (s, 2H)

### [Example 107] Synthesis of 6,7-dimethoxy-9-(2-(4-methylpiperidin-1-yl)pyrimidin-5-yl)naphtho[2,3-c]furan-(3H)-one hydrochloride

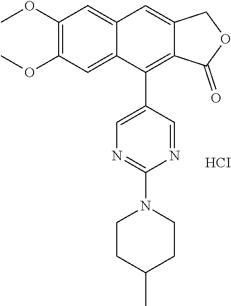

The compound of Example 107 was synthesized by the same method as that of the compound of Example 7 except for using 2-(4-methylpiperidin-1-yl)-5-(4,4,5,5-tetramethyl-1,3,2-dioxaborolan-2-yl)pyrimidine instead of 2-(4-methylpiperazin-1-yl)-5-(4,4,5,5-tetramethyl-1,3,2-dioxaborolan-2-yl)pyrimidine.

Mass (M+H⁺): 420.2

1H-NMR (500 Mhz, DMSO-d6): δ 0.93 (d, J=6.3 Hz, 3H), 1.11-1.13 (m, 2H), 1.65-1.72 (m, 3H), 2.92-2.97 (m, 2H), 3.74 (s, 3H), 3.92 (s, 3H), 4.71 (d, J=13.2 Hz, 2H), 5.42 (s, 2H), 7.09 (s, 1H), 7.50 (s, 1H), 7.93 (s, 1H), 8.39 (s, 2H)

### [Example 108] Synthesis of 6,7-dimethoxy-9-(2-(methylamino)pyrimidin-5-yl)naphtho[2,3-c]furan-1(3H)-one

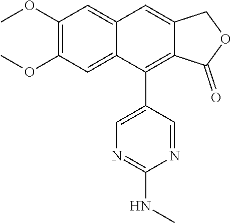 [SMILES: CNc1ncc(-c2c3c(cc4cc(OC)c(OC)cc24)COC3=O)cn1]

The compound of Example 108 was synthesized by the same method as that of the compound of Example 7 except for using N-methyl-5-(4,4,5,5-tetramethyl-1,3,2-dioxaborolan-2-yl)pyrimidin-2-amine instead of 2-(4-methylpiperazin-1-yl)-5-(4,4,5,5-tetramethyl-1,3,2-dioxaborolan-2-yl)pyrimidine.

Mass (M+H⁺): 352.1

1H-NMR (500 Mhz, DMSO-d6): δ 2.87 (d, J=4.85 Hz, 3H), 3.73 (s, 3H), 3.92 (s, 3H), 5.41 (s, 2H), 7.07 (s, 1H), 7.30 (m, 1H) 7.49 (s, 1H), 7.92 (s, 1H), 8.30 (br, 2H)

### [Example 109] Synthesis of 9-(2-((3,4-dimethylphenyl)(methyl)amino)pyrimidin-5-yl)-6,7-dimethoxynaphtho[2,3-c]furan-1(3H)-one

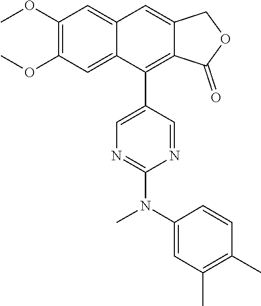

The compound of Example 109 was synthesized by the same method as that of the compound of Example 7 except for using N-(3,4-dimethylphenyl)-N-methyl-5-(4,4,5,5-tetramethyl-1,3,2-dioxaborolan-2-yl)pyrimidin-2-amine instead of 2-(4-methylpiperazin-1-yl)-5-(4,4,5,5-tetramethyl-1,3,2-dioxaborolan-2-yl)pyrimidine.

Mass (M+H⁺): 456.2

1H-NMR (500 Mhz, DMSO-d6): δ 2.20 (d, J=3.15 Hz, 6H), 3.49 (s, 3H), 3.74 (s, 3H), 3.92 (s, 3H), 5.42 (s, 2H), 7.05-7.16 (m, 4H), 7.50 (s, 1H), 7.94 (s, 1H), 8.38 (s, 2H)

### [Example 110] Synthesis of (R)-6,7-dimethoxy-9-(2-(2-(pyrrolidin-1-ylmethyl)pyrrolidin-1-yl)pyrimidin-5-yl)naphtho[2,3-c]furan-1(3H)-one

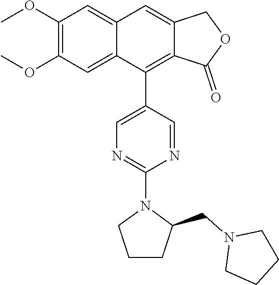 [SMILES: COc1cc2cc3c(c(-c4cnc(N5CCC[C@@H]5CN5CCCC5)nc4)c2cc1OC)C(=O)OC3]

The compound of Example 110 was synthesized by the same method as that of the compound of Example 7 except for using (R)-2-(2-(pyrrolidin-1-ylmethyl)pyrrolidin-1-yl)-5-(4,4,5,5-tetramethyl-1,3,2-dioxaborolan-2-yl)pyrimidine instead of 2-(4-methylpiperazin-1-yl)-5-(4,4,5,5-tetramethyl-1,3,2-dioxaborolan-2-yl)pyrimidine.

Mass (M+H⁺): 475.2

1H-NMR (500 Mhz, DMSO-d6): δ 1.64 (s, 4H), 1.92-2.07 (m, 4H), 2.45 (m, 9H), 3.50-3.58 (m, 2H), 3.73 (s, 3H), 3.92 (s, 3H), 5.42 (s, 2H), 7.07 (s, 1H), 7.50 (s, 1H), 7.93 (s, 1H), 8.36 (d, J=2.3 Hz, 2H)

### [Example 111] Synthesis of 6,7-dimethoxy-9-(2-(methyl(2-morpholinoethyl)amino)pyrimidin-5-yl)naphtho[2,3-c]furan-1(3H)-one hydrochloride

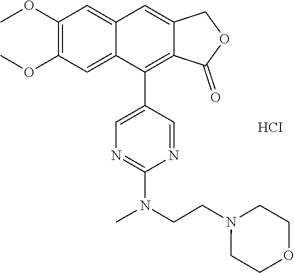

The compound of Example 111 was synthesized by the same method as that of the compound of Example 7 except for using N-methyl-N-(2-morpholinoethyl)-5-(4,4,5,5-tetramethyl-1,3,2-dioxaborolan-2-yl)pyrimidin-2-amine instead of 2-(4-methylpiperazin-1-yl)-5-(4,4,5,5-tetramethyl-1,3,2-dioxaborolan-2-yl)pyrimidine.

Mass (M+H⁺): 465.2

1H-NMR (500 Mhz, DMSO-d6): δ 2.43-2.57 (m, 6H), 3.18 (s, 3H), 3.52 (t, J=4.3 Hz, 4H), 3.73 (s, 3H), 3.80 (t, J=6.85 Hz, 2H), 3.92 (s, 3H), 5.41 (s, 2H), 7.06 (s, 1H), 7.49 (s, 1H), 7.92 (s, 1H), 8.36 (s, 2H)

### [Example 112] Synthesis of 6,7-dimethoxy-9-(2-(methyl(3-morpholinopropyl)amino)pyrimidin-5-yl)naphtho[2,3-c]furan-1(3H)-one dihydrochloride

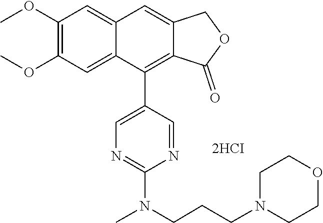

The compound of Example 112 was synthesized by the same method as that of the compound of Example 7 except for using N-methyl-N-(3-morpholinopropyl)-5-(4,4,5,5-tetramethyl-1,3,2-dioxaborolan-2-yl)pyrimidin-2-amine instead of 2-(4-methylpiperazin-1-yl)-5-(4,4,5,5-tetramethyl-1,3,2-dioxaborolan-2-yl)pyrimidine.

Mass (M+H⁺): 479.2

1H-NMR (500 Mhz, DMSO-d6): δ 2.03-2.09 (m, 2H), 3.02-3.07 (m, 2H), 3.13-3.21 (m, 4H), 3.42-3.44 (m, 2H), 3.69-3.76 (m, 7H), 3.92-3.94 (m, 6H), 5.43 (s, 2H), 7.08 (s, 1H), 7.51 (s, 1H), 7.95 (s, 1H), 8.42 (s, 2H), 10.34 (br, 1H)

### [Example 113] Synthesis of (R)-9-(2-(2-ethylpiperidin-1-yl)pyrimidin-5-yl)-6,7-dimethoxynaphtho[2,3-c]furan-1(3H)-one

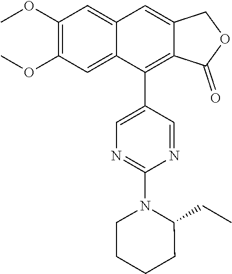 [SMILES: CC[C@@H]1CCCCN1c1ncc(-c2c3c(cc4cc(OC)c(OC)cc24)COC3=O)cn1]

The compound of Example 113 was synthesized by the same method as that of the compound of Example 7 except for using (R)-2-(2-ethylpiperidin-1-yl)-5-(4,4,5,5-tetramethyl-1,3,2-dioxaborolan-2-yl)pyrimidine instead of 2-(4-methylpiperazin-1-yl)-5-(4,4,5,5-tetramethyl-1,3,2-dioxaborolan-2-yl)pyrimidine.

Mass (M+H⁺): 434.2

1H-NMR (500 Mhz, DMSO-d6): δ 0.82 (t, J=7.45 Hz, 3H), 1.38-1.41 (m, 1H), 1.52-1.69 (m, 7H), 2.91-2.93 (m, 1H), 3.73 (s, 3H), 3.92 (s, 3H), 4.67-4.70 (m, 1H), 4.87-4.89 (m, 1H), 5.41 (s, 2H), 7.08 (s, 1H), 7.49 (s, 1H), 7.92 (s, 1H), 8.35 (s, 2H)

### [Example 114] Synthesis of 6,7-dimethoxy-9-(2-(2,2,6,6-tetramethylmorpholino)pyrimidin-5-yl)naphtho[2,3-c]furan-1(3H)-one

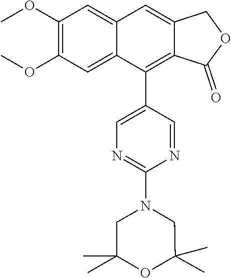 [SMILES: COc1cc2cc3c(c(-c4cnc(N5CC(C)(C)OC(C)(C)C5)nc4)c2cc1OC)C(=O)OC3]

The compound of Example 114 was synthesized by the same method as that of the compound of Example 7 except for using 2,2,6,6-tetramethyl-4-(5-(4,4,5,5-tetramethyl-1,3,2-dioxaborolan-2-yl)pyrimidin-2-yl)morpholine instead of 2-(4-methylpiperazin-1-yl)-5-(4,4,5,5-tetramethyl-1,3,2-dioxaborolan-2-yl)pyrimidine.

Mass (M+H⁺): 464.2

1H-NMR (500 Mhz, DMSO-d6): § 1.19 (s, 12H), 3.70 (s, 4H), 3.73 (s, 3H), 3.93 (s, 3H), 5.43 (s, 2H), 7.08 (s, 1H), 7.51 (s, 1H), 7.94 (s, 1H), 8.41 (s, 2H)

### [Example 115] Synthesis of 6,7-dimethoxy-9-(2-(methyl(tetrahydro-2H-pyran-4-yl)amino)pyrimidin-5-yl)naphtho[2,3-c]furan-1(3H)-one

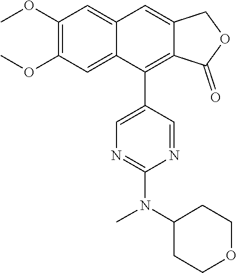 [SMILES: COc1cc2cc3c(c(-c4cnc(N(C)C5CCOCC5)nc4)c2cc1OC)C(=O)OC3]

The compound of Example 115 was synthesized by the same method as that of the compound of Example 7 except for using N-methyl-N-(tetrahydro-2H-pyran-4-yl)-5-(4,4,5,5-tetramethyl-1,3,2-dioxaborolan-2-yl)pyrimidin-2-amine instead of 2-(4-methylpiperazin-1-yl)-5-(4,4,5,5-tetramethyl-1,3,2-dioxaborolan-2-yl)pyrimidine.

Mass (M+H⁺): 436.2

1H-NMR (500 Mhz, DMSO-d6): δ 1.61 (d, J=11.45 Hz, 2H), 1.84-1.86 (m, 2H), 3.07 (s, 3H), 3.41-3.46 (m, 2H), 3.74 (s, 3H), 3.93-3.95 (m, 5H), 4.88-4.91 (m, 1H), 5.42 (s, 2H), 7.09 (s, 1H), 7.51 (s, 1H), 7.93 (s, 1H), 8.40 (s, 2H)

### [Example 116] Synthesis of 6,7-dimethoxy-9-(2-methylquinolin-6-yl)naphtho[2,3-c]furan-1(3H)-one

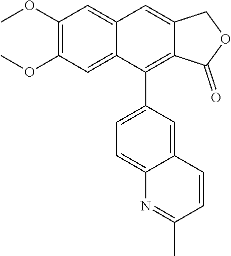 [SMILES: COc1cc2cc3c(c(-c4ccc5nc(C)ccc5c4)c2cc1OC)C(=O)OC3]

The compound of Example 116 was synthesized by the same method as that of the compound of Example 7 except for using 2-methyl-6-(4,4,5,5-tetramethyl-1,3,2-dioxaborolan-2-yl) quinoline instead of 2-(4-methylpiperazin-1-yl)-5-(4,4,5,5-tetramethyl-1,3,2-dioxaborolan-2-yl)pyrimidine.

Mass (M+H⁺): 386.1

1H-NMR (500 Mhz, DMSO-d6): δ 2.68 (s, 3H), 3.52 (s, 3H), 3.93 (s, 3H), 5.46 (s, 2H), 6.89 (s, 1H), 7.46 (d, J=8.0 Hz, 1H), 7.54 (s, 1H), 7.65 (d, J=8.0 Hz, 1H), 7.91 (s, 1H), 7.99-8.01 (m, 2H), 8.27 (d, J=8.55 Hz, 1H)

### [Example 117] Synthesis of 9-(4-hydroxyquinolin-6-yl)-6,7-dimethoxynaphtho[2,3-c]furan-1(3H)-one

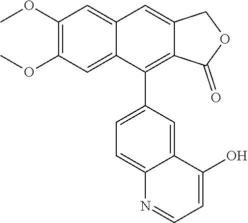 [SMILES: COc1cc2cc3c(c(-c4ccc5nccc(O)c5c4)c2cc1OC)C(=O)OC3]

The compound of Example 117 was synthesized by the same method as that of the compound of Example 7 except for using 6-(4,4,5,5-tetramethyl-1,3,2-dioxaborolan-2-yl) quinolin-4-ol instead of 2-(4-methylpiperazin-1-yl)-5-(4,4,5,5-tetramethyl-1,3,2-dioxaborolan-2-yl)pyrimidine.

Mass (M+H⁺): 388.1

1H-NMR (500 Mhz, DMSO-d6): δ 3.56 (s, 3H), 3.93 (s, 3H), 5.44 (s, 2H), 6.05-6.07 (m, 1H), 6.88 (s, 1H), 7.52 (s, 1H), 7.62-7.63 (m, 2H), 7.97-8.00 (m, 3H), 11.88 (br, 1H)

### [Example 118] Synthesis of 6,7-dimethoxy-9-(quinolin-7-yl)naphtho[2,3-c]furan-1(3H)-one

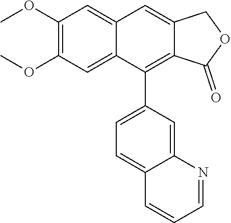 [SMILES: COc1cc2cc3c(c(-c4ccc5cccnc5c4)c2cc1OC)C(=O)OC3]

The compound of Example 118 was synthesized by the same method as that of the compound of Example 7 except for using 7-(4,4,5,5-tetramethyl-1,3,2-dioxaborolan-2-yl) quinoline instead of 2-(4-methylpiperazin-1-yl)-5-(4,4,5,5-tetramethyl-1,3,2-dioxaborolan-2-yl)pyrimidine.

Mass (M+H⁺): 372.1

1H-NMR (500 Mhz, DMSO-d6): δ 3.52 (s, 3H), 3.94 (s, 3H), 5.47 (s, 2H), 6.88 (s, 1H), 7.55-7.59 (m, 3H), 7.97 (s, 1H), 8.01 (s, 1H), 8.09 (d, J=8.0 Hz, 1H), 8.45 (d, J=8.0 Hz, 1H), 8.93 (s, 1H)

### [Example 119] Synthesis of 6,7-dimethoxy-9-(5-(trifluoromethyl) quinolin-8-yl)naphtho[2,3-c]furan-1(3H)-one

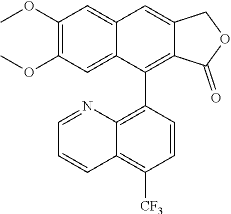 [SMILES: COc1cc2cc3c(c(-c4ccc(C(F)(F)F)c5cccnc45)c2cc1OC)C(=O)OC3]

The compound of Example 119 was synthesized by the same method as that of the compound of Example 7 except for using 8-(4,4,5,5-tetramethyl-1,3,2-dioxaborolan-2-yl)-5-(trifluoromethyl) quinoline instead of 2-(4-methylpiperazin-1-yl)-5-(4,4,5,5-tetramethyl-1,3,2-dioxaborolan-2-yl)pyrimidine.

Mass (M+H⁺): 440.1

1H-NMR (500 Mhz, DMSO-d6): δ 3.40 (s, 3H), 3.93 (s, 3H), 5.45-5.50 (m, 2H), 6.55 (s, 1H), 7.55 (s, 1H), 7.69-7.71 (m, 1H), 7.85 (d, J=7.45 Hz, 1H), 8.03 (s, 1H), 8.20 (d, J=7.45 Hz, 1H), 8.56-8.57 (m, 1H), 8.76 (s, 1H)

### [Example 120] Synthesis of 6,7-dimethoxy-9-(3-methylquinolin-6-yl)naphtho[2,3-c]furan-1(3H)-one

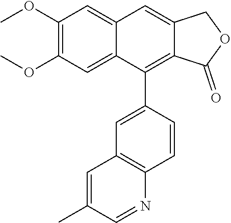 [SMILES: COc1cc2cc3c(c(-c4ccc5ncc(C)cc5c4)c2cc1OC)C(=O)OC3]

The compound of Example 120 was synthesized by the same method as that of the compound of Example 7 except for using 3-methyl-6-(4,4,5,5-tetramethyl-1,3,2-dioxaborolan-2-yl) quinoline instead of 2-(4-methylpiperazin-1-yl)-5-(4,4,5,5-tetramethyl-1,3,2-dioxaborolan-2-yl)pyrimidine.

Mass (M+H⁺): 386.1

1H-NMR (500 Mhz, DMSO-d6): δ 2.46 (s, 3H), 3.52 (s, 3H), 3.93 (s, 3H), 5.45 (s, 2H), 6.89 (s, 1H), 7.54 (s, 1H), 7.63-7.65 (m, 1H), 7.88 (s, 1H), 8.00 (s, 1H), 8.06 (d, J=8.0 Hz, 1H), 8.14 (s, 1H), 8.82 (s, 1H)

### [Example 121] Synthesis of 9-(6-((2S,6R)-2,6-dimethylmorpholino)pyridin-3-yl)-6,7-dimethoxynaphtho[2,3-c]furan-1(3H)-one

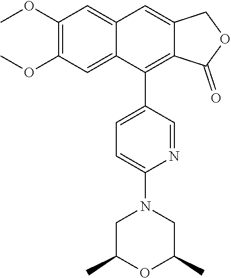 [SMILES: COc1cc2cc3c(c(-c4ccc(N5C[C@@H](C)O[C@@H](C)C5)nc4)c2cc1OC)C(=O)OC3]

The compound of Example 121 was synthesized by the same method as that of the compound of Example 7 except for using (2S,6R)-2,6-dimethyl-4-(5-(4,4,5,5-tetramethyl-1,3,2-dioxaborolan-2-yl)pyridin-2-yl)morpholine instead of 2-(4-methylpiperazin-1-yl)-5-(4,4,5,5-tetramethyl-1,3,2-dioxaborolan-2-yl)pyrimidine.

Mass (M+H⁺): 435.1

¹H-NMR (500 MHz, DMSO-d6): 1.16 (s, 6H), 3.63 (m, 2H), 3.66 (s, 3H), 3.91 (s, 3H), 4.22 (m, 2H), 5.39 (m, 2H), 6.94 (m, 1H), 7.04 (s, 1H), 7.47 (s, 1H), 7.58 (m, 1H), 7.89 (s, 1H), 8.09 (s, 1H).

### [Example 122] Synthesis of 9-(6-(4,4-difluoropiperidin-1-yl)pyridin-3-yl)-6,7-dimethoxynaphtho[2,3-c]furan-1(3H)-one

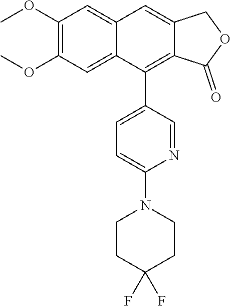 [SMILES: COc1cc2cc3c(c(-c4ccc(N5CCC(F)(F)CC5)nc4)c2cc1OC)C(=O)OC3]

The compound of Example 122 was synthesized by the same method as that of the compound of Example 7 except for using 2-(4,4-difluoropiperidin-1-yl)-5-(4,4,5,5-tetramethyl-1,3,2-dioxaborolan-2-yl)pyridine instead of 2-(4-methylpiperazin-1-yl)-5-(4,4,5,5-tetramethyl-1,3,2-dioxaborolan-2-yl)pyrimidine.

Mass (M+H⁺): 441.0

¹H-NMR (500 MHZ, DMSO-d6): 2.03 (brm, 4H), 3.66 (s, 3H), 3.76 (brm, 4H), 3.91 (s, 3H), 5.40 (s, 2H), 7.03 (m, 2H), 7.47 (s, 1H), 7.60 (m, 1H), 7.90 (s, 1H), 8.01 (s, 1H).

### [Example 123] Synthesis of 6,7-dimethoxy-9-(6-(piperidin-1-yl)pyridin-3-yl)naphtho[2,3-c]furan-1(3H)-one

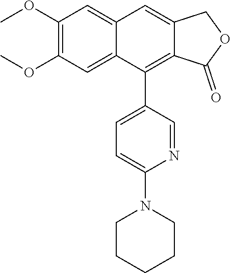 [SMILES: COc1cc2cc3c(c(-c4ccc(N5CCCCC5)nc4)c2cc1OC)C(=O)OC3]

The compound of Example 123 was synthesized by the same method as that of the compound of Example 7 except for using 2-(piperidin-1-yl)-5-(4,4,5,5-tetramethyl-1,3,2-dioxaborolan-2-yl)pyridine instead of 2-(4-methylpiperazin-1-yl)-5-(4,4,5,5-tetramethyl-1,3,2-dioxaborolan-2-yl)pyrimidine.

Mass (M+H⁺): 405.1

¹H-NMR (500 MHz, DMSO-d6): 1.58 (m, 6H), 3.58 (m, 4H), 3.67 (s, 3H), 3.92 (s, 3H), 5.40 (s, 2H), 6.90 (d, 1H), 7.06 (s, 1H), 7.47 (s, 1H), 7.52 dd, 1H), 7.89 (s, 1H), 8.06 (s, 1H).

### [Example 124] Synthesis of 6,7-dimethoxy-9-(6-morpholinopyridin-3-yl)naphtho[2,3-c]furan-1(3H)-one

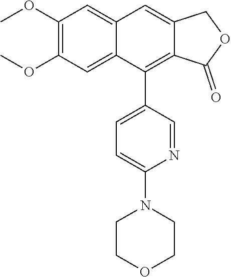 [SMILES: COc1cc2cc3c(c(-c4ccc(N5CCOCC5)nc4)c2cc1OC)C(=O)OC3]

The compound of Example 124 was synthesized by the same method as that of the compound of Example 7 except for using 4-(5-(4,4,5,5-tetramethyl-1,3,2-dioxaborolan-2-yl)pyridin-2-yl)morpholine instead of 2-(4-methylpiperazin-1-yl)-5-(4,4,5,5-tetramethyl-1,3,2-dioxaborolan-2-yl)pyrimidine.

Mass (M+H⁺): 407.1

¹H-NMR (500 MHz, DMSO-d6): 3.53 (brm, 4H), 3.67 (s, 3H), 3.72 (s, 3H), 3.92 (brm, 4H), 5.41 (s, 2H), 6.94 (m, 1H), 7.04 (s, 1H), 7.49 (s, 1H), 7.60 (m, 1H), 7.91 (s, 1H), 8.11 (s, 1H).

### [Example 125] Synthesis of 6,7-dimethoxy-9-(quinolin-8-yl)naphtho[2,3-c]furan-1(3H)-one

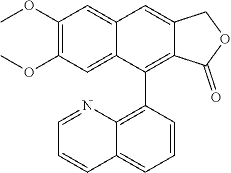 [SMILES: COc1cc2cc3c(c(-c4cccc5cccnc45)c2cc1OC)C(=O)OC3]

The compound of Example 125 was synthesized by the same method as that of the compound of Example 7 except for using 8-(4,4,5,5-tetramethyl-1,3,2-dioxaborolan-2-yl) quinoline instead of 2-(4-methylpiperazin-1-yl)-5-(4,4,5,5-tetramethyl-1,3,2-dioxaborolan-2-yl)pyrimidine.

Mass (M+H⁺):

1H-NMR (500 Mhz, DMSO-d6): δ 3.34 (s, 3H), 3.92 (s, 3H), 5.43-5.48 (m, 2H), 6.54 (s, 1H), 7.48-7.52 (m, 2H), 7.66-7.72 (m, 2H), 7.98 (s, 1H), 8.09-8.10 (m, 1H), 8.43-8.44 (m, 1H), 8.61 (s, 1H)

### [Example 126] Synthesis of 6,7-dimethoxy-9-(6-(3-methylpiperidin-1-yl)pyridin-3-yl)naphtho[2,3-c]furan-1(3H)-one

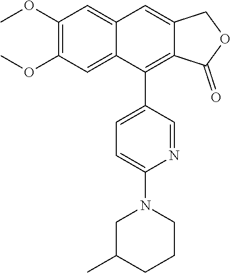 [SMILES: COc1cc2cc3c(c(-c4ccc(N5CCCC(C)C5)nc4)c2cc1OC)C(=O)OC3]

The compound of Example 126 was synthesized by the same method as that of the compound of Example 7 except for using 2-(3-methylpiperidin-1-yl)-5-(4,4,5,5-tetramethyl-1,3,2-dioxaborolan-2-yl)pyridine instead of 2-(4-methylpiperazin-1-yl)-5-(4,4,5,5-tetramethyl-1,3,2-dioxaborolan-2-yl)pyrimidine.

Mass (M+H⁺): 419.1

¹H-NMR (500 MHz, DMSO-d6): 0.91 (d, 3H), 1.14 (m, 1H), 1.48 (m, 1H), 1.60 (m, 1H), 1.70 (m, 1H), 1.79 (m, 1H), 2.51 (m, 1H), 2.80 (m, 1H), 3.67 (s, 3H), 3.92 (s, 3H), 4.28 (m, 2H), 5.40 (s, 2H), 6.91 (d, 1H), 7.06 (s, 1H), 7.47 (s, 1H), 7.51 (d, 1H), 7.88 (s, 1H), 8.06 (s, 1H).

### [Example 127] Synthesis of 9-(6-(3,5-dimethylpiperidin-1-yl)pyridin-3-yl)-6,7-dimethoxynaphtho[2,3-c]furan-1(3H)-one

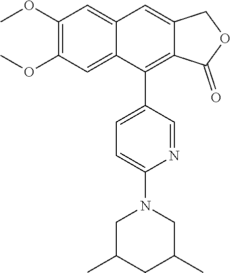 [SMILES: COc1cc2cc3c(c(-c4ccc(N5CC(C)CC(C)C5)nc4)c2cc1OC)C(=O)OC3]

The compound of Example 127 was synthesized by the same method as that of the compound of Example 7 except for using 2-(3,5-dimethylpiperidin-1-yl)-5-(4,4,5,5-tetramethyl-1,3,2-dioxaborolan-2-yl)pyridine instead of 2-(4-methylpiperazin-1-yl)-5-(4,4,5,5-tetramethyl-1,3,2-dioxaborolan-2-yl)pyrimidine.

Mass (M+H⁺): 433.1

¹H-NMR (500 MHz, DMSO-d6): 0.79 (m, 1H), 0.93 (d, 6H), 1.60 (m, 2H), 1.80 (m, 1H), 2.31 (m, 2H), 3.67 (s, 3H), 3.92 (s, 3H), 4.38 (m, 2H), 5.40 (s, 2H), 6.93 (d, 1H), 7.07 (s, 1H), 7.48 (s, 1H), 7.52 (d, 1H), 7.89 (s, 1H), 8.06 (s, 1H).

### [Example 128] Synthesis of 6,7-dimethoxy-9-(piperazin-1-yl)naphtho[2,3-c]furan-1(3H)-one

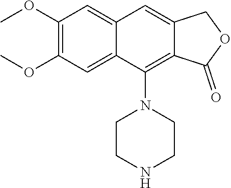 [SMILES: COc1cc2cc3c(c(N4CCNCC4)c2cc1OC)C(=O)OC3]

After 3 g (7.6 mmol) of 6,7-dimethoxy-3-oxo-1,3-dihydronaphtho[2,3-c]furan-4-yl trifluoromethanesulfonate, 1.6 g (8.7 mmol) of tert-butyl piperazin-1-carboxylate, and 3.2 g (22.8 mmol) of anhydrous potassium carbonate were injected into 100 ml of acetonitrile, the resulting mixture was stirred under reflux for 18 hours, and then stirred again with the addition of purified water to filter the crystallized solid, which was then recrystallized with ethyl acetate and purified by silica gel column chromatography to obtain 1.37 g (yield: 42%) of tert-butyl 4-(6,7-dimethoxy-3-oxo-1,3-dihydronaphtho[2,3-c]furan-4-yl) piperazin-1-carboxylate. After that, the resulting solid was dissolved in dichloromethane, and then TFA was injected thereto and stirred at room temperature for 18 hours. The reactant was dried under reduced pressure and then recrystallized with methanol/ester to obtain 0.77 g (2.34 mmol, 74%) of 6,7-dimethoxy-9-(piperazin-1-yl)naphtho[2,3-c]furan-1(3H)-one as a title compound.

Mass (M+H⁺): 329.1

¹H-NMR (500 MHz, DMSO-d6): 2.92 (brm, 4H), 5.21 (brm, 4H), 3.89 (s, 6H), 5.36 (s, 2H), 7.35 (s, 1H), 7.59 (s, 1H), 7.80 (s, 1H).

### [Example 129] Synthesis of 9-(4-(5-bromopyridin-2-yl) piperazin-1-yl)-6,7-dimethoxynaphtho[2,3-c]furan-1(3H)-one

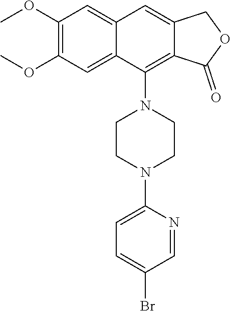 [SMILES: COc1cc2cc3c(c(N4CCN(c5ccc(Br)cn5)CC4)c2cc1OC)C(=O)OC3]

For the compound of Example 129, 6,7-dimethoxy-9-(piperazin-1-yl)naphto[2,3-c]furan-1(3H)-one (300 mg, 0.91 mmol), 192 mg of 5-bromo-2-fluoropyridine, and 253 mg of anhydrous potassium carbonate were injected into 5 ml of dimethylformamide, stirred under reflux for 18 hours, filtered with the injection of purified water, and purified by silica gel column chromatography to obtain 137 mg (0.28 mmol, 31%) of 9-(4-(5-bromopyridin-2-yl) piperazin-1-yl)-6,7-dimethoxycinaphto[2,3-c]furan-1(3H)-one as a title compound.

Mass (M+H⁺): 485.1

¹H-NMR (500 MHz, DMSO-d6): 3.39 (brm, 4H), 3.90˜3.99 (s+brm, 10H), 5.39 (s, 2H), 6.91 (d, 1H), 7.39 (s, 1H), 7.66 (s, 1H), 7.69 (d, 1H), 7.87 (s, 1H), 8.18 (s, 1H).

### [Example 130] Synthesis of 9-(4-(6-chloro-2-(methylthio)pyrimidin-4-yl) pilerazin-1-yl)-6,7-dimethoxynaphtho[2,3-c]furan-1(3H)-one

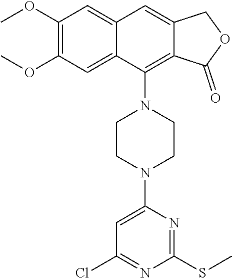

The compound of Example 130 was synthesized by the same method as that of the compound of Example 129 except for using 4,6-dichloro-2-(methylthio)pyrimidine instead of 5-bromo-2-fluoropyridine.

Mass (M+H⁺): 487.1

¹H-NMR (500 MHz, DMSO-d6): 2.42 (s, 3H), 2.47 (brm, 4H), 3.34 (brm, 4H), 3.90 (s, 3H), 3.92 (s, 3H), 5.39 (s, 2H), 6.76 (s, 1H), 7.40 (s, 1H), 7.68 (s, 1H), 7.86 (s, 1H).

### [Example 131] Synthesis of 9-(4-(5-bromopyrimidin-2-yl) piperazin-1-yl)-6,7-dimethoxynaphtho[2,3-c]furan-1(3H)-one

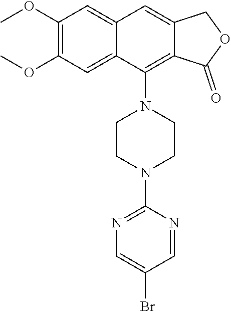 [SMILES: COc1cc2cc3c(c(N4CCN(c5ncc(Br)cn5)CC4)c2cc1OC)C(=O)OC3]

The compound of Example 131 was synthesized by the same method as that of the compound of Example 129 except for using 5-bromo-2-chloropyrimidine instead of 5-bromo-2-fluoropyridine.

Mass (M+H⁺): 486.1

¹H-NMR (500 MHZ, DMSO-d6): 2.46 (brm, 4H), 3.27 (brm, 4H), 3.90 (s, 3H), 3.92 (s, 3H), 5.38 (s, 2H), 7.39 (s, 1H), 7.67 (s, 1H), 7.88 (s, 1H), 8.47 (s, 2H).

### [Example 132] Synthesis of 6,7-dimethoxy-9-(quinolin-5-yl)naphtho[2,3-c]furan-1(3H)-one

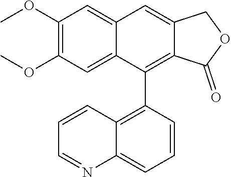 [SMILES: COc1cc2cc3c(c(-c4cccc5ncccc45)c2cc1OC)C(=O)OC3]

5 g (12.7 mmol) of 6,7-dimethoxy-3-oxo-1,3-dihydronaphtho[2,3-c]furan-4-yl trifluoromethanesulfonate, 3.88 g (15.2 mmol) of bis(pinacolato)diborane, 3.75 g (38.2 mmol) of potassium acetate, and 1.04 g (1.27 mmol) of Pd(dppf)Cl2-CH2Cl2 were added to 1,4-dioxane. After bubbling the reaction solvent with nitrogen, a reaction temperature was raised up to 80° C., and then the reaction was performed for three hours. After cooling to 25° C., 20 ml of ethyl acetate was injected and then stirred. The reaction solution was passed through a celite pad, filtered and distilled under reduced pressure. The residue was dissolved in dichloromethane, and then activated carbon was injected thereto, stirred, filtered, and distilled under reduced pressure. Methanol was injected into the distilled residue, stirred, filtered, and washed to obtain 4.01 g (10.83 mmol, 85.0%) of 6,7-dimethoxy-9-(4,4,5,5-tetramethyl-1,3,2-dioxaborolan-2-yl)naphtho[2,3-c]furan-1(3H)-one as an intermediate.

As the intermediate, 2.0 g (5.4 mmol) of 6,7-dimethoxy-9-(4,4,5,5-tetramethyl-1,3,2-dioxaboran-2-yl)naphtho[2,3-c]furan-1(3H)-one, 1.12 g (5.4 mmol) of 5-bromoquinoline and 1.49 g (10.78 mmol) of anhydrous potassium carbonate were added to 1,4-dioxane. After bubbling the reaction solvent with nitrogen, 0.18 g (0.256 mmol) of Pd(PPh₃)Cl₂ was injected thereto, and a reaction temperature was raised to 110° C., and then the reaction was performed for 18 hours. After cooling to 25° C., 20 ml of ethyl acetate was injected and then stirred. The reaction solution was passed through a celite pad, filtered and distilled under reduced pressure. The residue was dissolved in dichloromethane, and then activated carbon was injected thereto, stirred, filtered, and distilled under reduced pressure. The distilled residue was purified by column chromatography to obtain 1.34 g (3.62 mmol, 67.0%) of 6,7-dimethoxy-9-(quinolin-5-yl)naphtho[2,3-c]furan-1(3H)-one as a title compound.

Mass (M+H⁺): 372.1

¹H-NMR (500 MHz, DMSO-d6): 3.35 (s, 3H), 3.93 (s, 3H), 5.49 (s, 2H), 6.49 (s, 1H), 7.32 (dd, J=8.6, 4.0 Hz, 1H), 7.45-7.46 (m, 1H), 7.53 (dd, J=7.15, 1.15 Hz, 1H), 7.57 (s, 1H), 7.86-7.89 (m, 1H), 8.07 (s, 1H), 8.14-8.16 (m, 1H), 8.88-8.89 (m, 1H).

### [Example 133] Synthesis of 6,7-dimethoxy-9-(3-methoxyisoquinolin-8-yl)naphtho[2,3-c]furan-1(3H)-one

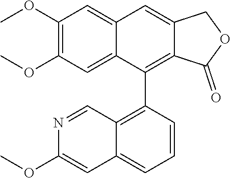 [SMILES: COc1cc2cccc(-c3c4c(cc5cc(OC)c(OC)cc35)COC4=O)c2cn1]

The compound of Example 133 was synthesized by the same method as that of the compound of Example 132 except for using 8-bromo-3-methoxyisoquinoline instead of 5-bromoquinoline.

Mass (M+H⁺): 402.1

¹H-NMR (500 MHz, DMSO-d6): 2.47 (d, J=1.75 Hz, 1H), 3.39 (s, 3H), 3.86 (s, 3H), 3.93 (s, 3H), 5.49-5.53 (m, 2H), 6.56 (s, 1H), 7.28 (d, J=0.55 Hz, 1H), 7.29-7.31 (m, 1H), 7.56 (s, 1H), 7.74-7.76 (m, 1H), 7.93-7.95 (m, 1H), 8.06 (s, 1H), 8.16 (s, 1H).

### [Example 134] Synthesis of 9-(8-fluoro-2-methylquinolin-4-yl)-6,7-dimethoxynaphtho[2,3-c]furan-1(3H)-one

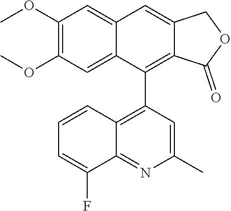 [SMILES: COc1cc2cc3c(c(-c4cc(C)nc5c(F)cccc45)c2cc1OC)C(=O)OC3]

The compound of Example 134 was synthesized by the same method as that of the compound of Example 132 except for using 4-bromo-8-fluoro-2-methylquinoline instead of 5-bromoquinoline.

Mass (M+H⁺): 404.1

¹H-NMR (500 MHz, DMSO-d6): 2.73 (s, 3H), 3.4 (s, 3H), 3.93 (s, 3H), 5.47-5.54 (m, 2H), 6.48 (s, 1H), 6.86 (d, 8.3 Hz, 1H), 7.26-7.28 (m, 1H), 7.48-7.49 (m, 2H), 7.50-7.60 (m, 1H), 7.58 (s, 1H), 8.10 (s, 1H).

### [Example 135] Synthesis of 6,7-dimethoxy-9-(4-(piperazin-1-yl) quinolin-6-yl)naphtho[2,3-c]furan-1(3H)-one

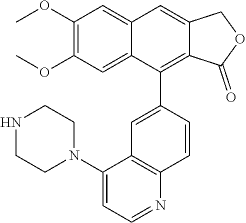 [SMILES: COc1cc2cc3c(c(-c4ccc5nccc(N6CCNCC6)c5c4)c2cc1OC)C(=O)OC3]

The compound of Example 135 was synthesized by the same method as that of the compound of Example 132 except for using 6-bromo-4-(piperazin-1-yl) quinoline instead of 5-bromoquinoline.

Mass (M+H⁺): 456.2

¹H-NMR (500 MHz, DMSO-d6): 2.89-2.97 (m, 3H), 3.08-3.12 (m, 1H), 3.24-3.34 (m, 4H), 3.54 (s, 3H), 3.93 (s, 3H), 5.44-5.47 (m, 2H), 6.93 (s, 1H), 7.03 (d, J=5.15 Hz, 1H), 7.54 (s, 1H), 7.65-7.67 (m, 1H), 8.00-8.02 (m, 2H), 8.04 (d, J=8.6 Hz, 1H), 8.74 (d, J=5.15 Hz, 1H).

### [Example 136] Synthesis of 6,7-dimethoxy-9-(2-methyl-8-(trifluoromethyl) quinolin-4-yl)naphtho[2,3-c]furan-1(3H)-one

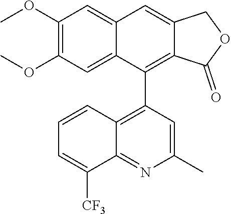 [SMILES: COc1cc2cc3c(c(-c4cc(C)nc5c(C(F)(F)F)cccc45)c2cc1OC)C(=O)OC3]

The compound of Example 136 was synthesized by the same method as that of the compound of Example 132 except for using 4-bromo-2-methyl-8-(trifluoromethyl) quinoline instead of 5-bromoquinoline.

Mass (M+H⁺): 454.1

¹H-NMR (500 MHz, DMSO-d6): 2.75 (s, 3H), 3.42 (s, 3H), 3.94 (s, 3H), 5.48-5.54 (m, 2H), 6.51 (s, 1H), 7.36 (d, J=8.55 Hz, 1H), 7.43 (t, J=7.45 Hz, 1H), 7.54 (s, 1H), 7.60 (s, 1H), 8.09-8.12 (m, 2H).

### [Example 137] Synthesis of 9-(8-fluoroquinolin-4-yl)-6,7-dimethoxynaphtho[2,3-c]furan-1(3H)-one

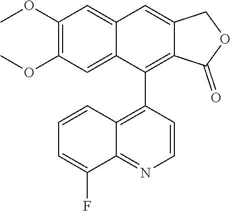 [SMILES: COc1cc2cc3c(c(-c4ccnc5c(F)cccc45)c2cc1OC)C(=O)OC3]

The compound of Example 137 was synthesized by the same method as that of the compound of Example 132 except for using 4-bromo-8-fluoroquinoline instead of 5-bromoquinoline.

Mass (M+H⁺): 390.1

¹H-NMR (500 MHz, DMSO-d6): 3.39 (s, 3H), 3.93 (s, 3H), 5.51-5.55 (m, 2H), 6.46 (s, 1H), 6.93 (d, J=8.6 Hz, 1H), 7.34-7.37 (m, 1H), 7.55-7.57 (m, 1H), 7.59-7.60 (m, 2H), 8.11 (s, H), 9.07 (d, J=4.3 Hz, 1H).

### [Example 138] Synthesis of 9-(4-chloroquinolin-6-yl)-6,7-dimethoxynaphtho[2,3-c]furan-1(3H)-one

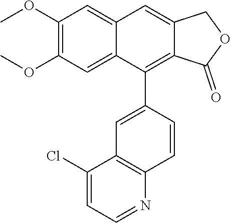 [SMILES: COc1cc2cc3c(c(-c4ccc5nccc(Cl)c5c4)c2cc1OC)C(=O)OC3]

The compound of Example 138 was synthesized by the same method as that of the compound of Example 132 except for using 6-bromo-4-chloroquinoline instead of 5-bromoquinoline.

Mass (M+H⁺): 406.1

¹H-NMR (500 MHz, DMSO-d6): 3.54 (s, 3H), 3.94 (s, 3H), 5.47 (s, 2H), 6.88 (s, 1H), 7.56 (s, 1H), 7.82 (d, J=4.55 Hz, 1H), 7.87 (dd, J=8.5 Hz, 1.7 Hz, 1H), 8.03 (s, 1H), 8.16 (d, J=1.45 Hz, 1H), 8.21 (d, J=8.6 Hz, 1H), 8.91 (d, J=4.9 Hz, 1H).

### [Example 139] Synthesis of 9-(6,8-difluoro-2-methylquinolin-4-yl)-6,7-dimethoxynaphtho[2,3-c]furan-1(3H)-one

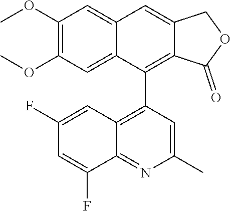 [SMILES: COc1cc2cc3c(c(-c4cc(C)nc5c(F)cc(F)cc45)c2cc1OC)C(=O)OC3]

The compound of Example 139 was synthesized by the same method as that of the compound of Example 132 except for using 4-bromo-6,8-difluoro-2-methylquinoline instead of 5-bromoquinoline.

Mass (M+H⁺): 422.1

¹H-NMR (500 MHz, DMSO-d6): 2.72 (s, 3H), 3.45 (s, 3H), 3.93 (s, 3H), 5.55-5.53 (m, 2H), 6.50 (s, 1H), 6.59-6.61 (m, 1H), 7.53 (s, 1H), 7.59 (s, 1H), 7.64-7.68 (m, 1H), 8.10 (s, 1H).

### [Example 140] Synthesis of 6,7-dimethoxy-9-(8-methoxyquinolin-4-yl)naphtho[2,3-c]furan-1(3H)-one

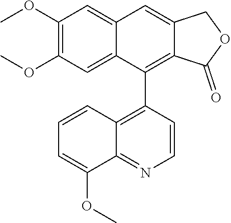 [SMILES: COc1cc2cc3c(c(-c4ccnc5c(OC)cccc45)c2cc1OC)C(=O)OC3]

The compound of Example 140 was synthesized by the same method as that of the compound of Example 132 except for using 4-bromo-8-methoxyquinoline instead of 5-bromoquinoline.

Mass (M+H⁺): 402.1

¹H-NMR (500 MHz, DMSO-d6): 3.36 (s, 3H), 3.93 (s, 3H), 3.98 (s, 3H), 5.47-5.54 (m, 2H), 6.45 (s, 1H), 6.60 (dd, J=8.6 Hz, 1.15 Hz, 1H), 7.15 (d, J=7.15 Hz, 1H), 7.27 (t, J=8.05 Hz, 1H), 7.47 (d, J=4.3 Hz, 1H), 7.58 (s, 1H), 8.09 (s, 1H), 8.95 (d, J=4.3 Hz, 1H).

### [Example 141] Synthesis of 6,7-dimethoxy-9-(7-methoxy-2-methylquinolin-4-yl)naphtho[2,3-c]furan-1(3H)-one

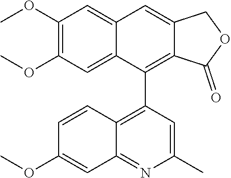 [SMILES: COc1ccc2c(-c3c4c(cc5cc(OC)c(OC)cc35)COC4=O)cc(C)nc2c1]

The compound of Example 141 was synthesized by the same method as that of the compound of Example 132 except for using 4-bromo-7-methoxy-2-methylquinoline instead of 5-bromoquinoline.

Mass (M+H⁺): 416.2

¹H-NMR (500 MHz, DMSO-d6): 2.66 (s, 3H), 3.38 (s, 3H), 3.87 (s, 3H), 3.93 (s, 3H), 5.46-5.50 (m, 2H), 6.49 (s, 1H), 6.91-6.93 (m, 2H), 7.19 (s, 1H), 7.41 (d, J=1.75 Hz, 1H), 7.56 (s, 1H), 8.07 (s, 1H).

### [Example 142] Synthesis of 9-(6-(4-chloropiperidin-1-yl)pyridin-3-yl)-6,7-dimethoxynaphtho[2,3-c]furan-1(3H)-one

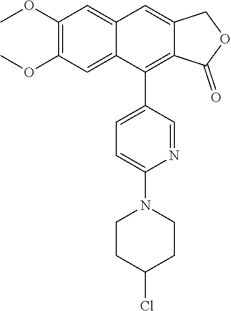 [SMILES: COc1cc2cc3c(c(-c4ccc(N5CCC(Cl)CC5)nc4)c2cc1OC)C(=O)OC3]

The compound of Example 142 was synthesized by the same method as that of the compound of Example 132 except for using 5-bromo-2-(4-chloropiperidin-1-yl)pyridine instead of 5-bromoquinoline.

Mass (M+H⁺): 439.2

¹H-NMR (500 MHz, DMSO-d6): 1.78 (m, 2H), 2.13 (m, 2H), 3.41 (m, 2H), 3.67 (s, 3H), 3.92 (s, 3H), 4.00 (m, 2H), 4.45 (m, 1H), 5.40 (s, 2H), 6.97 (d, 1H), 7.04 (s, 1H), 7.48 (s, 1H), 7.56 (d, 1H), 7.90 (s, 1H), 8.09 (s, 1H).

### [Example 143] Synthesis of 6,7-dimethoxy-9-(4-(2-morpholinoethyl) piperazin-1-yl)naphtho[2,3-c]furan-1(3H)-one

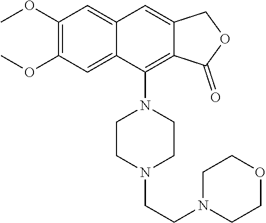 [SMILES: COc1cc2cc3c(c(N4CCN(CCN5CCOCC5)CC4)c2cc1OC)C(=O)OC3]

For the compound of Example 143, 6,7-dimethoxy-9-(piperazin-1-yl)naphto[2,3-c]furan-1(3H)-one (300 mg, 0.91 mmol), chloroethyl morpholine (340 mg, 1.83 mmol), and anhydrous potassium carbonate (503 mg, 3.64 mmol) were injected into 10 ml of dimethylformamide, stirred under reflux for 18 hours, filtered with the injection of purified water, and purified by silica gel column chromatography to obtain 223 mg (0.505 mmol, 56%) of 6,7-dimethoxy-9-(4-(2-morpholinoethyl) piperazin-1-yl)naphtho[2,3-c]furan-1(3H)-one as a title compound.

Mass (M+H⁺): 442.2

¹H-NMR (500 MHz, DMSO-d6): 2.40 (m, 4H), 2.46 (m, 4H), 2.54 (t, 2H), 3.27 (brm, 2H), 3.54 (t, 2H), 3.60 (brm, 2H), 3.89 (s, 3H), 3.91 (s, 3H), 4.14 (t, 2H), 5.38 (s, 2H), 7.38 (s, 1H), 7.66 (s, 1H), 7.81 (s, 1H).

### [Example 144] Synthesis of 9-(4-(2-hydroxyethyl) piperazin-1-yl)-6,7-dimethoxynaphtho[2,3-c]furan-1(3H)-one

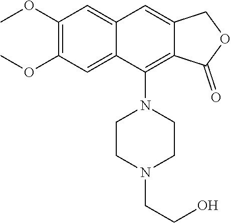 [SMILES: COc1cc2cc3c(c(N4CCN(CCO)CC4)c2cc1OC)C(=O)OC3]

After 1 g (2.5 mmol) of 6,7-dimethoxy-3-oxo-1,3-dihydronaphtho[2,3-c]furan-4-yl trifluoromethanesulfonate, 980 mg (7.5 mmol) of hydroxy ethyl piperazine, and 0.7 g (5.1 mmol) of anhydrous potassium carbonate were injected into 20 ml of acetonitrile, the resulting mixture was stirred under reflux for seven hours and stirred again with the addition of purified water to filter the crystallized solid, which was then recrystallized with ethyl acetate and purified by silica gel column chromatography to obtain 0.703 g (1.89 mmol, 76%) of 9-(4-(2-hydroxyethyl) piperazin-1-yl)-6,7-dimethoxynaphtho[2,3-c]furan-1(3H)-one as a title compound.

Mass (M+H⁺): 373.1

¹H-NMR (500 MHz, DMSO-d6): 2.46 (m, 2H), 2.53 (brm, 4H), 3.30 (brm, 4H), 3.53 (m, 2H), 3.88 (s, 3H), 3.89 (s, 3H), 4.41 (t, 1H), 5.36 (s, 2H), 7.35 (s, 1H), 7.60 (s, 1H), 7.76 (s, 1H).

### [Example 145] Synthesis of 9-(4-(2-hydroxyethyl) piperazin-1-yl)-6,7-dimethoxynaphtho[2,3-c]furan-1(3H)-one

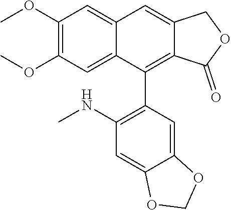 [SMILES: CNc1cc2c(cc1-c1c3c(cc4cc(OC)c(OC)cc14)COC3=O)OCO2]

After 1 g (2.5 mmol) of 6,7-dimethoxy-3-oxo-1,3-dihydronaphtho[2,3-c]furan-4-yl trifluoromethanesulfonate, 980 mg (7.5 mmol) of hydroxy ethyl piperazine, and 0.7 g (5.1 mmol) of anhydrous potassium carbonate were injected into 20 ml of acetonitrile, the resulting mixture was stirred under reflux for seven hours and stirred again with the addition of purified water to filter the crystallized solid, which was then recrystallized with ethyl acetate and purified by silica gel column chromatography to obtain 0.703 g (1.89 mmol, 76%) of 9-(4-(2-hydroxyethyl) piperazin-1-yl)-6,7-dimethoxynaphtho[2,3-c]furan-1(3H)-one as a title compound.

Mass (M+H⁺): 373.1

¹H-NMR (500 MHz, DMSO-d6): 2.46 (m, 2H), 2.53 (brm, 4H), 3.30 (brm, 4H), 3.53 (m, 2H), 3.88 (s, 3H), 3.89 (s, 3H), 4.41 (t, 1H), 5.36 (s, 2H), 7.35 (s, 1H), 7.60 (s, 1H), 7.76 (s, 1H).

### [Example 146] Synthesis of 9-(4-hydroxy-2,8-dimethylquinolin-7-yl)-6,7-dimethoxynaphtho[2,3-c]furan-1(3H)-one

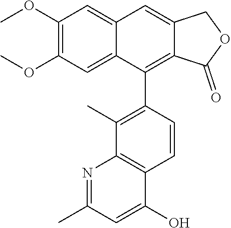 [SMILES: COc1cc2cc3c(c(-c4ccc5c(O)cc(C)nc5c4C)c2cc1OC)C(=O)OC3]

The compound of Example 146 was synthesized by the same method as that of the compound of Example 132 except for using 7-bromo-2,8-dimethylquinolin-4-ol instead of 5-bromoquinoline.

Mass (M+H⁺): 416.1

¹H-NMR (500 MHz, DMSO-d6): 2.06 (s, 3H), 2.41 (s, 3H), 3.51 (s, 3H), 3.93 (s, 3H), 5.47-5.48 (m, 2H), 5.99 (s, 1H), 6.59 (s, 1H), 7.02 (d, J=8.3 Hz, 1H), 7.54 (s, 1H), 7.98-8.00 (m, 2H), 10.38 (s, 1H).

### [Example 147] Synthesis of 9-(4-hydroxy-2-methylquinolin-6-yl)-6,7-dimethoxynaphtho[2,3-c]furan-1(3H)-one

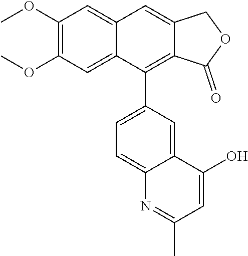 [SMILES: COc1cc2cc3c(c(-c4ccc5nc(C)cc(O)c5c4)c2cc1OC)C(=O)OC3]

The compound of Example 147 was synthesized by the same method as that of the compound of Example 132 except for using 6-bromo-2-methylquinolin-4-ol instead of 5-bromoquinoline.

Mass (M+H⁺): 402.1

¹H-NMR (500 MHz, DMSO-d6): 2.36 (s, 3H), 3.54 (s, 3H), 3.92 (s, 3H), 5.44 (s, 2H), 5.93 (s, 1H), 6.86 (s, 1H), 7.51 (s, 1H), 7.58 (s, 2H), 7.95 (d, J=7.45 Hz, 2H), 11.69 (s, 1H).

### [Example 148] Synthesis of 9-(6-fluoroquinolin-4-yl)-6,7-dimethoxynaphtho[2,3-c]furan-1(3H)-one

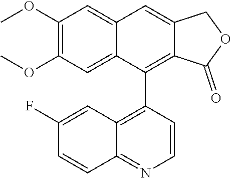 [SMILES: COc1cc2cc3c(c(-c4ccnc5ccc(F)cc45)c2cc1OC)C(=O)OC3]

The compound of Example 148 was synthesized by the same method as that of the compound of Example 132 except for using 4-bromo-6-fluoroquinoline instead of 5-bromoquinoline.

Mass (M+H⁺): 390.1

¹H-NMR (500 MHz, DMSO-d6) 3.40 (s, 3H), 3.94 (s, 3H), 5.50-5.54 (m, 2H), 6.46 (s, 1H), 6.79 (d, J=9.75 Hz, 1H), 7.53 (d, J=4.3 Hz, 1H), 7.59 (s, 1H), 7.63-7.67 (m, 1H), 8.11 (s, 1H), 8.19-8.21 (m, 1H), 9.02 (d, J=4.3 Hz, 1H).

### [Example 149] Synthesis of 6,7-dimethoxy-9-(8-methoxy-2-methylquinolin-4-yl)naphtho[2,3-c]furan-1(3H)-one

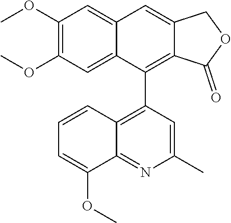 [SMILES: COc1cc2cc3c(c(-c4cc(C)nc5c(OC)cccc45)c2cc1OC)C(=O)OC3]

The compound of Example 149 was synthesized by the same method as that of the compound of Example 132 except for using 4-bromo-8-methoxy-2-methylquinoline instead of 5-bromoquinoline.

Mass (M+H⁺): 416.1

¹H-NMR (500 MHz, DMSO-d6) 2.68 (s, 3H), 3.37 (s, 3H), 3.93 (s, 3H), 3.96 (s, 3H), 5.46-5.53 (m, 2H), 6.46 (s, 1H), 6.55 (d, J=1.15 Hz, 1H), 7.09-7.11 (m, 1H), 7.17-7.19 (m, 1H), 7.35 (s, 1H), 7.57 (s, 1H), 8.07 (s, 1H).

### [Example 150] Synthesis of 6,7-dimethoxy-9-(7-methoxyquinolin-4-yl)naphtho[2,3-c]furan-1(3H)-one

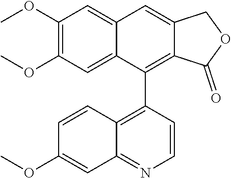 [SMILES: COc1ccc2c(-c3c4c(cc5cc(OC)c(OC)cc35)COC4=O)ccnc2c1]

The compound of Example 150 was synthesized by the same method as that of the compound of Example 132 except for using 4-bromo-7-methoxyquinoline instead of 5-bromoquinoline.

Mass (M+H⁺): 402.1

¹H-NMR (500 MHz, DMSO-d6) 3.37 (s, 3H), 3.90 (s, 3H), 3.93 (s, 3H), 5.47-5.54 (m, 2H), 6.46 (s, 1H), 6.98-7.02 (m, 2H), 7.31 (d, J=4.6 Hz, 1H), 7.58 (s, 1H), 8.09 (s, 1H), 8.93 (d, J=4.15 Hz, 1H).

### [Example 151] Synthesis of (S)-6,7-dimethoxy-9-(2-(1-phenyl-3,4-dihydroisoquinolin-2 (1H)-yl)pyrimidin-5-yl)naphtho[2,3-c]furan-1(3H)-one

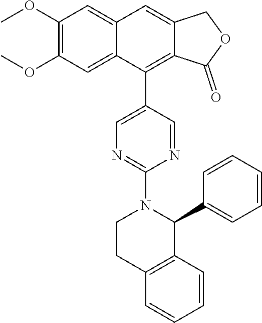 [SMILES: COc1cc2cc3c(c(-c4cnc(N5CCc6ccccc6[C@@H]5c5ccccc5)nc4)c2cc1OC)C(=O)OC3]

The compound of Example 151 was synthesized by the same method as that of the compound of Example 132 except for using (S)-2-(5-bromopyrimidin-2-yl)-1-phenyl-1,2,3,4-tetrahydroisoquinoline instead of 5-bromoquinoline.

Mass (M+H⁺): 530.2

¹H-NMR (500 MHz, DMSO-d6) 2.84-2.88 (m, 2H), 2.98-3.05 (m, 1H), 3.64-3.69 (m, 1H), 3.71 (s, 3H), 3.92 (s, 3H), 4.37-4.40 (m, 1H), 5.43 (s, 2H), 7.08 (s, 2H), 7.22-7.28 (m, 9H), 7.51 (s, 1H), 7.58 (s, 1H), 7.94 (s, 1H), 8.48 (d, J=1.75 Hz, 1H).

### [Example 152] Synthesis of 6,7-dimethoxy-9-(4-(piperazin-1-yl) quinolin-7-yl)naphtho[2,3-c]furan-1(3H)-one

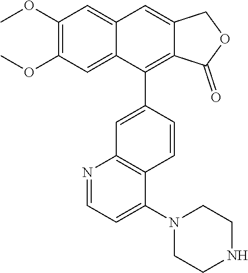 [SMILES: COc1cc2cc3c(c(-c4ccc5c(N6CCNCC6)ccnc5c4)c2cc1OC)C(=O)OC3]

The compound of Example 152 was synthesized by the same method as that of the compound of Example 132 except for using 7-bromo-4-(piperazin-1-yl) quinoline instead of 5-bromoquinoline.

Mass (M+H⁺): 456.2

¹H-NMR (500 MHz, DMSO-d6) 1.75 (s, 4H), 2.94-2.98 (m, 4H), 3.52 (s, 3H), 3.93 (s, 3H), 5.46 (s, 2H), 6.90 (s, 1H), 7.01 (d, J=5.15 Hz, 1H), 7.49 (dd, J=8.6 Hz, 1.7 Hz, 1H), 7.54 (s, 1H), 7.87 (d, J=1.4 Hz, 1H), 8.00 (s, 1H), 8.11 (d, J=8.55 Hz, 1H), 8.70 (d, J=4.9 Hz, 1H).

### [Example 153] Synthesis of 9-(8-hydroxy-6-(trifluoromethyl) naphthalen-2-yl)-6,7-dimethoxynaphtho[2,3-c]furan-1(3H)-one

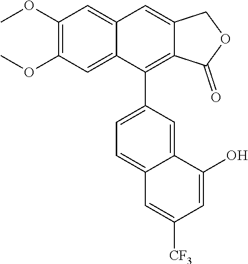 [SMILES: COc1cc2cc3c(c(-c4ccc5cc(C(F)(F)F)cc(O)c5c4)c2cc1OC)C(=O)OC3]

The compound of Example 153 was synthesized by the same method as that of the compound of Example 132 except for using 7-bromo-3-(trifluoromethyl) naphthalen-1-ol instead of 5-bromoquinoline.

Mass (M+H⁺): 456.1

¹H-NMR (500 MHz, DMSO-d6) 3.54 (s, 3H), 3.93 (s, 3H), 5.46 (s, 2H), 6.86 (s, 1H), 7.54 (s, 1H), 7.81 (d, J=8.3 Hz, 1H), 8.01 (s, 1H), 8.10-8.14 (m, 4H).

### [Example 154] Synthesis of 9-(4-hydroxy-8-methylquinolin-7-yl)-6,7-dimethoxynaphtho[2,3-c]furan-1(3H)-one

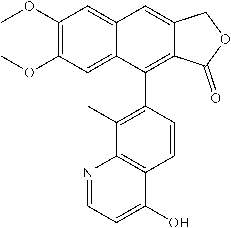 [SMILES: COc1cc2cc3c(c(-c4ccc5c(O)ccnc5c4C)c2cc1OC)C(=O)OC3]

The compound of Example 154 was synthesized by the same method as that of the compound of Example 132 except for using 7-bromo-8-methylquinolin-4-ol instead of 5-bromoquinoline.

Mass (M+H⁺): 402.1

¹H-NMR (500 MHz, DMSO-d6) 2.02 (s, 3H), 3.52 (s, 3H), 3.93 (s, 3H), 5.48 (s, 2H), 6.12 (d, J=7.4 Hz, 1H), 6.59 (s, 1H), 7.06 (d, J=8.3 Hz, 1H), 7.54 (s, 1H), 7.84-7.85 (m, 1H), 8.00-8.04 (m, 2H), 11.13 (d, J=3.8 Hz, 1H).

### [Example 155] Synthesis of 6,7-dimethoxy-9-(6-methoxy-2-methylquinolin-4-yl)naphtho[2,3-c]furan-1(3H)-one

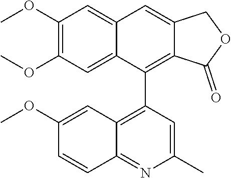 [SMILES: COc1ccc2nc(C)cc(-c3c4c(cc5cc(OC)c(OC)cc35)COC4=O)c2c1]

The compound of Example 155 was synthesized by the same method as that of the compound of Example 132 except for using 4-bromo-6-methoxy-2-methylquinoline instead of 5-bromoquinoline.

Mass (M+H⁺): 416.1

¹H-NMR (500 MHz, DMSO-d6) 2.64 (s, 3H), 3.39 (s, 3H), 3.44 (s, 3H), 3.93 (s, 3H), 5.47-5.51 (m, 2H), 6.32 (d, J=2.85 Hz, 1H), 6.52 (s, 1H), 7.29 (s, 1H), 7.36 (dd, J=12.0 Hz, 2.9 Hz, 1H), 7.57 (s, 1H), 7.94 (d, J=9.15 Hz, 1H), 8.08 (s, 1H).

### [Example 156] Synthesis of 9-(6-(6,7-dihydrothien[3,2-c]pyridin-5 (4H)-yl)pyridin-3-yl)-6,7-dimethoxynaphtho[2,3-c]furan-1(3H)-one

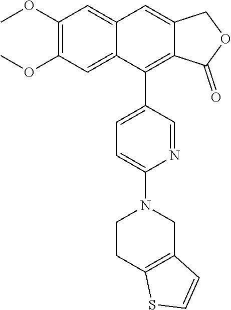 [SMILES: COc1cc2cc3c(c(-c4ccc(N5CCc6sccc6C5)nc4)c2cc1OC)C(=O)OC3]

The compound of Example 156 was synthesized by the same method as that of the compound of Example 7 except for using 5-(5-(4,4,5,5-tetramethyl-1,3,2-dioxaborolan-2-yl)pyridin-2-yl)-4,5,6,7-tetrahydrothien[3,2-c]pyridine instead of 2-(4-methylpiperazin-1-yl)-5-(4,4,5,5-tetramethyl-1,3,2-dioxaborolan-2-yl)pyrimidine.

Mass (M+H⁺): 459.1

¹H-NMR (500 MHZ, DMSO-d6) 2.91 (t, J=5.45 Hz, 2H), 3.67 (s, 3H), 3.92 (s, 3H), 4.00-4.03 (m, 2H), 663-4.71 (m, 2H) 5.41 (s, 2H), 6.94 (d, J=5.15 Hz, 1H), 7.02 (d, J=8.85 Hz, 1H), 7.06 (s, 1H), 7.33 (d, J=5.15 Hz, 1H), 7.49 (s, 1H), 7.59 (dd, J=8.85 Hz, 2.55 Hz, 1H), 7.90 (s, 1H), 8.11 (d, J=0.6 Hz, 1H).

### [Example 157] Synthesis of 9-(4-(4,6-dimethoxypyrimidin-5-yl)phenyl)-6,7-dimethoxynaphtho[2,3-c]furan-1(3H)-one

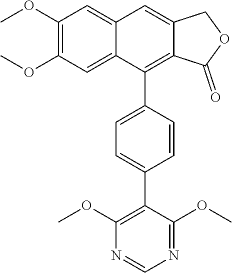

The compound of Example 157 was synthesized by the same method as that of the compound of Example 132 except for using 5-(4-bromophenyl)-4,6-dimethoxypyrimidine instead of 5-bromoquinoline.

Mass (M+H⁺): 459.2

¹H-NMR (500 MHz, DMSO-d6) 3.62 (s, 3H), 3.90 (s, 6H), 3.92 (s, 3H), 5.44 (s, 2H), 6.94 (s, 1H), 7.37-6.39 (m, 2H), 7.50-7.52 (m, 3H), 7.95 (s, 1H), 8.51 (s, 1H).

### [Example 158] Synthesis of 9-(2-((2-(1,3-dioxolan-2-yl)ethyl)(methyl)amino)pyrimidin-5-yl)-6,7-dimethoxynaphtho[2,3-c]furan-1(3H)-one

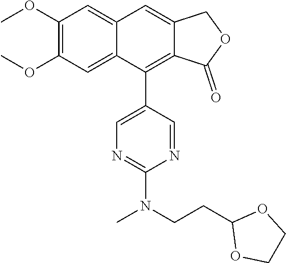 [SMILES: COc1cc2cc3c(c(-c4cnc(N(C)CCC5OCCO5)nc4)c2cc1OC)C(=O)OC3]

The compound of Example 158 was synthesized by the same method as that of the compound of Example 132 except for using N-(2-(1,3-oxolan-2-yl)ethyl)-5-bromo-N-methylpyrimidin-2-amine instead of 5-bromoquinoline.

Mass (M+H⁺): 452.2

¹H-NMR (500 MHZ, CDCl₃) 2.09-2.12 (m, 2H), 3.29 (s, 3H), 3.87-3.89 (m, 7H), 3.98-4.00 (m, 2H), 4.05 (s, 3H), 5.00 (t, J=4.6 Hz, 1H), 5.39 (d, J=0.85 Hz, 2H), 7.19-7.20 (m, 2H), 7.71 (s, 1H), 8.41 (s, 2H).

### [Example 159] Synthesis of 6,7-dimethoxy-9-(6-(4-methylpiperidin-1-yl)pyridin-3-yl)naphtho[2,3-c]furan-1(3H)-one

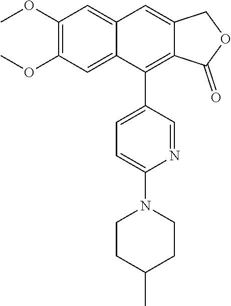 [SMILES: COc1cc2cc3c(c(-c4ccc(N5CCC(C)CC5)nc4)c2cc1OC)C(=O)OC3]

The compound of Example 159 was synthesized by the same method as that of the compound of Example 132 except for using 5-bromo-2-(4-methylpiperidin-1-yl)pyridine instead of 5-bromoquinoline.

Mass (M+H⁺): 419.1

¹H-NMR (500 MHZ, DMSO-d6): 0.94 (s, 3H), 1.14 (m, 2H), 1.62 (m, 1H), 1.69 (m, 1H), 2.84 (m, 2H), 2.84 (s, 2H), 3.68 (s, 3H), 3.93 (s, 3H), 4.35 (t, 2H), 5.40 (s, 2H), 6.90 (d, 1H), 7.07 (s, 1H), 7.49 (dd, 2H), 7.90 (d, 1H), 8.01 (d, 1H).

### [Example 160] Synthesis of 9-(6-(diethylamino)pyridin-3-yl)-6,7-dimethoxynaphtho[2,3-c]furan-1(3H)-one

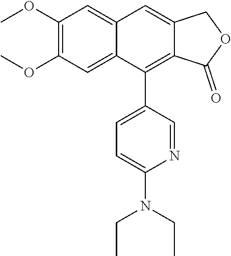 [SMILES: CCN(CC)c1ccc(-c2c3c(cc4cc(OC)c(OC)cc24)COC3=O)cn1]

The compound of Example 160 was synthesized by the same method as that of the compound of Example 132 except for using 5-bromo-N,N-diethylpyridin-2-amine instead of 5-bromoquinoline.

Mass (M+H⁺): 393.1

¹H-NMR (500 MHz, DMSO-d6): 1.15 (m, 6H), 3.55 (m, 4H), 3.68 (s, 3H), 3.92 (s, 3H), 5.40 (s, 2H), 6.67 (t, 1H), 7.11 (s, 1H), 7.47 (t, 2H), 7.88 (s, 1H), 8.05 (d, 1H).

### [Example 161] Synthesis of 9-(6-((2-(1,3-dioxolan-2-yl)ethyl)(methyl)amino)pyridin-3-yl)-6,7-dimethoxynaphtho[2,3-c]furan-1(3H)-one

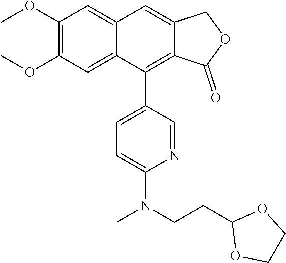 [SMILES: COc1cc2cc3c(c(-c4ccc(N(C)CCC5OCCO5)nc4)c2cc1OC)C(=O)OC3]

The compound of Example 161 was synthesized by the same method as that of the compound of Example 132 except for using N-(2-(1,3-dioxolan-2-yl)ethyl)-5-bromo-N-methylpyridin-2-amine instead of 5-bromoquinoline.

Mass (M+H⁺): 451.1

¹H-NMR (500 MHz, DMSO-d6): 1.90 (q, 2H), 3.05 (s, 3H), 3.59 (m, 2H), 3.68 (s, 3H), 3.76 (m, 2H), 3.89 (m, 2H), 3.92 (s, 3H), 4.87 (m, 1H), 5.41 (s, 2H), 6.70 (d, 1H), 7.08 (s, 1H), 7.48 (s, 1H), 7.53 (d, 1H), 7.89 (s, 1H), 8.06 (s, 1H).

### [Example 162] Synthesis of 9-(6-((2,2-dimethoxyethyl)(methyl)amino)pyridin-3-yl)-6,7-dimethoxynaphtho[2,3-c]furan-1(3H)-one

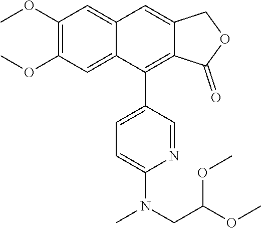

The compound of Example 162 was synthesized by the same method as that of the compound of Example 132 except for using 5-bromo-N-(2,2-dimethoxyethyl)-N-methylpyridin-2-amine instead of 5-bromoquinoline.

Mass (M+H⁺): 439.2

¹H-NMR (500 MHz, DMSO-d6): 3.08 (s, 3H), 3.30 (s, 6H), 3.67 (s, 3H), 3.69 (m, 2H), 3.92 (s, 3H), 4.59 (t, 1H), 5.40 (s, 2H), 6.84 (d, 1H), 7.06 (s, 1H), 7.48 (s, 1H), 7.54 (d, 1H), 7.89 (s, 1H), 8.06 (s, 1H).

### [Example 163] Synthesis of 9-(6-(dimethylamino)pyridin-3-yl)-6,7-dimethoxynaphtho[2,3-c]furan-1(3H)-one

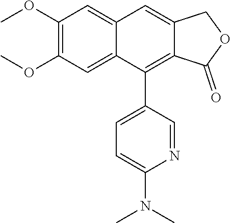 [SMILES: COc1cc2cc3c(c(-c4ccc(N(C)C)nc4)c2cc1OC)C(=O)OC3]

The compound of Example 163 was synthesized by the same method as that of the compound of Example 132 except for using 5-bromo-N,N-dimethylpyridin-2-amine instead of 5-bromoquinoline.

Mass (M+H⁺): 365.2

¹H-NMR (500 MHz, DMSO-d6): 3.09 (s, 6H), 3.67 (s, 3H), 3.91 (s, 3H), 5.39 (s, 2H), 6.74 (d, 1H), 7.06 (S, 1H) 7.47 (S, 1H), 7.53 (t, 1H), 7.88 (s, 1H), 8.06 (d, 1H).

### [Example 164] Synthesis of (S)-6,7-dimethoxy-9-(6-((5-methoxy-1,2,3,4-tetrahydronaphthalen-2-yl)(methyl)amino)pyridin-3-yl)naphtho[2,3-c]furan-1(3H)-one

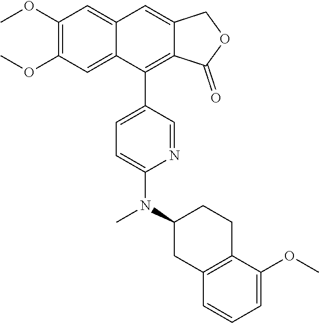

The compound of Example 164 was synthesized by the same method as that of the compound of Example 132 except for using (S)-5-bromo-N-(5-methoxy-1,2,3,4-tetrahydronaphthalen-2-yl)-N-methylpyridin-2-amine instead of 5-bromoquinoline.

Mass (M+H⁺): 511.2

¹H-NMR (500 MHz, DMSO-d6): 1.95 (m, 2H), 2.60 (m, 1H), 2.80 (m, 1H), 2.92 (m, 2H), 2.97 (s, 3H), 3.68 (s, 3H), 3.74 (s, 3H), 4.00 (s, 3H), 4.85 (m, 1H), 5.39 (s, 2H), 6.74 (d, 1H), 6.77 (d, 1H), 6.79 (d, 1H), 7.06 (m, 1H), 7.09 (d, 1H), 7.47 (s, 1H), 7.53 (q, 1H), 7.83 (s, 1H), 8.05 (d, 1H).

### [Example 165] Synthesis of 9-(6-(4-hydroxypiperidin-1-yl)pyridin-3-yl)-6,7-dimethoxynaphtho[2,3-c]furan-1(3H)-one

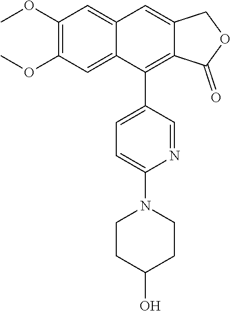 [SMILES: COc1cc2cc3c(c(-c4ccc(N5CCC(O)CC5)nc4)c2cc1OC)C(=O)OC3]

The compound of Example 165 was synthesized by the same method as that of the compound of Example 7 except for using 1-(5-(4,4,5,5-tetramethyl-1,3,2-dioxaborolan-2-yl)pyridin-2-yl) piperidin-4-ol instead of 2-(4-methylpiperazin-1-yl)-5-(4,4,5,5-tetramethyl-1,3,2-dioxaborolan-2-yl)pyrimidine.

Mass (M+H⁺): 421.0

¹H-NMR (500 MHz, DMSO-d6): 1.41 (m, 2H), 1.80 (m, 2H), 3.13 (m, 2H), 3.67 (s, 3H), 3.71 (m, 1H), 3.92 (s, 3H), 4.08 (m, 2H), 4.70 (d, 1H), 5.40 (s, 2H), 6.92 (d, 1H), 7.06 (s, 1H), 7.47 (s, 1H), 7.53 (d, 1H), 7.89 (s, 1H), 8.07 (d, 1H).

### [Example 166] Synthesis of 9-(6-(1,4-dioxa-8-azaspiro[4.5]decan-8-yl)pyridin-3-yl)-6,7-dimethoxynaphtho[2,3-c]furan-1(3H)-one

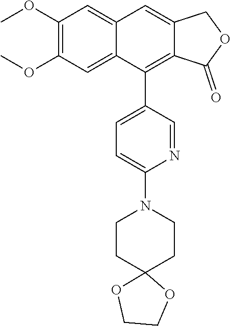 [SMILES: COc1cc2cc3c(c(-c4ccc(N5CCC6(CC5)OCCO6)nc4)c2cc1OC)C(=O)OC3]

The compound of Example 166 was synthesized by the same method as that of the compound of Example 132 except for using 8-(5-bromopyridin-2-yl)-1,4-dioxa-8-azaspiro[4.5]decane instead of 5-bromoquinoline.

Mass (M+H⁺): 463.2

¹H-NMR (500 MHz, DMSO-d6): 1.68 (m, 4H), 3.67 (s, 3H), 3.70 (m, 4H), 3.91 (s+m, 7H), 5.40 (s, 2H), 6.97 (d, 1H), 7.05 (s, 1H), 7.48 (s, 1H), 7.55 (d, 1H), 7.89 (s, 1H), 8.08 (d, 1H).

### [Example 167] Synthesis of 6,7-dimethoxy-9-(6-(4-methylpiperazin-1-yl)pyridin-3-yl)naphtho[2,3-c]furan-1(3H)-one

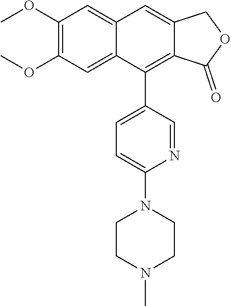 [SMILES: COc1cc2cc3c(c(-c4ccc(N5CCN(C)CC5)nc4)c2cc1OC)C(=O)OC3]

The compound of Example 167 was synthesized by the same method as that of the compound of Example 132 except for using 1-(5-bromopyridin-2-yl)-4-methylpiperazine instead of 5-bromoquinoline.

Mass (M+H⁺): 420.1

¹H-NMR (500 MHz, DMSO-d6): 2.22 (s, 3H), 2.42 (m, 4H), 3.56 (m, 4H), 3.67 (s, 3H), 3.92 (s, 3H), 5.40 (s, 2H), 6.92 (d, 1H), 7.04 (d, 1H), 7.48 (s, 1H), 7.56 (d, 1H), 7.90 (s, 1H), 8.08 (d, 1H).

### [Example 168] Synthesis of 6,7-dimethoxy-9-(6-((2-methoxyethyl)(methyl)amino)pyridin-3-yl)naphtho[2,3-c]furan-1(3H)-one hydrochloride

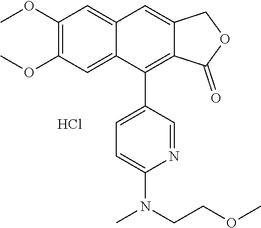

The compound of Example 168 was synthesized by the same method as that of the compound of Example 132 except for using 5-bromo-N-(2-methoxyethyl)-N-methylpyridin-2-amine instead of 5-bromoquinoline.

Mass (M+H⁺): 409.2

¹H-NMR (500 MHz, DMSO-d6): 3.27 (s, 3H), 3.50 (s, 3H), 3.61 (t, 2H), 3.73 (s, 3H), 3.92 (s, 2H), 3.93 (s, 3H), 5.45 (s, 2H), 7.02 (s, 1H), 7.28 (br, 1H), 7.54 (s, 1H), 7.95 (br, 1H), 7.99 (s, 1H), 8.07 (s, 1H).

### [Example 169] Synthesis of 6-(6,7-dimethoxy-3-oxo-1,3-dihydronaphtho[2,3-c]furan-4-yl)benzo[d][1,3]dioxol-5-yl picolate

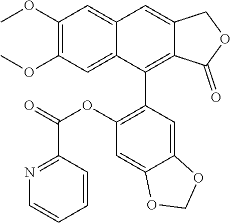 [SMILES: COc1cc2cc3c(c(-c4cc5c(cc4OC(=O)c4ccccn4)OCO5)c2cc1OC)C(=O)OC3]

After 5 g (13.14 mmol) of 9-(6-hydroxybenzo[d][1,3]dioxol-5-yl)-6,7-dimethoxynaphtho[2,3-c]furan-1(3H)-one, 1.7 g (13.8 mmol) of picolic acid, 0.8 g (6.54 mmol) of DMAP, and 3.0 g (15.6 mmol) of EDC-HCl were injected into 30 ml of dichloromethane, the resulting mixture was stirred at room temperature for two hours. After 20 ml of purified water was injected into the reactant and separated in layers, 20% hydrochloric acid was diluted 50 times and injected in the organic layer, and separated in layers. The organic layer was dried over anhydrous sodium sulfate, filtered, washed, and distilled under reduced pressure. The residue was dissolved in methanol, 5 ml of 35% hydrochloric acid was injected thereto, and the resulting mixture was stirred for three hours. The reactant was distilled under reduced pressure and the residue was dissolved in dichloromethane, after which tert-butylmethyl ester was slowly added dropwise thereto and stirred, and then the resulting solid was filtered and washed to obtain 2.7 g (5.56 mmol, 42.3%) of 6-(6,7-dimethoxy-3-oxo-1,3-dihydronaphtho[2,3-c]furan-4-yl)benzo[d][1,3]dioxol-5-yl picolate as a title compound.

Mass (M+H⁺): 486.1

¹H-NMR (500 MHz, DMSO-d6): 3.96 (s, 3H), 3.84 (s, 3H), 5.41 (s, 2H), 6.19 (s, 2H), 6.94 (s, 1H), 6.95 (s, 1H), 7.17 (s, 1H), 7.35-7.37 (m, 2H), 7.47-7.48 (m, 1H), 7.84 (s, 1H), 8.50-8.51 (m, 1H).

### [Example 170] Synthesis of 6,7-dimethoxy-9-(1-methyl-1,2,3,4-tetrahydroquinolin-7-yl)naphtho[2,3-c]furan-1(3H)-one

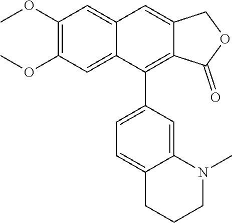

1 g (4.80 mmol) of 7-bromoquinoline and 4.33 g of paraformaldehyde were dissolved in 30 ml of acetic acid. 1.5 g of NaBH4CN was slowly added dropwise to the reaction solution, and the resulting mixture was stirred for 20 minutes. The reaction solution was neutralized with sodium hydroxide and extracted with dichloromethane. The organic layer was washed with brine, dried over anhydrous sodium sulfate, filtered, washed, and distilled under reduced pressure to obtain 1.05 g (4.64 mmol, m/z (M+H+): 228.0, 96.6%) of 7-bromo-1-methyl-1,2,3,4-tetrahydroquinoline as an intermediate. The compound of Example 170 was synthesized by the same method as that of the compound of Example 132 except for using 7-bromo-1-methyl-1,2,3,4-tetrahydroquinoline instead of 5-bromoquinoline.

Mass (M+H⁺): 390.1

¹H-NMR (500 MHz, DMSO-d6): 1.95-1.99 (m, 2H), 2.80-2.83 (m, 5H), 3.28 (br, 2H), 3.63 (s, 3H), 3.91 (s, 3H), 5.40 (s, 2H), 6.66-6.70 (br, 2H), 7.06 (s, 2H), 7.47 (s, 1H), 7.90 (s, 1H).

### [Example 171] Synthesis of 9-(6-(cyclopropyl(methyl)amino)pyridin-3-yl)-6,7-dimethoxynaphtho[2,3-c]furan-1(3H)-one

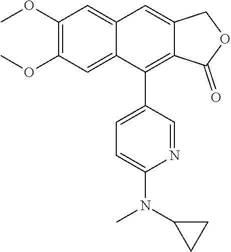 [SMILES: COc1cc2cc3c(c(-c4ccc(N(C)C5CC5)nc4)c2cc1OC)C(=O)OC3]

The compound of Example 171 was synthesized by the same method as that of the compound of Example 132 except for using 5-bromo-N-cyclopropyl-N-methylpyridin-2-amine instead of 5-bromoquinoline.

Mass (M+H⁺): 391.2

¹H-NMR (500 MHz, DMSO-d6): 0.65 (m, 2H), 0.90 (m, 2H), 2.59 (m, 1H), 3.11 (s, 3H), 3.66 (s, 3H), 3.91 (s, 3H), 5.39 (s, 2H), 7.05 (s, 1H), 7.10 (d, 1H), 7.46 (s, 1H), 7.57 (t, 3H), 7.88 (s, 1H), 8.09 (d, 1H).

### [Example 172] Synthesis of (S)-6,7-dimethoxy-9-(6-((5-methoxy-1,2,3,4-tetrahydronaphthalen-2-yl)amino)pyridin-3-yl)naphtho[2,3-c]furan-1(3H)-one

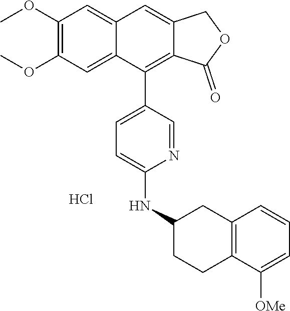

The compound of Example 172 was synthesized by the same method as that of the compound of Example 132 except for using (S)-5-bromo-N-(5-methoxy-1,2,3,4-tetrahydronaphthalen-2-yl)pyridin-2-amine instead of 5-bromoquinoline.

Mass (M+H⁺): 497.2

¹H-NMR (500 MHz, DMSO-d6): 2.69 (m, 2H), 2.85 (m, 2H), 3.75 (s, 3H), 3.83 (s, 3H), 3.93 (s, 3H), 4.20 (m, 1H), 5.44 (s, 2H), 6.78 (d, 2H), 7.10 (t, 2H) 7.32 (d, 2H), 7.56 (d, 1H), 8.03 (d, 2H)

### [Example 173] Synthesis of 6,7-dimethoxy-9-(5-(2-methoxyethoxy)pyridin-3-yl)naphtho[2,3-c]furan-1(3H)-one

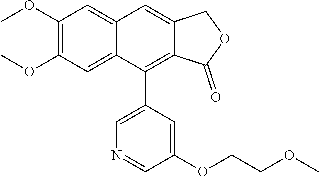

The compound of Example 173 was synthesized by the same method as that of the compound of Example 132 except for using 3-bromo-5-(2-methoxyethoxy)pyridine instead of 5-bromoquinoline.

Mass (M+H⁺): 396.1

¹H-NMR (500 MHz, DMSO-d6): 2.63 (s, 3H), 3.05-3.17 (m, 3H), 3.27 (s, 1H), 3.61 (s, 3H), 3.92 (s, 3H), 5.41 (s, 2H), 6.93 (s, 1H), 7.11 (s, 1H), 7.18-7.20 (m, 1H), 7.28-7.30 (m, 1H), 7.49 (s, 1H), 7.93 (s, 1H).

### [Example 174] Synthesis of 9-(6-(bis(2-methoxyethyl)amino)pyridin-3-yl)-6,7-dimethoxynaphtho[2,3-c]furan-1(3H)-one

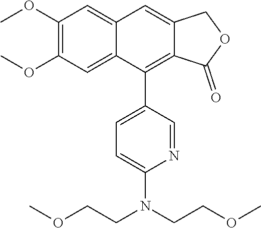

The compound of Example 174 was synthesized by the same method as that of the compound of Example 132 except for using 5-bromo-N,N-bis(2-methoxyethyl)pyridin-2-amine instead of 5-bromoquinoline.

Mass (M+H⁺): 453.2

¹H-NMR (500 MHz, DMSO-d6): 3.28 (s, 6H), 3.55 (m, 4H), 3.68 (t, 3H), 3.72 (m, 4H), 3.91 (s, 3H), 5.40 (s, 2H), 6.77 (d, 1H), 7.08 (s, 1H), 7.47 (s, 1H), 7.52 (d, 1H), 8.04 (s, 1H), 8.26 (d, 1H).

### [Example 175] Synthesis of 9-(6-(cyclohexyl(methyl)amino)pyridin-3-yl)-6,7-dimethoxynaphtho[2,3-c]furan-1(3H)-one

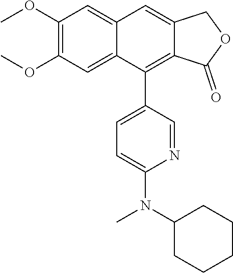 [SMILES: COc1cc2cc3c(c(-c4ccc(N(C)C5CCCCC5)nc4)c2cc1OC)C(=O)OC3]

The compound of Example 175 was synthesized by the same method as that of the compound of Example 132 except for using 5-bromo-N-cyclohexyl-N-methylpyridin-2-amine instead of 5-bromoquinoline.

Mass (M+H⁺): 432.51

¹H-NMR (500 MHz, DMSO-d6): 1.25 (m, 2H), 1.38 (m, 2H), 1.65 (m, 2H), 1.77 (m, 2H), 1.79 (m, 2H), 2.90 (s, 3H), 3.68 (s, 3H), 3.92 (s, 3H), 4.44 (m, 1H), 5.43 (s, 2H), 6.71 (d, 1H), 7.09 (s, 1H), 7.48 (s, 1H), 7.51 (d, 1H), 7.88 (s, 1H), 8.06 (d, 1H).

### [Example 176] Synthesis of 6,7-dimethoxy-9-(6-(methyl(2-methylbutyl)amino)pyridin-3-yl)naphtho[2,3-c]furan-1(3H)-one

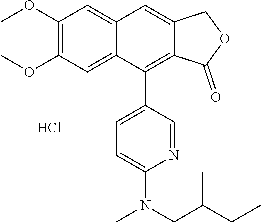 [SMILES: CCC(C)CN(C)c1ccc(-c2c3c(cc4cc(OC)c(OC)cc24)COC3=O)cn1 ; Cl]

The compound of Example 176 was synthesized by the same method as that of the compound of Example 132 except for using 5-bromo-N-methyl-N-(2-methylbutyl)pyridin-2-amine instead of 5-bromoquinoline.

Mass (M+H⁺): 421.2

¹H-NMR (500 MHz, DMSO-d6): 0.65 (m, 2H), 0.90 (m, 6H), 1.19 (m, 1H), 1.52 (m, 1H), 1.88 (m, 1H), 3.27 (s, 3H), 3.58 (m, 1H), 3.73 (s, 3H), 3.88 (s, 3H), 3.93 (s, 3H), 5.44 (s, 2H), 7.01 (s, 1H), 7.31 (d, 1H), 7.58 (s, 1H), 8.00 (m, 2H), 8.04 (d, 1H).

### [Example 177] Synthesis of 9-(6-(cyclopentyl(methyl)amino)pyridin-3-yl)-6,7-dimethoxynaphtho[2,3-c]furan-1(3H)-one

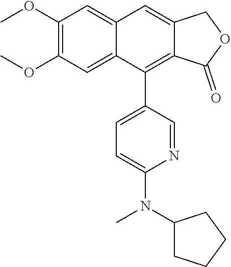 [SMILES: COc1cc2cc3c(c(-c4ccc(N(C)C5CCCC5)nc4)c2cc1OC)C(=O)OC3]

The compound of Example 177 was synthesized by the same method as that of the compound of Example 132 except for using 5-bromo-N-cyclopentyl-N-methylpyridin-2-amine instead of 5-bromoquinoline.

Mass (M+H⁺): 419.2

¹H-NMR (500 MHz, DMSO-d6): 1.58 (m, 4H), 1.70 (m, 2H), 1.81 (m, 2H), 2.90 (s, 3H), 3.69 (s, 3H), 3.92 (s, 3H), 5.06 (t, 1H), 5.40 (s, 2H), 6.73 (d, 1H), 7.10 (s, 1H), 7.48 (s, 1H), 7.52 (dd, 1H), 7.89 (s, 1H), 8.05 (d, 1H).

### [Example 178] Synthesis of 9-(6-(ethyl(methyl)amino)pyridin-3-yl)-6,7-dimethoxynaphtho[2,3-c]furan-1(3H)-one

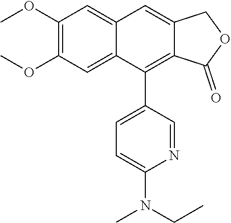 [SMILES: CCN(C)c1ccc(-c2c3c(cc4cc(OC)c(OC)cc24)COC3=O)cn1]

The compound of Example 178 was synthesized by the same method as that of the compound of Example 132 except for using 5-bromo-N-ethyl-N-methylpyridin-2-amine instead of 5-bromoquinoline.

Mass (M+H⁺): 379.2

¹H-NMR (500 MHz, DMSO-d6): 1.12 (t, 3H), 3.04 (s, 3H), 3.61 (q, 2H), 3.68 (s, 3H), 3.92 (s, 3H), 5.40 (s, 2H), 6.71 (d, 1H), 7.09 (s, 1H), 7.48 (s, 1H), 7.51 (dd, 1H), 7.89 (s, 1H), 8.05 (d, 1H).

### [Example 179] Synthesis of 6,7-dimethoxy-9-(6-(methyl((5-methylfuran-2-yl)methyl)amino)pyridin-3-yl)naphtho[2,3-c]furan-1(3H)-one

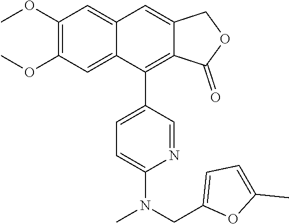

The compound of Example 179 was synthesized by the same method as that of the compound of Example 132 except for using 5-bromo-N-methyl-N-((5-methylfuran-2-yl)methyl)pyridin-2-amine instead of 5-bromoquinoline.

Mass (M+H⁺): 445.2

¹H-NMR (500 MHz, DMSO-d6): 2.20 (s, 3H), 3.07 (s, 3H), 3.67 (s, 3H), 3.92 (s, 3H), 4.74 (s, 3H), 5.41 (s, 2H), 5.96 (d, 1H), 6.17 (d, 1H), 6.83 (d, 1H), 7.06 (s, 1H), 7.49 (s, 1H), 7.55 (dd, 1H), 7.90 (s, 1H), 8.09 (d, 1H).

### [Example 180] Synthesis of 9-(6-(butyl(methyl)amino)pyridin-3-yl)-6,7-dimethoxynaphtho[2,3-c]furan-1(3H)-one

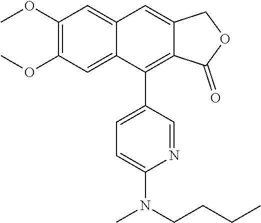 [SMILES: CCCCN(C)c1ccc(-c2c3c(cc4cc(OC)c(OC)cc24)COC3=O)cn1]

The compound of Example 180 was synthesized by the same method as that of the compound of Example 132 except for using 5-bromo-N-butyl-N-methylpyridin-2-amine instead of 5-bromoquinoline.

Mass (M+H⁺): 407.2

¹H-NMR (500 MHz, DMSO-d6): 0.91 (t, 3H), 1.31 (m, 2H), 1.56 (m, 2H), 3.05 (s, 3H), 3.55 (m, 2H), 3.68 (s, 3H), 3.92 (s, 3H), 5.40 (s, 2H), 6.69 (d, 1H), 7.08 (s, 1H), 7.48 (s, 1H), 7.50 (dd, 1H), 7.89 (s, 1H), 8.05 (d, 1H).

### [Example 181] Synthesis of 9-(6-(isobutyl(methyl)amino)pyridin-3-yl)-6,7-dimethoxynaphtho[2,3-c]furan-1(3H)-one

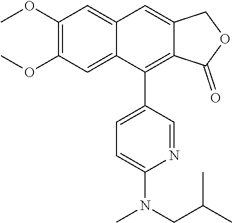 [SMILES: COc1cc2cc3c(c(-c4ccc(N(C)CC(C)C)nc4)c2cc1OC)C(=O)OC3]

The compound of Example 181 was synthesized by the same method as that of the compound of Example 132 except for using 5-bromo-N-isobutyl-N-methylpyridin-2-amine instead of 5-bromoquinoline.

Mass (M+H⁺): 407.2

¹H-NMR (500 MHz, DMSO-d6): 0.89 (d, 6H), 2.06 (m, 1H), 3.07 (s, 3H), 3.39 (m, 2H), 3.67 (s, 3H), 3.92 (s, 3H), 5.41 (s, 2H), 6.70 (d, 1H), 7.08 (s, 1H), 7.48 (s, 1H), 7.51 (dd, 1H), 7.89 (s, 1H), 8.05 (d, 1H).

### [Example 182] Synthesis of 9-(6-(ethyl(isopropyl)amino)pyridin-3-yl)-6,7-dimethoxynaphtho[2,3-c]furan-1(3H)-one

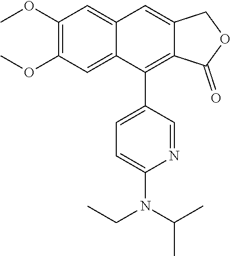 [SMILES: CCN(c1ccc(-c2c3c(cc4cc(OC)c(OC)cc24)COC3=O)cn1)C(C)C]

The compound of Example 182 was synthesized by the same method as that of the compound of Example 132 except for using 5-bromo-N-ethyl-N-isopropylpyridin-2-amine instead of 5-bromoquinoline.

Mass (M+H⁺): 407.2

¹H-NMR (500 MHz, DMSO-d6): 1.16 (t, 3H), 1.18 (d, 6H), 3.41 (q, 2H), 3.69 (s, 3H), 3.92 (s, 3H), 4.84 (m, 1H), 5.40 (s, 2H), 6.68 (d, 1H), 7.13 (s, 1H), 7.48 (s, 1H), 7.52 (dd, 1H), 7.88 (s, 1H), 8.07 (d, 1H).

### [Example 183] Synthesis of 9-(6-((cyclopropylmethyl)(methyl)amino)pyridin-3-yl)-6,7-dimethoxynaphtho[2,3-c]furan-1(3H)-one

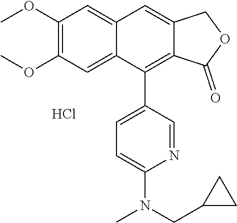 [SMILES: COc1cc2cc3c(c(-c4ccc(N(C)CC5CC5)nc4)c2cc1OC)C(=O)OC3 ; Cl]

The compound of Example 183 was synthesized by the same method as that of the compound of Example 132 except for using 5-bromo-N-(cyclopropylmethyl)-N-methylpyridin-2-amine instead of 5-bromoquinoline.

Mass (M+H⁺): 405.2

¹H-NMR (500 MHz, DMSO-d6): 1.22 (t, 3H), 3.72 (m, 5H), 3.74 (s, 3H), 3.94 (s, 3H), 5.45 (s, 2H), 7.03 (d, 1H), 7.30 (br, 1H), 7.55 (s, 1H), 8.01 (m, 2H), 8.05 (d, 1H).

### [Example 184] Synthesis of 9-(2-chloropyridin-4-yl)-6,7-dimethoxynaphtho[2,3-c]furan-1(3H)-one

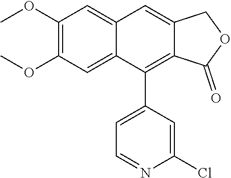 [SMILES: COc1cc2cc3c(c(-c4ccnc(Cl)c4)c2cc1OC)C(=O)OC3]

The compound of Example 184 was synthesized by the same method as that of the compound of Example 132 except for using 4-bromo-2-chloropyridine instead of 5-bromoquinoline.

Mass (M+H⁺): 356.0

¹H-NMR (500 MHz, DMSO-d6): 3.66 (s, 3H), 3.93 (s, 3H), 5.46 (s, 2H), 6.77 (s, 1H), 7.44-7.45 (m, 1H), 7.54 (s, 1H), 7.57 (s, 1H), 8.03 (s, 1H), 8.54 (d, J=5.15 Hz, 1H).

### [Example 185] Synthesis of 9-(6-chloropyridin-3-yl)-6,7-dimethoxynaphtho[2,3-c]furan-1(3H)-one

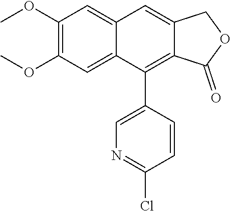 [SMILES: COc1cc2cc3c(c(-c4ccc(Cl)nc4)c2cc1OC)C(=O)OC3]

The compound of Example 185 was synthesized by the same method as that of the compound of Example 132 except for using 5-bromo-2-chloropyridine instead of 5-bromoquinoline.

Mass (M+H⁺): 356.0

¹H-NMR (500 MHz, DMSO-d6): 3.67 (s, 3H), 3.93 (s, 3H), 5.45 (s, 2H), 6.84 (s, 1H), 7.54 (s, 1H), 7.66 (dd, J=8.0 Hz, 0.55 Hz, 1H), 7.91 (dd, J=8.05 Hz, 2.6 Hz, 1H), 8.02 (s, 1H), 8.41 (t, J=0.85 Hz, 1H).

### [Example 186] Synthesis of 9-(2-fluoropyridin-4-yl)-6,7-dimethoxynaphtho[2,3-c]furan-1(3H)-one

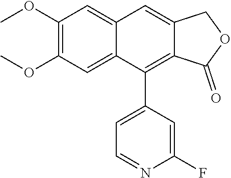 [SMILES: COc1cc2cc3c(c(-c4ccnc(F)c4)c2cc1OC)C(=O)OC3]

The compound of Example 186 was synthesized by the same method as that of the compound of Example 132 except for using 4-bromo-2-fluoropyridine instead of 5-bromoquinoline.

Mass (M+H⁺): 340.0

¹H-NMR (500 MHz, DMSO-d6): 3.65 (s, 3H), 3.93 (s, 3H), 5.46 (s, 2H), 6.78 (s, 1H), 7.26 (s, 1H), 7.36-7.37 (m, 1H), 7.54 (s, 1H), 8.03 (s, 1H), 8.37 (d, J=5.15 Hz, 1H).

### [Example 187] Synthesis of 9-(5-(2-(dimethylamino) ethoxy)pyridin-3-yl)-6,7-dimethoxynaphtho[2,3-c]furan-1(3H)-one

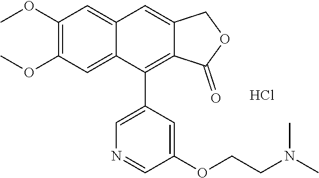

The compound of Example 187 was synthesized by the same method as that of the compound of Example 132 except for using 2-((5-bromopyridin-3-yl)oxy)-N,N-dimethylethan-1-amine instead of 5-bromoquinoline.

Mass (M+H⁺): 409.2

¹H-NMR (500 MHz, DMSO-d6): 2.81-2.82 (m, 6H), 3.51-3.53 (m, 2H), 3.68 (s, 3H), 3.93 (s, 3H), 4.48-4.51 (m, 2H), 5.47 (s, 2H), 6.90 (s, 1H), 7.56 (s, 1H), 7.71-7.75 (m, 1H), 8.04 (s, 1H), 8.34 (s, 1H), 8.56 (s, 1H).

### [Example 188] Synthesis of 9-(6-(isopropyl(methyl)amino)pyridin-3-yl)-6,7-dimethoxynaphtho[2,3-c]furan-1(3H)-one

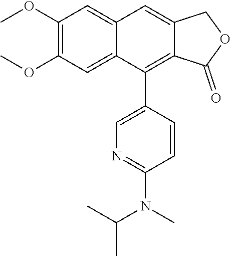 [SMILES: COc1cc2cc3c(c(-c4ccc(N(C)C(C)C)nc4)c2cc1OC)C(=O)OC3]

The compound of Example 188 was synthesized by the same method as that of the compound of Example 132 except for using 5-bromo-N-isopropyl-N-methylpyridin-2-amine instead of 5-bromoquinoline.

Mass (M+H⁺): 393.1

¹H-NMR (500 MHz, DMSO-d6): 1.15 (d, J=6.85 Hz, 6H), 2.86 (s, 3H), 3.68 (s, 3H), 3.92 (s, 3H), 4.89-4.93 (m, 1H), 5.40 (s, 2H), 6.71 (d, J=8.85 Hz, 1H), 7.09 (s, 1H), 7.47 (s, 1H), 7.51-7.53 (m, 1H), 7.88 (s, 1H), 8.05 (t, J=2.0 Hz, 1H).

### [Example 189] Synthesis of 9-(6-(3,4-dihydroisoquinolin-2 (1H)-yl)pyridin-3-yl)-6,7-dimethoxynaphtho[2,3-c]furan-1(3H)-one

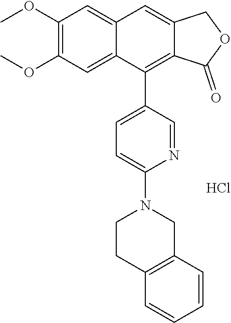 [SMILES: COc1cc2cc3c(c(-c4ccc(N5CCc6ccccc6C5)nc4)c2cc1OC)C(=O)OC3 ; Cl]

The compound of Example 189 was synthesized by the same method as that of the compound of Example 132 except for using 2-(5-bromopyridin-2-yl)-1,2,3,4-tetrahydroisoquinoline instead of 5-bromoquinoline.

Mass (M+H⁺): 453.1

¹H-NMR (500 MHz, DMSO-d6): 3.03 (t, J=5.7 Hz, 2H), 3.71 (s, 3H), 3.91-3.92 (m, 2H), 3.93 (s, 3H), 4.87 (s, 2H), 5.44 (s, 2H), 7.04 (s, 1H), 7.24-7.27 (m, 4H), 7.41 (br, 1H), 7.54 (s, 1H), 7.99-8.01 (m, 2H), 8.15 (d, J=2.0 Hz, 1H).

### [Example 190] Synthesis of 6,7-dimethoxy-9-(2-methyl-1,2,3,4-tetrahydroisoquinolin-7-yl)naphtho[2,3-c]furan-1(3H)-one

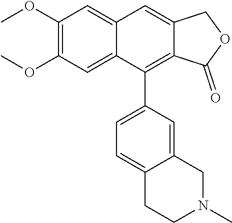

The compound of Example 190 was synthesized by the same method as that of the compound of Example 132 except for using 7-bromo-2-methyl-1,2,3,4-tetrahydroisoquinoline instead of 5-bromoquinoline.

Mass (M+H⁺): 390.2

¹H-NMR (500 MHz, DMSO-d6): 3.61-3.81 (m, 6H), 3.93 (s, 3H), 4.17-4.20 (m, 2H), 5.45 (s, 2H), 6.86 (s, 1H), 7.43-7.44 (m, 1H), 7.53 (s, 1H), 7.98 (s, 1H), 8.12 (d, J=7.45 Hz, 1H), 8.38 (d, J=2.9 Hz, 1H).

### [Example 191] Synthesis of 9-(6-(cyclopentyl(ethyl)amino)pyridin-3-yl)-6,7-dimethoxynaphtho[2,3-c]furan-1(3H)-one

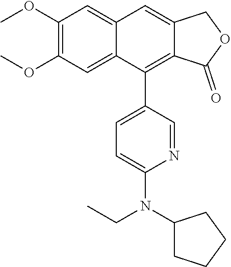 [SMILES: CCN(c1ccc(-c2c3c(cc4cc(OC)c(OC)cc24)COC3=O)cn1)C1CCCC1]

The compound of Example 191 was synthesized by the same method as that of the compound of Example 132 except for using 5-bromo-N-cyclopentyl-N-ethylpyridin-2-amine instead of 5-bromoquinoline.

Mass (M+H⁺): 433.1

¹H-NMR (500 MHZ, DMSO-d6): 1.18 (t, 3H), 1.57 (m, 4H), 1.71 (m, 2H), 1.87 (m, 2H), 3.45 (q, 2H), 3.70 (s, 3H), 3.92 (s, 3H), 4.80 (m, 1H), 5.40 (s, 2H), 6.73 (d, 1H), 7.12 (s, 1H), 7.48 (s, 1H), 7.52 (dd, 1H), 7.89 (s, 1H), 8.06 (d, 1H).

### [Example 192] Synthesis of 6,7-dimethoxy-9-(6-(methyl((tetrahydrofuran-2-yl)methyl)amino)pyridin-3-yl)naphtho[2,3-c]furan-1(3H)-one

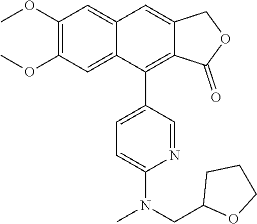 [SMILES: COc1cc2cc3c(c(-c4ccc(N(C)CC5CCCO5)nc4)c2cc1OC)C(=O)OC3]

The compound of Example 192 was synthesized by the same method as that of the compound of Example 132 except for using 5-bromo-N-methyl-N-((tetrahydrofuran-2-yl)methyl)pyridin-2-amine instead of 5-bromoquinoline.

Mass (M+H⁺): 435.1

¹H-NMR (500 MHZ, DMSO-d6): 1.55 (m, 1H), 1.79 (m, 1H), 1.86 (m, 1H), 1.94 (m, 1H), 3.11 (s, 3H), 3.51 (m, 1H), 3.62 (m, 1H), 3.68 (s, 3H), 3.78 (m, 2H), 3.92 (s, 3H), 4.10 (m, 1H), 5.40 (s, 2H), 6.73 (d, 1H), 7.08 (s, 1H), 7.48 (s, 1H), 7.52 (dd, 1H), 7.89 (s, 1H), 8.05 (d, 1H).

### [Example 193] Synthesis of 9-(6-(ethyl((tetrahydrofuran-2-yl)methyl)amino)pyridin-3-yl)-6,7-dimethoxynaphtho[2,3-c]furan-1(3H)-one

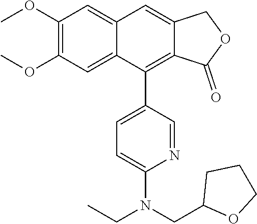 [SMILES: CCN(CC1CCCO1)c1ccc(-c2c3c(cc4cc(OC)c(OC)cc24)COC3=O)cn1]

The compound of Example 193 was synthesized by the same method as that of the compound of Example 132 except for using 5-bromo-N-ethyl-N-((tetrahydrofuran-2-yl)methyl)pyridin-2-amine instead of 5-bromoquinoline.

Mass (M+H⁺): 449.1

¹H-NMR (500 MHz, DMSO-d6): 1.14 (t, 3H), 1.55 (m, 1H), 1.79 (m, 1H), 1.86 (m, 1H), 1.94 (m, 1H), 3.43 (m, 1H), 3.63 (m, 3H), 3.69 (s, 3H), 3.78 (m, 2H), 3.92 (s, 3H), 4.10 (m, 1H), 5.40 (s, 2H), 6.72 (d, 1H), 7.10 (s, 1H), 7.48 (s, 1H), 7.52 (dd, 1H), 7.89 (s, 1H), 8.05 (d, 1H).

### [Example 194] Synthesis of 9-(6-((2,3-dihydro-1H-indan-2-yl)(methyl)amino)pyridin-3-yl)-6,7-dimethoxynaphtho[2,3-c]furan-1(3H)-one

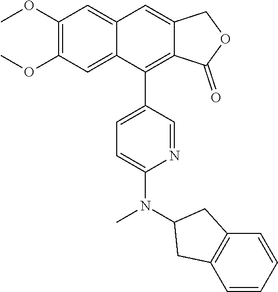 [SMILES: COc1cc2cc3c(c(-c4ccc(N(C)C5Cc6ccccc6C5)nc4)c2cc1OC)C(=O)OC3]

The compound of Example 194 was synthesized by the same method as that of the compound of Example 132 except for using 5-bromo-N-(2,3-dihydro-1H-indan-2-yl)-N-methylpyridin-2-amine instead of 5-bromoquinoline.

Mass (M+H⁺): 467.2

¹H-NMR (500 MHz, DMSO-d6): 2.90 (s, 3H), 3.07 (m, 2H), 3.14 (m, 2H), 3.69 (s, 3H), 3.93 (s, 3H), 5.45 (s, 2H), 6.56 (m, 1H), 6.83 (d, 1H), 7.10 (s, 1H), 7.14 (d, 1H), 7.25 (d, 2H), 7.48 (s, 1H), 7.57 (q, 1H), 7.89 (s, 1H), 8.09 (d, 1H).

### [Example 195] Synthesis of 9-(6-((2,3-dihydro-1H-indan-2-yl)(ethyl)amino)pyridin-3-yl)-6,7-dimethoxynaphtho[2,3-c]furan-1(3H)-one

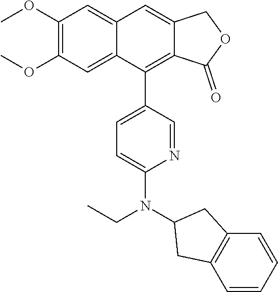 [SMILES: CCN(c1ccc(-c2c3c(cc4cc(OC)c(OC)cc24)COC3=O)cn1)C1Cc2ccccc2C1]

The compound of Example 195 was synthesized by the same method as that of the compound of Example 132 except for using 5-bromo-N-(2,3-dihydro-1H-indan-2-yl)-N-ethylpyridin-2-amine instead of 5-bromoquinoline.

Mass (M+H⁺): 481.2

¹H-NMR (500 MHz, DMSO-d6): 1.16 (t, 3H), 3.07 (m, 2H), 3.15 (m, 2H), 3.27 (s, 3H), 3.52 (m, 2H), 3.69 (s, 3H), 3.92 (s, 3H), 5.38 (s, 2H), 5.42 (m, 1H), 6.78 (d, 1H), 7.14 (m, 3H), 7.25 (d, 2H), 7.48 (s, 1H), 7.57 (q, 1H), 7.89 (s, 1H), 8.09 (d, 1H).

### [Example 196] Synthesis of 6,7-dimethoxy-9-(6-(methyl(2-(1-methyl-1H-indol-3-yl)ethyl)amino)pyridin-3-yl)naphtho[2,3-c]furan-1(3H)-one

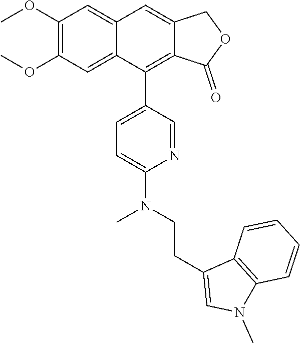 [SMILES: COc1cc2cc3c(c(-c4ccc(N(C)CCc5cn(C)c6ccccc56)nc4)c2cc1OC)C(=O)OC3]

The compound of Example 196 was synthesized by the same method as that of the compound of Example 132 except for using 5-bromo-N-methyl-N-(2-(1-methyl-1H-indol-3-yl)ethyl)pyridin-2-amine instead of 5-bromoquinoline.

Mass (M+H⁺): 508.2

¹H-NMR (500 MHz, DMSO-d6): 2.98 (m, 2H), 3.08 (s, 3H), 3.70 (d, 6H), 3.71 (m, 2H), 3.92 (s, 3H), 5.41 (s, 2H), 6.75 (d, 1H), 6.99 (m, 1H), 7.08 (d, 1H), 7.11 (m, 2H), 7.17 (s, 1H), 7.36 (d, 1H), 7.48 (s, 1H), 7.61 (d, 1H), 7.89 (s, 1H), 8.12 (s, 1H).

### [Example 197] Synthesis of 9-(6-(ethyl(2-(1-ethyl-1H-indol-3-yl)ethyl)amino)pyridin-3-yl)-6,7-dimethoxynaphtho[2,3-c]furan-1(3H)-one

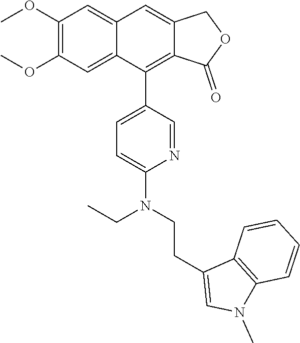 [SMILES: CCN(CCc1cn(C)c2ccccc12)c1ccc(-c2c3c(cc4cc(OC)c(OC)cc24)COC3=O)cn1]

The compound of Example 197 was synthesized by the same method as that of the compound of Example 132 except for using 5-bromo-N-ethyl-N-(2-(1-ethyl-1H-indol-3-yl)ethyl)pyrimidin-2-amine instead of 5-bromoquinoline.

Mass (M+H⁺): 536.3

¹H-NMR (500 MHz, DMSO-d6): 3.00 (m, 5H), 3.09 (m, 4H), 3.67 (d, 6H), 3.77 (m, 2H), 3.92 (s, 3H), 5.72 (s, 2H), 6.74 (d, 2H), 7.11 (d, 2H), 7.48 (s, 1H), 7.72 (d, 2H), 7.85 (d, 1H), 7.89 (s, 1H), 8.06 (s, 1H), 8.35 (d, 1H).

### [Example 198] Synthesis of 6,7-dimethoxy-9-(6-(methyl(thiophen-2-ylmethyl)amino)pyridin-3-yl)naphtho[2,3-c]furan-1(3H)-one

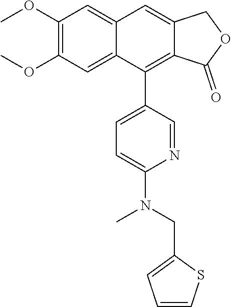 [SMILES: COc1cc2cc3c(c(-c4ccc(N(C)Cc5cccs5)nc4)c2cc1OC)C(=O)OC3]

The compound of Example 198 was synthesized by the same method as that of the compound of Example 132 except for using 5-bromo-N-methyl-N-(thiophen-2-ylmethyl)pyridin-2-amine instead of 5-bromoquinoline.

Mass (M+H⁺): 447.2

¹H-NMR (500 MHz, DMSO-d6): 3.05 (s, 3H), 3.68 (s, 1H), 3.92 (s, 3H), 5.05 (q, 2H), 5.43 (s, 2H), 6.81 (d, 1H), 6.95 (q, 1H), 7.06 (s, 2H), 7.34 (d, 1H), 7.48 (s, 1H), 7.60 (d, 1H), 7.90 (s, 1H), 8.13 (d, 1H).

### [Example 199] Synthesis of 9-(6-(ethyl(thiophen-2-ylmethyl)amino)pyridin-3-yl)-6,7-dimethoxynaphtho[2,3-c]furan-1(3H)-one

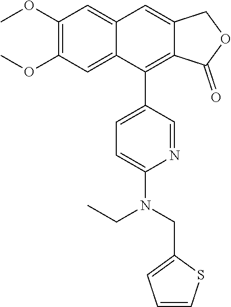 [SMILES: CCN(Cc1cccs1)c1ccc(-c2c3c(cc4cc(OC)c(OC)cc24)COC3=O)cn1]

The compound of Example 199 was synthesized by the same method as that of the compound of Example 132 except for using 5-bromo-N-ethyl-N-(thiophen-2-ylmethyl)pyridin-2-amine instead of 5-bromoquinoline.

Mass (M+H⁺): 461.0

¹H-NMR (500 MHz, DMSO-d6): 1.15 (t, 3H), 3.57 (m, 2H), 3.68 (s, 3H), 3.92 (s, 3H), 4.99 (q, 2H), 5.41 (s, 2H), 6.79 (d, 1H), 6.94 (q, 1H), 7.08 (s, 2H), 7.34 (d, 1H), 7.48 (s, 1H), 7.60 (d, 1H), 7.89 (s, 1H), 8.12 (d, 1H).

### [Example 200] Synthesis of 9-(5-isopropoxypyridin-3-yl)-6,7-dimethoxynaphtho[2,3-c]furan-1(3H)-one

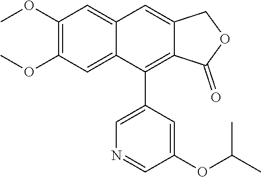 [SMILES: COc1cc2cc3c(c(-c4cncc(OC(C)C)c4)c2cc1OC)C(=O)OC3]

The compound of Example 200 was synthesized by the same method as that of the compound of Example 132 except for using 3-bromo-5-isopropoxypyridine instead of 5-bromoquinoline.

Mass (M+H⁺): 380.1

¹H-NMR (500 MHz, DMSO-d6): 1.31 (dd, J=6.0 Hz, 2.0 Hz, 6H), 3.68 (s, 3H), 3.93 (s, 3H), 4.82-4.85 (m, 1H), 5.47 (s, 2H), 6.91 (s, 1H), 7.56 (s, 1H), 7.95-7.96 (br, 1H), 8.05 (s, 1H), 8.42 (br, 1H), 8.60 (br, 1H).

### [Example 201] Synthesis of 9-(6-chloro-5-(dimethylamino)pyridin-3-yl)-6,7-dimethoxynaphtho[2,3-c]furan-1(3H)-one

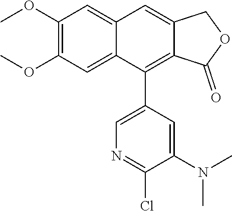

The compound of Example 201 was synthesized by the same method as that of the compound of Example 132 except for using 5-bromo-2-chloro-N,N-dimethylpyridin-3-amine instead of 5-bromoquinoline.

Mass (M+H⁺): 399.1

¹H-NMR (500 MHz, DMSO-d6): 2.77 (s, 6H), 3.68 (s, 3H), 3.93 (s, 3H), 5.44 (s, 2H), 6.93 (s, 1H), 7.53 (s, 1H), 7.58 (d, J=2.0 Hz, 1H), 8.00-8.01 (m, 2H).

### [Example 202] Synthesis of 6,7-dimethoxy-9-(6-(3-(m-tolyl) piperidin-1-yl)pyridin-3-yl)naphtho[2,3-c]furan-1(3H)-one

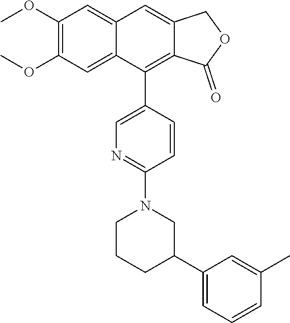

The compound of Example 202 was synthesized by the same method as that of the compound of Example 132 except for using 5-bromo-2-(3-(m-tolyl) piperidin-1-yl)pyridine instead of 5-bromoquinoline.

Mass (M+H⁺): 395.3

¹H-NMR (500 MHz, DMSO-d6): 1.61-1.93 (m, 5H), 2.28 (s, 3H), 2.71-2.74 (m, 1H), 2.87-2.93 (m, 2H), 3.68 (s, 3H), 3.92 (s, 3H), 4.40-4.51 (m, 1H), 5.40 (s, 2H), 6.96 (d, J=8.55 Hz, 1H), 7.02 (d, J=7.45 Hz, 1H), 7.07 (s, 1H), 7.10-7.134 (m, 2H), 7.19 (t, J=7.45 Hz, 1H), 7.48 (s, 1H), 7.54 (dd, J=8.9 Hz, 0.23 Hz, 1H), 7.89 (s, 1H), 8.08 (d, J=2.0 Hz, 1H).

### [Example 203] Synthesis of 6,7-dimethoxy-9-(3-(morpholinomethyl)-1H-indol-6-yl)naphtho[2,3-c]furan-1(3H)-one

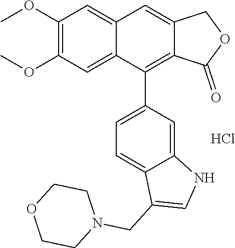 [SMILES: COc1cc2cc3c(c(-c4ccc5c(CN6CCOCC6)c[nH]c5c4)c2cc1OC)C(=O)OC3 ; Cl]

The compound of Example 203 was synthesized by the same method as that of the compound of Example 132 except for using 4-((6-bromo-1H-indol-3-yl)methyl)morpholine instead of 5-bromoquinoline.

Mass (M+H⁺): 459.3

¹H-NMR (500 MHz, DMSO-d6): 3.13-3.17 (m, 2H), 3.38-3.41 (m, 2H), 3.53 (s, 3H), 3.69 (t, J=12.2 Hz, 2H), 3.92-3.97 (m, 5H), 4.51-4.55 (m, 2H), 5.43 (s, 2H), 6.89 (s, 1H), 7.08 (d, J=8.25 Hz, 1H), 7.42 (s, 1H), 7.50 (s, 1H), 7.69 (s, 1H), 7.90 (d, J=7.95 Hz, 1H), 7.93 (s, 1H), 10.35 (br, 1H), 11.60 (s, 1H).

### [Example 204] Synthesis of 9-(5-(1H-benzo[d]imidazol-2-yl)-6-methoxypyridin-3-yl)-6,7-dimethoxynaphtho[2,3-c]furan-1(3H)-one

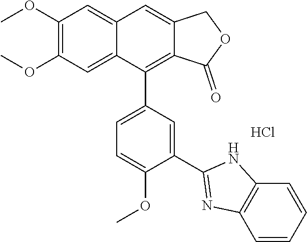 [SMILES: COc1cc2cc3c(c(-c4ccc(OC)c(-c5nc6ccccc6[nH]5)c4)c2cc1OC)C(=O)OC3 ; Cl]

The compound of Example 204 was synthesized by the same method as that of the compound of Example 132 except for using 2-(5-bromo-2-methoxypyridin-3-yl)-1H-benzo[d]imidazole instead of 5-bromoquinoline.

Mass (M+H⁺): 468.1

¹H-NMR (500 MHz, DMSO-d6): 3.69 (s, 3H), 3.95 (s, 3H), 4.25 (s, 3H), 5.49 (s, 2H), 7.01 (s, 1H), 7.45-7.47 (m, 2H), 7.58 (s, 1H), 7.77-7.79 (m, 2H), 8.05 (s, 1H), 8.51 (s, 1H), 8.68 (d, J=2.3 Hz, 1H)

### [Example 205] Synthesis of 9-(2-(4-(dimethylamino) piperidin-1-yl)pyrimidin-5-yl)-6,7-dimethoxynaphtho[2,3-c]furan-1(3H)-one

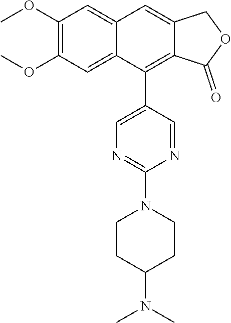 [SMILES: COc1cc2cc3c(c(-c4cnc(N5CCC(N(C)C)CC5)nc4)c2cc1OC)C(=O)OC3]

The compound of Example 205 was synthesized by the same method as that of the compound of Example 132 except for using 1-(5-bromopyrimidin-2-yl)-N,N-dimethylpiperidin-4-amine instead of 5-bromoquinoline.

Mass (M+H⁺): 449.2

¹H-NMR (500 MHz, DMSO-d6): 1.35-1.37 (m, 2H), 1.83-1.86 (m, 2H), 2.18 (s, 6H), 2.32-2.42 (m, 1H), 2.94-2.99 (m, 2H), 3.73 (s, 3H), 3.92 (s, 3H), 4.72 (d, J=13.45 Hz, 2H), 5.42 (s, 2H), 7.08 (s, 1H), 7.50 (s, 1H), 7.93 (s, 1H), 8.37 (s, 2H).

### [Example 206] Synthesis of 6,7-dimethoxy-9-(6-(methyl(1-methyl-1H-1,2,4-triazol-3-yl)amino)pyridin-3-yl)naphtho[2,3-c]furan-1(3H)-one

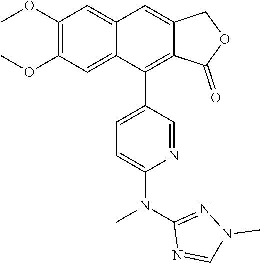

The compound of Example 206 was synthesized by the same method as that of the compound of Example 132 except for using 5-bromo-N-methyl-N-(1-methyl-1H-1,2,4-triazol-3-yl)pyridin-2-amine instead of 5-bromoquinoline.

Mass (M+H⁺): 432.1

¹H-NMR (500 MHz, DMSO-d6): 3.00 (s, 6H), 3.67 (s, 3H), 3.94 (s, 3H), 5.46 (s, 2H), 6.94 (s, 1H), 7.55 (s, 1H), 7.82 (d, 1H), 8.02 (s, 1H), 8.04 (d, 1H), 8.44 (s, 1H), 9.09 (s, 1H).

### [Example 207] Synthesis of 9-(6-(ethyl(1-ethyl-1H-1,2,4-triazol-3-yl)amino)pyridin-3-yl)-6,7-dimethoxynaphtho[2,3-c]furan-1(3H)-one

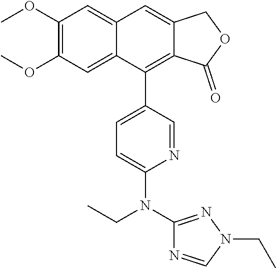 [SMILES: CCN(c1ccc(-c2c3c(cc4cc(OC)c(OC)cc24)COC3=O)cn1)c1ncn(CC)n1]

The compound of Example 207 was synthesized by the same method as that of the compound of Example 132 except for using 5-bromo-N-ethyl-N-(1-ethyl-1H-1,2,4-triazol-3-yl)pyridin-2-amine instead of 5-bromoquinoline.

Mass (M+H⁺): 460.1

¹H-NMR (500 MHz, DMSO-d6): 1.14 (t, 6H), 3.46 (q, 4H), 3.67 (s, 3H), 3.94 (s, 3H), 5.46 (s, 2H), 6.94 (s, 1H), 7.55 (s, 1H), 7.80 (d, 1H), 8.00 (s, 1H), 8.01 (d, 1H), 8.43 (s, 1H), 9.06 (s, 1H).

### [Example 208] Synthesis of 6,7-dimethoxy-9-(6-(methyl(2-(trifluoromethyl)benzyl)amino)pyridin-3-yl)naphtho[2,3-c]furan-1(3H)-one

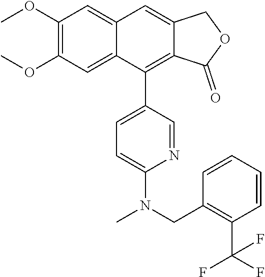 [SMILES: COc1cc2cc3c(c(-c4ccc(N(C)Cc5ccccc5C(F)(F)F)nc4)c2cc1OC)C(=O)OC3]

The compound of Example 208 was synthesized by the same method as that of the compound of Example 132 except for using 5-bromo-N-methyl-N-(2-(trifluoromethyl)benzyl)pyridin-2-amine instead of 5-bromoquinoline.

Mass (M+H⁺): 509.2

¹H-NMR (500 MHz, DMSO-d6): 3.15 (s, 3H), 3.67 (s, 3H), 3.92 (s, 3H), 5.06 (s, 2H), 5.41 (s, 2H), 6.78 (d, 1H), 7.04 (s, 1H), 7.27 (d, 1H), 7.46 (m, 2H), 7.60 (m, 2H), 7.74 (d, 1H), 7.90 (s, 1H), 8.06 (d, 1H).

### [Example 209] Synthesis of 9-(6-(ethyl(2-(trifluoromethyl)benzyl)amino)pyridin-3-yl)-6,7-dimethoxynaphtho[2,3-c]furan-1(3H)-one

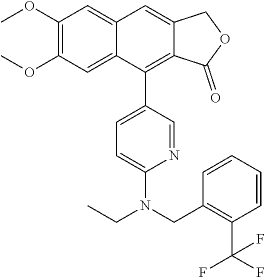 [SMILES: CCN(Cc1ccccc1C(F)(F)F)c1ccc(-c2c3c(cc4cc(OC)c(OC)cc24)COC3=O)cn1]

The compound of Example 209 was synthesized by the same method as that of the compound of Example 132 except for using 5-bromo-N-ethyl-N-(2-(trifluoromethyl)benzyl)pyridin-2-amine instead of 5-bromoquinoline.

Mass (M+H⁺): 523.1

¹H-NMR (500 MHZ, DMSO-d6): 1.19 (t, 3H), 3.65 (q, 4H), 3.67 (s, 3H), 3.92 (s, 3H), 4.99 (s, 2H), 5.41 (s, 2H), 6.73 (d, 1H), 7.05 (s, 1H), 7.32 (d, 1H), 7.46 (m, 2H), 7.58 (m, 2H), 7.74 (d, 1H), 7.89 (s, 1H), 8.06 (d, 1H).

### [Example 210] Synthesis of 6,7-dimethoxy-9-(4-(2,2,2-trifluoroacetyl)phenyl)naphtho[2,3-c]furan-1(3H)-one

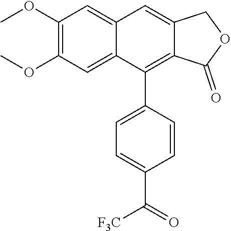 [SMILES: COc1cc2cc3c(c(-c4ccc(C(=O)C(F)(F)F)cc4)c2cc1OC)C(=O)OC3]

The compound of Example 210 was synthesized by the same method as that of the compound of Example 132 except for using 1-(4-bromophenyl)-2,2,2-trifluoroethan-1-one instead of 5-bromoquinoline.

Mass (M+H⁺): 417.1

¹H-NMR (500 MHz, DMSO-d6): 3.60 (s, 2H), 3.93 (s, 3H), 5.46 (s, 2H), 7.64 (s, 1H), 7.68 (d, 2H), 8.01 (s, 1H), 8.17 (d, 2H).

### [Example 211] Synthesis of (S)-4-benzyl-3-(5-(6,7-dimethoxy-3-oxo-1,3-dihydronaphtho[2,3-c]furan-4-yl)pyridin-2-yl) oxazolidin-2-one

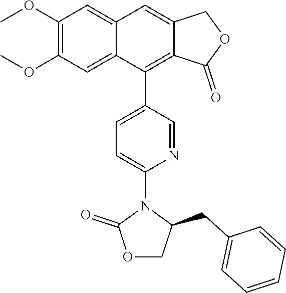 [SMILES: COc1cc2cc3c(c(-c4ccc(N5C(=O)OC[C@@H]5Cc5ccccc5)nc4)c2cc1OC)C(=O)OC3]

The compound of Example 211 was synthesized by the same method as that of the compound of Example 132 except for using (S)-4-benzyl-3-(5-bromopyridin-2-yl) oxazolidin-2-one instead of 5-bromoquinoline.

Mass (M+H⁺): 407.2

1H-NMR (500 MHz, DMSO-d6): 3.20 (m, 2H), 3.68 (s, 3H), 4.00 (s, 3H), 4.30 (m, 1H), 4.48 (m, 1H), 5.15 (m, 1H), 5.45 (s, 2H), 6.96 (d, 1H), 7.26 (m, 5H), 7.53 (s, 1H), 7.93 (d, 1H), 7.99 (s, 1H), 8.19 (d, 1H), 8.47 (d, 1H).

### [Example 212] Synthesis of 1-(6-hydroxybenzo[d][1,3]dioxol-5-yl)-3-(hydroxymethyl)-6,7-dimethoxy-2-naphthoate sodium (I)

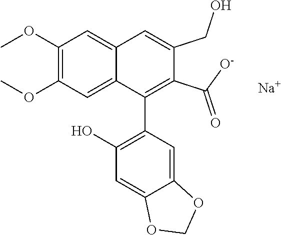 [SMILES: COc1cc2cc(CO)c(C(=O)[O-])c(-c3cc4c(cc3O)OCO4)c2cc1OC ; [Na+]]

After 2 g (5.26 mmol) of 9-(6-hydroxybenzo[d][1,3]dioxol-5-yl)-6,7-dimethoxynaphtho[2,3-c]furan-1(3H)-one and 20 ml of 2N sodium hydroxide were injected into dichloromethane, the resulting mixture was stirred at 40° C. for two hours and separated in layers. After 20 ml of ethyl acetate was injected into an aqueous layer of the reactant, the resulting mixture was stirred at 40° C. for two hours to precipitate a solid, which was then filtered, washed and dried under reduced pressure to obtain 1.8 g (4.28 mmol, 81%) of 1-(6-hydroxybenzo[d][1,3]dioxol-5-yl)-3-(hydroxymethyl)-6,7-dimethoxy-2-naphthoate sodium (1) as a title compound.

Mass (M+H⁺): 421.2

¹H-NMR (500 MHz, DMSO-d6): 3.54 (s, 3H), 3.86 (s, 3H), 4.37 (d, 1H), 4.67 (d, 1H), 4.48 (m, 1H), 5.46 (br, 1H), 5.89 (s, 1H), 5.97 (s, 1H), 6.52 (d, 2H), 6.56 (s, 1H), 7.23 (s, 1H), 7.65 (s, 1H), 11.48 (s, 1H).

### [Example 213] Synthesis of 9-(2-((3,4-dimethylphenyl)(methyl)amino)pyrimidin-5-yl)-6,7-dimethoxynaphtho[2,3-c]furan-1(3H)-one

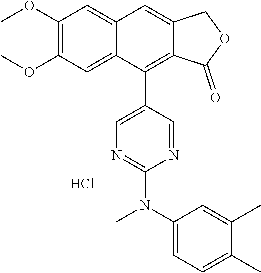

The compound of Example 213 was synthesized by the same method as that of the compound of Example 132 except for using 5-bromo-N-(3,4-dimethylphenyl)-N-methylpyrimidin-2-amine instead of 5-bromoquinoline.

Mass (M+H⁺): 456.1

¹H-NMR (500 MHz, DMSO-d6): 2.21 (d, J=2.85 Hz, 6H), 3.50 (s, 3H), 3.74 (s, 3H), 3.92 (s, 3H), 5.42 (s, 2H), 7.07 (s, 1H), 7.10-7.13 (m, 1H), 7.15-7.17 (m, 2H), 7.50 (s, 1H), 7.94 (s, 1H), 8.41 (s, 2H).

### [Example 214] Synthesis of 6,7-dimethoxy-9-(6-(methyl(5-methyl-1,3,4-thiadiazol-2-yl)amino)pyridin-3-yl)naphtho[2,3-c]furan-1(3H)-one

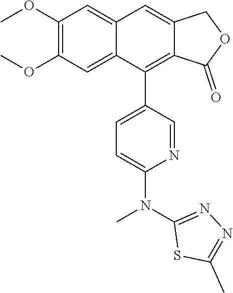 [SMILES: COc1cc2cc3c(c(-c4ccc(N(C)c5nnc(C)s5)nc4)c2cc1OC)C(=O)OC3]

The compound of Example 214 was synthesized by the same method as that of the compound of Example 132 except for using N-(5-bromopyridin-2-yl)-N,5-dimethyl-1,3,4-thiadiazol-2-amine instead of 5-bromoquinoline.

Mass (M+H⁺): 449.1

¹H-NMR (500 MHz, DMSO-d6): 2.42 (s, 3H), 3.64 (s, 3H), 3.79 (s, 3H), 3.93 (s, 3H), 5.44 (s, 2H), 6.97 (s, 1H), 7.16 (dd, J=8.3, 0.55 Hz, 1H), 7.51 (s, 1H), 7.72 (dd, J=8.3, 2.25 Hz, 1H), 7.95 (s, 1H), 8.32 (t, J=2.3 Hz, 1H).

### [Example 215] Synthesis of 6,7-dimethoxy-9-(6-(methyl(1-methyl-5-(methylthio)-1H-1,2,4-triazol-3-yl)amino)pyridin-3-yl)naphtho[2,3-c]furan-1(3H)-one

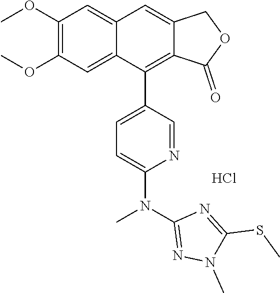

The compound of Example 215 was synthesized by the same method as that of the compound of Example 132 except for using 5-bromo-N-methyl-N-(1-methyl-5-(methylthio)-1H-1,2,4-triazol-3-yl)pyridin-2-amine instead of 5-bromoquinoline.

Mass (M+H⁺): 478.1

¹H-NMR (500 MHz, DMSO-d6): 2.65 (s, 3H), 3.60 (s, 3H), 3.67 (s, 3H), 3.70 (s, 3H), 3.93 (s, 3H), 5.43 (s, 2H), 7.00 (s, 1H), 7.51 (s, 1H), 7.73-7.75 (m, 1H), 7.96 (s, 1H), 8.04-8.05 (m, 1H), 8.27-8.28 (m, 1H).

### [Example 216] Synthesis of 6,7-dimethoxy-9-(2-(methyl(3-(trifluoromethyl)phenyl)amino)pyrimidin-5-yl)naphtho[2,3-c]furan-1(3H)-one

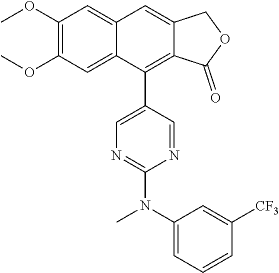 [SMILES: COc1cc2cc3c(c(-c4cnc(N(C)c5cccc(C(F)(F)F)c5)nc4)c2cc1OC)C(=O)OC3]

The compound of Example 216 was synthesized by the same method as that of the compound of Example 132 except for using 5-bromo-N-methyl-N-(3-(trifluoromethyl)phenyl)pyrimidin-2-amine instead of 5-bromoquinoline.

Mass (M+H⁺): 495.2

¹H-NMR (500 MHz, DMSO-d6): 3.51 (s, 3H), 3.69 (s, 3H), 3.92 (s, 3H), 5.42 (s, 2H), 6.84 (dd, J=8.6 Hz, 0.6 Hz, 1H), 7.05 (s, 1H), 7.49 (s, 1H), 7.51-7.53 (m, 1H), 7.59 (dd, J=8.6 Hz, 2.3 Hz, 1H), 7.64 (t, J=7.75 Hz, 1H), 7.69-7.71 (m, 2H), 7.93 (s, 1H), 8.17 (dd, J=2.3 Hz, 0.85 Hz, 1H).

### [Example 217] Synthesis of 9-(4-(dimethylamino)-3-nitrophenyl)-6,7-dimethoxynaphtho[2,3-c]furan-1(3H)-one

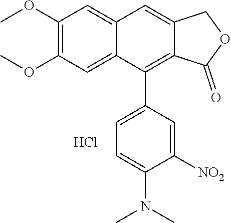 [SMILES: COc1cc2cc3c(c(-c4ccc(N(C)C)c([N+](=O)[O-])c4)c2cc1OC)C(=O)OC3 ; Cl]

The compound of Example 217 was synthesized by the same method as that of the compound of Example 132 except for using 4-bromo-N,N-dimethyl-2-nitroaniline instead of 5-bromoquinoline.

Mass (M+H⁺): 409.1

¹H-NMR (500 MHz, DMSO-d6): 2.90 (s, 6H), 3.67 (s, 3H), 3.92 (s, 3H), 5.41 (s, 2H), 7.01 (s, 1H), 7.26 (d, J=8.6 Hz, 1H), 7.50 (s, 1H), 7.52 (d, J=2.3 Hz, 1H), 7.77 (d, J=2.3 Hz, 1H), 7.93 (s, 1H).

### [Example 218] Synthesis of 4-((5-(6,7-dimethoxy-3-oxo-1,3-dihydronaphtho[2,3-c]furan-4-yl)pyridin-2-yl)(methyl)amino)benzonitrile

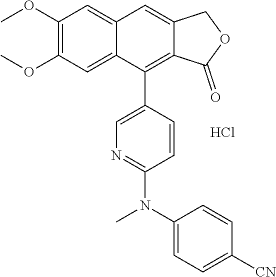 [SMILES: COc1cc2cc3c(c(-c4ccc(N(C)c5ccc(C#N)cc5)nc4)c2cc1OC)C(=O)OC3 ; Cl]

The compound of Example 218 was synthesized by the same method as that of the compound of Example 132 except for using 4-((5-bromopyridin-2-yl)(methyl)amino)benzonitrile instead of 5-bromoquinoline.

Mass (M+H⁺): 452.2

¹H-NMR (500 MHz, DMSO-d6): 3.57 (s, 3H), 3.70 (s, 3H), 3.93 (s, 3H), 5.43 (s, 2H), 7.01 (s, 1H), 7.14 (dd, J=8.3 Hz, 0.55 Hz, 1H), 7.52 (s, 1H), 7.54 (s, 1H), 7.56 (s, 1H), 7.76 (dd, J=8.9 Hz, 2.6 Hz, 1H), 7.83 (s, 1H), 7.85 (s, 1H), 7.97 (s, 1H), 8.24 (dd, J=1.7 Hz, 0.85 Hz, 1H).

### [Example 219] Synthesis of 6,7-dimethoxy-9-(6-morpholinopyridazin-3-yl)naphtho[2,3-c]furan-1(3H)-one

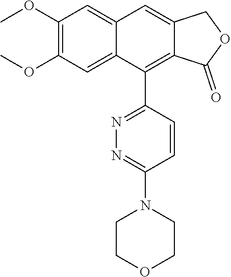 [SMILES: COc1cc2cc3c(c(-c4ccc(N5CCOCC5)nn4)c2cc1OC)C(=O)OC3]

The compound of Example 219 was synthesized by the same method as that of the compound of Example 132 except for using 4-(6-bromopyridazin-3-yl)morpholine instead of 5-bromoquinoline.

Mass (M+H⁺): 408.1

¹H-NMR (500 MHz, DMSO-d6): 3.60-3.61 (m, 2H), 3.64-3.66 (m, 5H), 3.71-3.72 (m, 2H), 3.75-3.77 (m, 2H), 3.93 (s, 3H), 5.47 (s, 2H), 6.99 (s, 1H), 7.37 (s, 1H), 7.52 (s, 1H), 7.54 (d, J=9.45 Hz, 1H), 8.01 (s, 1H).

### [Example 220] Synthesis of 6,7-dimethoxy-9-(6-morpholinoimidazo[1,2-b]pyridazin-3-yl)naphtho[2,3-c]furan-1(3H)-one

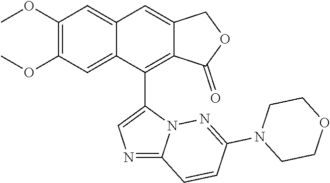 [SMILES: COc1cc2cc3c(c(-c4cnc5ccc(N6CCOCC6)nn45)c2cc1OC)C(=O)OC3]

The compound of Example 220 was synthesized by the same method as that of the compound of Example 132 except for using 4-(3-bromoimidazo[1,2-b]pyridazin-6-yl)morpholine instead of 5-bromoquinoline.

Mass (M+H⁺): 477.2

¹H-NMR (500 MHz, DMSO-d6): 3.12-3.14 (m, 4H), 3.56 (t, J=4.6 Hz, 4H), 3.67 (s, 3H), 3.93 (s, 3H), 5.46 (q, J=14.6 Hz, 2H), 7.18 (s, 1H), 7.19 (d, J=10.05 Hz, 1H), 7.52 (s, 1H), 7.83 (s, 1H), 7.97 (d, J=10.0 Hz, 1H), 8.01 (s, 1H).

### [Example 221] Synthesis of 9-(2,2-difluorobenzo[d][1,3]dioxol-5-yl)-6,7-dimethoxynaphtho[2,3-c]furan-1(3H)-one

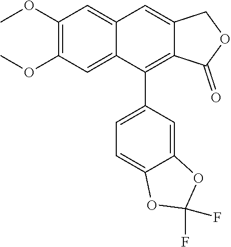 [SMILES: COc1cc2cc3c(c(-c4ccc5c(c4)OC(F)(F)O5)c2cc1OC)C(=O)OC3]

The compound of Example 221 was synthesized by the same method as that of the compound of Example 132 except for using 5-bromo-2,2-difluorobenzo[d][1,3]dioxole instead of 5-bromoquinoline.

Mass (M+H⁺): 401.0

¹H-NMR (500 MHz, DMSO-d6): 3.64 (s, 3H), 3.92 (s, 3H), 5.43 (s, 2H), 6.88 (s, 1H), 7.17 (dd, J=8.3, 1.45 Hz, 1H), 7.45 (d, J=1.75 Hz, 1H), 7.50 (s, 1H), 7.52 (s, 1H), 7.96 (s, 1H).

### [Example 222] Synthesis of 6,7-dimethoxy-9-(6-(methyl(phenyl)amino)pyridin-3-yl)naphtho[2,3-c]furan-1(3H)-one

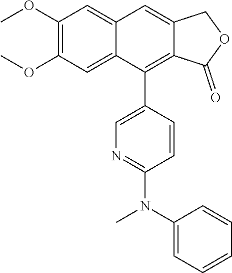 [SMILES: COc1cc2cc3c(c(-c4ccc(N(C)c5ccccc5)nc4)c2cc1OC)C(=O)OC3]

The compound of Example 222 was synthesized by the same method as that of the compound of Example 132 except for using 5-bromo-N-methyl-N-phenylpyridin-2-amine instead of 5-bromoquinoline.

Mass (M+H⁺): 427.1

¹H-NMR (500 MHz, DMSO-d6): 3.47 (s, 3H), 3.69 (s, 3H), 3.92 (s, 3H), 5.41 (s, 2H), 6.63 (dd, J=10.6, 0.85 Hz, 1H), 7.06 (s, 1H), 7.22-7.25 (m, 1H), 7.36-7.40 (m, 2H), 7.44-7.48 (m, 4H), 7.91 (s, 1H), 8.15 (dd, J=2.25, 0.9 Hz, 1H).

### [Example 223] Synthesis of 9-(6-(3-(dimethylamino)-5-(methylthio)-1H-1,2,4-triazol-1-yl)pyridin-3-yl)-6,7-dimethoxynaphtho[2,3-c]furan-1(3H)-one

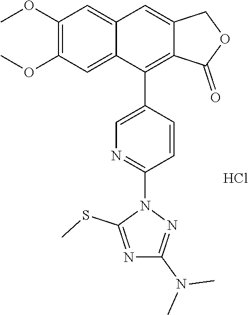 [SMILES: COc1cc2cc3c(c(-c4ccc(-n5nc(N(C)C)nc5SC)nc4)c2cc1OC)C(=O)OC3 ; Cl]

The compound of Example 223 was synthesized by the same method as that of the compound of Example 132 except for using 1-(5-bromopyridin-2-yl)-N,N-dimethyl-5-(methylthio)-1H-1,2,4-triazol-3-amine instead of 5-bromoquinoline.

Mass (M+H⁺): 478.1

¹H-NMR (500 MHz, DMSO-d6): 2.45-2.47 (m, 3H), 3.41 (s, 3H), 3.55 (s, 3H), 3.67 (s, 3H), 3.92 (s, 3H), 5.42 (s, 2H), 6.92 (d, J=8.6 Hz, 1H), 6.98 (s, 1H), 7.51 (s, 1H), 7.75 (dd, J=8.85, 2.55 Hz, 1H), 7.95 (s, 1H), 8.17 (d, J=1.75 Hz, 1H).

### [Example 224] Synthesis of 1-(6-hydroxybenzo[d][1,3]dioxol-5-yl)-3-(hydroxymethyl)-6,7-dimethoxy-2-naphthoyl chloride

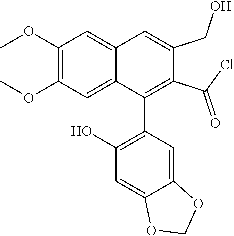 [SMILES: COc1cc2cc(CO)c(C(=O)Cl)c(-c3cc4c(cc3O)OCO4)c2cc1OC]

After 2 g (5.26 mmol) of 9-(6-hydroxybenzo[d][1,3]dioxol-5-yl)-6,7-dimethoxynaphtho[2,3-c]furan-1(3H)-one and 20 ml of 2N sodium hydroxide were injected into dichloromethane, the resulting mixture was stirred at 40° C. for two hours and separated in layers. After 20 ml of ethyl acetate was injected into an aqueous layer of the reactant, the resulting mixture was stirred at 40° C. for two hours to precipitate a solid, which was then filtered, washed and dried under reduced pressure to obtain 1.8 g (4.28 mmol, 81%) of 1-(6-hydroxybenzo[d][1,3]dioxol-5-yl)-3-(hydroxymethyl)-6,7-dimethoxy-2-naphthoic acid sodium salt as an intermediate.

After 1 g (2.38 mmol) of the intermediate was injected into 5 ml of toluene and 1 ml of dimethylformamide, 0.7 ml of thionyl chloride was slowly added dropwise thereto, stirred for 17 hours, crystallized in methanol, and then filtered and washed to obtain 0.79 g (1.89 mmol) of 1-(6-hydroxybenzo[d][1,3]dioxol-5-yl)-3-(hydroxymethyl)-6,7-dimethoxy-2-naphthoyl chloride as a title compound.

Mass (M+H⁺): 415.2

¹H-NMR (500 MHz, DMSO-d6): 3.64 (s, 3H), 3.95 (s, 3H), 5.38 (s, 2H), 5.98 (d, 2H), 6.61 (d, 2H), 6.93 (s, 1H), 7.43 (s, 1H), 7.85 (s, 1H), 9.09 (s, 1H)

### [Example 225] Synthesis of 9-(6-(benzo[d]thiazol-2-yl(methyl)amino)pyridin-3-yl)-6,7-dimethoxynaphtho[2,3-c]furan-1(3H)-one

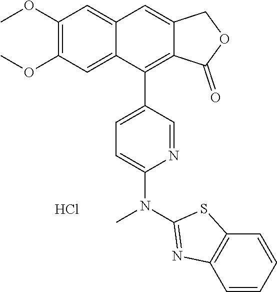 [SMILES: COc1cc2cc3c(c(-c4ccc(N(C)c5nc6ccccc6s5)nc4)c2cc1OC)C(=O)OC3 ; Cl]

The compound of Example 225 was synthesized by the same method as that of the compound of Example 132 except for using N-(5-bromopyridin-2-yl)-N-methylbenzo[d]thiazol-2-amine instead of 5-bromoquinoline.

Mass (M+H⁺): 484.1

¹H-NMR (500 MHz, DMSO-d6): 3.67 (s, 3H), 3.87 (s, 3H), 3.97 (s, 3H), 5.46 (s, 2H), 6.98 (s, 1H), 7.26 (m, 1H), 7.47 (m, 2H), 7.56 (m, 2H), 7.84 (d, 1H), 7.92 (d, 1H), 7.99 (s, 1H), 8.45 (d, 1H).

### [Example 226] Synthesis of 9-(6-(ethyl(pyridin-4-ylmethyl)amino)pyridin-3-yl)-6,7-dimethoxynaphtho[2,3-c]furan-1(3H)-one

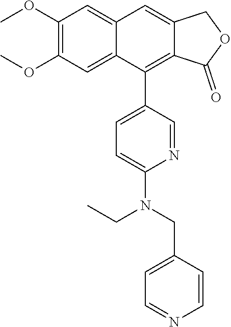 [SMILES: CCN(Cc1ccncc1)c1ccc(-c2c3c(cc4cc(OC)c(OC)cc24)COC3=O)cn1]

The compound of Example 226 was synthesized by the same method as that of the compound of Example 132 except for using 5-bromo-N-ethyl-N-(pyridin-4-ylmethyl)pyridin-2-amine instead of 5-bromoquinoline.

Mass (M+H⁺): 456.2

¹H-NMR (500 MHz, DMSO-d6): 3.62 (m, 2H), 3.67 (s, 3H), 3.91 (s, 3H), 4.84 (d, 2H), 5.40 (s, 1H), 6.73 (d, 1H), 7.05 (s, 1H), 7.25 (d, 2H), 7.47 (s, 1H), 7.55 (d, 1H), 7.89 (s, 1H), 8.06 (s, 1H), 8.47 (d, 2H).

### [Example 227] Synthesis of 9-(4-(5-bromopyrimidin-2-yl) piperazin-1-yl)-6,7-dimethoxynaphtho[2,3-c]furan-1(3H)-one

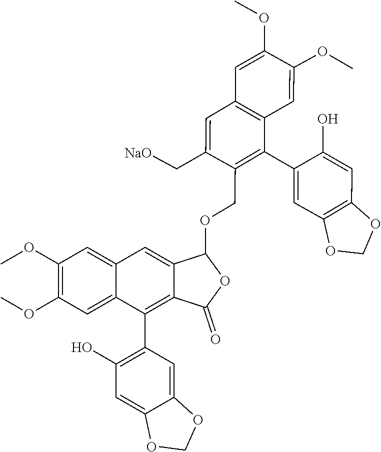

After 2 g (5.26 mmol) of 9-(6-hydroxybenzo[d][1,3]dioxol-5-yl)-6,7-dimethoxynaphtho[2,3-c]furan-1(3H)-one and 20 ml of 2N sodium hydroxide were injected into dichloromethane, the resulting mixture was stirred at 40° C. for two hours and separated in layers. After 20 ml of ethyl acetate was injected into an aqueous layer of the reactant, the resulting mixture was stirred at 40° C. for two hours to precipitate a solid, which was then filtered, washed and dried under reduced pressure to obtain 1.8 g (4.28 mmol, 81%) of 1-(6-hydroxybenzo[d][1,3]dioxol-5-yl)-3-(hydroxymethyl)-6,7-dimethoxy-2-naphthoic acid sodium salt.

After 1 g (2.38 mmol) of the intermediate, 545 mg (2.85 mmol) of EDC HCl, 386 mg (2.85 mmol) of HOBt, and 2.0 ml of DIEA were injected into 10 ml of dichloromethane, 0.3 ml of cis-2,6-dimethyl morpholine was slowly injected thereto, after which the resulting mixture was stirred at room temperature for 24 hours, and then ethyl acetate and purified water were injected thereto, filtered, washed, and distilled under reduced pressure. The residue was purified by column chromatography to obtain 0.85 g of the title compound (1.08 mmol, 43.1%).

Mass (M+H⁺): 785.2

¹H-NMR (500 MHz, DMSO-d6): 3.64 (s, 3H), 3.90 (s, 3H), 3.94 (d, 6H), 5.03 (d, 2H), 5.31 (t, 1H), 5.39 (s. 2H), 5.98 (d, 2H), 6.16 (s, 2H), 6.55 (s, 1H), 6.61 (s, 1H), 6.93 (s, 1H), 7.15 (s, 1H), 7.43 (s, 1H), 7.51 (s, 1H), 7.85 (s, 1H), 7.94 (s, 1H), 7.98 (s, 1H), 8.11 (s, 1H), 8.97 (s, 2H).

### [Example 228] Synthesis of 1-(6-hydroxybenzo[d][1,3]dioxol-5-yl)-3-(hydroxymethyl)-N-isopropyl-6,7-dimethoxy-2-naphthamide

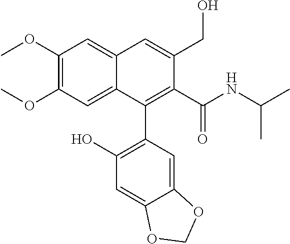 [SMILES: COc1cc2cc(CO)c(C(=O)NC(C)C)c(-c3cc4c(cc3O)OCO4)c2cc1OC]

After 2 g (5.26 mmol) of 9-(6-hydroxybenzo[d][1,3]dioxol-5-yl)-6,7-dimethoxynaphtho[2,3-c]furan-1(3H)-one and 20 ml of 2N sodium hydroxide were injected into dichloromethane, the resulting mixture was stirred at 40° C. for two hours and separated in layers. After 20 ml of ethyl acetate was injected into an aqueous layer of the reactant, the resulting mixture was stirred at 40° C. for two hours to precipitate a solid, which was then filtered, washed and dried under reduced pressure to obtain 1.8 g (4.28 mmol, 81%) of 1-(6-hydroxybenzo[d][1,3]dioxol-5-yl)-3-(hydroxymethyl)-6,7-dimethoxy-2-naphthoic acid sodium salt as an intermediate.

After 500 mg (1.2 mmol) of 1-(6-hydroxybenzo[d][1,3]dioxol-5-yl)-3-(hydroxymethyl)-6,7-dimethoxy-2-naphthoic acid sodium salt, 0.18 ml of tetramethyl orthosilicate, and 0.1 ml of isopropylamine were injected into 10 ml of toluene, the resulting mixture was stirred under reflux for eight hours. Ethyl acetate and purified water were injected and separated in layers, after which the separated organic layer was distilled under reduced pressure. The distilled residue was purified by column chromatography to obtain 150 mg (0.34 mmol, 28.3%) of 1-(6-hydroxybenzo[d][1,3]dioxol-5-yl)-3-(hydroxymethyl)-N-isopropyl-6,7-dimethoxy-2-naphthoamide as a title compound.

Mass (M+H⁺): 438.0

¹H-NMR (500 MHz, DMSO-d6): 0.91 (q, 6H), 3.58 (s, 3H), 3.77 (m, 1H), 3.85 (s, 3H), 4.61 (m, 2H), 5.15 (t, 1H), 5.94 (d, 2H), 6.53 (s, 1H), 6.59 (s, 1H), 6.65 (s, 1H), 7.31 (s, 1H), 7.37 (d, 1H), 7.81 (s, 1H), 8.82 (s, 1H)

### [Example 229] Synthesis of 6,7-dimethoxy-9-(6-(methylamino)pyridin-3-yl)naphtho[2,3-c]furan-1(3H)-one

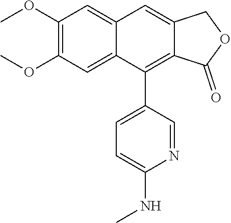 [SMILES: CNc1ccc(-c2c3c(cc4cc(OC)c(OC)cc24)COC3=O)cn1]

The compound of Example 230 was synthesized by the same method as that of the compound of Example 132 except for using 5-bromo-N-methylpyridin-2-amine instead of 5-bromoquinoline.

Mass (M+H⁺): 351.2

¹H-NMR (500 MHz, DMSO-d6): 2.83 (d, 3H), 3.68 (s, 3H), 3.93 (s, 3H), 5.40 (s, 2H), 6.54 (d, 1H), 7.67 (d, 1H), 7.09 (s, 1H), 7.41 (d, 1H), 7.48 (s, 1H), 7.88 (s, 1H), 7.96 (d, 1H).

### [Example 230] Synthesis of N-(5-(6,7-dimethoxy-3-oxo-1,3-dihydronaphtho[2,3-c]furan-4-yl)pyridin-2-yl)-N-methylacetamide

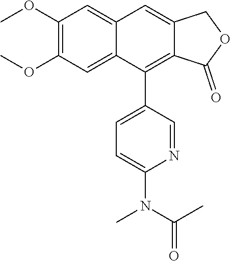 [SMILES: COc1cc2cc3c(c(-c4ccc(N(C)C(C)=O)nc4)c2cc1OC)C(=O)OC3]

The compound of Example 230 was synthesized by the same method as that of the compound of Example 132 except for using N-(5-bromopyridin-2-yl)-N-methylacetamide instead of 5-bromoquinoline.

Mass (M+H⁺): 393.1

¹H-NMR (500 MHz, DMSO-d6): 2.13 (s, 3H), 3.39 (s, 3H), 3.66 (s, 3H), 3.94 (s, 3H), 5.46 (s, 2H), 6.90 (s, 1H), 7.54 (s, 1H), 7.72 (d, 1H), 7.89 (d, 1H), 8.01 (s, 1H), 8.44 (s, 1H).

### [Example 231] Synthesis of 6,7-dimethoxy-9-(6-(methyl(5-methyl-1,3,4-oxadiazol-2-yl)amino)pyridin-3-yl)naphtho[2,3-c]furan-1(3H)-one

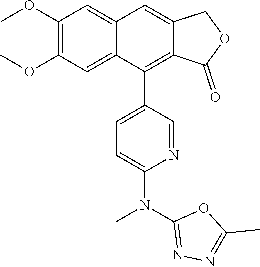

The compound of Example 231 was synthesized by the same method as that of the compound of Example 132 except for using N-(5-bromopyridin-2-yl)-N,5-dimethyl-1,3,4-oxadiazol-2-amine instead of 5-bromoquinoline.

Mass (M+H⁺): 433.0

¹H-NMR (500 MHz, DMSO-d6): 3.28 (s, 3H), 3.66 (m, 6H), 3.94 (s, 3H), 5.45 (s, 2H), 6.95 (s, 1H), 7.53 (s, 1H), 7.88 (d, 1H), 7.99 (s, 1H), 8.10 (d, 1H), 8.37 (s, 1H).

### [Example 232] Synthesis of 9-(6-((2,4-dimethoxybenzyl)(methyl)amino)pyridin-3-yl)-6,7-dimethoxynaphtho[2,3-c]furan-1(3H)-one

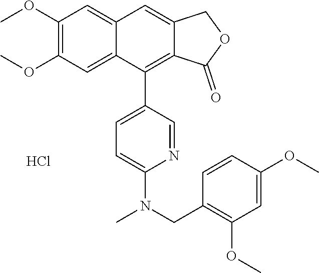

The compound of Example 232 was synthesized by the same method as that of the compound of Example 132 except for using 5-bromo-N-(2,4-dimethoxybenzyl)-N-methylpyridin-2-amine instead of 5-bromoquinoline.

Mass (M+H⁺): 501.2

¹H-NMR (500 MHZ, DMSO-d6): 3.13 (s, 3H), 3.72 (S, 3H), 3.73 (s, 3H), 3.76 (s, 3H), 3.93 (s, 3H), 4.78 (d, 2H), 5.44 (s, 2H), 6.49 (dd, 1H), 6.59 (d, 1H), 7.03 (s, 1H), 7.10 (br, 1H), 7.30 (d, 1H), 7.53 (s, 1H), 7.57 (s, 1H), 7.97 (d, 1H), 8.10 (s, 1H).

### [Example 233] Synthesis of N-(5-(6,7-dimethoxy-3-oxo-1,3-dihydronaphtho[2,3-c]furan-4-yl)pyridin-2-yl)-N-methylpivalamide

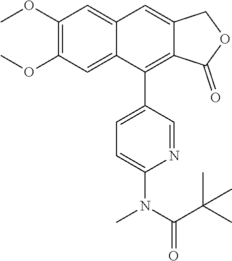 [SMILES: COc1cc2cc3c(c(-c4ccc(N(C)C(=O)C(C)(C)C)nc4)c2cc1OC)C(=O)OC3]

The compound of Example 233 was synthesized by the same method as that of the compound of Example 132 except for using N-(5-bromopyridin-2-yl)-N-methylpivalamide instead of 5-bromoquinoline.

Mass (M+H⁺): 435.0

¹H-NMR (500 MHz, DMSO-d6): 3.24 (s, 3H), 3.62 (s, 3H), 3.94 (s, 3H), 5.47 (s, 2H), 6.82 (s, 1H), 7.55 (s, 1H), 7.59 (d, 1H), 7.94 (d, 1H), 8.02 (s, 1H), 8.44 (s, 1H).

### [Example 234] Synthesis of 6-chloro-N-(5-(6,7-dimethoxy-3-oxo-1,3-dihydronaphtho[2,3-c]furan-4-yl)pyridin-2-yl)-N-methylnicotinamide

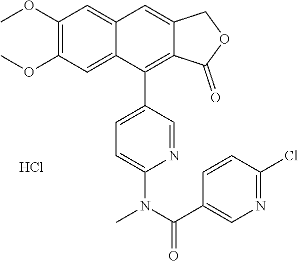 [SMILES: COc1cc2cc3c(c(-c4ccc(N(C)C(=O)c5ccc(Cl)nc5)nc4)c2cc1OC)C(=O)OC3 ; Cl]

The compound of Example 234 was synthesized by the same method as that of the compound of Example 132 except for using N-(5-bromopyridin-2-yl)-6-chloro-N-methylnicotinamide instead of 5-bromoquinoline.

Mass (M+H⁺): 490.1

¹H-NMR (500 MHz, DMSO-d6): 3.55 (s, 3H), 3.66 (s, 3H), 3.93 (s, 3H), 5.44 (s, 2H), 6.69 (s, 1H), 7.42 (m, 1H), 7.46 (m, 1H), 7.53 (s, 1H), 7.79 (d, 1H), 7.99 (s, 1H), 8.28 (s, 1H), 8.34 (s, 1H).

### [Example 235] Synthesis of N-(5-(6,7-dimethoxy-3-oxo-1,3-dihydronaphtho[2,3-c]furan-4-yl)pyridin-2-yl)-N-methylmorpholin-4-carboxamide

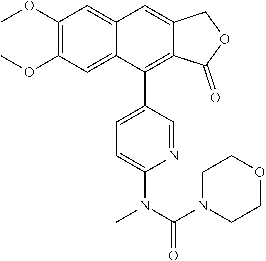 [SMILES: COc1cc2cc3c(c(-c4ccc(N(C)C(=O)N5CCOCC5)nc4)c2cc1OC)C(=O)OC3]

The compound of Example 235 was synthesized by the same method as that of the compound of Example 132 except for using N-(5-bromopyridin-2-yl)-N-methylmorpholin-4-carboxamide instead of 5-bromoquinoline.

Mass (M+H⁺): 464.2

¹H-NMR (500 MHz, DMSO-d6): 3.26 (s, 3H), 3.31 (m, 4H), 3.55 (m, 4H), 3.68 (s, 3H), 3.93 (s, 3H), 5.44 (s, 2H), 6.97 (s, 1H), 7.17 (d, 1H), 7.52 (s, 1H), 7.72 (d, 1H), 7.97 (s, 1H), 8.27 (s, 1H).

### [Example 236] Synthesis of N-(4-((5-(6,7-dimethoxy-3-oxo-1,3-dihydronaphtho[2,3-c]furan-4-yl)pyridin-2-yl)(methyl)amino)phenyl)-N-methylacetamide

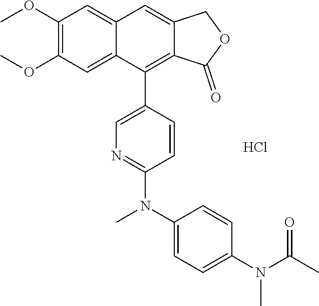 [SMILES: COc1cc2cc3c(c(-c4ccc(N(C)c5ccc(N(C)C(C)=O)cc5)nc4)c2cc1OC)C(=O)OC3 ; Cl]

The compound of Example 236 was synthesized by the same method as that of the compound of Example 132 except for using N-(4-((5-bromopyridin-2-yl)(methyl)amino)phenyl)-N-methylacetamide instead of 5-bromoquinoline.

Mass (M+H⁺): 498.2

¹H-NMR (500 MHz, DMSO-d6): 3.50 (s, 3H), 3.69 (s, 3H), 3.92 (s, 3H), 5.42 (s, 2H), 6.87-6.89 (m, 1H), 7.4 (s, 1H), 7.50 (s, 1H), 7.59-7.61 (m, 3H), 7.73-7.75 (m, 1H), 7.85-7.86 (m, 1H), 7.93 (s, 1H), 8.17 (t, J=0.85 Hz, 1H).

### [Example 237] Synthesis of 3-((5-(6,7-dimethoxy-3-oxo-1,3-dihydronaphtho[2,3-c]furan-4-yl)pyridin-2-yl)(methyl)amino)benzonitrile

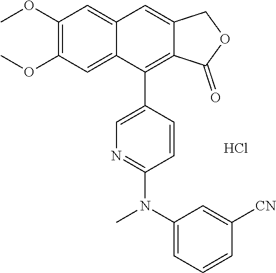 [SMILES: COc1cc2cc3c(c(-c4ccc(N(C)c5cccc(C#N)c5)nc4)c2cc1OC)C(=O)OC3 ; Cl]

The compound of Example 237 was synthesized by the same method as that of the compound of Example 132 except for using 3-((5-bromopyridin-2-yl)(methyl)amino)benzonitrile instead of 5-bromoquinoline.

Mass (M+H⁺): 452.1

¹H-NMR (500 MHZ, DMSO-d6): 3.50 (s, 3H), 3.70 (s, 3H), 3.93 (s, 3H), 5.44 (s, 2H), 6.89 (d, J=8.6 Hz, 1H), 7.04 (s, 1H), 7.50 (s, 1H), 7.59-7.62 (m, 3H), 7.74-7.75 (m, 1H), 7.87 (s, 1H), 7.93 (s, 1H), 8.17 (d, J=2.3 Hz, 1H).

### [Example 238] Synthesis of 9-(6-(4-(dimethylamino) piperidin-1-yl)pyridin-3-yl)-6,7-dimethoxynaphtho[2,3-c]furan-1(3H)-one

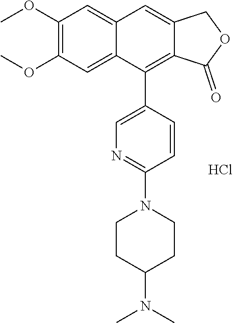 [SMILES: COc1cc2cc3c(c(-c4ccc(N5CCC(N(C)C)CC5)nc4)c2cc1OC)C(=O)OC3 ; Cl]

The compound of Example 238 was synthesized by the same method as that of the compound of Example 132 except for using 1-(5-bromopyridin-2-yl)-N,N-dimethylpiperidin-4-amine instead of 5-bromoquinoline.

Mass (M+H⁺): 448.2

¹H-NMR (500 MHz, DMSO-d6): 1.72-1.76 (m, 2H), 2.16-2.19 (m, 2H), 2.73 (d, J=5.15 Hz, 6H), 2.99-3.03 (m, 2H), 3.40-3.44 (m, 1H), 3.70 (s, 3H), 3.93 (s, 3H), 4.55-4.57 (m, 2H), 5.43 (s, 2H), 7.03 (s, 1H), 7.28 (br, 1H), 7.52 (s, 1H), 7.96 (s, 1H), 8.11 (s, 1H), 10.62 (br, 1H).

### [Example 239] Synthesis of 9-(6-((4-mercaptophenyl)(methyl)amino)pyridin-3-yl)-6,7-dimethoxynaphtho[2,3-c]furan-1(3H)-one

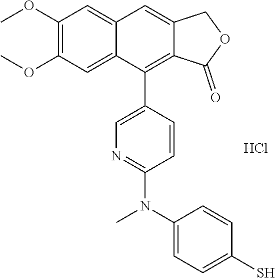 [SMILES: COc1cc2cc3c(c(-c4ccc(N(C)c5ccc(S)cc5)nc4)c2cc1OC)C(=O)OC3 ; Cl]

The compound of Example 239 was synthesized by the same method as that of the compound of Example 132 except for using 4-((5-bromopyridin-2-yl)(methyl)amino)benzenethiol instead of 5-bromoquinoline.

Mass (M+H⁺): 459.0

¹H-NMR (500 MHz, DMSO-d6): 2.71 (s, 3H), 3.67 (s, 3H), 3.92 (s, 3H), 5.42 (s, 2H), 6.71 (d, J=8.6 Hz, 2H), 6.85 (d, J=8.3 Hz, 1H), 6.89 (s, 1H), 7.40 (d, J=8.85 Hz, 2H), 7.51 (s, 1H), 7.66 (dd, J=8.3, 2.3 Hz, 1H), 7.97 (s, 1H), 8.36 (d, J=0.55 Hz, 1H).

### [Example 240] Synthesis of 6,7-dimethoxy-9-(6-(3-(trifluoromethyl)-5,6-dihydro-[1,2,4]triazolo[4,3-a]pyrazin-7 (8H)-yl) pyri din-3-yl)naphtho[2,3-c]furan-1(3H)-one

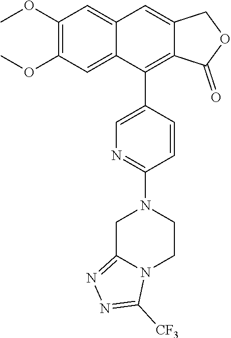 [SMILES: COc1cc2cc3c(c(-c4ccc(N5CCn6c(nnc6C(F)(F)F)C5)nc4)c2cc1OC)C(=O)OC3]

The compound of Example 240 was synthesized by the same method as that of the compound of Example 132 except for using 7-(5-bromopyridin-2-yl)-3-(trifluoromethyl)-5,6,7,8-tetrahydro-[1,2,4]triazolo[4,3-a]pyrazine instead of 5-bromoquinoline.

Mass (M+H⁺): 512.2

¹H-NMR (500 MHz, DMSO-d6): 3.66 (s, 3H), 3.92 (s, 3H), 4.21-4.22 (m, 2H), 4.29-4.30 (m, 2H), 5.06 (s, 2H), 5.42 (s, 2H), 7.02 (s, 1H), 7.19 (d, J=8.35 Hz, 1H), 7.50 (s, 1H), 7.70 (dd, J=8.6, 2.3 Hz, 1H), 7.93 (s, 1H), 8.18 (d, J=1.75 Hz, 1H).

### [Example 241] Synthesis of 3-((5-(6,7-dimethoxy-3-oxo-1,3-dihydronaphtho[2,3-c]furan-4-yl)pyridin-2-yl)(methyl)amino)-1-methyl-1H-pyrazol-4-carbonitrile

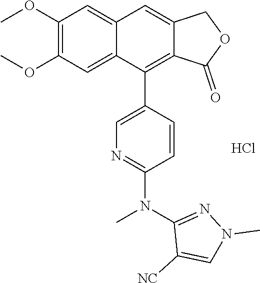

The compound of Example 241 was synthesized by the same method as that of the compound of Example 132 except for using 3-((5-bromopyridin-2-yl)(methyl)amino)-1-methyl-1H-pyrazol-4-carbonitrile instead of 5-bromoquinoline.

Mass (M+H⁺): 456.2

¹H-NMR (500 MHz, DMSO-d6): 3.67 (s, 3H), 3.92 (s, 3H), 4.16 (d, J=8.05 Hz, 6H), 5.51 (s, 2H), 7.00 (s, 1H), 7.60 (s, 1H), 8.11 (s, 1H), 8.40 (d, J=9.15 Hz, 1H), 8.72 (dd, J=9.15, 2.0 Hz, 1H), 9.22 (s, 1H), 9.27 (dd, J=1.7 Hz, 1H).

### [Example 242] Synthesis of 6,7-dimethoxy-9-(4-(piperidin-1-yl)phenyl)naphtho[2,3-c]furan-1(3H)-one

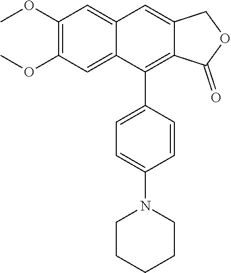 [SMILES: COc1cc2cc3c(c(-c4ccc(N5CCCCC5)cc4)c2cc1OC)C(=O)OC3]

After 4 g (23.2 mmol) of 4-bromoaniline, 9.6 g (69.8 mmol) of anhydrous potassium carbonate, and 3.4 ml (24.4 mmol) of 1,5-dibromopentane were injected into 60 ml of dimethylformamide, the resulting mixture was stirred at 100° C. for 18 hours, cooled, injected into ethyl acetate and purified water, separated in layers, added purified water and then separated in layers, dried over anhydrous sodium carbonate, filtered, washed, and distilled under reduced pressure. The residue was purified by column chromatography to obtain 3.8 g (15.8 mmol, 68%) of 1-(4-bromophenyl) piperidine as an intermediate.

After 1.6 g (6.7 mmol) of 1-(4-bromophenyl) piperidine, 550 mg (0.7 mmol) of PdCl2(dppf)CH2Cl2, 2.5 g (10.0 mmol) of bis(pinacolato)diborane, and 1.98 g (20.1 mmol) of potassium acetate were injected into 20 ml of 1,4-dioxane, the resulting mixture was heated at 100° C., stirred for 18 hours, distilled under reduced pressure, and purified by column chromatography to obtain 1.2 g (4.17 mmol, 62%) of 1-(4-(4,4,5,5-tetramethyl-1,3,2-dioxaborolan-2-yl)phenyl) piperidine as an intermediate.

After 1.5 g (3.8 mmol) of 6,7-dimethoxy-3-oxo-1,3-dihydronaphtho[2,3-c]furan-4-yl trifluoromethanesulfonate, 1.2 g (4.2 mmol) of 1-(4-(4,4,5,5-tetramethyl-1,3,2-dioxaborolan-2-yl)phenyl) piperidine, 310 mg (0.38 mmol) of PdCl2(dppf)CH2Cl2, and 3.2 g (15.2 mmol) of K3PO4 were injected into 30 ml of 1,4-dioxane, the resulting mixture was stirred at 80 to 90° C. for 20 hours, distilled under reduced pressure, separated in layers with ethyl acetate and purified water, dried over anhydrous sodium sulfate, filtered and washed. The reactant was distilled under reduced pressure, after which the residue was recrystallized with methanol to obtain 1.2 g (2.97 mmol, 78%) of 6,7-dimethoxy-9-(4-(piperidin-1-yl)phenyl)naphtho[2,3-c]furan-1(3H)-one as a title compound.

Mass (M+H⁺): 404.2

¹H-NMR (500 MHz, DMSO-d6): 1.54 (m, 2H), 1.63 (m, 4H), 3.22 (t, 4H), 3.62 (s, 3H), 3.91 (s, 3H), 5.38 (s, 2H), 7.00 (d, 2H), 7.05 (s, 1H), 7.17 (d, 2H), 7.45 (s, 1H), 7.86 (s, 1H).

### [Example 243] Synthesis of 6,7-dimethoxy-9-(3-(piperidin-1-yl)phenyl)naphtho[2,3-c]furan-1(3H)-one

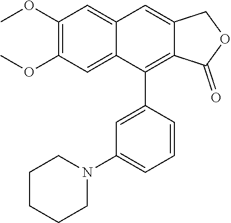 [SMILES: COc1cc2cc3c(c(-c4cccc(N5CCCCC5)c4)c2cc1OC)C(=O)OC3]

The compound of Example 243 was synthesized by the same method as that of the compound of Example 242 except for using 3-bromoaniline instead of 4-bromoaniline.

Mass (M+H⁺): 404.2

¹H-NMR (500 MHz, DMSO-d6): 1.52 (m, 2H), 1.57 (m, 4H), 3.13 (t, 4H), 3.59 (s, 3H), 3.91 (s, 3H), 5.40 (s, 2H), 6.70 (d, 1H), 6.84 (s, 1H), 6.98 (s, 1H), 7.00 (d, 1H), 7.29 (t, 1H), 7.47 (s, 1H), 7.90 (s, 1H).

### [Example 244] Synthesis of 6,7-dimethoxy-9-(3-morpholinophenyl)naphtho[2,3-c]furan-1(3H)-one

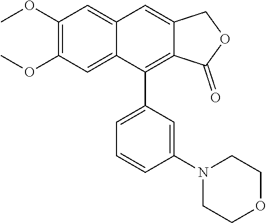 [SMILES: COc1cc2cc3c(c(-c4cccc(N5CCOCC5)c4)c2cc1OC)C(=O)OC3]

The compound of Example 244 was synthesized by the same method as that of the compound of Example 242 except for using bis(2-bromoethyl) ester instead of 1,5-dibromopentane.

Mass (M+H⁺): 406.2

¹H-NMR (500 MHZ, DMSO-d6): 3.10 (t, 4H), 3.59 (s, 3H), 3.69 (t, 4H), 3.91 (s, 3H), 5.41 (s, 2H), 6.77 (d, 1H), 6.88 (s, 1H), 6.96 (s, 1H), 7.03 (s, 1H), 7.33 (t, 1H), 7.47 (s, 1H), 7.91 (s, 1H).

### [Example 245] Synthesis of 6,7-dimethoxy-9-(4-morpholinophenyl)naphtho[2,3-c]furan-1(3H)-one

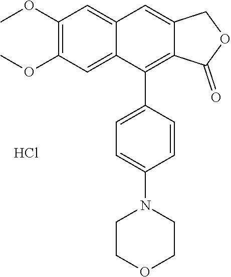 [SMILES: COc1cc2cc3c(c(-c4ccc(N5CCOCC5)cc4)c2cc1OC)C(=O)OC3 ; Cl]

The compound of Example 245 was synthesized by the same method as that of the compound of Example 242 except for using bis(2-bromoethyl) ester instead of 1,5-dibromopentane.

Mass (M+H⁺): 406.2

¹H-NMR (500 MHz, DMSO-d6): 3.20 (t, 4H), 3.61 (s, 3H), 3.75 (t, 4H), 3.91 (s, 3H), 5.39 (s, 2H), 7.02 (s, 1H), 7.03 (d, 2H), 7.21 (d, 2H), 7.46 (s, 1H), 7.87 (s, 1H).

### [Example 246] Synthesis of 6,7-dimethoxy-9-(4-(methylamino)phenyl)naphtho[2,3-c]furan-1(3H)-one

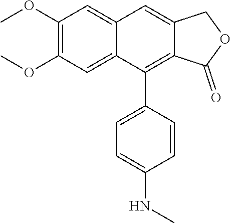 [SMILES: CNc1ccc(-c2c3c(cc4cc(OC)c(OC)cc24)COC3=O)cc1]

The compound of Example 246 was synthesized by the same method as that of the compound of Example 242 except for using iodomethane instead of 1,5-dibromopentane.

Mass (M+H⁺): 350.2

¹H-NMR (500 MHz, DMSO-d6): 2.73 (d, 3H), 3.63 (s, 3H), 3.90 (s, 3H), 5.37 (s, 2H), 5.85 (q, 1H), 6.62 (d, 1H), 7.07 (d, 2H), 7.11 (s, 1H), 7.43 (s, 1H), 7.82 (s, 1H).

### [Example 247] Synthesis of 9-(4-(dimethylamino)phenyl)-6,7-dimethoxynaphtho[2,3-c]furan-1(3H)-one

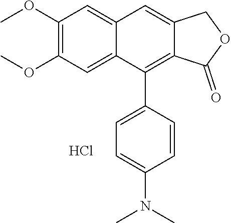 [SMILES: COc1cc2cc3c(c(-c4ccc(N(C)C)cc4)c2cc1OC)C(=O)OC3 ; Cl]

The compound of Example 247 was synthesized by the same method as that of the compound of Example 242 except for using iodomethane instead of 1,5-dibromopentane.

Mass (M+H⁺): 364.1

¹H-NMR (500 MHz, DMSO-d6): 3.08 (s, 6H), 3.62 (s, 3H), 3.91 (s, 3H), 5.41 (s, 2H), 6.96 (s, 1H), 7.36 (brm, 4H), 7.48 (s, 1H), 7.91 (s, 1H).

### [Example 248] Synthesis of 9-(4-(ethyl(methyl)amino)phenyl)-6,7-dimethoxynaphtho[2,3-c]furan-1(3H)-one

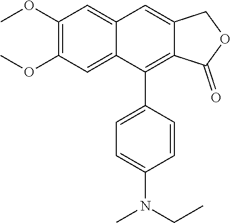 [SMILES: CCN(C)c1ccc(-c2c3c(cc4cc(OC)c(OC)cc24)COC3=O)cc1]

### Step 1) Synthesis of 4-bromo-N,N-diethylaniline and 4-bromo-N-ethylaniline

After 10 g (58.1 mmol) of 4-bromoaniline, 32 g (231.5 mmol) of anhydrous potassium carbonate, and 14 ml (173.4 mmol) of iodoethane were injected into acetonitrile, the resulting mixture was heated and stirred at 60° C. for 18 hours. The reactant was filtered, washed and distilled under reduced pressure. The resulting product was purified by column chromatography to obtain 4.3 g (18.8 mmol, 30%) of 4-bromo-N,N-diethylaniline as intermediate 1, and 5.28 g (26.4 mmol, 46%) of 4-bromo-N-ethylaniline as intermediate 2.

### Step 2) Synthesis of 4-bromo-N-ethyl-N-methylaniline

After 2 g (9.99 mmol) of 4-bromo-N-ethylaniline as intermediate 2 and 0.62 ml (14.98 mmol) of iodomethane were injected into 30 ml of tetrahydrofuran, 1.3 g (30.0 mmol) of sodium hydride (55% mineral oil) was slowly injected thereto. The reactant was stirred for five hours, after which methanol was injected and distilled under reduced pressure. Dichloromethane and purified water were injected into the concentrate to separate in layers, and then the organic layer was dried over anhydrous sodium sulfate, filtered, washed, and concentrated under reduced pressure. The concentrate was purified by column chromatography to obtain 2.03 g (9.49 mmol, 95%) of 4-bromo-N-ethyl-N-methylaniline as an intermediate.

### Step 3) Synthesis of N-ethyl-N-methyl-4-(4,4,5,5-tetramethyl-1,3,2-dioxaborolan-2-yl) aniline

After 2.03 g (9.49 mmol) of 4-bromo-N-ethyl-N-methylaniline as an intermediate, 774 mg (0.95 mmol) of PdCl2(dppf)CH2Cl2, 2.89 g (11.38 mmol) of bis(pinacolato)diborane, and 2.77 g (28.5 mmol) of potassium acetate were injected into 20 ml of 1,4-dioxane, the resulting mixture was heated at 100° C., stirred for 18 hours, distilled under reduced pressure, and purified by column chromatography to obtain 1.77 g (6.78 mmol, 72%) of N-ethyl-N-methyl-4-(4,4,5,5-tetramethyl-1,3,2-dioxaborolan-2-yl) aniline as an intermediate.

### Step 4) Synthesis of 9-(4-(ethyl(methyl)amino)phenyl)-6,7-dimethoxynaphtho[2,3-c]furan-1(3H)-one

After 2.65 g (6.75 mmol) of 6,7-dimethoxy-3-oxo-1,3-dihydronaphtho[2,3-c]furan-4-yl trifluoromethanesulfonate, 1.77 g (6.78 mmol) of N-ethyl-N-methyl-4-(4,4,5,5-tetramethyl-1,3,2-dioxaborolan-2-yl) aniline, 551 mg (0.67 mmol) of PdCl2(dppf)CH2Cl2, and 5.73 g (27.0 mmol) of K3PO4 were injected into 30 ml of 1,4-dioxane, the resulting mixture was stirred at 80-90° C. for 20 hours, distilled under reduced pressure, separated in layers with ethyl acetate and purified water, dried over anhydrous sodium sulfate, filtered and washed. The reactant was distilled under reduced pressure, and the residue was purified by column chromatography to obtain 2.06 g (5.46 mmol, 81%) of 9-(4-(ethyl(methyl)amino)phenyl)-6,7-dimethoxynaphtho[2,3-c]furan-1(3H)-one as a title compound.

Mass (M+H⁺): 378.2

¹H-NMR (500 MHz, DMSO-d6): 1.09 (t, 3H), 2.92 (s, 3H), 3.44 (q, 2H), 3.62 (s, 3H), 3.91 (s, 3H), 5.37 (s, 2H), 6.77 (d, 2H), 7.11 (s, 1h), 7.15 (d, 2H), 7.44 (s, 1H), 7.83 (s, 1H).

### [Example 249] Synthesis of 9-(4-(diethylamino)phenyl)-6,7-dimethoxynaphtho[2,3-c]furan-1(3H)-one

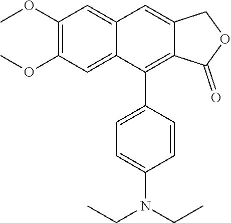 [SMILES: CCN(CC)c1ccc(-c2c3c(cc4cc(OC)c(OC)cc24)COC3=O)cc1]

The compound of Example 249 was synthesized by the same method as that of Step 3, 4) of Example 248 using 4-bromo-N,N-diethylaniline as intermediate 1 produced in Step 1) of Example 248.

Mass (M+H⁺): 392.1

¹H-NMR (500 MHz, DMSO-d6): 1.31 (t, 6H), 3.39 (q, 4H), 3.63 (s, 3H), 3.91 (s, 3H), 5.37 (s, 2H), 6.73 (d, 2H), 7.13 (s, 1H), 7.15 (d, 2H), 7.44 (s, 1H), 7.82 (s, 1H).

### [Example 250] Synthesis of 9-(4-(ethylamino)phenyl)-6,7-dimethoxynaphtho[2,3-c]furan-1(3H)-one

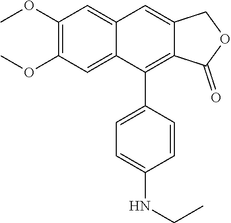 [SMILES: CCNc1ccc(-c2c3c(cc4cc(OC)c(OC)cc24)COC3=O)cc1]

The compound of Example 250 was synthesized by the same method as that of Step 3, 4) of Example 248 using 4-bromo-N-ethylaniline as intermediate 2 produced in Step 1) of Example 248.

Mass (M+H⁺): 364.2

¹H-NMR (500 MHz, DMSO-d6): 1.19 (t, 3H), 3.07 (q, 2H), 3.62 (s, 3H), 3.90 (s, 3H), 5.37 (s, 2H), 5.79 (brm, 1H), 6.64 (d, 2H), 7.06 (d, 2H), 7.11 (s, 1H), 7.43 (s, 1H), 7.82 (s, 1H).

### [Example 251] Synthesis of 6,7-dimethoxy-9-(4-(pyrrolidin-1-yl)phenyl)naphtho[2,3-c]furan-1(3H)-one

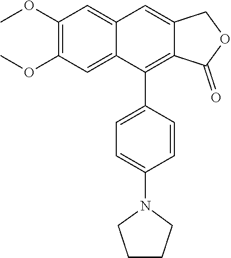 [SMILES: COc1cc2cc3c(c(-c4ccc(N5CCCC5)cc4)c2cc1OC)C(=O)OC3]

The compound of Example 251 was synthesized by the same method as that of the compound of Example 242 except for using 1,4-dibromobutane instead of 1,5-dibromopentane.

Mass (M+H⁺): 390.1

¹H-NMR (500 MHz, DMSO-d6): 1.97 (t, 4H), 3.29 (t, 4H), 3.62 (s, 3H), 3.92 (s, 3H), 5.35 (s, 2H), 6.62 (d, 2H), 7.12 (s, 1H), 7.15 (d, 2H), 7.44 (s, 1H), 7.83 (s, 1H).

### [Example 252] Synthesis of 6,7-dimethoxy-9-(3-(pyrrolidin-1-yl)phenyl)naphtho[2,3-c]furan-1(3H)-one

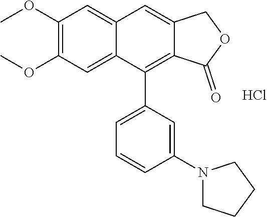 [SMILES: COc1cc2cc3c(c(-c4cccc(N5CCCC5)c4)c2cc1OC)C(=O)OC3 ; Cl]

The compound of Example 252 was synthesized by the same method as that of the compound of Example 242 except for using 1,4-dibromobutane instead of 1,5-dibromopentane and using 3-bromoaniline instead of 4-bromoaniline.

Mass (M+H⁺): 390.2

¹H-NMR (500 MHz, DMSO-d6): 1.93 (m, 4H), 3.24 (m, 4H), 3.59 (s, 3H), 3.91 (s, 3H), 5.41 (s, 2H), 6.54 (m, 1H), 6.59 (d, 1H), 6.71 (m, 1H), 7.02 (s, 1H), 7.29 (t, 1H), 7.47 (s, 1H), 7.90 (s, 1H).

### [Example 253] Synthesis of 9-(4-(ethyl(2-methoxyethyl)amino)phenyl)-6,7-dimethoxynaphtho[2,3-c]furan-1(3H)-one

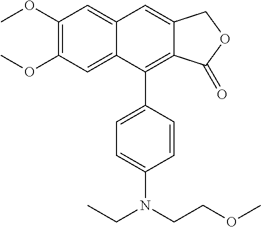

The compound of Example 253 was synthesized by the same method as that of Step 3, 4) of Example 248 using 4-bromo-N-ethylaniline as intermediate 2 produced in Step 1) of Example 248 and using 2-bromoethyl methyl ester instead of iodomethane of Step 2) of Example 248.

Mass (M+H⁺): 422.1

¹H-NMR (500 MHz, DMSO-d6): 1.15 (dt, 3H), 3.28 (dq, 2H), 3.28 (m, 2H), 3.51 (m, 2H), 3.63 (d, 3H), 3.91 (d, 3H), 5.38 (d, 2H), 6.64 (d, 1H), 6.75 (d, 1H), 7.08 (d, 1H), 7.13 (m, 2H), 7.44 (d, 1H), 7.83 (d, 1H).

### [Example 254] Synthesis of 9-(4-(ethyl(pyridin-3-ylmethyl)amino)phenyl)-6,7-dimethoxynaphtho[2,3-c]furan-1(3H)-one

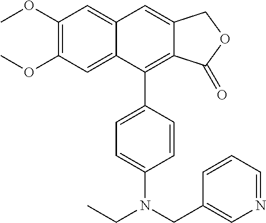 [SMILES: CCN(Cc1cccnc1)c1ccc(-c2c3c(cc4cc(OC)c(OC)cc24)COC3=O)cc1]

300 mg (0.86 mmol) of 9-(4-(ethylamino)phenyl)-6,7-dimethoxynaphtho[2,3-c]furan-1(3H)-one, 360 mg (2.58 mmol) of anhydrous potassium carbonate, and 650 mg (2.58 mmol) of 3-bromomethyl pyridine bromate were injected into 10 ml of dimethylformamide. The resulting mixture was stirred at 80-90° C. for eight hours, after which ethyl acetate and purified water were injected to separate in layers, and then the organic layer was dried with anhydrous sodium sulfate, filtered, and washed. The filtered reaction solution was distilled under reduced pressure, and the residue was recrystallized with methanol to obtain 184 mg (0.417 mmol, 49%) of 9-(4-(ethyl(pyridin-3-ylmethyl)amino)phenyl)-6,7-dimethoxynaphtho[2,3-c]furan-1(3H)-one as a title compound.

Mass (M+H⁺): 455.2

¹H-NMR (500 MHz, DMSO-d6): 1.17 (t, 3H), 3.53 (q, 2H), 3.60 (s, 3H), 3.90 (s, 3H), 4.64 (s, 2H), 5.37 (s, 2H), 6.79 (s, 2H), 7.06 (s, 1H), 7.14 (d, 2H), 7.33 (m, 1H), 7.44 (s, 1H), 7.66 (d, 1H), 7.83 (s, 1H), 8.43 (d, 1H), 8.51 (s, 1H).

### [Example 255] Synthesis of 6,7-dimethoxy-9-(4-(methyl(pyridin-3-ylmethyl)amino)phenyl)naphtho[2,3-c]furan-1(3H)-one

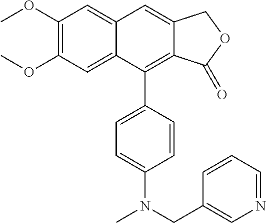 [SMILES: COc1cc2cc3c(c(-c4ccc(N(C)Cc5cccnc5)cc4)c2cc1OC)C(=O)OC3]

The compound of Example 255 was synthesized by the same method as that of the compound of Example 254 except for using 6,7-dimethoxy-9-(4-(methylamino)phenyl)naphtho[2,3-c]furan-1(3H)-one instead of 9-(4-(ethylamino)phenyl)-6,7-dimethoxynaphtho[2,3-c]furan-1(3H)-one.

Mass (M+H⁺): 441.2

¹H-NMR (500 MHz, DMSO-d6): 3.07 (s, 3H), 3.60 (s, 3H), 3.90 (s, 3H), 4.68 (s, 2H), 5.38 (s, 2H), 6.86 (d, 2H), 7.04 (s, 1H), 7.16 (d, 2H), 7.33 (m, 1H), 7.44 (s, 1H), 7.65 (d, 1H), 7.84 (s, 1H), 8.44 (d, 1H), 8.51 (s, 1H).

### [Example 256] Synthesis of 6,7-dimethoxy-9-(4-((2-methoxyethyl)(methyl)amino)phenyl)naphtho[2,3-c]furan-1(3H)-one

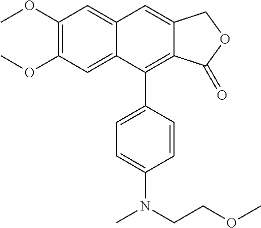

The compound of Example 256 was synthesized by the same method as that of the compound of Example 255 except for using 2-bromodethyl methyl ester instead of 3-bromomethyl pyridine bromate.

Mass (M+H⁺): 408.21

¹H-NMR (500 MHz, DMSO-d6): 2.97 (s, 3H), 3.26 (s, 3H), 3.53 (m, 4H), 3.63 (s, 3H), 3.91 (s, 3H), 5.38 (s, 2H), 6.78 (d, 2H), 7.10 (s, 1H), 7.15 (d, 2H), 7.44 (s, 1H), 7.84 (s, 1H).

### [Example 257] Synthesis of 6,7-dimethoxy-9-(3-(methylamino)phenyl)naphtho[2,3-c]furan-1(3H)-one

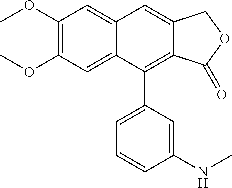

The compound of Example 257 was synthesized by the same method as that of the compound of Example 242 except for using iodomethane instead of 1,5-dibromopentane and using 3-bromoaniline instead of 4-bromoaniline.

Mass (M+H⁺): 350.2

¹H-NMR (500 MHz, DMSO-d6): 2.65 (d, 3H), 3.59 (s, 3H), 3.91 (s, 3H), 5.40 (s, 2H), 5.68 (q, 1H), 6.44 (d, 1H), 6.47 (d, 1H), 6.61 (d, 1H), 7.00 (s, 1H), 7.18 (t, 1H), 7.46 (s, 1H), 7.89 (s, 1H).

### [Example 258] Synthesis of 9-(3-(ethylamino)phenyl)-6,7-dimethoxynaphtho[2,3-c]furan-1(3H)-one

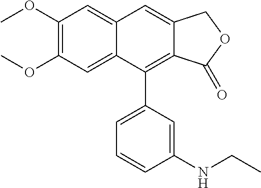 [SMILES: CCNc1cccc(-c2c3c(cc4cc(OC)c(OC)cc24)COC3=O)c1]

The compound of Example 258 was synthesized by the same method as that of the compound of Example 242 except for using iodoethane instead of 1,5-dibromopentane and using 3-bromoaniline instead of 4-bromoaniline.

Mass (M+H⁺): 364.2

¹H-NMR (500 MHz, DMSO-d6): 1.13 (t, 1H), 3.01 (q, 2H), 3.59 (s, 3H), 3.91 (s, 3H), 5.40 (s, 2H), 5.58 (m, 1H), 6.46 (d, 2H), 6.63 (d, 1H), 7.00 (s, 1H), 7.17 (t, 1H), 7.46 (s, 1H), 7.89 (s, 1H).

### [Example 259] Synthesis of 4-(2,2-difluorobenzo[d][1,3]dioxol-5-yl)-6,7,9-trimethoxynaphtho[2,3-c]furan-1(3H)-one

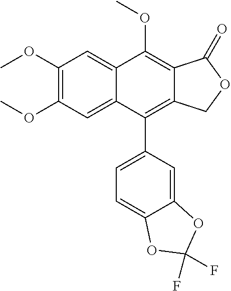 [SMILES: COc1cc2c(OC)c3c(c(-c4ccc5c(c4)OC(F)(F)O5)c2cc1OC)COC3=O]

### Step 1) Synthesis of 6,7,9-trimethoxynaphtho[2,3-c]furan-1(3H)-one

After 20 g (50.98 mmol) of 9-(6-hydroxybenzo[d][1,3]dioxol-5-yl)-6,7-dimethoxynaphtho[2,3-c]furan-1(3H)-one and 6.8 g (152.9 mmol) of sodium hydride (55% mineral oil) were injected into 30 ml of methanol, the resulting mixture was stirred for 30 minutes, and then the pH was adjusted to 1.0 with 20% hydrochloric acid and stirred at room temperature for one hour. The resulting solid was filtered, washed and dried under reduced pressure to obtain 13.2 g (50.7 mmol, 99.4%) of 9-hydroxy-6,7-dimethoxynaphtho[2,3-c]furan-1(3H)-one as an intermediate. After 13.2 g (50.7 mmol) of 9-hydroxy-6,7-dimethoxynaphtho[2,3-c]furan-1(3H)-one, 28.3 g (168.5 mmol) of anhydrous potassium carbonate, and 9.6 ml (153.4 mmol) of iodomethane were slowly added dropwise, the resulting mixture was stirred under reflux for 20 hours. The reactant was cooled, filtered, washed, distilled under reduced pressure, and recrystallized with methanol and purified water to obtain a solid, which was then dried under reduced pressure to obtain 13.43 g (48.9 mmol, 96.5%) of 6,7,9-trimethoxynaphtho[2,3-c]furan-1(3H)-one as an intermediate.

### Step 2) Synthesis of 4-bromo-6,7,9-trimethoxynaphtho[2,3-c]furan-1(3H)-one

After 13.43 g (48.9 mmol) of 6,7,9-trimethoxynaphtho[2,3-c]furan-1(3H)-one and 26.3 g (146.9 mmol) of N-brorosuccinimide were injected into 30 ml of dimethylformamide, the resulting mixture was stirred for four hours. After an aqueous sodium hydrogen carbonate solution was injected into the reactant, the resulting mixture was stirred to precipitate a solid, which was then filtered and washed. After the resulting solid was stirred in a methanol solvent, the resulting mixture was filtered and washed to obtain 14.91 g (42.2 mmol, 86.2%) of 4-bromo-6,7,9-trimethoxynaphtho[2,3-c]furan-1(3H)-one as an intermediate.

### Step 3) Synthesis of 6,7,9-trimethoxy-4-(4,4,5,5-tetramethyl-1,3,2-dioxaborolan-2-yl)naphtho[2,3-c]furan-1(3H)-one

14.91 g (42.2 mmol) of 4-bromo-6,7,9-trimethoxynaphtho[2,3-c]furan-1(3H)-one, 12.87 g (50.6 mmol) of bis(pinacolado)diborane, and 12.43 g (126.6 mmol) of potassium acetate were injected into 60 ml of 1,4-dioxane. The reactant was bubbled with nitrogen gas for ten minutes, after which 1.72 g (4.22 mmol) of Pd(dppf)Cl2 was injected thereto, stirred under reflux for 18 hours, filtered through a celite pad, washed, distilled under reduced pressure, and purified by column chromatography to obtain 11.3 g (28.2 mmol, 67.0%) of 6,7,9-trimethoxy-4-(4,4,5,5-tetramethyl-1,3,2-dioxaborolan-2-yl)naphtho[2,3-c]furan-1(3H)-one as an intermediate.

### Step 4) Synthesis of 4-(2,2-difluorobenzo[d][1,3]dioxol-5-yl)-6,7,9-trimethoxynaphtho[2,3-c]furan-1(3H)-one

After 3.56 g (15.0 mmol) of 5-bromo-2,2-difluorobenzo[d][1,3]dioxole, 6.02 g (15.0 mmol) of 6,7,9-trimethoxy-4-(4,4,5,5-tetramethyl-1,3,2-dioxaborolan-2-yl)naphtho[2,3-c]furan-1(3H)-one, and 4.15 g (30.0 mmol) of anhydrous potassium carbonate were injected into 30 ml of 1,4-dioxane, nitrogen bubbling was performed for ten minutes, and then 0.53 g (0.755 mmol) of Pd(PPh3)Cl2 was injected thereto and stirred under reflux for 24 hours. The reactant was cooled, filtered through a celite pad, washed, distilled under reduced pressure, and recrystallized in a methanol solvent to obtain 4.92 g (11.4 mmol, 76.1%) of 4-(2,2-difluorobenzo[d][1,3]dioxol-5-yl)-6,7,9-trimethoxynaphtho[2,3-c]furan-1(3H)-one as a title compound.

Mass (M+H⁺): 431.2

¹H-NMR (500 MHz, DMSO-d6): 3.70 (s, 3H), 3.92 (s, 3H), 4.21 (s, 3H), 5.15-5.28 (m, 2H), 6.92 (s, 1H), 7.27 (dd, J=8.3, 1.7 Hz, 1H), 7.55-7.57 (m, 2H), 7.61 (s, 1H).

### [Example 260] Synthesis of 4-(6-(1,4-dioxa-8-azaspiro[4.5]decan-8-yl)pyridin-3-yl)-6,7,9-trimethoxynaphtho[2,3-c]furan-1(3H)-one

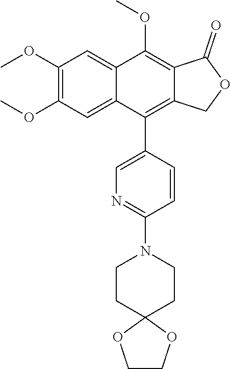 [SMILES: COc1cc2c(OC)c3c(c(-c4ccc(N5CCC6(CC5)OCCO6)nc4)c2cc1OC)COC3=O]

The compound of Example 260 was synthesized by the same method as that of the compound of Example 259 except for using 8-(5-bromopyridin-2-yl)-1,4-dioxa-8-azaspiro[4.5]decane instead of 5-bromo-2,2-difluorobenzo[d][1,3]dioxole.

Mass (M+H⁺): 493.2

¹H-NMR (500 MHz, DMSO-d6): 1.67 (t, J=5.7 Hz, 4H), 3.67-3.69 (m, 4H), 3.72 (s, 3H), 3.91 (s, 4H), 3.92 (s, 3H), 4.19 (s, 3H), 5.21-5.32 (m, 2H), 7.00-7.01 (m, 2H), 7.60 (s, 1H), 7.61-7.63 (m, 1H), 8.18 (d, J=2.05 Hz, 1H).

### [Example 261] Synthesis of 4-(6-(butyl(methyl)amino)pyridin-3-yl)-6,7,9-trimethoxynaphtho[2,3-c]furan-1(3H)-one

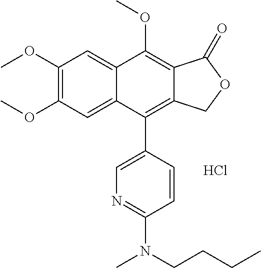 [SMILES: CCCCN(C)c1ccc(-c2c3c(c(OC)c4cc(OC)c(OC)cc24)C(=O)OC3)cn1 ; Cl]

The compound of Example 261 was synthesized by the same method as that of the compound of Example 259 except for using 5-bromo-N-butyl-N-methylpyridin-2-amine instead of 5-bromo-2,2-difluorobenzo[d][1,3]dioxole.

Mass (M+H⁺): 437.0

¹H-NMR (500 MHz, DMSO-d6): 0.91 (t, J=7.15 Hz, 3H), 1.32-1.36 (m, 2H), 1.57-1.60 (m, 2H), 3.18 (s, 3H), 3.61 (t, J=7.7 Hz, 2H), 3.75 (s, 3H), 3.92 (s, 3H), 4.21 (s, 3H), 5.22-5.34 (m, 2H), 6.95 (s, 1H), 7.62 (s, 1H), 7.90 (br, 1H), 8.qq (d, J=2.3 Hz, 1H).

### Example 262 Synthesis of 4-(benzo[d][1,3]dioxol-5-yl)-6,7,9-trimethoxynaphtho[2,3-c]furan-1(3H)-one

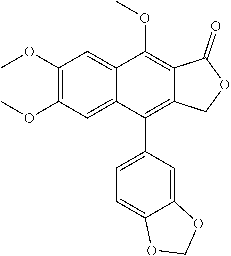 [SMILES: COc1cc2c(OC)c3c(c(-c4ccc5c(c4)OCO5)c2cc1OC)COC3=O]

The compound of Example 262 was synthesized by the same method as that of the compound of Example 259 except for using 5-bromobenzo[d][1,3]dioxole instead of 5-bromo-2,2-difluorobenzo[d][1,3]dioxole.

Mass (M+H⁺): 395.0

¹H-NMR (500 MHZ, DMSO-d6): 3.69 (s, 3H), 3.92 (s, 3H), 4.19 (s, 3H), 5.14-5.26 (m, 2H), 6.08-6.11 (m, 2H), 6.89 (dd, J=7.7, 1.7 Hz, 1H), 6.99 (s, 1H), 7.04 (d, J=1.7 Hz, 1H), 7.05-7.06 (m, 1H), 7.60 (s, 1H)

### [Example 263] Synthesis of 6,7,9-trimethoxy-4-(6-(methyl(3-(trifluoromethyl)phenyl)amino)pyridin-3-yl)naphtho[2,3-c]furan-1(3H)-one

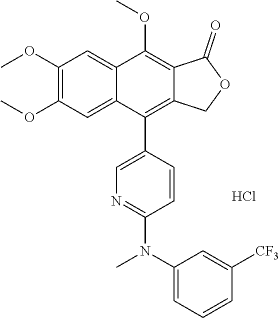 [SMILES: COc1cc2c(OC)c3c(c(-c4ccc(N(C)c5cccc(C(F)(F)F)c5)nc4)c2cc1OC)COC3=O ; Cl]

The compound of Example 263 was synthesized by the same method as that of the compound of Example 259 except for using 5-bromo-N-methyl-N-(3-(trifluoromethyl)phenyl)pyridin-2-amine instead of 5-bromo-2,2-difluorobenzo[d][1,3]dioxole.

Mass (M+H⁺): 525.1

¹H-NMR (500 MHz, DMSO-d6): 3.51 (s, 3H), 3.74 (s, 3H), 3.92 (s, 3H), 4.20 (s, 3H), 5.18-5.35 (m, 2H), 6.88 (d, J=8.85 Hz, 1H), 6.99 (s, 1H), 7.57-7.59 (m, 1H), 7.61 (s, 1H), 7.67 (t, J=7.7 Hz, 1H), 7.71-7.72 (m, 2H), 7.75 (s, 1H), 8.27 (t, J=1.75 Hz, 1H).

### [Example 264] Synthesis of 3-(dimethylamino)-1-(5-(6,7,9-trimethoxy-1-oxo-1,3-dihydronaphtho[2,3-c]furan-4-yl)pyridin-2-yl)-1H-pyrazol-4-carbonitrile

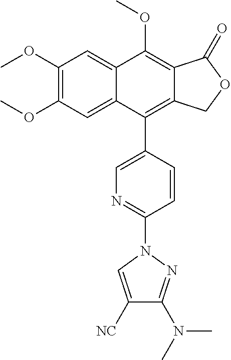 [SMILES: COc1cc2c(OC)c3c(c(-c4ccc(-n5cc(C#N)c(N(C)C)n5)nc4)c2cc1OC)COC3=O]

The compound of Example 264 was synthesized by the same method as that of the compound of Example 259 except for using 1-(5-bromopyridin-2-yl)-3-(dimethylamino)-1H-pyrazol-4-carbonitrile instead of 5-bromo-2,2-difluorobenzo[d][1,3]dioxole.

Mass (M+H⁺): 486.2

¹H-NMR (500 MHz, DMSO-d6): 3.04 (s, 6H), 3.71 (s, 3H), 3.93 (s, 3H), 4.23 (s, 3H), 5.19-5.37 (m, 2H), 6.91 (s, 1H), 7.64 (s, 1H), 7.95 (d, J=8.3 Hz, 1H), 8.12 (dd, J=8.55, 2.3 Hz, 1H), 8.57 (d, J=2.25 Hz, 1H), 9.22 (s, 1H).

### [Example 265] Synthesis of 6,7,9-trimethoxy-4-(6-((2-methoxyethyl)(methyl)amino)pyridin-3-yl)naphtho[2,3-c]furan-1(3H)-one

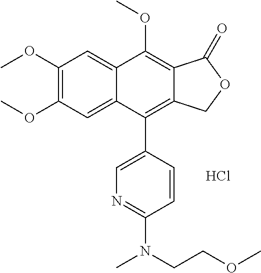

The compound of Example 265 was synthesized by the same method as that of the compound of Example 259 except for using 5-bromo-N-(2-methoxyethyl)-N-methylpyridin-2-amine instead of 5-bromo-2,2-difluorobenzo[d][1,3]dioxole.

Mass (M+H⁺): 439.1

¹H-NMR (500 MHZ, DMSO-d6): 3.19 (s, 3H), 3.27 (s, 3H), 3.58 (t, J=5.45 Hz, 2H), 3.75 (s, 3H), 3.81-3.83 (m, 2H), 3.93 (s, 3H), 4.21 (s, 3H), 5.22-5.31 (m, 2H), 6.95 (s, 1H), 7.19 (br, 1H), 7.62 (s, 1H), 7.89 (br, 1H), 8.12 (d, J=2.0 Hz, 1H).

### [Example 266] Synthesis of (S)-6,7,9-trimethoxy-4-(6-((5-methoxy-1,2,3,4-tetrahydronaphthalen-2-yl)(methyl)amino)pyridin-3-yl)naphtho[2,3-c]furan-1(3H)-one

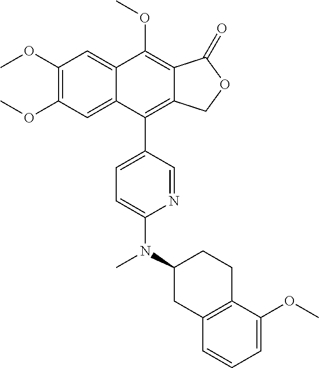

The compound of Example 266 was synthesized by the same method as that of the compound of Example 259 except for using (S)-5-bromo-N-(5-methoxy-1,2,3,4-tetrahydronaphthalen-2-yl)-N-methylpyridin-2-amine instead of 5-bromo-2,2-difluorobenzo[d][1,3]dioxole.

Mass (M+H⁺): 541.3

¹H-NMR (500 MHz, DMSO-d6): 1.94 (m, 2H), 2.92 (m, 2H), 2.97 (m, 2H), 3.26 (s, 3H), 3.73 (s, 3H), 3.74 (s, 3H), 3.92 (s, 3H), 4.19 (s, 3H), 4.83 (m, 1H), 5.20 (d, 2H), 6.69 (d, 1H), 6.73 (d, 1H), 6.80 (d, 1H), 7.04 (s, 1H), 7.07 (m, 1H), 7.61 (s, 1H), 7.62 (d, 1H), 8.16 (s, 1H).

### [Example 267] Synthesis of 6,7,9-trimethoxy-4-(6-morpholinopyridin-3-yl)naphtho[2,3-c]furan-1(3H)-one

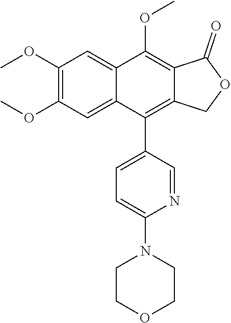 [SMILES: COc1cc2c(OC)c3c(c(-c4ccc(N5CCOCC5)nc4)c2cc1OC)COC3=O]

The compound of Example 267 was synthesized by the same method as that of the compound of Example 259 except for using 4-(5-bromopyridin-2-yl)morpholine instead of 5-bromo-2,2-difluorobenzo[d][1,3]dioxole.

Mass (M+H⁺): 437.2

¹H-NMR (500 MHz, DMSO-d6): 3.52 (m, 4H), 3.71 (m, 4H), 3.72 (s, 3H), 3.93 (s, 3H), 4.20 (s, 3H), 5.24 (d, 2H), 6.97 (m, 2H), 7.61 (s, 1H), 7.67 (d, 1H), 8.22 (s, 1H).

### [Example 268] Synthesis of 4-(6-((2,3-dihydro-1H-indan-2-yl)(methyl)amino)pyridin-3-yl)-6,7,9-trimethoxynaphtho[2,3-c]furan-1(3H)-one

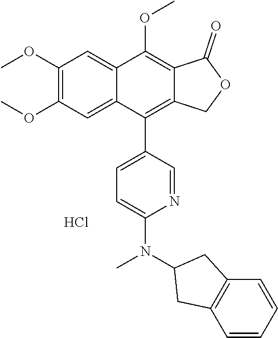 [SMILES: COc1cc2c(OC)c3c(c(-c4ccc(N(C)C5Cc6ccccc6C5)nc4)c2cc1OC)COC3=O ; Cl]

The compound of Example 268 was synthesized by the same method as that of the compound of Example 259 except for using 5-bromo-N-(2,3-dihydro-1H-indan-2-yl)-N-methylpyridin-2-amine instead of 5-bromo-2,2-difluorobenzo[d][1,3]dioxole.

Mass (M+H⁺): 497.2

¹H-NMR (500 MHZ, DMSO-d6): 2.95 (s, 3H), 3.10 (m, 2H), 3.18 (m, 2H), 3.75 (s, 3H), 4.00 (s, 3H), 4.20 (s, 3H), 5.27 (d, 2H), 5.53 (br, 1H), 7.02 (s, 1H), 7.15 (s, 1H), 7.16 (s, 1H), 7.24 (s, 1H), 7.25 (s, 1H), 7.62 (s, 1H), 7.79 (br, 1H), 8.19 (d, 1H).

### [Example 269] Synthesis of 6-(6,7-dimethoxy-3-oxo-1,3-dihydronaphtho[2,3-c]furan-4-yl)benzo[d][1,3]dioxol-5-yl L-valinate hydrochloride

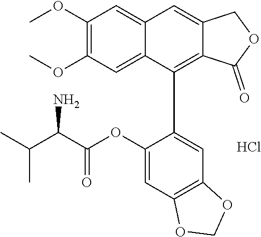 [SMILES: COc1cc2cc3c(c(-c4cc5c(cc4OC(=O)[C@H](N)C(C)C)OCO5)c2cc1OC)C(=O)OC3 ; Cl]

After 2.85 g (7.49 mmol) of 9-(6-hydroxybenzo[d][1,3]dioxol-5-yl)-6,7-dimethoxynaphtho[2,3-c]furan-1(3H)-one, 1.95 g (8.97 mmol) of Boc-L-valine, 0.64 g (5.32 mmol) of DMAP, and 2.15 g (11.21 mmol) of EDC-HCl were injected into 30 ml of dichloromethane, the resulting mixture was stirred at room temperature for two hours. After 20 ml of purified water was injected into the reactant and separated in layers, 20% hydrochloric acid was diluted 50 times and injected in the organic layer, and separated in layers. The organic layer was dried over anhydrous sodium sulfate, filtered, washed, and distilled under reduced pressure. The residue was dissolved in methanol, 5 ml of 35% hydrochloric acid was injected thereto, and the resulting mixture was stirred for three hours. The reactant was distilled under reduced pressure and the residue was dissolved in dichloromethane, after which tert-butylmethyl ester was slowly added dropwise thereto and stirred, and then the resulting solid was filtered and washed to obtain 3.07 g (5.94 mmol, 79.5%) of 6-(6,7-dimethoxy-3-oxo-1,3-dihydronaphtho[2,3-c]furan-4-yl)benzo[d][1,3]dioxol-5-yl L-valinate hydrochloride as a title compound.

Mass (M+H⁺): 480.2

¹H-NMR (500 Mhz, DMSO-d6): δ 0.02-0.17 (m, 6H), 0.85-0.95 (m, 1H), 1.24-1.38 (m, 1H), 3.65 (d, J=2.6 Hz, 3H), 3.90 (d, J=3.15 Hz, 3H), 5.36-5.44 (m, 2H), 6.17-6.19 (m, 2H), 6.83 (d, J=11.75 Hz, 1H), 6.90 (d, J=3.15 Hz, 1H), 7.05 (d, J=41.8 Hz, 1H), 7.46 (s, 1H), 7.95 (d, J=4.3 Hz, 1H), 8.37 (br, 2H)

### [Example 270] Synthesis of 6-(6,7-dimethoxy-3-oxo-1,3-dihydronaphtho[2,3-c]furan-4-yl)benzo[d][1,3]dioxol-5-yl L-isoleucinate

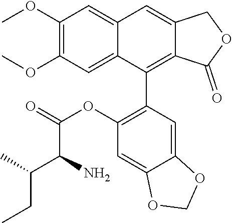 [SMILES: CC[C@H](C)[C@H](N)C(=O)Oc1cc2c(cc1-c1c3c(cc4cc(OC)c(OC)cc14)COC3=O)OCO2]

The compound of Example 270 was synthesized by the same method as that of the compound of Example 269 except for using N-Boc-isoleucine instead of N-Boc-valine.

Mass (M+H⁺): 494.2

¹H-NMR (500 MHz, CDCl₃): 3.66-3.73 (m, 2H), 3.81 (s, 3H), 3.84 (s, 3H), 4.01 (s, 3H), 4.04 (s, 3H), 5.98-5.99 (m, 1H), 6.04-6.06 (m, 2H), 6.10 (s, 2H), 6.64 (s, 1H), 6.94 (d=J=3.7 Hz, 1H), 7.14 (s, 2H), 7.74 (s, 1H).

### [Example 271] Synthesis of 6-(6,7-dimethoxy-3-oxo-1,3-dihydronaphtho[2,3-c]furan-4-yl)benzo[d][1,3]dioxol-5-yl isonicoinate

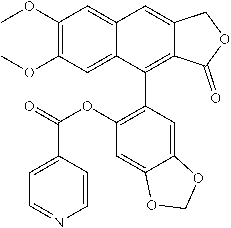 [SMILES: COc1cc2cc3c(c(-c4cc5c(cc4OC(=O)c4ccncc4)OCO5)c2cc1OC)C(=O)OC3]

The compound of Example 271 was synthesized by the same method as that of the compound of Ex ample 269 except for using isonicotinic acid instead of N-Boc-valine.

Mass (M+H⁺): 486.1

¹H-NMR (500 MHz, DMSO-d6): 3.67 (s, 3H), 3.84 (s, 3H), 5.39 (s, 2H), 6.19 (s, 2H), 6.90 (s, 1H), 6.98 (s, 1H), 7.19-7.20 (m, 3H), 7.37 (s, 1H), 7.86 (s, 1H), 8.56 (dd, J=4.3, 1.75 Hz, 2H).

### [Example 272] Synthesis of 6-(6,7-dimethoxy-3-oxo-1,3-dihydronaphtho[2,3-c]furan-4-yl)benzo[d][1,3]dioxol-5-yl nicotinate

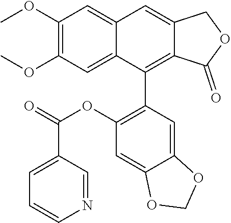 [SMILES: COc1cc2cc3c(c(-c4cc5c(cc4OC(=O)c4cccnc4)OCO5)c2cc1OC)C(=O)OC3]

The compound of Example 272 was synthesized by the same method as that of the compound of Example 269 except for using nicotinic acid instead of N-Boc-valine.

Mass (M+H⁺): 486.1

¹H-NMR (500 MHz, DMSO-d6): 3.67 (s, 3H), 3.84 (s, 3H), 5.35-5.42 (m, 2H), 6.19 (s, 2H), 6.92 (s, 1H), 6.97 (s, 1H), 7.19 (s, 1H), 7.32-7.35 (m, 1H), 7.37 (s, 1H), 7.70-7.72 (m, 1H), 7.86 (s, 1H), 8.39 (dd, J=1.45, 0.85 Hz, 1H), 8.63 (dd, J=4.85, 1.70 Hz, 1H).

### [Example 273] Synthesis of 6-(6,7-dimethoxy-3-oxo-1,3-dihydronaphtho[2,3-c]furan-4-yl)benzo[d][1,3]dioxol-5-yl L-prolinate

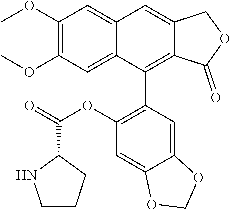 [SMILES: COc1cc2cc3c(c(-c4cc5c(cc4OC(=O)[C@@H]4CCCN4)OCO5)c2cc1OC)C(=O)OC3]

The compound of Example 273 was synthesized by the same method as that of the compound of Example 269 except for using N-Boc-proline instead of N-Boc-valine.

Mass (M+H⁺): 478.1

¹H-NMR (500 MHz, DMSO-d6): 0.21-0.23 (m, 1H), 0.66-0.79 (m, 1H), 1.03-1.07 (m, 1H), 1.41-1.52 (m, 1H), 2.73-2.78 (m, 1H), 2.89-2.91 (m, 1H), 3.65 (d, J=3.7 Hz, 3H), 3.91 (d, J=4.0 Hz, 3H), 4.05-4.19 (m, 1H), 5.40-5.45 (m, 2H), 6.18-6.20 (m, 2H), 6.80 (s, 1H), 6.95 (d, J=4.3 Hz, 1H), 7.01 (s, 1H), 7.04 (s, 1H), 7.49 (d, J=2.85 Hz, 1H), 7.97 (d, J=6.3 Hz, 1H).

### [Example 274] Synthesis of 6-(6,7-dimethoxy-3-oxo-1,3-dihydronaphtho[2,3-c]furan-4-yl)benzo[d][1,3]dioxol-5-yl acetate

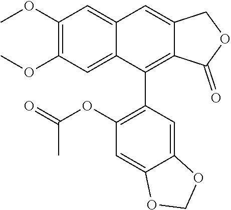 [SMILES: COc1cc2cc3c(c(-c4cc5c(cc4OC(C)=O)OCO5)c2cc1OC)C(=O)OC3]

The compound of Example 274 was synthesized by the same method as that of the compound of Example 269 except for using acetic acid instead of N-Boc-valine.

Mass (M+H⁺): 423.1

¹H-NMR (500 MHz, DMSO-d6): 1.56 (s, 3H), 3.63 (s, 3H), 3.91 (s, 3H), 5.38-5.46 (m, 2H), 6.14 (d, J=2.0 Hz, 2H), 6.83 (s, 1H), 6.84 (s, 1H), 6.94 (s, 1H), 7.46 (s, 1H), 7.92 (s, 1H).

### [Example 275] Synthesis of 6-(6,7-dimethoxy-3-oxo-1,3-dihydronaphtho[2,3-c]furan-4-yl)benzo[d][1,3]dioxol-5-yl isobutrate

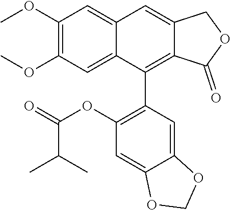 [SMILES: COc1cc2cc3c(c(-c4cc5c(cc4OC(=O)C(C)C)OCO5)c2cc1OC)C(=O)OC3]

The compound of Example 275 was synthesized by the same method as that of the compound of Example 269 except for using isobutyric acid instead of N-Boc-valine.

Mass (M+H⁺): 451.1

¹H-NMR (500 MHz, DMSO-d6): 0.25 (d, J=7.15 Hz, 3H), 0.52 (d, J=6.85 Hz, 3H), 2.11-2.15 (m, 1H), 3.64 (s, 3H), 3.90 (s, 3H), 5.41 (s, 2H), 6.15 (s, 2H), 6.81 (s, 1H), 6.88 (s, 1H), 6.92 (s, 1H), 7.45 (s, 1H), 7.92 (s, 1H).

### [Example 276] Synthesis of 6-(6,7-dimethoxy-3-oxo-1,3-dihydronaphtho[2,3-c]furan-4-yl)benzo[d][1,3]dioxol-5-yl pivalate

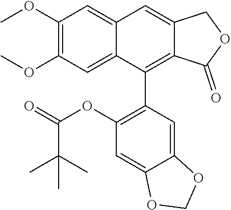 [SMILES: COc1cc2cc3c(c(-c4cc5c(cc4OC(=O)C(C)(C)C)OCO5)c2cc1OC)C(=O)OC3]

The compound of Example 276 was synthesized by the same method as that of the compound of Example 269 except for using pivalic acid instead of N-Boc-valine.

Mass (M+H⁺): 465.2

¹H-NMR (500 MHz, DMSO-d6): 0.51 (s, 9H), 3.64 (s, 3H), 3.90 (s, 3H), 5.41 (s, 2H), 6.15 (s, 2H), 6.82 (s, 1H), 6.89 (s, 1H), 6.91 (s, 1H), 7.45 (s, 1H), 7.92 (s, 1H).

### [Example 277] Synthesis of 6-(6,7-dimethoxy-3-oxo-1,3-dihydronaphtho[2,3-c]furan-4-yl)benzo[d][1,3]dioxol-5-yl diethyl phosphate

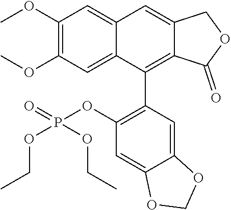 [SMILES: CCOP(=O)(OCC)Oc1cc2c(cc1-c1c3c(cc4cc(OC)c(OC)cc14)COC3=O)OCO2]

After 0.6 g (1.58 mmol) of 9-(6-hydroxybenzo[d][1,3]dioxol-5-yl)-6,7-dimethoxynaphtho[2,3-c]furan-1(3H)-one and 0.5 ml (3.48 mmol) of diethylchlorophosphate were injected into 10 ml of dichloromethane in a reactor, the resulting mixture was cooled to 0-5° C., and then 0.44 ml (6.32 mmol) of triethylamine was slowly added dropwise thereto, heated to room temperature, and stirred for 17 hours. After 10 ml of purified water was injected and separated in layers, 10 ml of purified water was injected again, separated in layers, dried over anhydrous sodium sulfate, filtered, washed, and distilled under reduced pressure. The residue was purified by column chromatography to obtain 480 mg (0.92 mmol, 59%) of 6-(6,7-dimethoxy-3-oxo-1,3-dihydronaphtho[2,3-c]furan-4-yl)benzo[d][1,3]dioxol-5-yl diethyl phosphate as a title compound.

Mass (M+H⁺): 517.1

¹H-NMR (500 MHz, DMSO-d6): 0.67 (t, 3H), 0.95 (t, 3H), 3.28 (q, 2H), 3.62 (q, 2H), 3.67 (s, 3H), 3.91 (s, 3H), 5.42 (s, 2H), 6.14 (s, 2H), 6.87 (s, 2H), 6.95 (s, 1H), 7.48 (s, 1H), 7.94 (s, 1H).

### [Example 278] Synthesis of 6-(6,7-dimethoxy-3-oxo-1,3-dihydronaphtho[2,3-c]furan-4-yl)benzo[d][1,3]dioxol-5-yl phenyl isopropylphosphoramidate

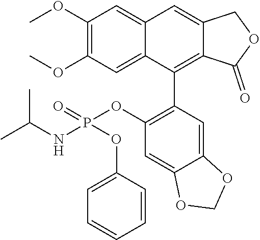 [SMILES: COc1cc2cc3c(c(-c4cc5c(cc4OP(=O)(NC(C)C)Oc4ccccc4)OCO5)c2cc1OC)C(=O)OC3]

After 0.65 g (1.71 mmol) of 9-(6-hydroxybenzo[d][1,3]dioxol-5-yl)-6,7-dimethoxynaphtho[2,3-c]furan-1(3H)-one and 0.3 ml (2.05 mmol) of phenyl dichlorophosphate were injected into 10 ml of dichloromethane in a reactor, the resulting mixture was cooled to 0-5° C., and then 0.7 ml (5.13 mmol) of triethylamine was slowly added dropwise thereto, and heated to room temperature, after which 0.8 ml (8.6 mmol) of isopropylamine was injected and stirred for 17 hours. After 10 ml of purified water was injected and separated in layers, 10 ml of purified water was injected again, separated in layers, dried over anhydrous sodium sulfate, filtered, washed, and distilled under reduced pressure. The residue was purified by column chromatography to obtain 890 mg (1.54 mmol, 90%) of 6-(6,7-dimethoxy-3-oxo-1,3-dihydronaphtho[2,3-c]furan-4-yl)benzo[d][1,3]dioxol-5-yl phenyl isopropylphosphoramidate as a title compound.

Mass (M+H⁺): 578.1

¹H-NMR (500 MHz, DMSO-d6): 0.54 (d, 3H), 0.58 (d, 2H), 2.22 (m, 1H), 3.44 (s, 3H), 3.92 (s, 3H), 5.39 (m, 1H), 5.41 (m, 2H), 6.13 (s, 2H), 6.51 (d, 2H), 6.85 (m, 2H), 7.10 (m, 2H), 7.14 (s, 1H), 7.47 (s, 1H), 7.93 (s, 1H).

### [Example 279] Synthesis of 6-(6,7-dimethoxy-3-oxo-1,3-dihydronaphtho[2,3-c]furan-4-yl)benzo[d][1,3]dioxol-5-yl phenyl (2-morpholinoethyl)phosphoramidate

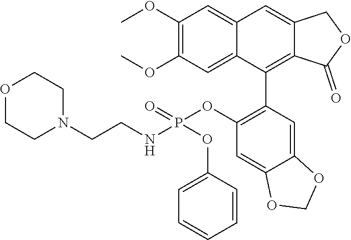 [SMILES: COc1cc2cc3c(c(-c4cc5c(cc4OP(=O)(NCCN4CCOCC4)Oc4ccccc4)OCO5)c2cc1OC)C(=O)OC3]

The compound of Example 279 was synthesized by the same method as that of the compound of Example 278 except for using 4-(2-aminoethyl)morpholine instead of isopropylamine.

Mass (M+H⁺): 649.2

¹H-NMR (500 MHz, DMSO-d6): 1.88 (m, 2H), 2.04 (m, 4H), 2.22 (m, 2H), 3.44 (m, 4H), 3.48 (s, 3H), 3.92 (s, 3H), 5.36 (m, 1H), 5.40 (dd, 2H), 6.13 (s, 2H), 6.54 (d, 2H), 6.83 (d, 2H), 7.02 (t, 1H), 7.08 (m, 2H), 7.21 (s, 1H), 7.46 (s, 1H), 7.92 (s, 1H).

### [Example 280] Synthesis of 6-(6,7-dimethoxy-3-oxo-1,3-dihydronaphtho[2,3-c]furan-4-yl)benzo[d][1,3]dioxol-5-yl N-(2-morpholinoethyl)-P-phenylphosphoramidate

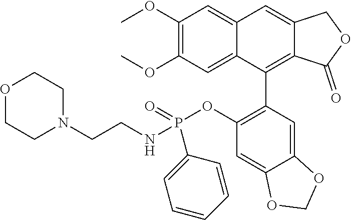 [SMILES: COc1cc2cc3c(c(-c4cc5c(cc4OP(=O)(NCCN4CCOCC4)c4ccccc4)OCO5)c2cc1OC)C(=O)OC3]

The compound of Example 280 was synthesized by the same method as that of the compound of Example 279 except for using phenyl phosphonic dichloride instead of phenyl dichlorophosphate.

Mass (M+H⁺): 633.2

¹H-NMR (500 MHz, DMSO-d6): 2.07 (m, 2H), 2.11 (m, 4H), 2.52 (m, 2H), 3.43 (m, 4H), 3.60 (s, 3H), 3.91 (s, 3H), 5.06 (m, 1H), 5.39 (dd, 2H), 6.12 (s, 2H), 6.77 (m, 2H), 6.83 (s, 1H), 6.85 (s, 1H), 7.01 (m, 2H), 7.27 (m, 2H), 7.44 (s, 1H), 7.92 (s, 1H).

### [Example 281] Synthesis of 6-(6,7-dimethoxy-3-oxo-1,3-dihydronaphtho[2,3-c]furan-4-yl)benzo[d][1,3]dioxol-5-yl N-isopropyl-P-phenylphosphoramidate

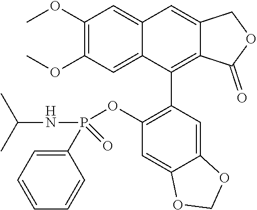 [SMILES: COc1cc2cc3c(c(-c4cc5c(cc4OP(=O)(NC(C)C)c4ccccc4)OCO5)c2cc1OC)C(=O)OC3]

The compound of Example 281 was synthesized by the same method as that of the compound of Example 278 except for using phenyl phosphonic dichloride instead of phenyl dichlorophosphate.

Mass (M+H⁺): 562.2

¹H-NMR (500 MHz, DMSO-d6): 0.75 (d, 3H), 0.80 (d, 3H), 2.79 (m, 1H), 3.56 (s, 3H), 3.92 (s, 3H), 5.06 (t, 1H), 5.39 (dd, 2H), 6.11 (d, 2H), 6.75 (s, 1H), 6.81 (m, 3H), 6.99 (m, 2H), 7.20 (s, 1H), 7.27 (t, 1H), 7.44 (s, 1H), 7.91 (s, 1H).

### Experimental Example 1. Comparative Experiment for Inhibitory Effect on SARS-COV-2 Infection in Vero Cell Line

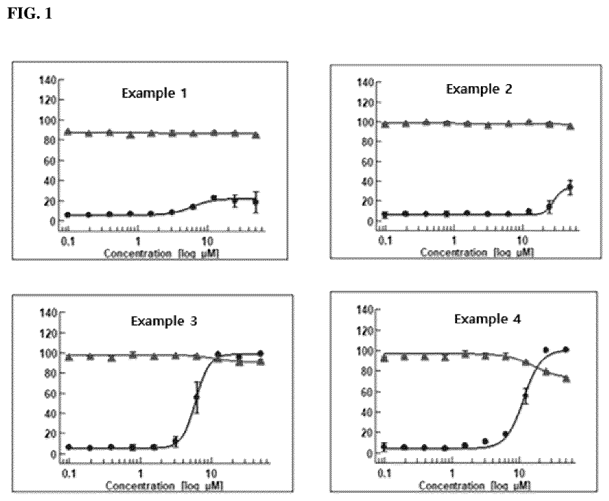

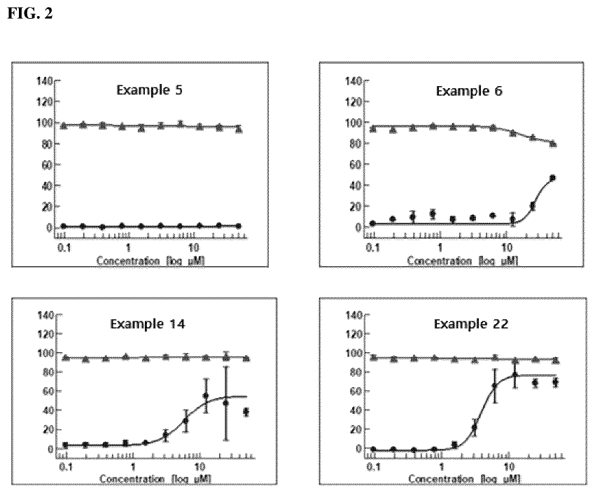

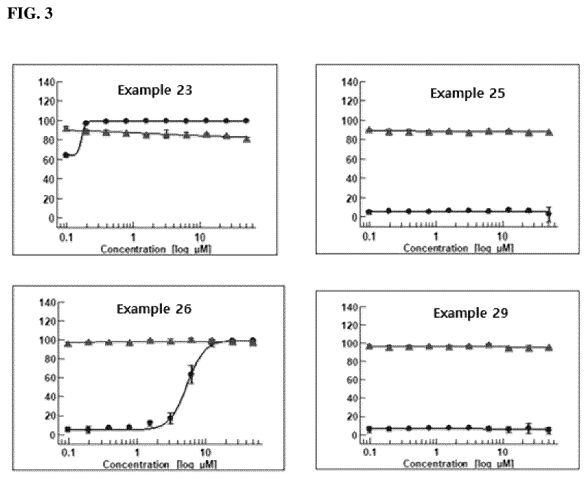

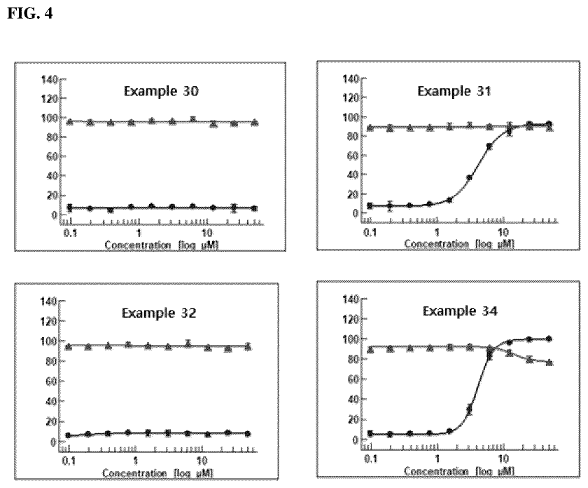

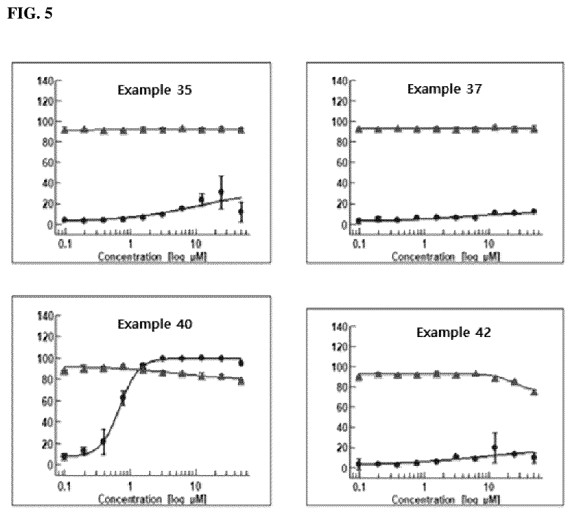

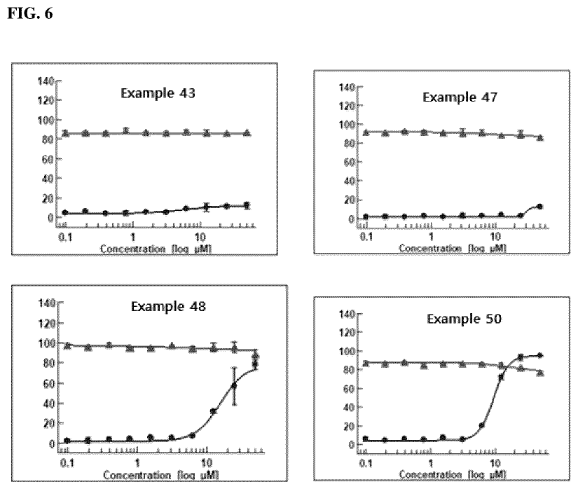

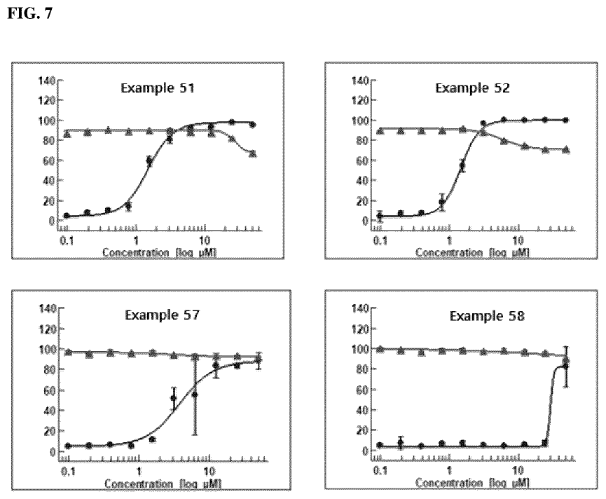

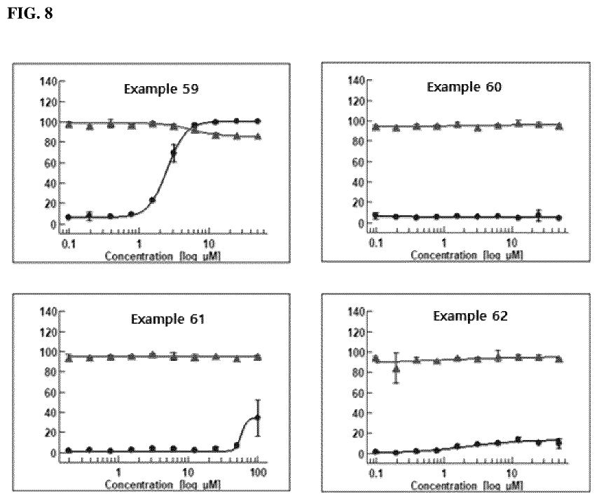

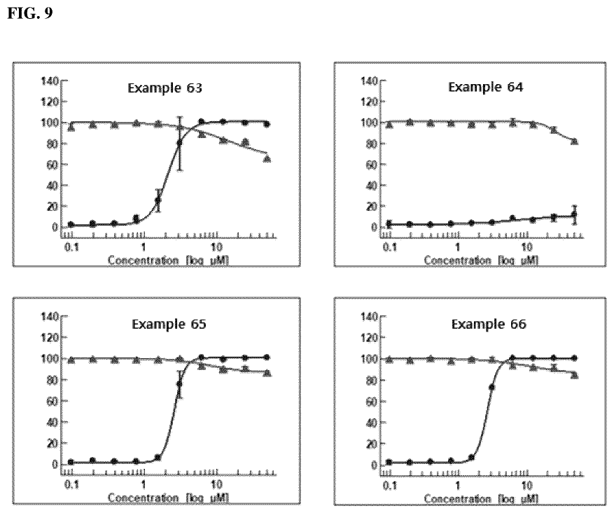

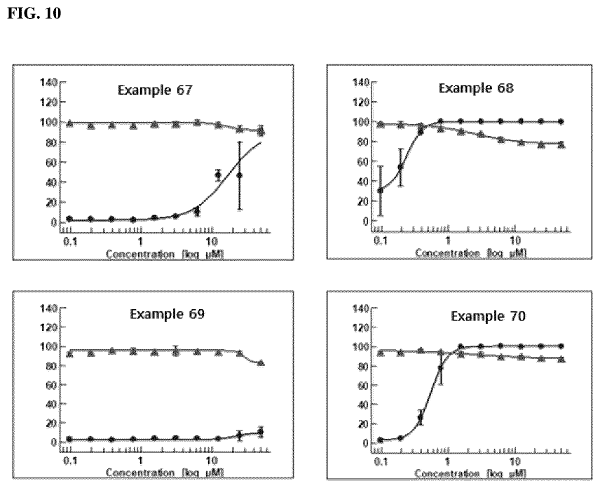

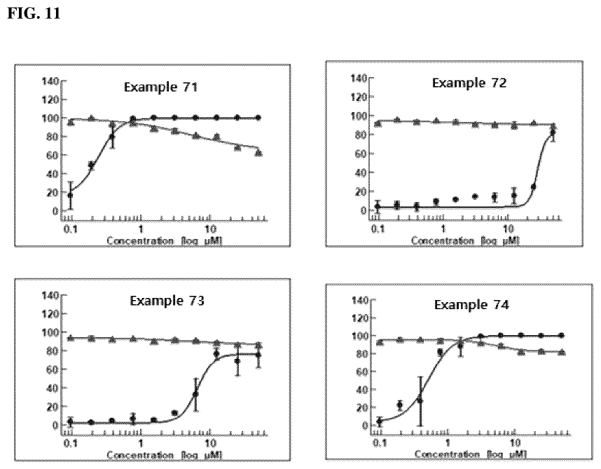

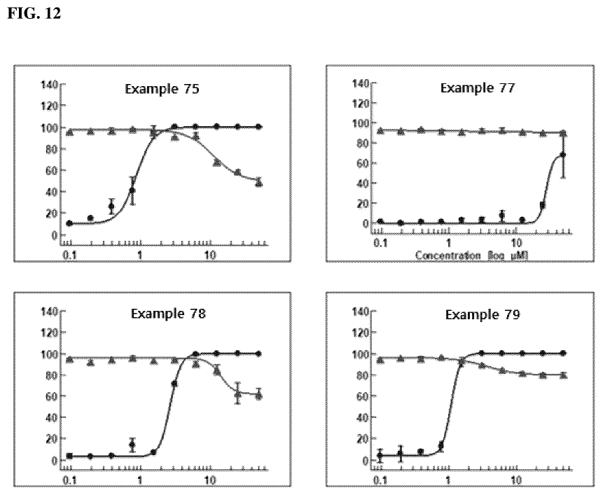

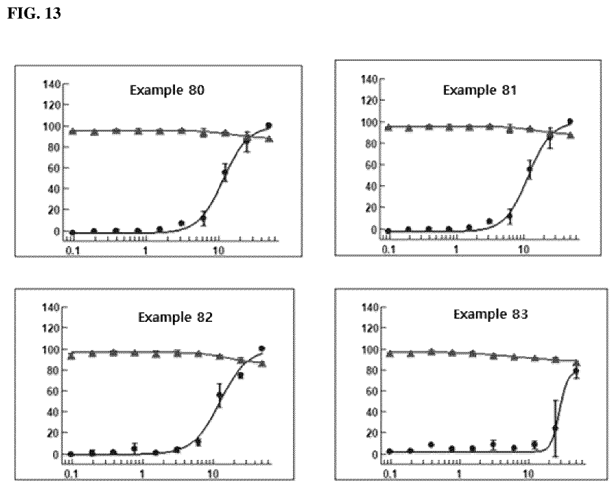

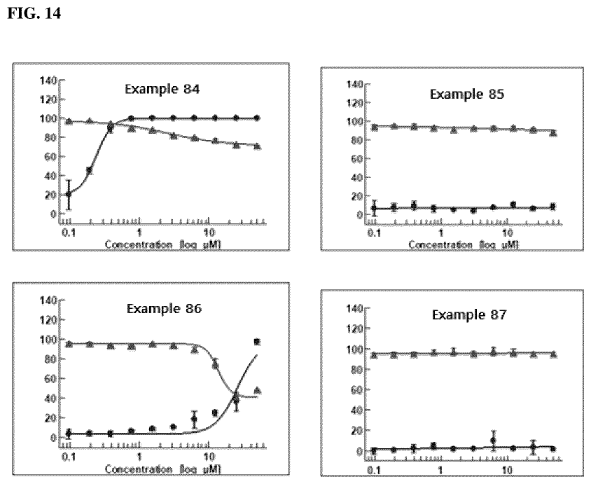

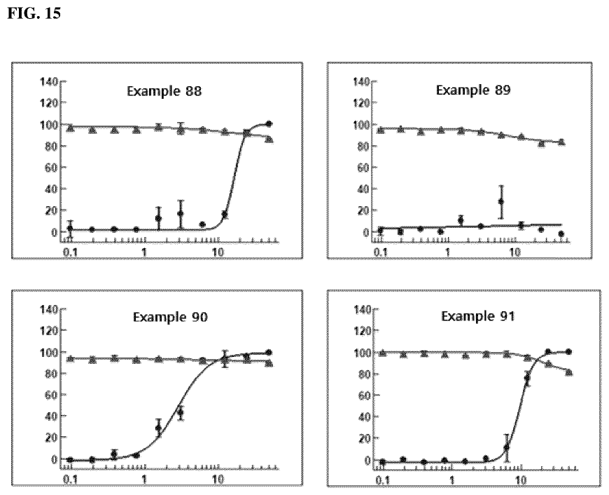

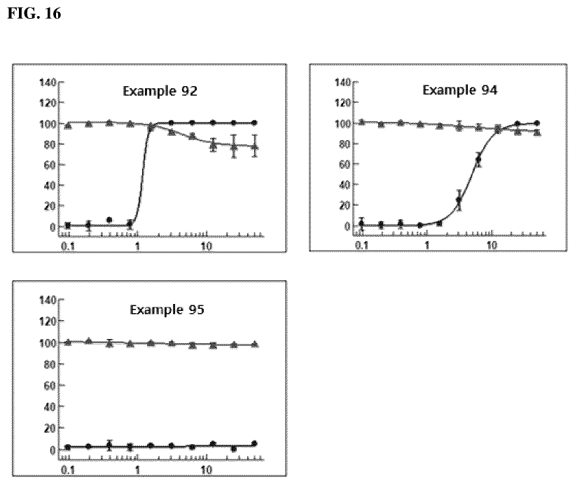

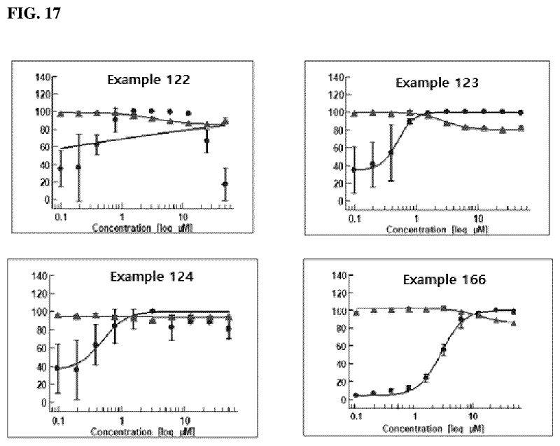

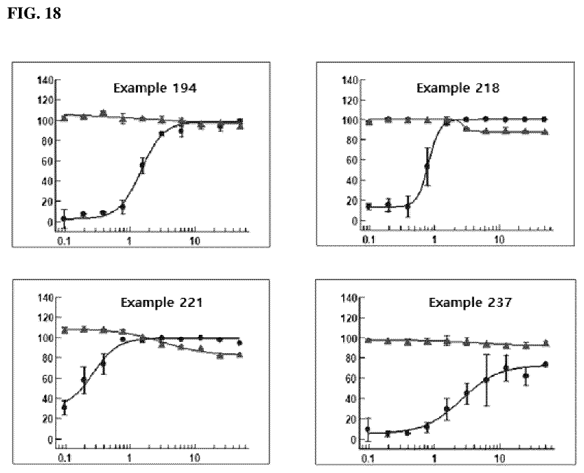

In order to confirm the inhibitory effect of the novel 6,7-dimethoxynaphtho[2,3-c]furan-1(3H)-one derivatives against SARS-COV-2 infection, the Vero cell line was cultured for 24 hours, dosed with example compounds at ten concentrations ranging from 0.1 micro M (micro gram) to 50 micro M, and infected with the SARS-COV-2 provided from the Korea Centers for Disease Control and Prevention (KCDC). Specifically, the Vero cells were seeded at 1.2×10⁴ per well into a 384-well tissue culture plate. On the next day, the compound at a concentration of 50 micro M was serially diluted two-fold to prepare compounds at ten concentrations and treated with Vero cells. Soon after, the cells treated with the compound were infected with SARS-COV-2 (COVID 19) and cultured at 37° C. for 24 hours. Then, the cells were fixed and permeabilized. After that, the cells were treated with anti-SARS-COV-2 Nucleocapsid (N) primary antibody and then stained by treatment with Alexa Fluor 488-conjugated IgG secondary antibody and Hoechst 33342. Fluorescent expression was obtained using an Operetta large image analyzer (Perkin Elmer). The resulting image were used with Columbus software to calculate the percentage of infected cells and measure the inhibitory efficacy of the drug, which were then summarized in Table 2, and the response curve and 50% inhibitory effect (IC₅₀) values according to the drug concentration are derived with XLFit (IDBS) software, and the results and cytotoxicity (CC₅₀) of the test substances are summarized in Table 1 and FIGS. 1 to 18. FIGS. 1 to 18 are graphs showing the SARS-COV inhibitory activity and cytotoxicity of some of the drugs of each Example listed in Table 1 below. In the graphs of FIGS. 1 to 18, a horizontal axis represents the drug concentration (μM) expressed by log values, and a vertical axis represents the SARS-COV-2 inhibition rate (%) and the cell survival rate (%) (in FIGS. 1 to 18. ▴ represents CC₅₀, and ● represents IC₅₀).

TABLE 1
SARS-COV-2 virus inhibitory ability
and cytotoxicity of compound

| | SARS-COV-2 inhibition | Cytotoxicity | SI |
|---|---|---|---|
| Compound | (IC₅₀, μM) | (CC₅₀, μM) | (μM) |
| Example 1 | >50 | >50 | 1.00 |
| Example 2 | >50 | >50 | 1.00 |
| Example 3 | 5.89 | >50 | 8.48 |
| Example 4 | 11.22 | >50 | 4.45 |
| Example 5 | >50 | >50 | 1.00 |
| Example 6 | >50 | >50 | 1.00 |
| Example 7 | 13.38 | >50 | 3.74 |
| Example 8 | 2.87 | >50 | 17.39 |
| Example 10 | 0.55 | >50 | 91.58 |
| Example 11 | 0.16 | >50 | 309.03 |
| Example 12 | 0.83 | >50 | 60.38 |
| Example 13 | >50 | >50 | 1.00 |
| Example 14 | 15.11 | >50 | 3.31 |
| Example 15 | 4.40 | >50 | 11.37 |
| Example 16 | 31.18 | >50 | 1.6 |
| Example 17 | 31.37 | >50 | 1.59 |
| Example 18 | 0.50 | >50 | 99.07 |
| Example 19 | 31.32 | >50 | 1.60 |
| Example 20 | >50 | >50 | 1.00 |
| Example 21 | >50 | >50 | 1.00 |
| Example 22 | 4.70 | >50 | 10.64 |
| Example 23 | 0.08 | >50 | 61.34 |
| Example 24 | >50 | >50 | 1.00 |
| Example 25 | >50 | >50 | 1.00 |
| Example 26 | 5.25 | >50 | 9.52 |
| Example 28 | >50 | >50 | 1.00 |
| Example 29 | >50 | >50 | 1.00 |
| Example 30 | >50 | >50 | 1.00 |
| Example 31 | 4.22 | >50 | 11.86 |
| Example 32 | >50 | >50 | 1.00 |
| Example 33 | >50 | >50 | 1.00 |
| Example 34 | 4.03 | >50 | 12.40 |
| Example 35 | >50 | >50 | 1.00 |
| Example 37 | >50 | >50 | 1.00 |
| Example 39 | 13.60 | >50 | 1.98 |
| Example 40 | 0.65 | >50 | 76.71 |
| Example 41 | 15.24 | >50 | 2.41 |
| Example 42 | >50 | >50 | 1.00 |
| Example 43 | >50 | >50 | 1.00 |
| Example 46 | 0.10 | >50 | 512.00 |
| Example 47 | >50 | >50 | 1.00 |
| Example 48 | 19.62 | >50 | 2.55 |
| Example 49 | >50 | >50 | 1.00 |
| Example 50 | 9.49 | >50 | 5.27 |
| Example 51 | 1.47 | >50 | 34.12 |
| Example 52 | 1.42 | >50 | 35.13 |
| Example 53 | 2.51 | >50 | 19.94 |
| Example 54 | >50 | >50 | 1.00 |
| Example 55 | 11.81 | >50 | 4.23 |
| Example 57 | 4.14 | >50 | 12.07 |
| Example 58 | 30.05 | >50 | 1.66 |
| Example 59 | 2.40 | >50 | 20.79 |
| Example 60 | >50 | >50 | 1.00 |
| Example 61 | >50 | >50 | 1.00 |
| Example 62 | >50 | >50 | 1.00 |
| Example 63 | 2.12 | >50 | 23.56 |
| Example 64 | >50 | >50 | 1.00 |
| Example 65 | 2.59 | >50 | 19.30 |
| Example 66 | 2.63 | >50 | 19.03 |
| Example 67 | 18.40 | >50 | 2.72 |
| Example 68 | 0.18 | >50 | 270.89 |
| Example 69 | >50 | >50 | 1.00 |
| Example 70 | 0.54 | >50 | 92.84 |
| Example 71 | 0.21 | >50 | 234.36 |
| Example 72 | 30.41 | >50 | 1.64 |
| Example 73 | 7.66 | >50 | 6.53 |
| Example 74 | 0.51 | >50 | 98.94 |
| Example 75 | 0.83 | >50 | 60.56 |
| Example 76 | 0.21 | >50 | 270.89 |
| Example 77 | 31.55 | >50 | 1.58 |
| Example 78 | 2.66 | >50 | 18.80 |
| Example 79 | 1.08 | >50 | 46.19 |
| Example 80 | 11.89 | >50 | 4.21 |
| Example 81 | 20.59 | >50 | 2.43 |
| Example 82 | 12.72 | >50 | 3.93 |
| Example 83 | 30.28 | >50 | 1.65 |
| Example 84 | 0.21 | >50 | 237.53 |
| Example 85 | >50 | >50 | 1.00 |
| Example 86 | 25.67 | 19.25 | 0.75 |
| Example 87 | >50 | >50 | 1.00 |
| Example 88 | 16.65 | >50 | 3.00 |
| Example 89 | >50 | >50 | 1.00 |
| Example 90 | 2.95 | >50 | 16.96 |
| Example 91 | 9.70 | >50 | 5.15 |
| Example 92 | 1.18 | >50 | 42.34 |
| Example 94 | 4.92 | >50 | 10.16 |
| Example 95 | >50 | >50 | 1.00 |
| Example 96 | 1.84 | >50 | 27.11 |
| Example 97 | 5.72 | >50 | 8.74 |
| Example 98 | 15.73 | >50 | 3.18 |
| Example 99 | 5.75 | >50 | 8.70 |
| Example 100 | 0.52 | >50 | 96.21 |
| Example 101 | 3.85 | >50 | 13.00 |
| Example 102 | 1.64 | >50 | 30.41 |
| Example 103 | 0.29 | >50 | 175.17 |
| Example 104 | >50 | >50 | 1.0 |
| Example 105 | 5.23 | >50 | 9.56 |
| Example 106 | 0.17 | >50 | 297.58 |
| Example 107 | 1.41 | >50 | 35.49 |
| Example 108 | 2.77 | >50 | 18.03 |
| Example 109 | 2.67 | >50 | 18.73 |
| Example 110 | 8.66 | >50 | 5.78 |
| Example 111 | 21.09 | >50 | 2.37 |
| Example 112 | 7.46 | >50 | 6.70 |
| Example 113 | 0.99 | >50 | 50.52 |
| Example 114 | >50 | >50 | 1.0 |
| Example 115 | 1.03 | >50 | 48.62 |
| Example 116 | 1.14 | >50 | 43.72 |
| Example 117 | >50 | >50 | 1.0 |
| Example 118 | 0.19 | >50 | 257.92 |
| Example 119 | 4.36 | >50 | 11.45 |
| Example 120 | 4.14 | >50 | 12.07 |
| Example 121 | 2.32 | >50 | 21.51 |
| Example 122 | 0.17 | >50 | 512.00 |
| Example 123 | 0.34 | >50 | 145.26 |
| Example 124 | 0.28 | >50 | 176.18 |
| Example 125 | >50 | >50 | 1.0 |
| Example 126 | 0.41 | >50 | 122.97 |
| Example 127 | 1.1 | >50 | 45.29 |
| Example 128 | 28.04 | >50 | 1.78 |
| Example 129 | 21.8 | >50 | 2.29 |
| Example 130 | >50 | >50 | 1.0 |
| Example 131 | 34.65 | >50 | 1.44 |
| Example 132 | >50 | >50 | 1.0 |
| Example 133 | >50 | >50 | 1.0 |
| Example 134 | >50 | >50 | 1.0 |
| Example 135 | 17.22 | >50 | 2.90 |
| Example 136 | >50 | >50 | 1.0 |
| Example 137 | >50 | >50 | 1.0 |
| Example 138 | 37.66 | >50 | 1.33 |
| Example 139 | >50 | >50 | 1.0 |
| Example 140 | >50 | >50 | 1.0 |
| Example 141 | 24.15 | >50 | 2.07 |
| Example 142 | 2.00 | >50 | 25.00 |
| Example 143 | >50 | >50 | 1.0 |
| Example 144 | >50 | >50 | 1.0 |
| Example 146 | >50 | >50 | 1.0 |
| Example 147 | >50 | >50 | 1.0 |
| Example 148 | >50 | >50 | 1.0 |
| Example 149 | >50 | >50 | 1.0 |
| Example 150 | >50 | >50 | 1.0 |
| Example 151 | >50 | >50 | 1.0 |
| Example 152 | 23.80 | >50 | 2.10 |
| Example 153 | >50 | >50 | 1.0 |
| Example 154 | >50 | >50 | 1.0 |
| Example 155 | >50 | >50 | 1.0 |
| Example 156 | 1.02 | >50 | 48.93 |
| Example 157 | >50 | >50 | 1.0 |
| Example 158 | 6.89 | >50 | 7.26 |
| Example 159 | 1.52 | >50 | 32.86 |
| Example 160 | 0.83 | >50 | 60.45 |
| Example 161 | 6.46 | >50 | 7.74 |
| Example 162 | 4.30 | >50 | 11.63 |
| Example 163 | 0.06 | >50 | 885.42 |
| Example 164 | 1.28 | >50 | 39.02 |
| Example 165 | 2.91 | 34 | 11.69 |
| Example 166 | 2.70 | >50 | 18.50 |
| Example 167 | 14.76 | >50 | 3.73 |
| Example 168 | 1.18 | >50 | 42.46 |
| Example 169 | 1.23 | >50 | 40.73 |
| Example 170 | 2.06 | >50 | 24.25 |
| Example 171 | 0.06 | >50 | 885.42 |
| Example 172 | >50 | >50 | 1.0 |
| Example 173 | >50 | >50 | 1.0 |
| Example 174 | 19.11 | >50 | 2.62 |
| Example 175 | 1.19 | >50 | 42.02 |
| Example 176 | 2.21 | >50 | 22.63 |
| Example 177 | 0.28 | >50 | 180.12 |
| Example 178 | 0.04 | >50 | 1323.43 |
| Example 179 | 1.02 | >50 | 49.02 |
| Example 180 | 0.68 | >50 | 73.59 |
| Example 181 | 0.93 | >50 | 53.67 |
| Example 182 | 0.66 | >50 | 75.67 |
| Example 183 | 0.40 | >50 | 124.83 |
| Example 184 | >50 | >50 | 1.0 |
| Example 185 | 0.33 | >50 | 149.26 |
| Example 186 | >50 | >50 | 1.0 |
| Example 187 | >50 | >50 | 1.0 |
| Example 188 | 0.04 | >50 | 1418.97 |
| Example 189 | 0.69 | >50 | 72.26 |
| Example 190 | 28.96 | >50 | 1.73 |
| Example 191 | 1.41 | >50 | 35.47 |
| Example 192 | 4.96 | >50 | 10.08 |
| Example 193 | 7.74 | >50 | 6.46 |
| Example 194 | 1.49 | >50 | 33.63 |
| Example 195 | 5.27 | >50 | 9.50 |
| Example 196 | 2.43 | >50 | 20.54 |
| Example 197 | 17.37 | >50 | 2.88 |
| Example 198 | 1.03 | >50 | 48.47 |
| Example 199 | 1.98 | >50 | 25.20 |
| Example 200 | 28.24 | >50 | 1.77 |
| Example 201 | 1.27 | >50 | 39.52 |
| Example 202 | 1.01 | 38.35 | 38.01 |
| Example 203 | 3.54 | 29.74 | 8.39 |
| Example 204 | 25.94 | 24.2 | 0.93 |
| Example 205 | 6.46 | >50 | 7.75 |
| Example 206 | 17.92 | >50 | 2.79 |
| Example 207 | 6.60 | >50 | 7.57 |
| Example 208 | 2.64 | >50 | 18.96 |
| Example 209 | 3.44 | >50 | 14.52 |
| Example 210 | 10.17 | >50 | 4.91 |
| Example 211 | 4.00 | >50 | 12.50 |
| Example 212 | 17.81 | >50 | 2.81 |
| Example 213 | 4.41 | >50 | 2.81 |
| Example 214 | >50 | >50 | 1.00 |
| Example 215 | 19.58 | >50 | 2.55 |
| Example 216 | 0.96 | >50 | 52.13 |
| Example 217 | 4.97 | >50 | 10.06 |
| Example 218 | 0.76 | >50 | 65.75 |
| Example 219 | >50 | >50 | 1.00 |
| Example 220 | >50 | >50 | 1.00 |
| Example 221 | 0.18 | >50 | 275.32 |
| Example 222 | 0.31 | >50 | 162.21 |
| Example 223 | 16.10 | >50 | 3.11 |
| Example 224 | 1.9 | >50 | 2.63 |
| Example 225 | >50 | 32.36 | 0.65 |
| Example 226 | 6.69 | >50 | 7.47 |
| Example 227 | 0.9 | >50 | 55.59 |
| Example 228 | 33.37 | >50 | 1.50 |
| Example 229 | 2.84 | >50 | 17.63 |
| Example 230 | 20.4 | >50 | 2.45 |
| Example 231 | >50 | >50 | 1.00 |
| Example 232 | 3.25 | >50 | 15.36 |
| Example 233 | >50 | >50 | 1.00 |
| Example 234 | >50 | >50 | 1.00 |
| Example 235 | >50 | >50 | 1.00 |
| Example 236 | 1.65 | >50 | 30.25 |
| Example 237 | 0.81 | >50 | 61.98 |
| Example 238 | 17.61 | >50 | 2.84 |
| Example 239 | >50 | >50 | 1.00 |
| Example 240 | >50 | >50 | 1.00 |
| Example 241 | >50 | >50 | 1.00 |
| Example 242 | 1.94 | 10.55 | 5.45 |
| Example 243 | 15.33 | >50 | 3.26 |
| Example 244 | >50 | >50 | 1.00 |
| Example 245 | >50 | >50 | 1.00 |
| Example 246 | 3.29 | >50 | 15.18 |
| Example 247 | 0.15 | >50 | 344.5 |
| Example 248 | <0.098 | >50 | 344.5 |
| Example 249 | 0.5 | >50 | 100.23 |
| Example 250 | 4.81 | >50 | 10.40 |
| Example 251 | 0.42 | >50 | 119.39 |
| Example 252 | >50 | >50 | 1 |
| Example 253 | 2.43 | >50 | 20.54 |
| Example 254 | 4.11 | >50 | 12.17 |
| Example 255 | 3.18 | >50 | 15.74 |
| Example 256 | 0.61 | >50 | 82.38 |
| Example 257 | 8.01 | >50 | 6.24 |
| Example 258 | 16.46 | >50 | 3.04 |
| Example 259 | >50 | >50 | 1.00 |
| Example 260 | >50 | >50 | 1.00 |
| Example 261 | 11.89 | >50 | 4.21 |
| Example 262 | 9.26 | >50 | 5.40 |
| Example 263 | >50 | >50 | 1.00 |
| Example 264 | >50 | >50 | 1.00 |
| Example 265 | 33.03 | >50 | 1.51 |
| Example 266 | >50 | >50 | 1.00 |
| Example 267 | >50 | >50 | 1.00 |
| Example 268 | >50 | >50 | 1.00 |
| Example 269 | 0.53 | >50 | 94.66 |
| Example 270 | 0.41 | >50 | 122.64 |
| Example 271 | 1.45 | >50 | 34.41 |
| Example 272 | 1.84 | >50 | 27.23 |
| Example 273 | 1.37 | >50 | 36.52 |
| Example 274 | 2.55 | >50 | 19.61 |
| Example 275 | 28.44 | >50 | 1.76 |
| Example 276 | >50 | >50 | 1.00 |
| Example 277 | >50 | >50 | 1.00 |
| Example 278 | >50 | >50 | 1.00 |
| Example 279 | 6.09 | >50 | 8.21 |
| Example 280 | 1.96 | >50 | 25.56 |
| Example 281 | 14.50 | >50 | 3.45 |

**Selectivity Index (SI): a value obtained by dividing a value of cytotoxicity (CC₅₀) (assumed to be 50, if >50) by a value of SARS-COV-2 inhibition (IC₅₀) (assumed to be 50, if >50).

As confirmed in Table 1 and FIGS. 1 to 18, the Example compounds show excellent SARS-COV-2 inhibitory activity and low toxicity. Thus, it is confirmed that the compound of one Example exhibits an excellent anti-viral effect against SARS-COV-2, and may exhibit excellent preventive or therapeutic effects against SARS-COV-2 infection disease.

### Experimental Example 2. Comparative Experiment for Inhibition of IL-5 Expression in Th2 Cells

In order to confirm the inhibitory effect of the novel 6,7-dimethoxynaphtho[2,3-c]furan-1(3H)-one derivatives against IL-5 expression, Th2 cells were cultured using mouse splenocytes, and dosed with the test drugs prepared in Examples 1 to 95 at a concentration of 1 nM, so that IL-5 was measured through an ELISA test. Specifically, after spleens were collected from Balb/C mice, CD4+ T cells were isolated therefrom and were seeded at 1-2×10⁶ per well into a 48-well tissue culture plate coated with anti-CD3 (5 ug/ml) and anti-CD28 (1 ug/ml). At this time, the culture medium was incubated with IL-2 (20 ng/ml), IL-4 (100 ng/ml), anti-IFNγ (10 ug/ml), anti-IL12 (10 ug/ml), and a derivative of 1 nM for two days, transferred to a new tissue culture plate, and further cultured for three days to induce differentiation into Th2 cells. After replacing with a new medium containing anti-IFNγ (10 ug/ml), anti-IL12 (10 ug/ml) and a derivative of 1 nM, polarization of Th2 cells was induced using PMA and A23187. After two days of culture, the cells were centrifuged and the culture medium was taken to perform IL-5 ELISA. IL-5 ELISA was performed according to the test method of the kit manufacturer. As a result, an inhibitory ratio of IL-5 expression was derived with Th0 cells, which did not induce Th2 differentiation, as 100% inhibition and with Th2 cells, which were not treated with the novel derivative, as a control for 0% inhibition.

TABLE 2
Inhibitory Ability of Compound against IL-5 Expression (%)

| | Inhibitory | | Inhibitory |
|---|---|---|---|
| Compound | ability (%) | Compound | ability (%) |
| Example 1 | 61.9 | Example 13 | 67.7 |
| Example 14 | 58.9 | Example 15 | 82.9 |
| Example 16 | 70.8 | Example 17 | 55.2 |
| Example 18 | 72.3 | Example 19 | 92.3 |
| Example 20 | 40.8 | Example 22 | 59.9 |
| Example 23 | 68.2 | Example 24 | 72.3 |
| Example 25 | 56.8 | Example 37 | 56.0 |
| Example 38 | 62.8 | Example 39 | 47.7 |
| Example 40 | 65.1 | Example 41 | 66.0 |
| Example 42 | 64.8 | Example 43 | 65.9 |
| Example 44 | 58.3 | Example 45 | 64.2 |
| Example 46 | 50.4 | Example 47 | 61.0 |
| Example 48 | 58.1 | Example 50 | 61.5 |

As confirmed in Table 2, it was found that the Example compounds show excellent inhibitory activity against IL-5 expression. As a result, it was confirmed that the Example compounds show excellent an anti-allergic effect and an asthma treatment effect.

### Experimental Example 3. Comparative Experiment for Inhibition of Novel 6,7-dimethoxynaphtho[2,3-c]furan-1(3H)-one Derivatives Against IL-4 Secretion

In order to confirm the inhibitory effect of the novel 6,7-dimethoxynaphtho[2,3-c]furan-1(3H)-one derivatives against IL-4 expression, the RBL-2H3 cell line was used to measure the ability for inhibiting IL-4 secretion. The RBL-2H3 cell line was purchased from the American Type Culture Collection (ATCC #CRL-2256). The cells were diluted at a concentration of 1×10⁶ cells/ml in RPMI medium containing 15% fetal bovine serum (FBS) and antibiotics (penicillin at 100 U/mL, streptomycin at 100 μg/mL), distributed at 200 μl/well in a 48 well plate, and cultured in a 37° C., 5% CO₂ incubator for 24 hours. In 24 hours later, the cells were pre-treated one hour before with a test drug and AS1517499 (STAT6 inhibitor) as a control drug, and treated with 1 uM of A23187 for six hours to induce an immune response, after which a group not containing the drug was used as a control. In order to measure a secretion level of cytokine IL-4, the medium was collected from the well plate and centrifuged at 125×g for five minutes to remove a residue, and 100 μl of the medium was used to measure an amount of IL-4 secreted from the cells by using IL-4 enzyme-linked immunosorbent assay (ELISA, Komabiotech). A degree of inhibition of cell secretion by the drug was calculated using [Equation 1] below.

$$IL - 4 secretion inhibitory ability ( \% ) = 100 - \frac{Test group IL - 4 concentration}{Control group IL - 4 concentration} ⨯ 100 \qquad [ Equation 1 ]$$

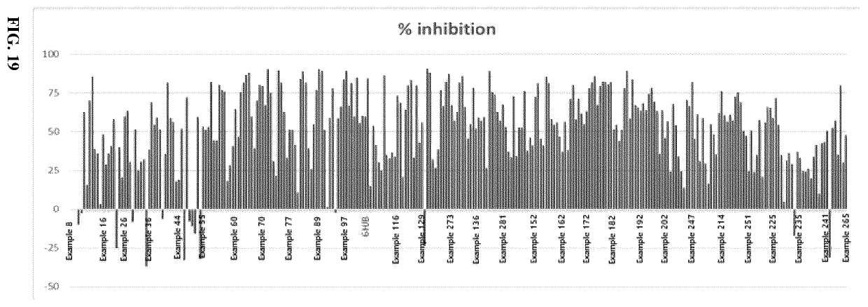

First of all, 288 kinds of materials were treated at a single concentration of 10 uM so as to select 60 kinds of materials which inhibit secretion of IL-4 by 75% or more (FIG. 19).

After that, a repeated experiment and a two-concentration (10, 3 uM) test were performed using the selected 60 kinds of materials so as to select the final eight kinds, and the IC₅₀ value was calculated and shown in Table 3.

TABLE 3

| compound | IC₅₀ (μM) |
|---|---|
| Example 59 | 2.35 |
| Example 63 | 4.23 |
| Example 65 | 2.18 |
| Example 76 | 4.05 |
| Example 73 | 3.19 |
| Example 86 | 2.21 |
| Example 84 | 5.23 |
| Example 169 | 2.34 |

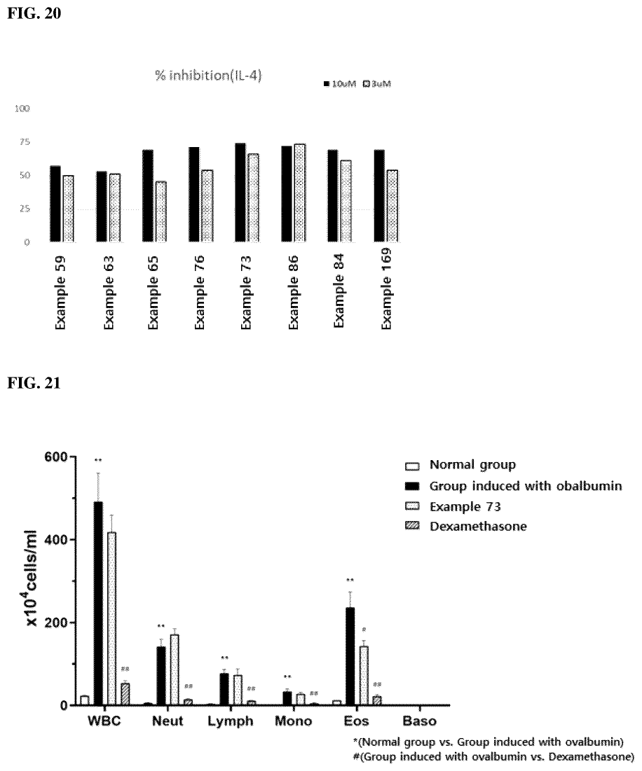

In addition, the IL-4 secretion inhibitory activity (%) in RBL-2H3 cells upon 10, 3 uM treatment is shown in FIG. 20.

### Experimental Example 4. Comparative Experiment for Inhibitory Effect of Novel 6,7-dimethoxynaphtho[2,3-c]furan-1(3H)-one Derivatives Against Influenza Virus Standard Strains (A/Puerto Rico/08/1934, H1N1)

In order to confirm the inhibitory effect of the novel 6,7-dimethoxynaphtho[2,3-c]furan-1(3H)-one derivatives against infection with influenza virus standard strain H1N1, MDCK cell line was cultured for 24 hours, and then the example compounds were administered at five concentrations ranging from 0.01 micro M to 100 micro M, and infected with A/Puerto Rico/08/1934 (A/PR/8/34; H1N1). Specifically, the MDCK cells were seeded at 2.0×10⁴ per well into a 96-well tissue culture plate. On the next day, the compound at a concentration of 100 micro M was serially diluted ten-fold to prepare compounds at five concentrations and treated with the MDCK cells. Soon after, the cells treated with the compound were infected with H1N1 and cultured at 37° C. for 66 hours. After that, the cells were treated with EZ-CYTOX and the survival of living cells was measured at UV450 nm in three to four hours later.

The results and the 50% half maximal effective concentration (EC₅₀) and cytotoxicity (CC₅₀) of the test materials are summarized in Table 4.

TABLE 4
Inhibitory activity and cytotoxicity of compound against
influenza virus standard strain H1N1 (A/PR/8/34; H1N1)

| | H1N1 inhibition | Cytotoxicity | SI |
|---|---|---|---|
| Compound | (EC₅₀, μM) | (CC₅₀, μM) | (μM) |
| Example 10 | 0.170 | 1.226 | 7.212 |
| Example 51 | 0.107 | 1.761 | 16.460 |
| Example 52 | 1.885 | 4.471 | 2.372 |
| Example 123 | 0.198 | 2.031 | 10.258 |
| Example 164 | 17.250 | >100 | 5.800 |
| Example 170 | 1.671 | 6.818 | 4.080 |
| Example 179 | 0.178 | 1.629 | 9.152 |
| Example 180 | —* | —* | —* |
| Example 221 | 0.249 | 0.752 | 3.020 |

*Not able to be determined

As confirmed in Table 4, it was found that the example compounds show excellent inhibitory activity against influenza virus standard strain H1N1. Thus, it is confirmed that the example compounds exhibit an excellent anti-viral effect against influenza virus standard strain H1N1. From this, it is confirmed that the compounds may exhibit excellent preventive or therapeutic effects against an influenza virus infection disease.

### Experimental Example 5. Comparative Experiment for Inhibitory Effect of Novel 6,7-dimethoxynaphtho[2,3-c]furan-1(3H)-one Derivatives Against Dengue Virus and Zika Virus

In order to confirm the inhibitory effect of the novel 6,7-dimethoxynaphtho[2,3-c]furan-1(3H)-one derivatives against infection with dengue virus, Vero E6 cell line was cultured for 24 hours, and then the compound of Example 221 was administered at eight concentrations ranging from 0.0625 micro M to 3 micro M, and infected with dengue virus. Specifically, the Vero cells were seeded at 2.5×10⁴ per well into a 96-well tissue culture plate. On the next day, a compound having a concentration of 0.0625 micro M was prepared from a compound having a concentration of 3 micro M and treated to Vero E6 cells, and then the cells treated with the compound were immediately infected with dengue virus (0.01 MOI) and cultured at 37° C. for five days.

In addition, in order to confirm the inhibitory effect against infection with Zika virus, Vero cell line was cultured for 24 hours, and then the compound of Example 221 was administered at eight concentrations ranging from 0.25 micro M to 5 micro M, and infected with Zika virus. Specifically, the Vero cells were seeded at 2.5×10⁴ per well into a 96-well tissue culture plate. On the next day, a compound having a concentration of 0.25 micro M was prepared from a compound having a concentration of 5 micro M and treated to Vero cells, and then the cells treated with the compound were immediately infected with Zika virus (0.02 MOI) and cultured at 37° C. for five days.

After that, the cells were treated with EZ-CYTOX and the survival of living cells was measured at UV450 nm in three to four hours later.

The results and the cytotoxicity (CC₅₀) of the test materials are summarized in Table 5.

TABLE 5
Inhibitory activity and cytotoxicity of compound of
Example 221 against dengue virus and Zika virus

| | Dengue virus | | | Zika virus | | |
|---|---|---|---|---|---|---|
| | Inhibitory | | | Inhibitory | | |
| | ability | Cytotoxicity | SI | ability | Cytotoxicity | SI |
| Compound | (EC₅₀, μM) | (CC₅₀, μM) | (μM) | (EC₅₀, μM) | (CC₅₀, μM) | (μM) |
| Example 221 | 0.079 | 0.292 | 3.70 | 0.484 | 7.584 | 15.67 |

It was confirmed that the compound of Example 221 protects from cytopathic effects induced by dengue virus and Zika virus, and this result suggests that the compound of Example 221 may be an anti-Flavivirus agent.

### Experimental Example 6. Comparative Experiment for Inhibitory Effect of Novel 6,7-dimethoxynaphtho[2,3-c]furan-1(3H)-one Derivatives Against Severe Fever with Thrombocytopenia Syndrome Virus

In order to confirm the inhibitory effect of the novel 6,7-dimethoxynaphtho[2,3-c]furan-1(3H)-one derivatives against virus infection with severe fever with thrombocytopenia syndrome (hereinafter, SFTS), the Vero cell line was cultured for 24 hours, dosed with example compounds at ten concentrations ranging from 0.5 micro M (micro gram) to 50 micro M, and infected with SFTS virus (KADGH; NCCP43261). Specifically, the Vero cells were seeded at 1.2×10⁴ per well into a 384-well tissue culture plate. On the next day, the compound at a concentration of 50 micro M was serially diluted two-fold to prepare compounds at ten concentrations and treated with Vero cells. Soon after, the cells treated with the compound were infected with SFTS virus and cultured at 37° C. for 24 hours. Then, the cells were fixed and permeabilized. After that, the cells were treated with an anti-Ab10 primary antibody and then stained by treatment with an goat anti-Human IgG (H+L) secondary antibody and Alexa Fluor 488. Fluorescent expression was obtained using an Operetta large image analyzer (Perkin Elmer). The response curve and 50% inhibitory effect (IC₅₀) values according to the drug concentration are derived with XLFit (IDBS) software, and the results and cytotoxicity (CC₅₀) of the test substances are summarized in Table 6.

TABLE 6
SFTS virus inhibitory ability and cytotoxicity of compound

| | SFTSV inhibition | Cytotoxicity | SI |
|---|---|---|---|
| Compound | (IC₅₀, μM) | (CC₅₀, μM) | (μM) |
| Example 194 | 0.965 | 47068 | 48785 |
| Example 221 | 0.040 | 457 | 11472 |
| Example 237 | 0.794 | 1015 | 1277 |
| Ribavirin | 64.8 | 1269 | 19.5 |
| Favipiravir | 277.5 | — | >1.8 |
| Chloroquine | 21.8 | 104 | 4.7 |

**Selectivity Index (SI): a value obtained by dividing a value of cytotoxicity (CC₅₀) (assumed to be 50, if >50) by a value of SFTSV inhibition (IC₅₀) (assumed to be 50, if >50).

As confirmed in Table 6, it was found that the example compounds show excellent inhibitory activity against SFTS virus as well as low toxicity. Thus, it is confirmed that the example compounds exhibit an excellent anti-viral effect against SFTS virus, and it is confirmed that the compounds may exhibit excellent preventive or therapeutic effects against a SFTS virus infection disease.

### Experimental Example 7. Comparative Experiment for Inhibition of Novel 6,7-dimethoxynaphtho[2,3-c]furan-1(3H)-one Derivatives Against Secretion of Th2 Inflammatory Cytokines IL-4 and IL-5 in Mouse Splenocytes

In order to confirm an inhibitory effect of novel 6, 7-dimethoxynaphtho[2,3-c]furan-1(3H)-one derivatives against inflammation, splenocytes were isolated by using a spleen which is isolated from Balb/c mice. The isolated splenocytes were diluted at a concentration of 5×10⁶ cells/mL in RPMI medium containing 10% fetal bovine serum (FBS) and antibiotics (penicillin at 100 U/mL, streptomycin at 100 μg/mL), distributed at 500 μl/well in a 24 well plate, and cultured in a 37° C., 5% CO₂ incubator for 48 hours. When splenocytes were distributed, an immune reaction was induced by treating with 5 μg/mL of Concanavalin A, and at this time, the splenocytes were treated with the materials selected in Experimental Example 3 at a concentration of 1 uM. First, in order to measure Th2 cytokines, the medium was collected from the 24-well plate, and only the supernatant was separated through centrifugation at 800×g for five minutes, after which 100 μl of the supernatant per well was used in an enzyme-linked immunosorbent assay (ELISA, Komabiotech) experiment so as to measure a degree of secretion of Th2 cytokines IL-4 and IL-5. A degree of inhibition against secretion of cytokines by the test material was calculated using [Equation 2] below, and the measurement results are shown in Table 7.

$$Secretion inhibitory ability ( \% ) = 100 - \frac{Test group value - Normal group value}{Control group value - Normal group value} ⨯ 100 \qquad [ Equation 2 ]$$

TABLE 7

| | |
|---|---|
| IL-4 | IL-5 |

| | | Secretion | | Secretion |
|---|---|---|---|---|
| Compound | | inhibitory | | inhibitory |
| (1 μM) | pg/ml | ability (%) | pg/ml | ability (%) |
| Normal group | 0.0 | — | 4.6 | — |
| Control group | 340.9 | — | 93.1 | — |
| Example 59 | 71.6 | 79.0 | 51.9 | 44.3 |
| Example 63 | 1.3 | 99.6 | 17.2 | 81.6 |
| Example 65 | 1.5 | 99.5 | 25.2 | 73.0 |
| Example 76 | 1.2 | 99.6 | 13.4 | 85.6 |
| Example 73 | 86.3 | 74.7 | 74.1 | 20.4 |
| Example 86 | 96.0 | 71.8 | 71.0 | 23.7 |
| Example 84 | 1.1 | 99.7 | 11.2 | 88.0 |
| Example 169 | 46.7 | 86.3 | 67.1 | 27.9 |

As confirmed in Table 7, the mouse splenocytes were treated with the derivatives at a concentration of 1 uM, and thus it was confirmed that IL-4 and IL-5 secretion are remarkably inhibited in most of the experimental groups compared to the normal group. In particular, it was observed that the secretion of the cytokine IL-4 is relatively low compared to IL-5. From the above results, it is considered that the novel 6,7-dimethoxynaphtho[2,3-c]furan-1(3H)-one derivatives have a medicinal effect on respiratory diseases such as asthma as well as an anti-inflammatory effect through the inhibition of actual cytokines.

### Experimental Example 8. Comparative Experiment for Inhibitory Effect of Novel 6,7-dimethoxynaphtho[2,3-c]furan-1(3H)-one Derivatives Against Asthma in Balb/c Mouse Asthma Model

The novel 6,7-dimethoxynaphtho[2,3-c]furan-1(3H)-one derivatives were used to measure changes in several indicators related to asthma, a type of respiratory disease, in the Balb/c mouse asthma model. In detail, five-week-old Balb/c mice were purchased and acclimated for one week, after which the mice were sensitized systemically by intraperitoneal injection with 0.1% ovalbumin (OVA 1 mg/mL, Al(OH)₃ 20 mg/mL) at 100 μl/mouse on days 0 and 14 from a week after the acclimation. From a week (day 21) after a final systemic sensitization, the compound of Example 73 was orally administered everyday at 10 mg/kg to mice for ten days, and dexamethasone was intraperitoneally administered at 1 mg/kg as a positive control group. In one hour later, 0.2% ovalbumin solution was sprayed with a nebulizer (PARIBoy SX, GmbH, Germany) so as to be inhaled for one hour. In five hours after the final sensitization (day 30), an autopsy was performed, and 1 mL of phosphate buffer solution was flowed through a tracheotomy to collect a bronchoalveolar lavage fluid (BALF). The collected BALF was centrifuged at 3000 rpm for ten minutes, and the supernatant was used to measure bioactive materials (IL-4, IL-5), and pellets were used to measure the number of inflammatory cells. In addition, an IgE concentration in the isolated BALF was measured using IgE enzyme-linked immunosorbent assay (ELISA, Komabiotech), and IL-4 and IL-5 were measured using their respective enzyme-linked immunosorbent assay (ELISA, IL-4: Enzo #ADI-900-043, IL-5: R&D system #M5000).

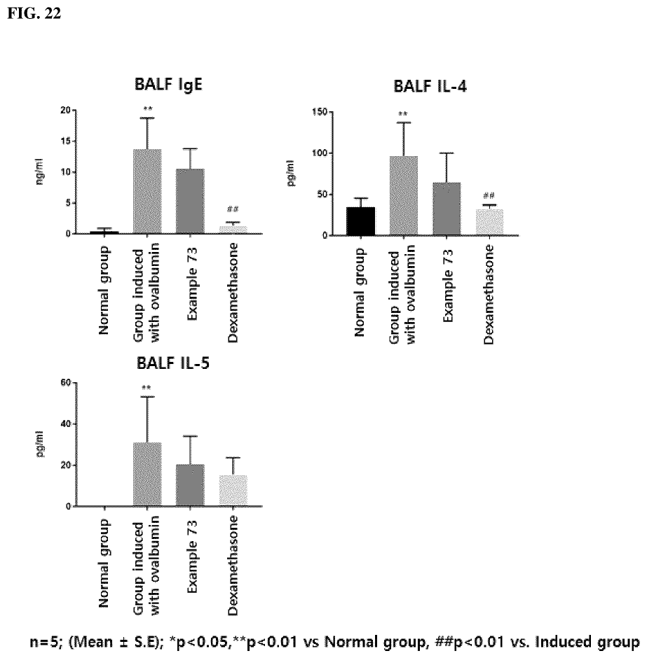

The results are shown in Table 8 and FIG. 22, and each secretion inhibitory activity was calculated in the same manner as in [Equation 2] of Experimental Example 4.

TABLE 8

| | BALF IgE | BALF IL-4 | BALF IL-5 |
|---|---|---|---|
| | ng/ml | pg/ml | pg/ml |
| | (Secretion | (Secretion | (Secretion |
| | inhibitory | inhibitory | inhibitory |
| Mean ± S.D. | ability %) | ability %) | ability %) |
| Normal group | 0.4 ± 0.526 | 34.6 ± 10.789 | 0 ± 0 |
| | (—) | (—) | (—) |
| Group induced | 13.8 ± 4.980** | 96.8 ± 40.197** | 31.2 ± 21.95** |
| with ovalbumin | (—) | (—) | (—) |
| Group treated | 10.5 ± 3.282 | 64.9 ± 35.025 | 20.7 ± 13.46 |
| with Examples 73 | (24.3%) | (51.3%) | (33.7%) |
| Dexamethasone | 1.3 ± 0.536 ^{##} | 32.9 ± 4.534 ^{##} | 15.6 ± 8.07 |
| (positive | (93.0%) | (102.7%) | (50.0%) |
| control group) | | | |

n = 5;
(Mean ± S.E);
*p < 0.05,
**p < 0.01 vs Normal,
^{##} p < 0.01 vs. Induced

In addition, the BALF pellets were re-dissolved by adding 0.5 mL of phosphate buffer solution, and then 0.1 mL for each thereof was added to a 96-well plate and centrifuged at 800 rpm for five minutes to attach cells to the bottom (three wells per sample). Then, the cells were stained with Diff Quik staining solution (Sysmex) and the number of cells was measured with a flow cytometer.

The results were shown in FIG. 21, the SPSS program was used for statistical processing, the Levene test was performed for equal-variance test, and the significance test was performed through one-way ANOVA.

As can be seen in Table 8, FIGS. 22 and 21, the compound of Example 73 reduced Th2 cytokines IL-4 and IL-5 as well as IgE in the BALF. In addition, it was observed that inflammatory cells are reduced as a whole and, in particular, the number of eosinophils highly associated with type 2 asthma is significantly reduced. From these results, it was confirmed that the compound of Example 73 has an excellent allergy inhibitory effect and is a material capable of effectively treating respiratory diseases such as asthma.

1. A compound of formula 1 below, a pharmaceutically acceptable salt thereof, a stereoisomer thereof, a hydrate thereof or a solvate thereof: 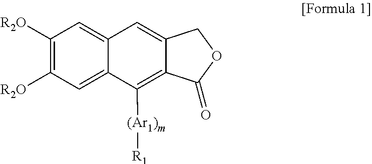
wherein in above formula 1,
Ar₁ is substituted or unsubstituted C₆-C₁₂ arylene group, or substituted or unsubstituted C₂-C₁₂ heteroarylene group comprising at least one of N, O and S as a heteroatom in a ring, and m is 0 or 1,
R₁ is hydrogen, halogen, substituted or unsubstituted C₁-C₁₀ linear or branched alkyl, substituted or unsubstituted C₂-C₂₀ heterocycloalkyl comprising at least one of N, O and S as a heteroatom in a ring (wherein a substituent of C₃-C₂₀ cycloalkyl or C₂-C₂₀ heterocycloalkyl may be each independently bonded to an adjacent group to form a ring), substituted or unsubstituted C₂-C₂₀ heteroaryl comprising at least one of N, O and S as a heteroatom in a ring, —NR₄R₅, —OR₆, —N═CHR₇ or 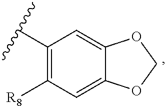
when R₁ is hydrogen or halogen, m is 1,
when C₂-C₂₀ heteroaryl is furan or thiophene and m is 0, the furan and thiophene each independently have at least one hydrogen substituted with C₁-C₁₀ linear or branched alkyl, substituted or unsubstituted C₆-C₂₀ aryl, substituted or unsubstituted C₂-C₂₀ heteroaryl, or —C(═O)Rₐ (wherein Rₐ is hydrogen, C₁-C₁₀ linear or branched alkyl, substituted or unsubstituted C₆-C₂₀ aryl, or substituted or unsubstituted C₂-C₂₀ heteroaryl),
R₂ and R₃ are each independently C₁-C₁₀ linear or branched alkyl,
R₄ to R₇ are each independently hydrogen, halogen, C₁-C₁₀ linear or branched alkyl (wherein at least one of the hydrogen of the alkyl may be substituted with OH, substituted or unsubstituted C₃-C₁₀ cycloalkyl, substituted or unsubstituted C₃-C₁₀ cycloalkenyl, substituted or unsubstituted C₂-C₁₀ heterocycloalkyl, substituted or unsubstituted C₆-C₂₀ aryl, substituted or unsubstituted C₂-C₂₀ heteroaryl, —C(═O)OR_{b}, linear or branched alkoxy, or amine), substituted or unsubstituted C₃-C₁₀ cycloalkyl (wherein a substituent of the cycloalkyl may be bonded to an adjacent group to form a ring), substituted or unsubstituted C₂-C₁₀ heterocycloalkyl comprising at least one of N, O and S as a heteroatom in a ring (wherein a substituent of the heterocycloalkyl may be bonded to an adjacent group to form a ring), substituted or unsubstituted C₆-C₂₀ aryl (wherein a substituent of the aryl may be bonded to an adjacent group to form a ring), substituted or unsubstituted C₂-C₂₀ heteroaryl comprising at least one of N, O and S as a heteroatom in a ring, or —C(═O)R_{c},
R_{b} is C₁-C₁₀ linear or branched alkyl, and Rₑ is C₁-C₁₀ linear or branched alkyl or C₁-C₁₀ linear or branched alkoxy, and
R₈ is —OC(═O)R_{d} or C₁-C₂₀ linear or branched alkoxy, and R_{d} is C₁-C₁₀ linear or branched alkyl, C₁-C₁₀ linear or branched alkoxy, or substituted or unsubstituted C₃-C₂₀ cycloalkoxy.

2. The compound, the pharmaceutically acceptable salt thereof, the stereoisomer thereof, the hydrate thereof or the solvate thereof according to claim 1, wherein Ar₁—R₁ is represented by formula 2 below: 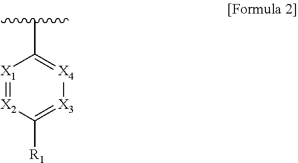
wherein in above formula 2,
X₁ to X₄ are each independently CH or N, and R₁ is same as defined in above formula 1.

3. The compound, the pharmaceutically acceptable salt thereof, the stereoisomer thereof, the hydrate thereof or the solvate thereof according to claim 2, wherein above formula 2 is represented by formulas 2-1 to 2-8 below: 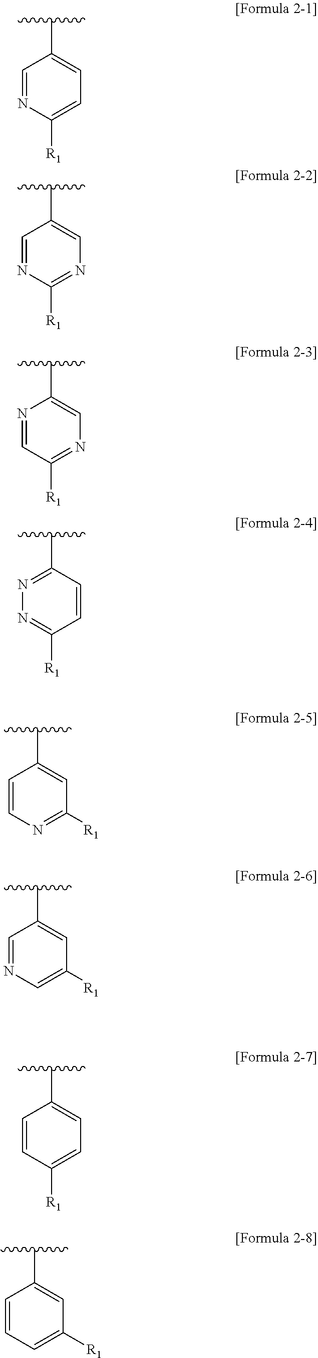
wherein in above formulas 2-1 to 2-8, R₁ is same as defined in above formula 2.

4. The compound, the pharmaceutically acceptable salt thereof, the stereoisomer thereof, the hydrate thereof or the solvate thereof according to claim 1, wherein R₁ is hydrogen, halogen, substituted or unsubstituted C₁-C₁₀ linear or branched alkyl, —NR₄R₅, —OR₆, —N═CHR₇, 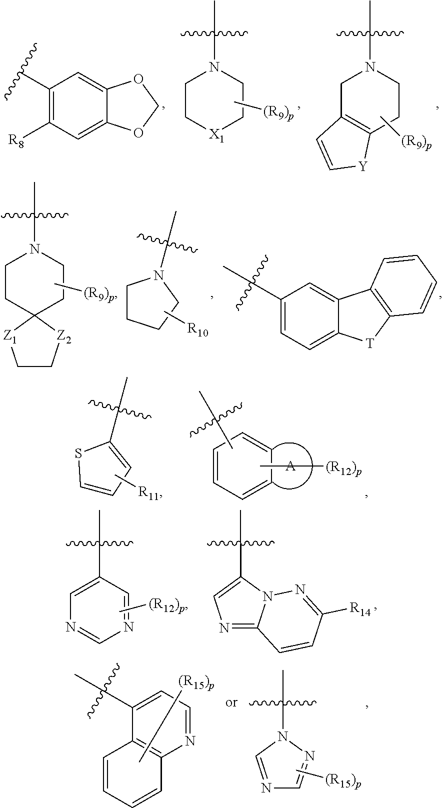
X₁ is —CHR₁₃, —CX₂ (wherein, X is halogen)-NR₁₃, —O— or —S—,
Y, Z₁, Z₂ and T are each independently —O— or —S—,
R₉ is each independently hydrogen, C₁-C₁₀ linear or branched alkyl (wherein at least one of the hydrogen of R₉ may be substituted with —OH, and R₉ may be bonded to an adjacent group to form a substituted or unsubstituted ring), substituted or unsubstituted C₆-C₂₀ aryl, amine, —OH, or —C(═O)ORₑ (Rₑ is hydrogen or C₁-C₁₀ linear or branched alkyl),
R₁₀ is hydrogen, C₁-C₁₀ linear or branched alkyl (wherein at least one of the hydrogen of the alkyl may be substituted with —OH, C₁-C₁₀ linear or branched alkoxy, or substituted or unsubstituted C₂-C₂₀ heterocycloalkyl), —C(═O)R_{f} (wherein R_{f} is —OH, C₁-C₁₀ linear or branched alkyl, C₁-C₁₀ linear or branched alkoxy, substituted or unsubstituted amine, substituted or unsubstituted C₂-C₂₀ heterocycloalkyl) or —OC(═O)R_{g} (wherein R_{g} is C₁-C₁₀ linear or branched alkyl and may be substituted with —OH),
R₁₁ is —C(═O)Rₐ (wherein Rₐ is hydrogen, C₁-C₁₀ linear or branched alkyl, substituted or unsubstituted C₆-C₂₀ aryl, or substituted or unsubstituted C₂-C₂₀ heteroaryl),
R₁₂ is each independently hydrogen, halogen, C₁-C₁₀ linear or branched alkyl (wherein at least one of the hydrogen of the alkyl may be substituted with substituted or unsubstituted C₂-C₁₀ heterocycloalkyl), substituted or unsubstituted C₂-C₂₀ heterocycloalkyl, C₁-C₁₀ linear or branched alkoxy, —CX₃ (wherein X is halogen), or —OH,
R₁₃ is hydrogen, halogen, substituted or unsubstituted C₁-C₁₀ linear or branched alkyl (wherein at least one of the hydrogen of the alkyl may be substituted with —OH, substituted or unsubstituted C₂-C₁₀ heterocycloalkyl, and R₁₃ may be bonded to an adjacent group to form a substituted or unsubstituted ring), substituted or unsubstituted C₃-C₂₀ cycloalkyl (wherein a substituent of the C₃-C₂₀ cycloalkyl may be bonded to an adjacent group to form a ring), substituted or unsubstituted C₆-C₂₀ aryl, substituted or unsubstituted C₃-C₂₀ heteroaryl (wherein at least one of the hydrogen of the C₃-C₂₀ heteroaryl may be substituted with C₁-C₁₀ alkylthio group or halogen), —OH, or amine,
R₁₄ is C₂-C₁₀ heterocycloalkyl comprising at least one of N and O as a heteroatom in a ring,
R₁₅ is each independently halogen, C₁-C₁₀ linear or branched alkyl, —CX₃ (wherein X is halogen), C₁-C₁₀ linear or branched alkoxy, an alkylthio group, or amine,
p is an integer of 0 or more and 5 or less,
A is substituted or unsubstituted C₂-C₁₀ heterocycloalkyl comprising at least one of N, O and S as a heteroatom in a ring, substituted or unsubstituted C₆-C₂₀ aryl, or substituted or unsubstituted C₂-C₂₀ heteroaryl comprising at least one N as a heteroatom in a ring, and
R₄ to R₈ are same as defined in formula 1.

5. The compound, the pharmaceutically acceptable salt thereof, the stereoisomer thereof, the hydrate thereof or the solvate thereof according to claim 1, wherein, R1 is hydrogen, fluroine, chlorine, —CH₃,

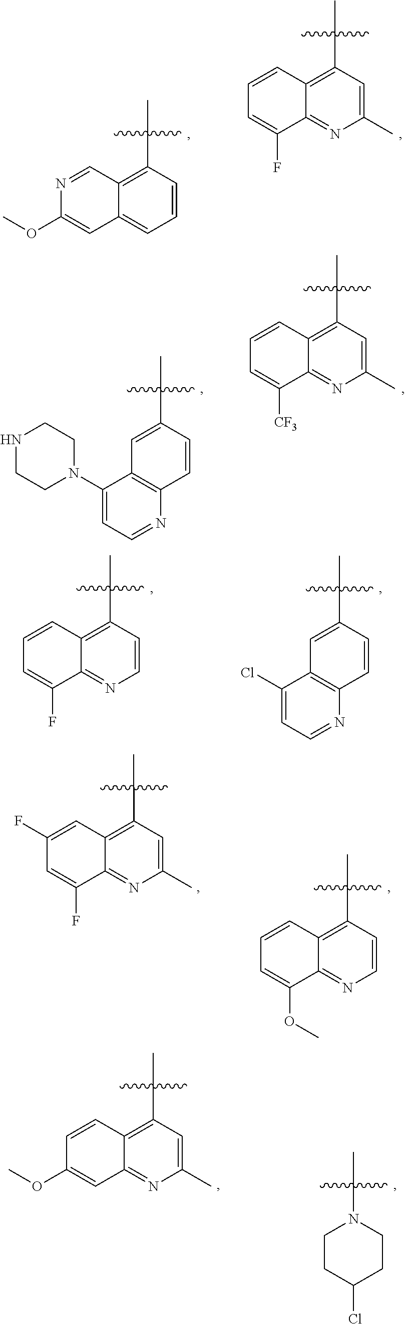

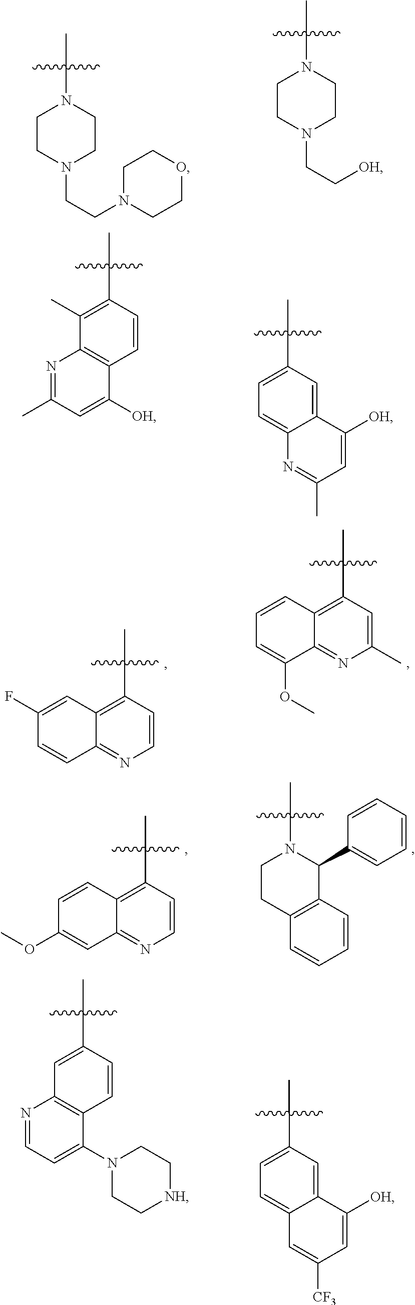

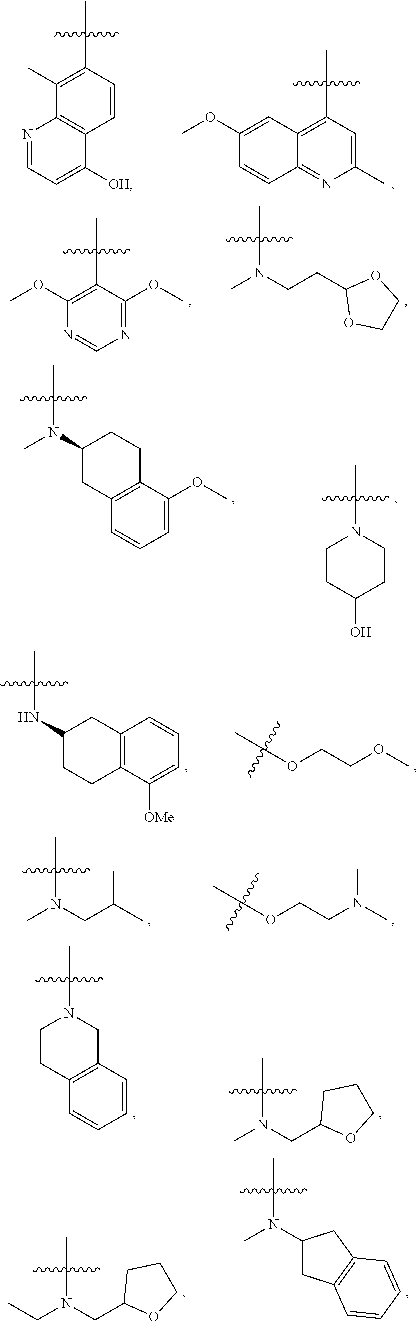

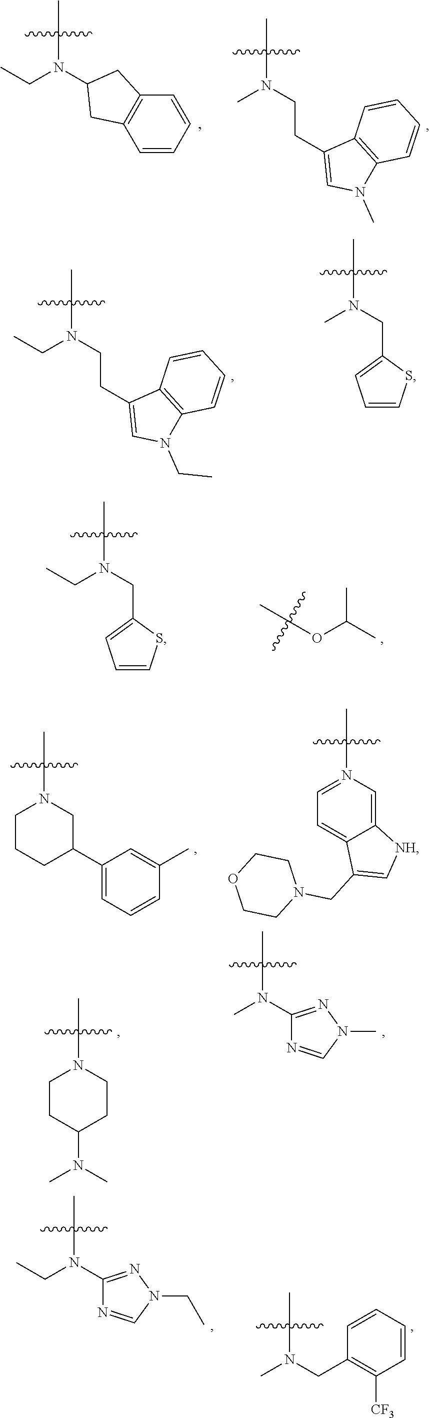

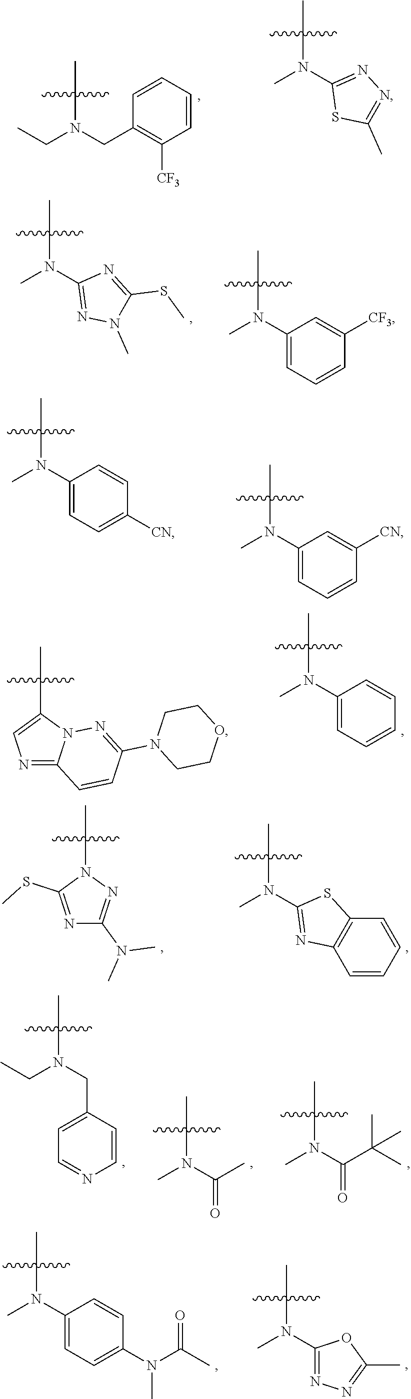

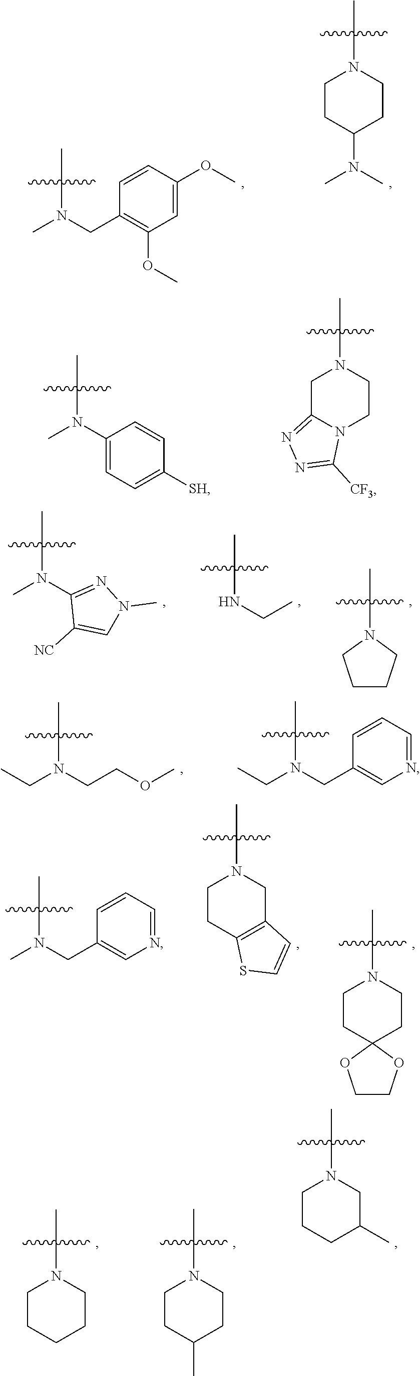

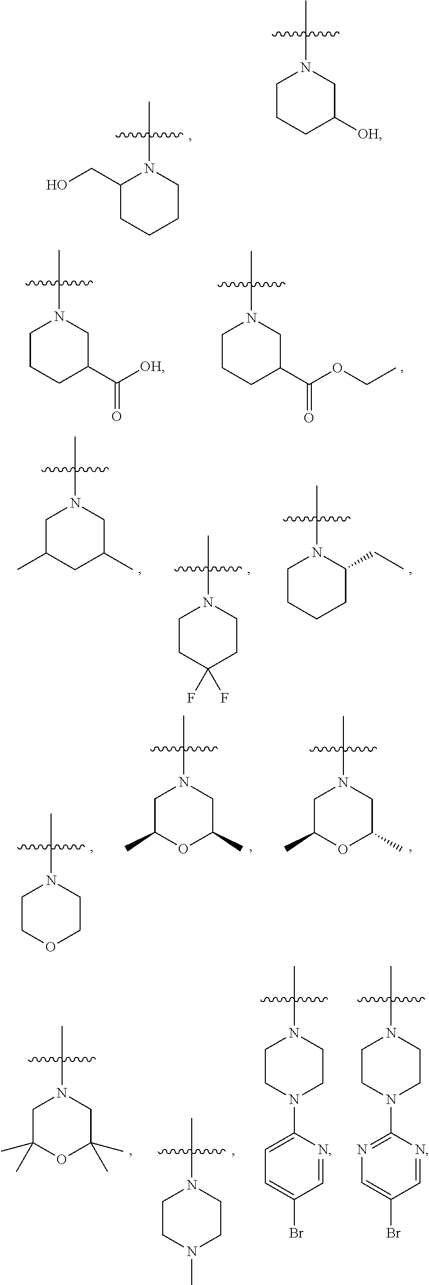

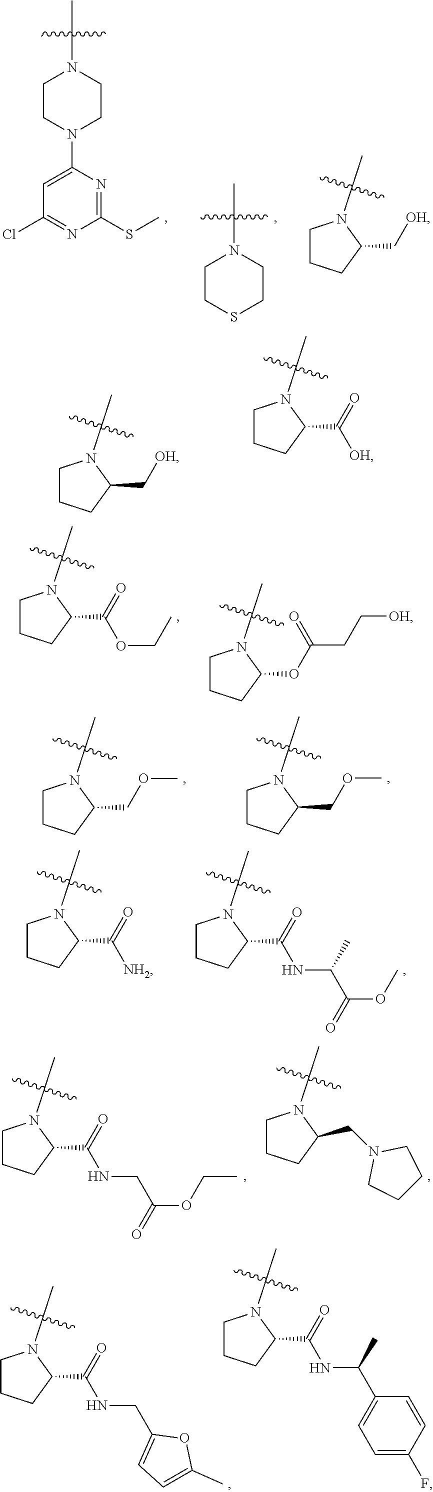

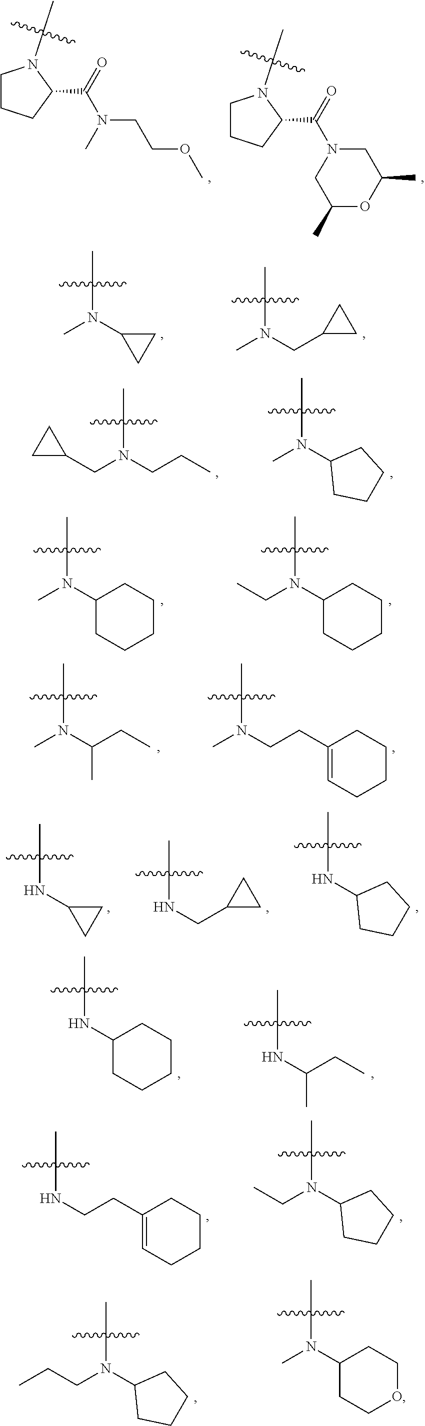

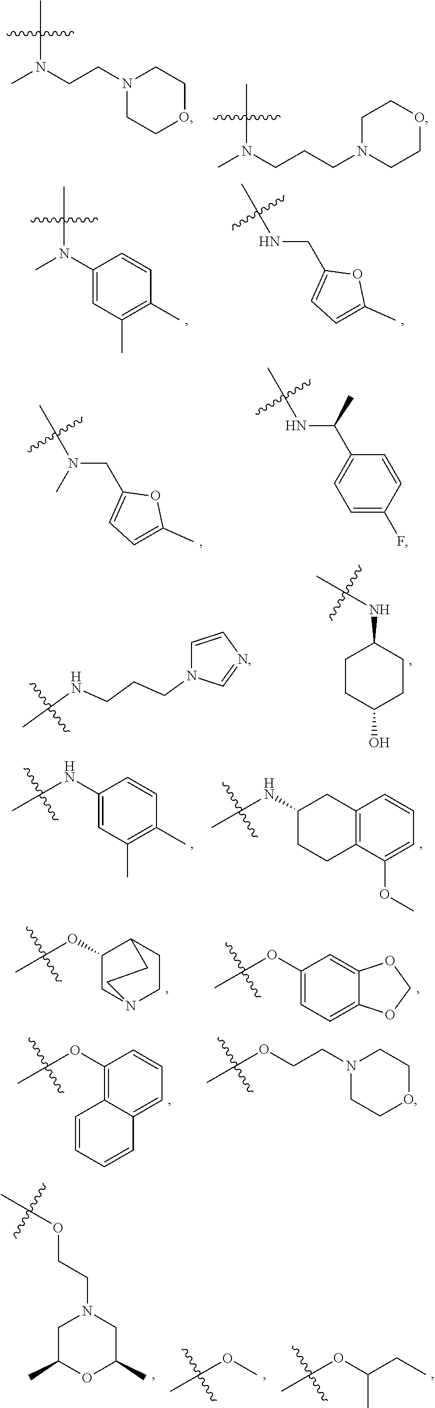

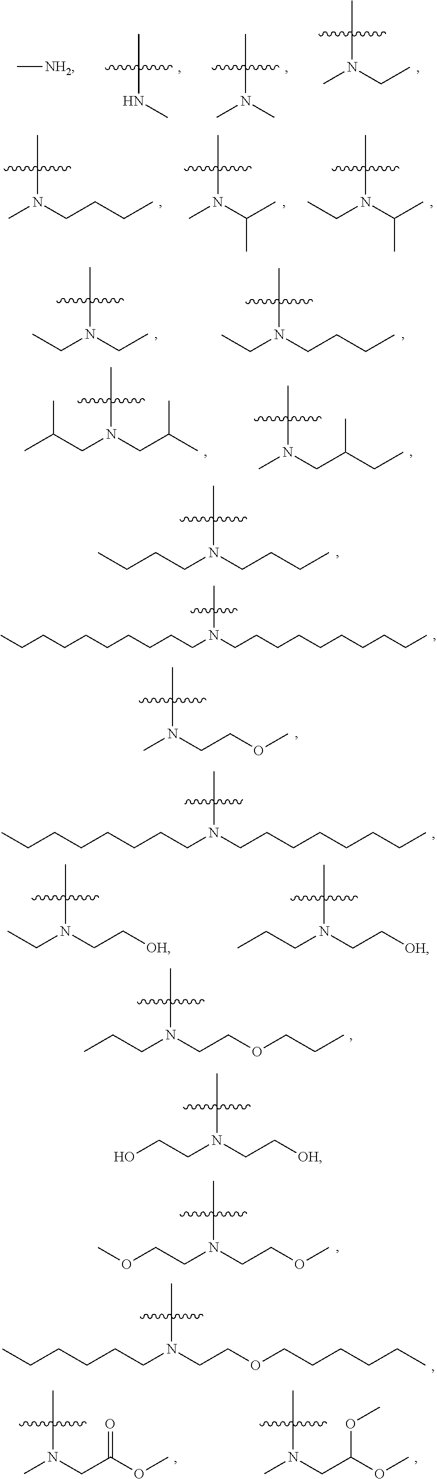

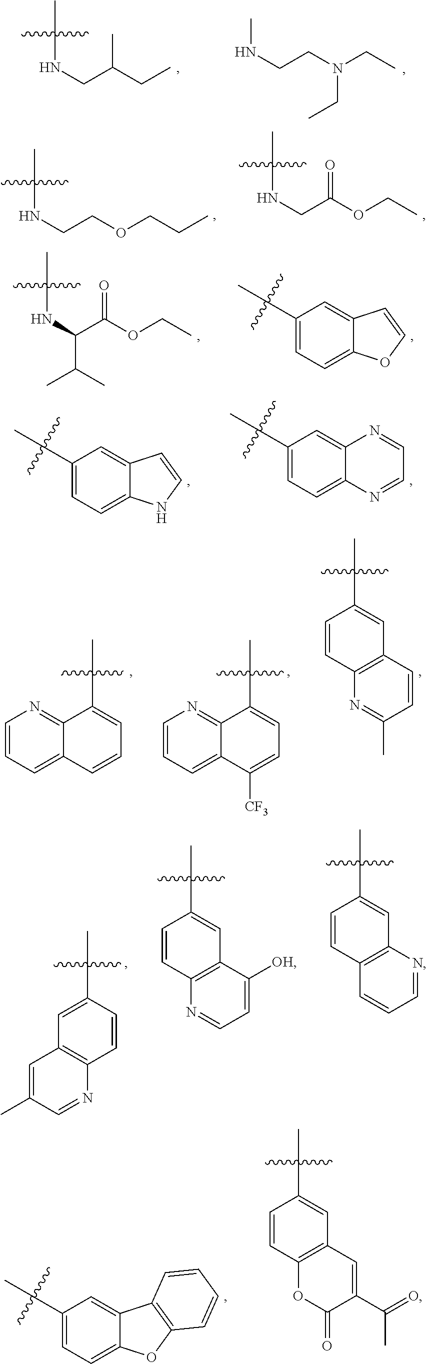

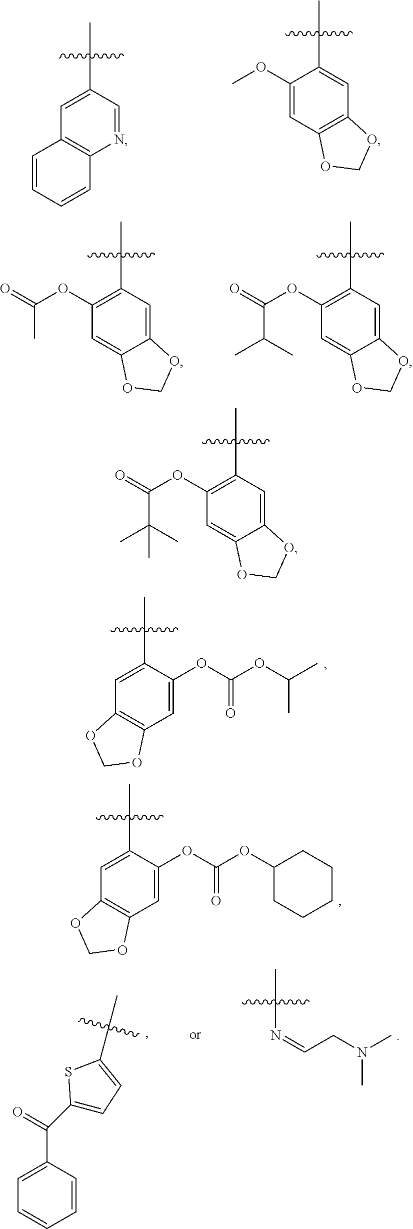

6. The compound, the pharmaceutically acceptable salt thereof, the stereoisomer thereof, the hydrate thereof and the solvate thereof according to claim 1, wherein the compound represented by above formula 1 is one selected from the group consisting of the following compounds:
[001] (S)-6,7-dimethoxy-9-((5-methoxy-1,2,3,4-tetrahydronaphthalen-2-yl)amino)naphtho[2,3-c]furan-1(3H)-one
[002] 9-((2S,6R)-2,6-dimethylmorpholino)-6,7-dimethoxynaphtho[2,3-c]furan-1(3H)-one
[003] 9-(6,7-dihydrothieno[3,2-c]pyridin-5 (4H)-yl)-6,7-dimethoxynaphtho[2,3-c]furan-1(3H)-one
[004] 9-(3,5-dimethylpiperidin-1-yl)-6,7-dimethoxynaphtho[2,3-c]furan-1(3H)-one
[005] 6,7-dimethoxy-9-((2-methoxyethyl)(methyl)amino)naphtho[2,3-c]furan-1(3H)-one
[006] 9-((2-(diethylamino)ethyl)amino)-6,7-dimethoxynaphtho[2,3-c]furan-1(3H)-one
[007] 9-(2-((2S,6R)-2,6-dimethylmorpholino)pyrimidin-5-yl)-6,7-dimethoxynaphtho[2,3-c]furan-1(3H)-one
[008] 9-(2-((2S,6R)-2,6-dimethylmorpholino)pyrimidin-5-yl)-6,7-dimethoxynaphtho[2,3-c]furan-1(3H)-one
[009] 6,7-dimethoxy-9-(6-thiomorpholinopyridin-3-yl)naphtho[2,3-c]furan-1(3H)-one
[010] 6,7-dimethoxy-9-(6-thiomorpholinopyridin-3-yl)naphtho[2,3-c]furan-1(3H)-one
[011] 9-(2-(dimethylamino)pyrimidin-5-yl)-6,7-dimethoxynaphtho[2,3-c]furan-1(3H)-one
[012] 9-(2-(6,7-dihydrothieno[3,2-c]pyridin-5 (4H)-yl)pyrimidin-5-yl)-6,7-dimethoxynaphtho[2,3-c]furan-1(3H)-one
[013] 6,7-dimethoxy-9-(2-(2-morpholinoethoxy)pyrimidin-5-yl)naphtho[2,3-c]furan-1(3H)-one
[014] 9-(2-((3,4-dimethylphenyl)amino)pyrimidin-5-yl)-6,7-dimethoxynaphtho[2,3-c]furan-1(3H)-one
[015] Ethyl 1-(5-(6,7-dimethoxy-3-oxo-1,3-dihydronaphtho[2,3-c]furan-4-yl)pyrimidin-2-yl) piperidin-3-carboxylate
[016] 9-(2-((3-(1H-imidazol-1-yl) propyl)amino)pyrimidin-5-yl)-6,7-dimethoxynaphtho[2,3-c]furan-1(3H)-one
[017] (R)-6,7-dimethoxy-9-(2-(quinuclidin-3-yloxy)pyrimidin-5-yl)naphtho[2,3-c]furan-1(3H)-one
[018] 6,7-dimethoxy-9-(2-((2-methoxyethyl)(methyl)amino)pyrimidin-5-yl)naphtho[2,3-c]furan-1(3H)-one
[019] (S)ethyl 1-(5-(6,7-dimethoxy-3-oxo-1,3-dihydronaphtho[2,3-c]furan-4-yl)pyrimidin-2-yl)pyrrolidin-2-carboxylate
[020] 9-(2-(benzo[d][1,3]dioxol-5-yloxy)pyrimidin-5-yl)-6,7-dimethoxynaphtho[2,3-c]furan-1(3H)-one
[021] 6,7-dimethoxy-9-(2-(naphthalen-1-yloxy)pyrimidin-5-yl)naphtho[2,3-c]furan-1(3H)-one
[022] (S)-6,7-dimethoxy-9-(2-((5-methoxy-1,2,3,4-tetrahydronaphthalen-2-yl)amino)pyrimidin-5-yl)naphtho[2,3-c]furan-1(3H)-one
[023] 9-(2-(isopropyl(methyl)amino)pyrimidin-5-yl)-6,7-dimethoxynaphtho[2,3-c]furan-1(3H)-one
[024] 9-(2-((S)-2-((2S,6R)-2,6-dimethylmorpholin-4-carbonyl)pyrrolidin-1-yl)pyrimidin-5-yl)-6,7-dimethoxynaphtho[2,3-c]furan-1(3H)-one
[025] 9-(2-(sec-butoxy)pyrimidin-5-yl)-6,7-dimethoxynaphtho[2,3-c]furan-1(3H)-one
[026] 6,7-dimethoxy-9-(2-methoxypyrimidin-5-yl)naphtho[2,3-c]furan-1(3H)-one
[027] Methyl (5-(6,7-dimethoxy-3-oxo-1,3-dihydronaphtho[2,3-c]furan-4-yl)pyrimidin-2-yl)-D-valinate
[028] 9-(2-((2-(cyclohex-1-en-1-yl)ethyl)amino)pyrimidin-5-yl)-6,7-dimethoxynaphtho[2,3-c]furan-1(3H)-one
[029] (Z)-9-(5-((2-(dimethylamino)ethyliden)amino)pyrazin-2-yl)-6,7-dimethoxynaphtho[2,3-c]furan-1(3H)-one
[030] 9-(5-aminopyrazin-2-yl)-6,7-dimethoxynaphtho[2,3-c]furan-1(3H)-one
[031] 9-(5-(dimethylamino)pyrazin-2-yl)-6,7-dimethoxynaphtho[2,3-c]furan-1(3H)-one
[032] 6,7-dimethoxy-9-(pyrazin-2-yl)naphtho[2,3-c]furan-1(3H)-one
[033] (S)-1-(5-(6,7-dimethoxy-3-oxo-1,3-dihydronaphtho[2,3-c]furan-4-yl)pyrimidin-2-yl)-N-(2-methoxyethyl)-N-methylpyrrolidin-2-carboxamide
[034] 9-(1H-indol-5-yl)-6,7-dimethoxynaphtho[2,3-c]furan-1(3H)-one
[035] 9-(6-fluoropyridin-3-yl)-6,7-dimethoxynaphtho[2,3-c]furan-1(3H)-one
[036] 2-hydroxyethyl(5-(6,7-dimethoxy-3-oxo-1,3-dihydronaphtho[2,3-c]furan-4-yl)pyrimidin-2-yl)-L-prolinate
[037] 6,7-dimethoxy-9-(2-((2-propoxyethyl)amino)pyrimidin-5-yl)naphtho[2,3-c]furan-1(3H)-one
[038] Methyl (5-(6,7-dimethoxy-3-oxo-1,3-dihydronaphtho[2,3-c]furan-4-yl)pyrimidin-2-yl)-L-prolyl-D-alaniate
[039] (S)-1-(5-(6,7-dimethoxy-3-oxo-1,3-dihydronaphtho[2,3-c]furan-4-yl)pyrimidin-2-yl)-N—((S)-1-(4-fluorophenyl)ethyl)pyrrolidin-2-carboxamide
[040] 6,7-dimethoxy-9-(quinoxalin-6-yl)naphtho[2,3-c]furan-1(3H)-one
[041] (S)-1-(5-(6,7-dimethoxy-3-oxo-1,3-dihydronaphtho[2,3-c]furan-4-yl)pyrimidin-2-yl)-N-((5-methylfuran-2-yl)methyl)pyrrolidin-2-carboxamide
[042] 6,7-dimethoxy-9-(2-((2-propoxyethyl)(propyl)amino)pyrimidin-5-yl)naphtho[2,3-c]furan-1(3H)-one
[043] 9-(dibenzo[b,d]furan-2-yl)-6,7-dimethoxynaphtho[2,3-c]furan-1(3H)-one
[044] Ethyl (5-(6,7-dimethoxy-3-oxo-1,3-dihydronaphtho[2,3-c]furan-4-yl)pyrimidin-2-yl)glycinate
[045] Ethyl (5-(6,7-dimethoxy-3-oxo-1,3-dihydronaphtho[2,3-c]furan-4-yl)pyrimidin-2-yl)-L-proglycinate
[046] 6,7-dimethoxy-9-(quinolin-3-yl)naphtho[2,3-c]furan-1(3H)-one
[047] 9-(2-(((1r,4r)-4-hydroxycyclohexyl)amino)pyrimidin-5-yl)-6,7-dimethoxynaphtho[2,3-c]furan-1(3H)-one carboxamide
[048] 9-(2-(hexyl(2-(hexyloxy)ethyl)amino)pyrimidin-5-yl)-6,7-dimethoxynaphtho[2,3-c]furan-1(3H)-one
[049] (S)-1-(5-(6,7-dimethoxy-3-oxo-1,3-dihydronaphtho[2,3-c]furan-4-yl)pyrimidin-2-yl)pyrrolidin-2-carboxamide
[050] 6,7-dimethoxy-9-(2-(((5-methylfuran-2-yl)methyl)amino)pyrimidin-5-yl)naphtho[2,3-c]furan-1(3H)-one
[051] 6,7-dimethoxy-9-(2-morpholinopyrimidin-5-yl)naphtho[2,3-c]furan-1(3H)-one carboxamide
[052] 9-(benzofuran-5-yl)-6,7-dimethoxynaphtho[2,3-c]furan-1(3H)-one carboxamide
[053] 9-(5-benzothiophen-2-yl)-6,7-dimethoxynaphtho[2,3-c]furan-1(3H)-one
[054] 9-(2-(2-((2S,6R)-2,6-dimethylmorpholino) ethoxy)pyrimidin-5-yl)-6,7-dimethoxynaphtho[2,3-c]furan-1(3H)-one
[055] (S)-9-(2-((1-(4-fluorophenyl)ethyl)amino)pyrimidin-5-yl)-6,7-dimethoxynaphtho[2,3-c]furan-1(3H)-one
[056] 9-(3-acetyl-2-oxo-2H-chromen-6-yl)-6,7-dimethoxynaphtho[2,3-c]furan-1(3H)-one
[057] 9-(2-(3,5-dimethylpiperidin-1-yl)pyrimidin-5-yl)-6,7-dimethoxynaphtho[2,3-c]furan-1(3H)-one
[058] 9-(2-((2-(diethylamino)ethyl)amino)pyrimidin-5-yl)-6,7-dimethoxynaphtho[2,3-c]furan-1(3H)-one carboxamide
[059] 6,7-dimethoxy-9-(2-(3-methylpiperidin-1-yl)pyrimidin-5-yl)naphtho[2,3-c]furan-1(3H)-one
[060] 1-(5-(6,7-dimethoxy-3-oxo-1,3-dihydronaphtho[2,3-c]furan-4-yl)pyrimidin-2-yl) piperidin-3-carboxylic acid
[061] (5-(6,7-dimethoxy-3-oxo-1,3-dihydronaphtho[2,3-c]furan-4-yl)pyrimidin-2-yl)-L-prolan
[062] 9-(2-((2S,6S)-2,6-dimethylmorpholino)pyrimidin-5-yl)-6,7-dimethoxynaphtho[2,3-c]furan-1(3H)-one
[063] 6,7-dimethoxy-9-(2-thiomorpholinopyrimidin-5-yl)naphtho[2,3-c]furan-1(3H)-one
[064] 6,7-dimethoxy-9-(2-((2-methylbutyl)amino)pyrimidin-5-yl)naphtho[2,3-c]furan-1(3H)-one
[065] 6,7-dimethoxy-9-(2-(methyl(2-methylbutyl)amino)pyrimidin-5-yl)naphtho[2,3-c]furan-1(3H)-one
[066] 9-(2-(butyl(ethyl)amino)pyrimidin-5-yl)-6,7-dimethoxynaphtho[2,3-c]furan-1(3H)-one
[067] 9-(2-((cyclopropylmethyl)amino)pyrimidin-5-yl)-6,7-dimethoxynaphtho[2,3-c]furan-1(3H)-one
[068] 9-(2-((cyclopropylmethyl)(methyl)amino)pyrimidin-5-yl)-6,7-dimethoxynaphtho[2,3-c]furan-1(3H)-one
[069] 9-(2-(cyclopropylamino)pyrimidin-5-yl)-6,7-dimethoxynaphtho[2,3-c]furan-1(3H)-one
[070] 9-(2-(cyclopropyl(methyl)amino)pyrimidin-5-yl)-6,7-dimethoxynaphtho[2,3-c]furan-1(3H)-one
[071] 9-(2-(butyl(methyl)amino)pyrimidin-5-yl)-6,7-dimethoxynaphtho[2,3-c]furan-1(3H)-one
[072] 9-(2-(bis(2-methoxyethyl)amino)pyrimidin-5-yl)-6,7-dimethoxynaphtho[2,3-c]furan-1(3H)-one
[073] 9-(2-(dibutylamino)pyrimidin-5-yl)-6,7-dimethoxynaphtho[2,3-c]furan-1(3H)-one
[074] 9-(2-(diethylamino)pyrimidin-5-yl)-6,7-dimethoxynaphtho[2,3-c]furan-1(3H)-one
[075] 9-(2-(ethyl(isopropyl)amino)pyrimidin-5-yl)-6,7-dimethoxynaphtho[2,3-c]furan-1(3H)-one hydrochloride
[076] 9-(2-(butyl(methyl)amino)pyrimidin-5-yl)-6,7-dimethoxynaphtho[2,3-c]furan-1(3H)-one hydrochloride
[077] 9-(2-((2-hydroxyethyl)(propyl)amino)pyrimidin-5-yl)-6,7-dimethoxynaphtho[2,3-c]furan-1(3H)-one hydrochloride
[078] 9-(2-(cyclohexyl(ethyl)amino)pyrimidin-5-yl)-6,7-dimethoxynaphtho[2,3-c]furan-1(3H)-one hydrochloride
[079] 9-(2-(cyclohexyl(methyl)amino)pyrimidin-5-yl)-6,7-dimethoxynaphtho[2,3-c]furan-1(3H)-one hydrochloride
[080] 9-(2-(3-hydroxy peridin-1-ylpyrimidin-5-yl)-6,7-dimethoxynaphtho[2,3-c]furan-1(3H)-one hydrochloride
[081] 9-(2-(ethyl(2-hydroxyethyl)amino)pyrimidin-5-yl)-6,7-dimethoxynaphtho[2,3-c]furan-1(3H)-one hydrochloride
[082] (R)-9-(2-(2-(hydroxymethyl)pyrrolidin-1-yl)pyrimidin-5-yl)-6,7-dimethoxynaphtho[2,3-c]furan-1(3H)-one hydrochloride
[083] (S)-9-(2-(2-(hydroxymethyl)pyrrolidin-1-yl)pyrimidin-5-yl)-6,7-dimethoxynaphtho[2,3-c]furan-1(3H)-one hydrochloride
[084] 9-(2-(cyclopentyl(methyl)amino)pyrimidin-5-yl)-6,7-dimethoxynaphtho[2,3-c]furan-1(3H)-one hydrochloride
[085] 9-(2-(bis(2-hydroxyethyl)amino)pyrimidin-5-yl)-6,7-dimethoxynaphtho[2,3-c]furan-1(3H)-one hydrochloride
[086] 9-(2-(diisobutylamino)pyrimidin-5-yl)-6,7-dimethoxynaphtho[2,3-c]furan-1(3H)-one hydrochloride
[087] 9-(2-(didecylamino)pyrimidin-5-yl)-6,7-dimethoxynaphtho[2,3-c]furan-1(3H)-one hydrochloride
[088] 6,7-dimethoxy-9-(6-methoxybenzo[d][1,3]dioxol-5-yl)naphtho[2,3-c]furan-1(3H)-one
[089] 9-(2-(1,4-dioxa-8-azaspiro[4.5]decan-8-yl)pyrimidin-5-yl)-6,7-dimethoxynaphtho[2,3-c]furan-1(3H)-one
[090] Methyl N-(5-(6,7-dimethoxy-3-oxo-1,3-dihydronaphtho[2,3-c]furan-4-yl)pyrimidin-2-yl)-N-methyl glycinate hydrochloride
[091] 9-(2-(cyclopentylamino)pyrimidin-5-yl)-6,7-dimethoxynaphtho[2,3-c]furan-1(3H)-one hydrochloride
[092] 9-(2-(cyclopentyl(ethyl)amino)pyrimidin-5-yl)-6,7-dimethoxynaphtho[2,3-c]furan-1(3H)-one hydrochloride
[093] 9-(2-(cyclopentyl(propyl)amino)pyrimidin-5-yl)-6,7-dimethoxynaphtho[2,3-c]furan-1(3H)-one hydrochloride
[094] 9-(2-((2,2-dimethoxyethyl)(methyl)amino)pyrimidin-5-yl)-6,7-dimethoxynaphtho[2,3-c]furan-1(3H)-one
[095] 9-(2-(dioctylamino)pyrimidin-5-yl)-6,7-dimethoxynaphtho[2,3-c]furan-1(3H)-one hydrochloride
[096] (S)-6,7-dimethoxy-9-(2-((5-methoxy-1,2,3,4-tetrahydronaphthalen-2-yl)(methyl)amino)pyrimidin-5-yl)naphtho[2,3-c]furan-1(3H)-one hydrochloride
[097] (S)-6,7-dimethoxy-9-(2-(2-(methoxymethyl)pyrrolidin-1-yl)pyrimidin-5-yl)naphtho[2,3-c]furan-1(3H)-one hydrochloride
[098] 6-(6,7-dimethoxy-3-oxo-1,3-dihydronaphtho[2,3-c]furan-4-yl)benzo[d][1,3]dioxol-5-yl isopropyl carbonate
[099] Cyclohexyl(6-(6,7-dimethoxy-3-oxo-1,3-dihydronaphtho[2,3-c]furan-4-yl)benzo[d][1,3]dioxol-5-yl)carbonate
[100] 9-(2-(sec-butyl(methyl)amino)pyrimidin-5-yl)-6,7-dimethoxynaphtho[2,3-c]furan-1(3H)-one hydrochloride
[101] 9-(2-((2-(cyclohex-1-en-1-yl)ethyl)(methyl)amino)pyrimidin-5-yl)-6,7-dimethoxynaphtho[2,3-c]furan-1(3H)-one hydrochloride
[102] (R)-6,7-dimethoxy-9-(2-(2-(methoxymethyl)pyrrolidin-1-yl)pyrimidin-5-yl)naphtho[2,3-c]furan-1(3H)-one hydrochloride
[103] 6,7-dimethoxy-9-(2-(methyl((5-methylfuran-2-yl)methyl)amino)pyrimidin-5-yl)naphtho[2,3-c]furan-1(3H)-one hydrochloride
[104] 9-(2-(2-(hydroxymethyl) piperidin-1-yl)pyrimidin-5-yl)-6,7-dimethoxynaphtho[2,3-c]furan-1(3H)-one hydrochloride
[105] 9-(2-((cyclopropylmethyl)(propyl)amino)pyrimidin-5-yl)-6,7-dimethoxynaphtho[2,3-c]furan-1(3H)-one hydrochloride
[106] 9-(2-(ethyl(methyl)amino)pyrimidin-5-yl)-6,7-dimethoxynaphtho[2,3-c]furan-1(3H)-one hydrochloride
[107] 6,7-dimethoxy-9-(2-(4-methylpiperidin-1-yl)pyrimidin-5-yl)naphtho[2,3-c]furan-(3H)-one hydrochloride
[108] 6,7-dimethoxy-9-(2-(methylamino)pyrimidin-5-yl)naphtho[2,3-c]furan-1(3H)-one
[109] 9-(2-((3,4-dimethylphenyl)(methyl)amino)pyrimidin-5-yl)-6,7-dimethoxynaphtho[2,3-c]furan-1(3H)-one
[110] (R)-6,7-dimethoxy-9-(2-(2-(pyrrolidin-1-ylmethyl)pyrrolidin-1-yl)pyrimidin-5-yl)naphtho[2,3-c]furan-1(3H)-one
[111] 6,7-dimethoxy-9-(2-(methyl(2-morpholinoethyl)amino)pyrimidin-5-yl)naphtho[2,3-c]furan-1(3H)-one hydrochloride
[112] 6,7-dimethoxy-9-(2-(methyl(3-morpholinopropyl)amino)pyrimidin-5-yl)naphtho[2,3-c]furan-1(3H)-one dihydrochloride
[113] (R)-9-(2-(2-ethylpiperidin-1-yl)pyrimidin-5-yl)-6,7-dimethoxynaphtho[2,3-c]furan-1(3H)-one
[114] 6,7-dimethoxy-9-(2-(2,2,6,6-tetramethylmorpholino)pyrimidin-5-yl)naphtho[2,3-c]furan-1(3H)-one
[115] 6,7-dimethoxy-9-(2-(methyl(tetrahydro-2H-pyran-4-yl)amino)pyrimidin-5-yl)naphtho[2,3-c]furan-1(3H)-one
[116] 6,7-dimethoxy-9-(2-methylquinolin-6-yl)naphtho[2,3-c]furan-1(3H)-one
[117] 9-(4-hydroxyquinolin-6-yl)-6,7-dimethoxynaphtho[2,3-c]furan-1(3H)-one
[118] 6,7-dimethoxy-9-(quinolin-7-yl)naphtho[2,3-c]furan-1(3H)-one
[119] 6,7-dimethoxy-9-(5-(trifluoromethyl) quinolin-8-yl)naphtho[2,3-c]furan-1(3H)-one
[120] 6,7-dimethoxy-9-(3-methylquinolin-6-yl)naphtho[2,3-c]furan-1(3H)-one
[121] 9-(6-((2S,6R)-2,6-dimethylmorpholino)pyridin-3-yl)-6,7-dimethoxynaphtho[2,3-c]furan-1(3H)-one
[122] 9-(6-(4,4-difluoropiperidin-1-yl)pyridin-3-yl)-6,7-dimethoxynaphtho[2,3-c]furan-1(3H)-one
[123] 6,7-dimethoxy-9-(6-(piperidin-1-yl)pyridin-3-yl)naphtho[2,3-c]furan-1(3H)-one
[124] 6,7-dimethoxy-9-(6-morpholinopyridin-3-yl)naphtho[2,3-c]furan-1(3H)-one
[125] 6,7-dimethoxy-9-(quinolin-8-yl)naphtho[2,3-c]furan-1(3H)-one
[126] 6,7-dimethoxy-9-(6-(3-methylpiperidin-1-yl)pyridin-3-yl)naphtho[2,3-c]furan-1(3H)-one
[127] 6,7-dimethoxy-9-(6-(3-methylpiperidin-1-yl)pyridin-3-yl)naphtho[2,3-c]furan-1(3H)-one
[128] 6,7-dimethoxy-9-(piperazin-1-yl)naphtho[2,3-c]furan-1(3H)-one
[129] 9-(4-(5-bromopyridin-2-yl) piperazin-1-yl)-6,7-dimethoxynaphtho[2,3-c]furan-1(3H)-one
[130] 9-(4-(6-chloro-2-(methylthio)pyrimidin-4-yl) piperazin-1-yl)-6,7-dimethoxynaphtho[2,3-c]furan-1(3H)-one
[131] 9-(4-(5-bromopyrimidin-2-yl) piperazin-1-yl)-6,7-dimethoxynaphtho[2,3-c]furan-1(3H)-one
[132] 6,7-dimethoxy-9-(quinolin-5-yl)naphtho[2,3-c]furan-1(3H)-one
[133] 6,7-dimethoxy-9-(3-methoxyisoquinolin-8-yl)naphtho[2,3-c]furan-1(3H)-one
[134] 9-(8-fluoro-2-methylquinolin-4-yl)-6,7-dimethoxynaphtho[2,3-c]furan-1(3H)-one
[135] 6,7-dimethoxy-9-(4-(piperazin-1-yl) quinolin-6-yl)naphtho[2,3-c]furan-1(3H)-one
[136] 6,7-dimethoxy-9-(2-methyl-8-(trifluoromethyl) quinolin-4-yl)naphtho[2,3-c]furan-1(3H)-one
[137] 9-(8-fluoroquinolin-4-yl)-6,7-dimethoxynaphtho[2,3-c]furan-1(3H)-one
[138] 9-(4-chloroquinolin-6-yl)-6,7-dimethoxynaphtho[2,3-c]furan-1(3H)-one
[139] 9-(6,8-difluoro-2-methylquinolin-4-yl)-6,7-dimethoxynaphtho[2,3-c]furan-1(3H)-one
[140] 6,7-dimethoxy-9-(8-methoxyquinolin-4-yl)naphtho[2,3-c]furan-1(3H)-one
[141] 6,7-dimethoxy-9-(7-methoxy-2-methylquinolin-4-yl)naphtho[2,3-c]furan-1(3H)-one
[142] 9-(6-(4-chloropiperidin-1-yl)pyridin-3-yl)-6,7-dimethoxynaphtho[2,3-c]furan-1(3H)-one
[143] 6,7-dimethoxy-9-(4-(2-morpholinoethyl) piperazin-1-yl)naphtho[2,3-c]furan-1(3H)-one
[144] 9-(4-(2-hydroxyethyl) piperazin-1-yl)-6,7-dimethoxynaphtho[2,3-c]furan-1(3H)-one
[146] 9-(4-hydroxy-2,8-dimethylquinolin-7-yl)-6,7-dimethoxynaphtho[2,3-c]furan-1(3H)-one
[147] 9-(4-hydroxy-2-methylquinolin-6-yl)-6,7-dimethoxynaphtho[2,3-c]furan-1(3H)-one
[148] 9-(6-fluoroquinolin-4-yl)-6,7-dimethoxynaphtho[2,3-c]furan-1(3H)-one
[149] 6,7-dimethoxy-9-(8-methoxy-2-methylquinolin-4-yl)naphtho[2,3-c]furan-1(3H)-one
[150] 6,7-dimethoxy-9-(7-methoxyquinolin-4-yl)naphtho[2,3-c]furan-1(3H)-one
[151] (S)-6,7-dimethoxy-9-(2-(1-phenyl-3,4-dihydroisoquinolin-2 (1H)-yl)pyrimidin-5-yl)naphtho[2,3-c]furan-1(3H)-one
[152] 6,7-dimethoxy-9-(4-(piperazin-1-yl) quinolin-7-yl)naphtho[2,3-c]furan-1(3H)-one
[154] 9-(4-hydroxy-8-methylquinolin-7-yl)-6,7-dimethoxynaphtho[2,3-c]furan-1(3H)-one
[155] 6,7-dimethoxy-9-(6-methoxy-2-methylquinolin-4-yl)naphtho[2,3-c]furan-1(3H)-one
[156] 9-(6-(6,7-dihydrothien[3,2-c]pyridin-5 (4H)-yl)pyridin-3-yl)-6,7-dimethoxynaphtho[2,3-c]furan-1(3H)-one
[157] 9-(4-(4,6-dimethoxypyrimidin-5-yl)phenyl)-6,7-dimethoxynaphtho[2,3-c]furan-1(3H)-one
[158] 9-(2-((2-(1,3-dioxolan-2-yl)ethyl)(methyl)amino)pyrimidin-5-yl)-6,7-dimethoxynaphtho[2,3-c]furan-1(3H)-one
[159] 6,7-dimethoxy-9-(6-(4-methylpiperidin-1-yl)pyridin-3-yl)naphtho[2,3-c]furan-1(3H)-one
[160] 9-(6-(diethylamino)pyridin-3-yl)-6,7-dimethoxynaphtho[2,3-c]furan-1(3H)-one
[161] 9-(6-((2-(1,3-dioxolan-2-yl)ethyl)(methyl)amino)pyridin-3-yl)-6,7-dimethoxynaphtho[2,3-c]furan-1(3H)-one
[162] 9-(6-((2,2-dimethoxyethyl)(methyl)amino)pyridin-3-yl)-6,7-dimethoxynaphtho[2,3-c]furan-1(3H)-one
[163] 9-(6-(dimethylamino)pyridin-3-yl)-6,7-dimethoxynaphtho[2,3-c]furan-1(3H)-one
[164] (S)-6,7-dimethoxy-9-(6-((5-methoxy-1,2,3,4-tetrahydronaphthalen-2-yl)(methyl)amino)pyridin-3-yl)naphtho[2,3-c]furan-1(3H)-one
[165] 9-(6-(4-hydroxypiperidin-1-yl)pyridin-3-yl)-6,7-dimethoxynaphtho[2,3-c]furan-1(3H)-one
[166] 9-(6-(1,4-dioxa-8-azaspiro[4.5]decan-8-yl)pyridin-3-yl)-6,7-dimethoxynaphtho[2,3-c]furan-1(3H)-one
[167] 6,7-dimethoxy-9-(6-(4-methylpiperazin-1-yl)pyridin-3-yl)naphtho[2,3-c]furan-1(3H)-one
[168] 6,7-dimethoxy-9-(6-((2-methoxyethyl)(methyl)amino)pyridin-3-yl)naphtho[2,3-c]furan-1(3H)-one hydrochloride
[170] 9-(6-(cyclopropyl(methyl)amino)pyridin-3-yl)-6,7-dimethoxynaphtho[2,3-c]furan-1(3H)-one
[172] (S)-6,7-dimethoxy-9-(6-((5-methoxy-1,2,3,4-tetrahydronaphthalen-2-yl)amino)pyridin-3-yl)naphtho[2,3-c]furan-1(3H)-one
[173] 6,7-dimethoxy-9-(5-(2-methoxyethoxy)pyridin-3-yl)naphtho[2,3-c]furan-1(3H)-one
[174] 9-(6-(bis(2-methoxyethyl)amino)pyridin-3-yl)-6,7-dimethoxynaphtho[2,3-c]furan-1(3H)-one
[175] 9-(6-(cyclohexyl(methyl)amino)pyridin-3-yl)-6,7-dimethoxynaphtho[2,3-c]furan-1(3H)-one
[176] 6,7-dimethoxy-9-(6-(methyl(2-methylbutyl)amino)pyridin-3-yl)naphtho[2,3-c]furan-1(3H)-one
[177] 9-(6-(cyclopentyl(methyl)amino)pyridin-3-yl)-6,7-dimethoxynaphtho[2,3-c]furan-1(3H)-one
[178] 9-(6-(ethyl(methyl)amino)pyridin-3-yl)-6,7-dimethoxynaphtho[2,3-c]furan-1(3H)-one
[179] 6,7-dimethoxy-9-(6-(methyl((5-methylfuran-2-yl)methyl)amino)pyridin-3-yl)naphtho[2,3-c]furan-1(3H)-one
[180] 9-(6-(butyl(methyl)amino)pyridin-3-yl)-6,7-dimethoxynaphtho[2,3-c]furan-1(3H)-one
[181] 9-(6-(isobutyl(methyl)amino)pyridin-3-yl)-6,7-dimethoxynaphtho[2,3-c]furan-1(3H)-one
[182] 9-(6-(ethyl(isopropyl)amino)pyridin-3-yl)-6,7-dimethoxynaphtho[2,3-c]furan-1(3H)-one
[183] 9-(6-((cyclopropylmethyl)(methyl)amino)pyridin-3-yl)-6,7-dimethoxynaphtho[2,3-c]furan-1(3H)-one
[184] 9-(2-chloropyridin-4-yl)-6,7-dimethoxynaphtho[2,3-c]furan-1(3H)-one
[185] 9-(6-chloropyridin-3-yl)-6,7-dimethoxynaphtho[2,3-c]furan-1(3H)-one
[186] 9-(2-fluoropyridin-4-yl)-6,7-dimethoxynaphtho[2,3-c]furan-1(3H)-one
[187] 9-(5-(2-(dimethylamino) ethoxy)pyridin-3-yl)-6,7-dimethoxynaphtho[2,3-c]furan-1(3H)-one
[188] 9-(6-(isopropyl(methyl)amino)pyridin-3-yl)-6,7-dimethoxynaphtho[2,3-c]furan-1(3H)-one
[189] 9-(6-(3,4-dihydroisoquinolin-2 (1H)-yl)pyridin-3-yl)-6,7-dimethoxynaphtho[2,3-c]furan-1(3H)-one
[191] 9-(6-(cyclopentyl(ethyl)amino)pyridin-3-yl)-6,7-dimethoxynaphtho[2,3-c]furan-1(3H)-one
[192] 6,7-dimethoxy-9-(6-(methyl((tetrahydrofuran-2-yl)methyl)amino)pyridin-3-yl)naphtho[2,3-c]furan-1(3H)-one
[193] 9-(6-(ethyl((tetrahydrofuran-2-yl)methyl)amino)pyridin-3-yl)-6,7-dimethoxynaphtho[2,3-c]furan-1(3H)-one
[194] 9-(6-((2,3-dihydro-1H-indan-2-yl)(methyl)amino)pyridin-3-yl)-6,7-dimethoxynaphtho[2,3-c]furan-1(3H)-one
[195] 9-(6-((2,3-dihydro-1H-indan-2-yl)(ethyl)amino)pyridin-3-yl)-6,7-dimethoxynaphtho[2,3-c]furan-1(3H)-one
[196] 6,7-dimethoxy-9-(6-(methyl(2-(1-methyl-1H-indol-3-yl)ethyl)amino)pyridin-3-yl)naphtho[2,3-c]furan-1(3H)-one
[197] 9-(6-(ethyl(2-(1-ethyl-1H-indol-3-yl)ethyl)amino)pyridin-3-yl)-6,7-dimethoxynaphtho[2,3-c]furan-1(3H)-one
[198] 6,7-dimethoxy-9-(6-(methyl(thiophen-2-ylmethyl)amino)pyridin-3-yl)naphtho[2,3-c]furan-1(3H)-one
[199] 9-(6-(ethyl(thiophen-2-ylmethyl)amino)pyridin-3-yl)-6,7-dimethoxynaphtho[2,3-c]furan-1(3H)-one
[200] 9-(5-isopropoxypyridin-3-yl)-6,7-dimethoxynaphtho[2,3-c]furan-1(3H)-one
[202] 6,7-dimethoxy-9-(6-(3-(m-tolyl) piperidin-1-yl)pyridin-3-yl)naphtho[2,3-c]furan-1(3H)-one
[203] 6,7-dimethoxy-9-(3-(morpholinomethyl)-1H-indol-6-yl)naphtho[2,3-c]furan-1(3H)-one
[205] 9-(2-(4-(dimethylamino) piperidin-1-yl)pyrimidin-5-yl)-6,7-dimethoxynaphtho[2,3-c]furan-1(3H)-one
[206] 6,7-dimethoxy-9-(6-(methyl(1-methyl-1H-1,2,4-triazol-3-yl)amino)pyridin-3-yl)naphtho[2,3-c]furan-1(3H)-one
[207] 9-(6-(ethyl(1-ethyl-1H-1,2,4-triazol-3-yl)amino)pyridin-3-yl)-6,7-dimethoxynaphtho[2,3-c]furan-1(3H)-one
[208] 6,7-dimethoxy-9-(6-(methyl(2-(trifluoromethyl)benzyl)amino)pyridin-3-yl)naphtho[2,3-c]furan-1(3H)-one
[209] 9-(6-(ethyl(2-(trifluoromethyl)benzyl)amino)pyridin-3-yl)-6,7-dimethoxynaphtho[2,3-c]furan-1(3H)-one
[213] 9-(2-((3,4-dimethylphenyl)(methyl)amino)pyrimidin-5-yl)-6,7-dimethoxynaphtho[2,3-c]furan-1(3H)-one
[214] 6,7-dimethoxy-9-(6-(methyl(5-methyl-1,3,4-thiadiazol-2-yl)amino)pyridin-3-yl)naphtho[2,3-c]furan-1(3H)-one
[215] 6,7-dimethoxy-9-(6-(methyl(1-methyl-5-(methylthio)-1H-1,2,4-triazol-3-yl)amino)pyridin-3-yl)naphtho[2,3-c]furan-1(3H)-one
[216] 6,7-dimethoxy-9-(2-(methyl(3-(trifluoromethyl)phenyl)amino)pyrimidin-5-yl)naphtho[2,3-c]furan-1(3H)-one
[218] 4-((5-(6,7-dimethoxy-3-oxo-1,3-dihydronaphtho[2,3-c]furan-4-yl)pyridin-2-yl)(methyl)amino)benzonitrile
[219] 6,7-dimethoxy-9-(6-morpholinopyridazin-3-yl)naphtho[2,3-c]furan-1(3H)-one
[220] 6,7-dimethoxy-9-(6-morpholinoimidazo[1,2-b]pyridazin-3-yl)naphtho[2,3-c]furan-1(3H)-one
[222] 6,7-dimethoxy-9-(6-(methyl(phenyl)amino)pyridin-3-yl)naphtho[2,3-c]furan-1(3H)-one
[223] 9-(6-(3-(dimethylamino)-5-(methylthio)-1H-1,2,4-triazol-1-yl)pyridin-3-yl)-6,7-dimethoxynaphtho[2,3-c]furan-1(3H)-one
[225] 9-(6-(benzo[d]thiazol-2-yl(methyl)amino)pyridin-3-yl)-6,7-dimethoxynaphtho[2,3-c]furan-1(3H)-one
[226] 9-(6-(ethyl(pyridin-4-ylmethyl)amino)pyridin-3-yl)-6,7-dimethoxynaphtho[2,3-c]furan-1(3H)-one
[229] 6,7-dimethoxy-9-(6-(methylamino)pyridin-3-yl)naphtho[2,3-c]furan-1(3H)-one
[230] N-(5-(6,7-dimethoxy-3-oxo-1,3-dihydronaphtho[2,3-c]furan-4-yl)pyridin-2-yl)-N-methylacetamide
[231] 6,7-dimethoxy-9-(6-(methyl(5-methyl-1,3,4-oxadiazol-2-yl)amino)pyridin-3-yl)naphtho[2,3-c]furan-1(3H)-one
[232] 9-(6-((2,4-dimethoxybenzyl)(methyl)amino)pyridin-3-yl)-6,7-dimethoxynaphtho[2,3-c]furan-1(3H)-one
[233] N-(5-(6,7-dimethoxy-3-oxo-1,3-dihydronaphtho[2,3-c]furan-4-yl)pyridin-2-yl)-N-methylpivalamide
[236] N-(4-((5-(6,7-dimethoxy-3-oxo-1,3-dihydronaphtho[2,3-c]furan-4-yl)pyridin-2-yl)(methyl)amino)phenyl)-N-methylacetamide
[237] 3-((5-(6,7-dimethoxy-3-oxo-1,3-dihydronaphtho[2,3-c]furan-4-yl)pyridin-2-yl)(methyl)amino)benzonitrile
[238] 9-(6-(4-(dimethylamino) piperidin-1-yl)pyridin-3-yl)-6,7-dimethoxynaphtho[2,3-c]furan-1(3H)-one
[239] 9-(6-((4-mercaptophenyl)(methyl)amino)pyridin-3-yl)-6,7-dimethoxynaphtho[2,3-c]furan-1(3H)-one
[240] 6,7-dimethoxy-9-(6-(3-(trifluoromethyl)-5,6-dihydro-[1,2,4]triazolo[4,3-a]pyrazin-7 (8H)-yl)pyridin-3-yl)naphtho[2,3-c]furan-1(3H)-one
[241] 3-((5-(6,7-dimethoxy-3-oxo-1,3-dihydronaphtho[2,3-c]furan-4-yl)pyridin-2-yl)(methyl)amino)-1-methyl-1H-pyrazol-4-carbonitrile
[242] 6,7-dimethoxy-9-(4-(piperidin-1-yl)phenyl)naphtho[2,3-c]furan-1(3H)-one
[243] 6,7-dimethoxy-9-(3-(piperidin-1-yl)phenyl)naphtho[2,3-c]furan-1(3H)-one
[244] 6,7-dimethoxy-9-(3-morpholinophenyl)naphtho[2,3-c]furan-1(3H)-one
[245] 6,7-dimethoxy-9-(4-morpholinophenyl)naphtho[2,3-c]furan-1(3H)-one
[246] 6,7-dimethoxy-9-(4-(methylamino)phenyl)naphtho[2,3-c]furan-1(3H)-one
[247] 9-(4-(dimethylamino)phenyl)-6,7-dimethoxynaphtho[2,3-c]furan-1(3H)-one
[248] 9-(4-(ethyl(methyl)amino)phenyl)-6,7-dimethoxynaphtho[2,3-c]furan-1(3H)-one
[249] 9-(4-(diethylamino)phenyl)-6,7-dimethoxynaphtho[2,3-c]furan-1(3H)-one
[250] 9-(4-(ethylamino)phenyl)-6,7-dimethoxynaphtho[2,3-c]furan-1(3H)-one
[251] 6,7-dimethoxy-9-(4-(pyrrolidin-1-yl)phenyl)naphtho[2,3-c]furan-1(3H)-one
[252] 6,7-dimethoxy-9-(3-(pyrrolidin-1-yl)phenyl)naphtho[2,3-c]furan-1(3H)-one
[253] 9-(4-(ethyl(2-methoxyethyl)amino)phenyl)-6,7-dimethoxynaphtho[2,3-c]furan-1(3H)-one
[254] 9-(4-(ethyl(pyridin-3-ylmethyl)amino)phenyl)-6,7-dimethoxynaphtho[2,3-c]furan-1(3H)-one
[255] 6,7-dimethoxy-9-(4-(methyl(pyridin-3-ylmethyl)amino)phenyl)naphtho[2,3-c]furan-1(3H)-one
[256] 6,7-dimethoxy-9-(4-((2-methoxyethyl)(methyl)amino)phenyl)naphtho[2,3-c]furan-1(3H)-one
[257] 6,7-dimethoxy-9-(3-(methylamino)phenyl)naphtho[2,3-c]furan-1(3H)-one
[258] 9-(3-(ethylamino)phenyl)-6,7-dimethoxynaphtho[2,3-c]furan-1(3H)-one
[274] 6-(6,7-dimethoxy-3-oxo-1,3-dihydronaphtho[2,3-c]furan-4-yl)benzo[d][1,3]dioxol-5-yl acetate
[275] 6-(6,7-dimethoxy-3-oxo-1,3-dihydronaphtho[2,3-c]furan-4-yl)benzo[d][1,3]dioxol-5-yl isobutrate
[276] 6-(6,7-dimethoxy-3-oxo-1,3-dihydronaphtho[2,3-c]furan-4-yl)benzo[d][1,3]dioxol-5-yl pivalate

7. A compound of formula 3 below, a pharmaceutically acceptable salt thereof, a stereoisomer thereof, a hydrate thereof or a solvate thereof: 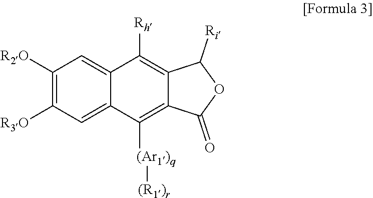
wherein in above formula 3,
Ar_{1′} is substituted or unsubstituted C₆-C₁₂ arylene group, or substituted or unsubstituted C₂-C₁₂ heteroarylene group comprising at least one of N, O and S as a heteroatom in a ring, and
q is 0 or 1,
R_{1′} is each independently halogen, substituted or unsubstituted C₆-C₂₀ aryl (wherein a substituent of the C₆-C₂₀ aryl may be bonded to an adjacent group to form a ring); —NR_{4′}R_{5′}; —OR_{6′}; —C(═O)CX₃ (wherein X is halogen); —NO₂; 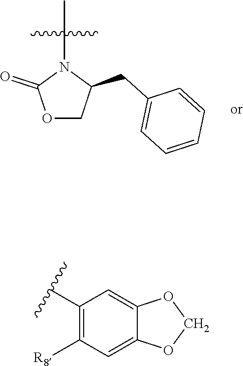
(wherein at least one of the hydrogen of 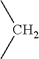
may be substituted with halogen), and r is an integer of 0 or more and 2 or less,
R_{2′} and R_{3′} are each independently C₁-C₁₀ linear or branched alkyl,
R_{4′} to R_{6′} are each independently C₁-C₁₀ linear or branched alkyl, or —C(═O)R_{c′},
R_{c′} is substituted or unsubstituted C₂-C₁₀ heterocycloalkyl comprising at least one of N, O and S as a heteroatom in a ring, or substituted or unsubstituted C₂-C₂₀ heteroaryl comprising at least one of N, O and S as a heteroatom in a ring,
R_{8′} is hydrogen, —OC(═O)R_{d′}, —OP(═O)(OR_{d′})₂, —OP(═O)(NHR_{d′})(OR_{e′}), —OP(═O)(NHR_{d′})(R_{e′}), —OH, —NHR_{d′} or C₁-C₂₀ linear or branched alkoxy,
R_{d′} and R_{e′} are each independently C₁-C₁₀ linear or branched alkyl (wherein at least one of the hydrogen of the alkyl may be substituted with C₂-C₁₀ heterocycloalkyl or amine), C₂-C₁₀ heterocycloalkyl, substituted or unsubstituted C₆-C₂₀ aryl, or substituted or unsubstituted C₂-C₂₀ heteroaryl, and
R_{h′} and R_{i′} are each independently hydrogen,

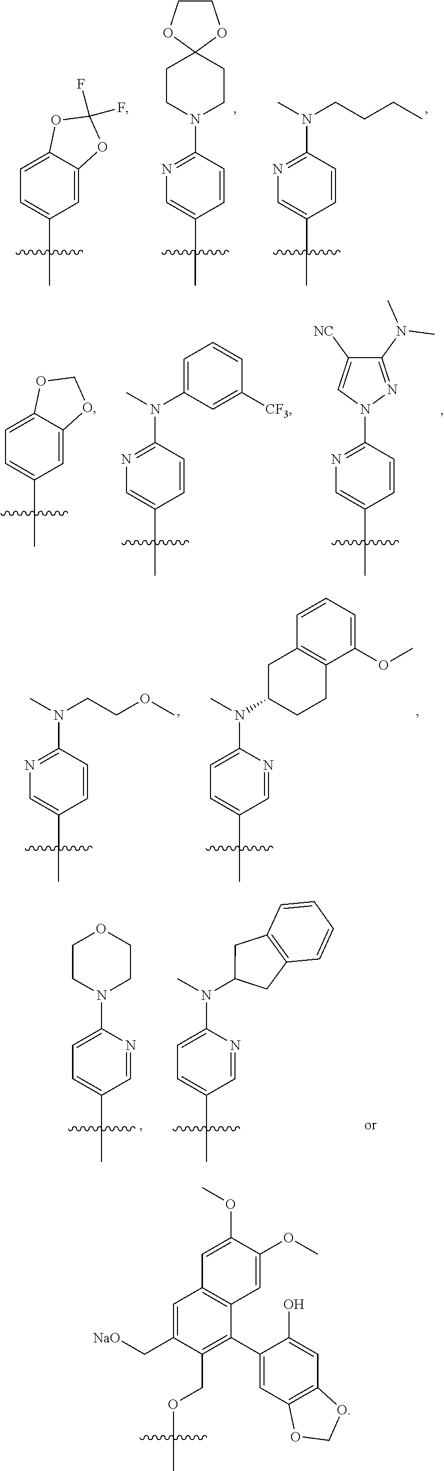

8. The compound, the pharmaceutically acceptable salt thereof, the stereoisomer thereof, the hydrate thereof or the solvate thereof according to claim 7, wherein (Ar_{1′})_{q}—(R_{1′})ᵣ is represented by formula 4 below: 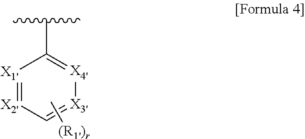
wherein in above formula 4,
X_{1′} to X_{4′} are each independently CH or N, and R_{1′} and r are same as defined in above formula 3.

9. The compound, the pharmaceutically acceptable salt thereof, the stereoisomer thereof, the hydrate thereof or the solvate thereof according to claim 8, wherein above formula 4 is represented by formulas 4-1 to 4-4 below: 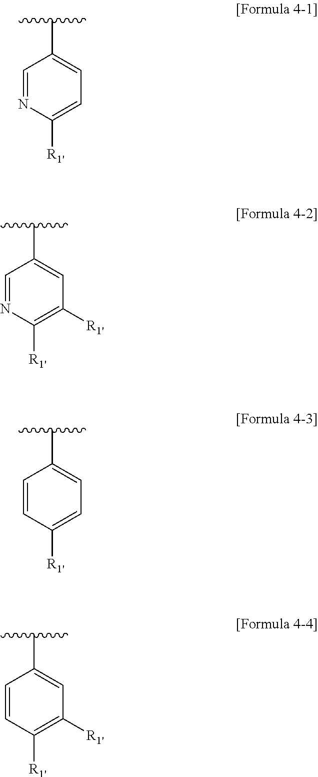
wherein in above formulas 4-1 to 4-4, R_{1′} is same as defined in above formula 3.

10. The compound, the pharmaceutically acceptable salt thereof, the stereoisomer thereof, the hydrate thereof or the solvate thereof according to claim 7, wherein R_{1′} is halogen, —NR_{4′}R_{5′}, —OR_{6′}, —C(═O)CX₃ (wherein X is halogen), —NO₂, 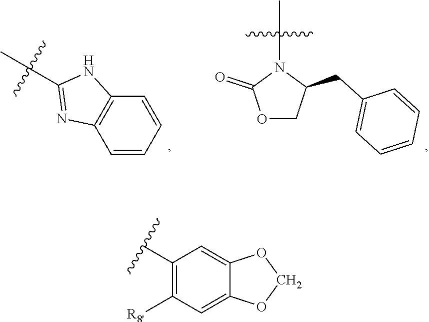
(wherein at least one of the hydrogen of 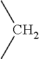
may be substituted with halogen), or 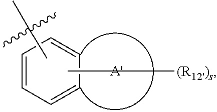
R_{12′} is each independently C₁-C₁₀ linear or branched alkyl, —CX₃ (wherein X is halogen) or —OH,
s is an integer of 0 or more and 5 or less,
A′ is C₆-C₁₂ aryl or C₂-C₁₀ heterocycloalkyl comprising at least one of N, O and S as a heteroatom in a ring, and
R_{4′} to R_{6′} and R_{8′} are same as defined in formula 3.

11. The compound, the pharmaceutically acceptable salt thereof, the stereoisomer thereof, the hydrate thereof or the solvate thereof according to claim 8, wherein Rr is chlorine,

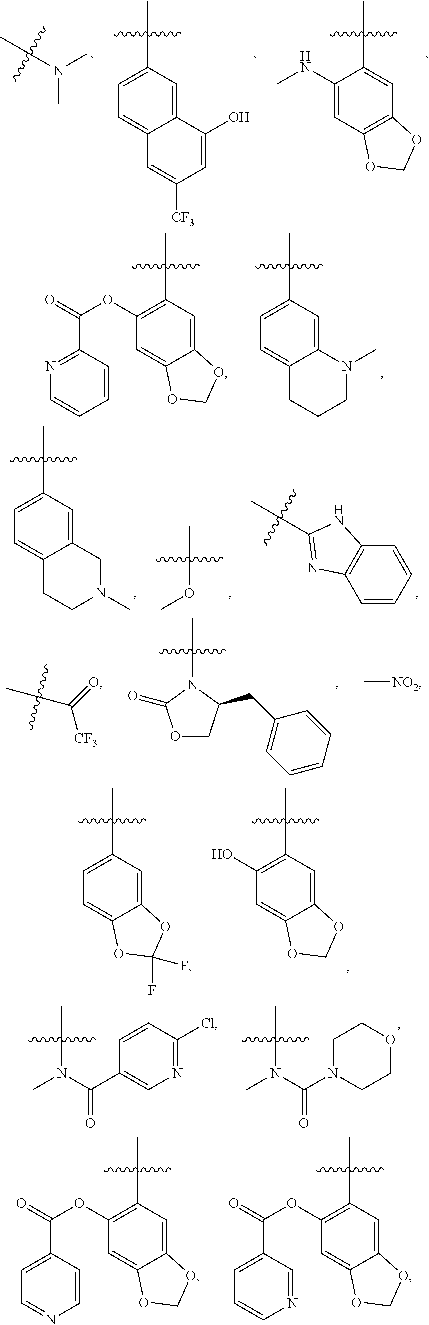

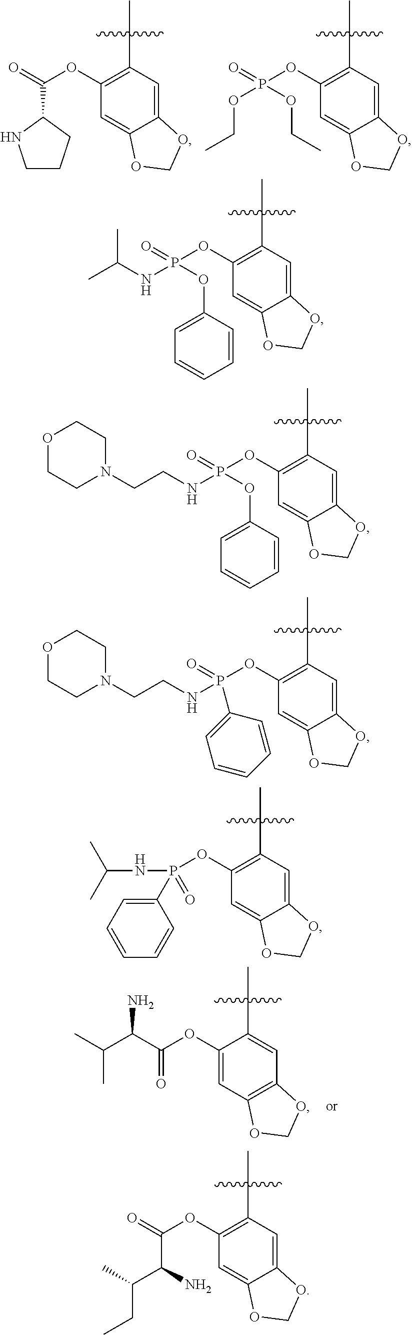

12. The compound, the pharmaceutically acceptable salt thereof, the stereoisomer thereof, the hydrate thereof and the solvate thereof according to claim 7, wherein the compound represented by above formula 3 is one selected from the group consisting of the following compounds:
[145] 9-(4-(2-hydroxyethyl) piperazin-1-yl)-6,7-dimethoxynaphtho[2,3-c]furan-1(3H)-one
[153] 9-(8-hydroxy-6-(trifluoromethyl) naphthalen-2-yl)-6,7-dimethoxynaphtho[2,3-c]furan-1(3H)-one
[169] 6-(6,7-dimethoxy-3-oxo-1,3-dihydronaphtho[2,3-c]furan-4-yl)benzo[d][1,3]dioxol-5-yl picolate
[170] 6,7-dimethoxy-9-(1-methyl-1,2,3,4-tetrahydroquinolin-7-yl)naphtho[2,3-c]furan-1(3H)-one
[190] 6,7-dimethoxy-9-(2-methyl-1,2,3,4-tetrahydroisoquinolin-7-yl)naphtho[2,3-c]furan-1(3H)-one
[201] 9-(6-chloro-5-(dimethylamino)pyridin-3-yl)-6,7-dimethoxynaphtho[2,3-c]furan-1(3H)-one
[204] 9-(5-(1H-benzo[d]imidazol-2-yl)-6-methoxypyridin-3-yl)-6,7-dimethoxynaphtho[2,3-c]furan-1(3H)-one
[210] 6,7-dimethoxy-9-(4-(2,2,2-trifluoroacetyl)phenyl)naphtho[2,3-c]furan-1(3H)-one
[211] (S)-4-benzyl-3-(5-(6,7-dimethoxy-3-oxo-1,3-dihydronaphtho[2,3-c]furan-4-yl)pyridin-2-yl) oxazolidin-2-one
[217] 9-(4-(dimethylamino)-3-nitrophenyl)-6,7-dimethoxynaphtho[2,3-c]furan-1(3H)-one
[221] 9-(2,2-difluorobenzo[d][1,3]dioxol-5-yl)-6,7-dimethoxynaphtho[2,3-c]furan-1(3H)-one
[227] 9-(4-(5-bromopyrimidin-2-yl) piperazin-1-yl)-6,7-dimethoxynaphtho[2,3-c]furan-1(3H)-one
[234] 6-chloro-N-(5-(6,7-dimethoxy-3-oxo-1,3-dihydronaphtho[2,3-c]furan-4-yl)pyridin-2-yl)-N-methylnicotinamide
[235] N-(5-(6,7-dimethoxy-3-oxo-1,3-dihydronaphtho[2,3-c]furan-4-yl)pyridin-2-yl)-N-methylmorpholin-4-carboxamide
[259] 4-(2,2-difluorobenzo[d][1,3]dioxol-5-yl)-6,7,9-trimethoxynaphtho[2,3-c]furan-1(3H)-one
[260] 4-(6-(1,4-dioxa-8-azaspiro[4.5]decan-8-yl)pyridin-3-yl)-6,7,9-trimethoxynaphtho[2,3-c]furan-1(3H)-one
[261] 4-(6-(butyl(methyl)amino)pyridin-3-yl)-6,7,9-trimethoxynaphtho[2,3-c]furan-1(3H)-one
[262] 4-(benzo[d][1,3]dioxol-5-yl)-6,7,9-trimethoxynaphtho[2,3-c]furan-1(3H)-one
[263] 6,7,9-trimethoxy-4-(6-(methyl(3-(trifluoromethyl)phenyl)amino)pyridin-3-yl)naphtho[2,3-c]furan-1(3H)-one
[264] 3-(dimethylamino)-1-(5-(6,7,9-trimethoxy-1-oxo-1,3-dihydronaphtho[2,3-c]furan-4-yl)pyridin-2-yl)-1H-pyrazol-4-carbonitrile
[265] 6,7,9-trimethoxy-4-(6-((2-methoxyethyl)(methyl)amino)pyridin-3-yl)naphtho[2,3-c]furan-1(3H)-one
[266] (S)-6,7,9-trimethoxy-4-(6-((5-methoxy-1,2,3,4-tetrahydronaphthalen-2-yl)(methyl)amino)pyridin-3-yl)naphtho[2,3-c]furan-1(3H)-one
[267] 6,7,9-trimethoxy-4-(6-morpholinopyridin-3-yl)naphtho[2,3-c]furan-1(3H)-one
[268] 4-(6-((2,3-dihydro-1H-indan-2-yl)(methyl)amino)pyridin-3-yl)-6,7,9-trimethoxynaphtho[2,3-c]furan-1(3H)-one
[269] 6-(6,7-dimethoxy-3-oxo-1,3-dihydronaphtho[2,3-c]furan-4-yl)benzo[d][1,3]dioxol-5-yl L-valinate hydrochloride
[270] 6-(6,7-dimethoxy-3-oxo-1,3-dihydronaphtho[2,3-c]furan-4-yl)benzo[d][1,3]dioxol-5-yl L-isoleucinate
[271] 6-(6,7-dimethoxy-3-oxo-1,3-dihydronaphtho[2,3-c]furan-4-yl)benzo[d][1,3]dioxol-5-yl isonicoinate
[272] 6-(6,7-dimethoxy-3-oxo-1,3-dihydronaphtho[2,3-c]furan-4-yl)benzo[d][1,3]dioxol-5-yl nicotinate
[273] 6-(6,7-dimethoxy-3-oxo-1,3-dihydronaphtho[2,3-c]furan-4-yl)benzo[d][1,3]dioxol-5-yl L-prolinate
[277] 6-(6,7-dimethoxy-3-oxo-1,3-dihydronaphtho[2,3-c]furan-4-yl)benzo[d][1,3]dioxol-5-yl diethyl phosphate
[278] 6-(6,7-dimethoxy-3-oxo-1,3-dihydronaphtho[2,3-c]furan-4-yl)benzo[d][1,3]dioxol-5-yl phenyl isopropylphosphoramidate
[279] 6-(6,7-dimethoxy-3-oxo-1,3-dihydronaphtho[2,3-c]furan-4-yl)benzo[d][1,3]dioxol-5-yl phenyl (2-morpholinoethyl)phosphoramidate
[280] 6-(6,7-dimethoxy-3-oxo-1,3-dihydronaphtho[2,3-c]furan-4-yl)benzo[d][1,3]dioxol-5-yl N-(2-morpholinoethyl)-P-phenylphosphoramidate
[281] 6-(6,7-dimethoxy-3-oxo-1,3-dihydronaphtho[2,3-c]furan-4-yl)benzo[d][1,3]dioxol-5-yl N-isopropyl-P-phenylphosphoramidate

13. A compound below, a pharmaceutically acceptable salt thereof, a stereoisomer thereof, a hydrate thereof or a solvate thereof:
[212] 1-(6-hydroxybenzo[d][1,3]dioxol-5-yl)-3-(hydroxymethyl)-6,7-dimethoxy-2-naphthoate sodium (I)
[224] 1-(6-hydroxybenzo[d][1,3]dioxol-5-yl)-3-(hydroxymethyl)-6,7-dimethoxy-2-naphthoyl chloride
[228] 1-(6-hydroxybenzo[d][1,3]dioxol-5-yl)-3-(hydroxymethyl)-N-isopropyl-6,7-dimethoxy-2-naphthamide

14. The compound, the pharmaceutically acceptable salt thereof, the stereoisomer thereof, the hydrate thereof or the solvate thereof according to claim 1, wherein the salt is acid addition salt formed by pharmaceutically acceptable free acid.

15. The compound, the pharmaceutically acceptable salt thereof, the stereoisomer thereof, the hydrate thereof or the solvate thereof according to claim 14, wherein the free acid is selected from the group consisting of citric acid, acetic acid, lactic acid, tartaric acid, maleic acid, fumaric acid, formic acid, propionic acid, oxalic acid, trifluoroacetic acid, benzoic acid, gluconic acid, methanesulfonic acid, glycolic acid, succinic acid, 4-toluenesulfonic acid, camphorsulfonic acid, glutamic acid, aspartic acid, silicylic acid, malonic acid, malic acid, benzenesulfonic acid, hydrochloric acid, bromic acid, nitric acid, sulfuric acid, and phosphoric acid.

16. A pharmaceutical composition for an anti-viral purpose comprising the compound, the pharmaceutically acceptable salt thereof, the stereoisomer thereof, the hydrate thereof or the solvate thereof according to claim 1 as active ingredients.

17. The pharmaceutical composition according to claim 16, wherein the composition is a composition for an anti-viral purpose with regard to one or more of SARS-CoV-2, influenza virus, dengue virus, Zika virus, and severe fever with thrombocytopenia syndrome virus (SFTS).

18. A pharmaceutical composition for preventing or treating a virus infection disease, comprising the compound, the pharmaceutically acceptable salt thereof, the stereoisomer thereof, the hydrate thereof or the solvate thereof according to claim 1 as active ingredients.

19. The pharmaceutical composition according to claim 18, wherein the virus infection disease is at least one of SARS-COV-2 infection disease, influenza virus infection disease, dengue virus infection disease, Zika virus infection disease, and severe fever with thrombocytopenia syndrome virus infection disease.

20. The pharmaceutical composition according to claim 19, wherein the SARS-CoV-2 infection disease is COVID-19.

21. The pharmaceutical composition according to claim 18, wherein the composition inhibits intracellular infection and proliferation of one or more of SARS-COV-2, influenza virus, dengue virus, Zika virus, and severe fever with thrombocytopenia syndrome virus.

22. A pharmaceutical composition for preventing or treating a respiratory disease, comprising the compound, the pharmaceutically acceptable salt thereof, the stereoisomer thereof, the hydrate thereof or the solvate thereof according to claim 1 as active ingredients.

23. The pharmaceutical composition according to claim 22, wherein the respiratory disease is at least one selected from the group consisting of asthma, cold, pneumonia, bronchitis, chronic obstructive pulmonary disease and rhinitis.

24. The pharmaceutical composition according to claim 22, wherein the composition inhibits expression of interleukin-5.

25. A pharmaceutical composition for preventing or treating an allergic disease, comprising the compound, the pharmaceutically acceptable salt thereof, the stereoisomer thereof, the hydrate thereof or the solvate thereof according to claim 1 as active ingredients.

26. The pharmaceutical composition of claim 25, wherein the allergic disease is at least one selected from the group consisting of rhinitis, asthma, atopic dermatitis, allergic conjunctivitis, allergic otitis media, allergic gastrointestinal infection, anaphylaxis and hives.

27. A method for preventing or treating a virus infection disease, comprising administering the compound, the pharmaceutically acceptable salt thereof, the stereoisomer thereof, the hydrate thereof or the solvate thereof according to claim 1 into a subject in need thereof.

28. A use of the compound, the pharmaceutically acceptable salt thereof, the stereoisomer thereof, the hydrate thereof or the solvate thereof according to claim 1 for preventing or treating a virus infection disease.

29. A use of the compound, the pharmaceutically acceptable salt thereof, the stereoisomer thereof, the hydrate thereof or the solvate thereof according to claim 1 for manufacturing a medicament for preventing or treating a virus infection disease.

30. A method for preventing or treating a respiratory disease, comprising administering the compound, the pharmaceutically acceptable salt thereof, the stereoisomer thereof, the hydrate thereof or the solvate thereof according to claim 1 into a subject in need thereof.

31. A use of the compound, the pharmaceutically acceptable salt thereof, the stereoisomer thereof, the hydrate thereof or the solvate thereof according to claim 1 for preventing or treating a respiratory disease.

32. A use of the compound, the pharmaceutically acceptable salt thereof, the stereoisomer thereof, the hydrate thereof or the solvate thereof according to claim 1 for manufacturing a medicament for preventing or treating a respiratory disease.

33. A method for preventing or treating an allergic disease, comprising administering the compound, the pharmaceutically acceptable salt thereof, the stereoisomer thereof, the hydrate thereof or the solvate thereof according to claim 1 into a subject in need thereof.

34. A use of the compound, the pharmaceutically acceptable salt thereof, the stereoisomer thereof, the hydrate thereof or the solvate thereof according to claim 1 for preventing or treating an allergic disease.

35. A use of the compound, the pharmaceutically acceptable salt thereof, the stereoisomer thereof, the hydrate thereof or the solvate thereof according to claim 1 for manufacturing a medicament for preventing or treating an allergic disease.
